(19) United States
(12) Patent Application Publication
(10) Pub. No.: US 2025/0235652 A1
(43) Pub. Date: Jul. 24, 2025

(54) PATIENT INTERFACE

(71) Applicant: Fisher & Paykel Healthcare Limited, Auckland (NZ)

(72) Inventors: Jae Yun LIM, Auckland (NZ); Roheet PATEL, Auckland (NZ); Craig Robert PRENTICE, Auckland (NZ); Thomas Mark RICHARDSON, Auckland (NZ); Simon MITTERMEIER, Auckland (NZ); Monika GANTERER, Auckland (NZ); Hamish Joshua ROSE, Auckland (NZ); Max Leon BETTERIDGE, Auckland (NZ); Jeremy Owen YOUNG, Auckland (NZ); Nigel TRINIDADE, Auckland (NZ); Mark Richard TOMLINSON, Auckland (NZ); Vitaly KAPELEVICH, Auckland (NZ); Campbell Neil Addison MARTIN, Auckland (NZ); Tony William SPEAR, Auckland (NZ); Matthew James PEDERSEN, Auckland (NZ)

(21) Appl. No.: 19/096,531

(22) Filed: Mar. 31, 2025

Related U.S. Application Data
(63) Continuation-in-part of application No. 16/977,277, filed on Sep. 1, 2020.
(63) Continuation of application No. 17/636,816, filed on Feb. 18, 2022.
(60) Provisional application No. 62/639,908, filed on Mar. 7, 2018.
(60) Provisional application No. 62/679,675, filed on Jun. 1, 2018.
(60) Provisional application No. 62/680,503, filed on Jun. 4, 2018.
(60) Provisional application No. 62/768,765, filed on Nov. 16, 2018.
(60) Provisional application No. 62/892,429, filed on Aug. 27, 2019.
(60) Provisional application No. 62/896,454, filed on Sep. 5, 2019.

(51) Int. Cl. A61M 16/06; A61M 16/00; A61M 16/08; A61M 16/16

(52) U.S. Cl. CPC A61M 16/0622; A61M 16/0683; A61M 16/0816; A61M 16/022; A61M 16/16

(57) ABSTRACT

A respiratory mask includes a frame and a cushion module. The frame can include a front wall having a vent and/or a gas inlet opening and a collar extending away from the front wall. The collar surrounds the vent and/or the gas inlet opening. The cushion module comprises a cushion and a housing, which is made of a material more rigid than the cushion. The housing defines a connection opening. A friction coupling selectively couples the cushion module to the frame and comprises an elastomeric friction member coupled to a portion of the housing that defines the connection opening. In some arrangements, an outer surface of the friction member is exposed when the cushion module is coupled to the frame. In some arrangements, the housing extends through the peripheral surface.

### PRIORITY AND INCORPORATION BY REFERENCE

Any and all applications for which a foreign or domestic priority claim is identified in connection with the present application are hereby incorporated by reference herein and made a part of the present disclosure.

### BACKGROUND

### Field

The present disclosure relates to respiratory therapy systems. In particular, the disclosure relates to interface assemblies for use in respiratory therapy and portions thereof.

### Description of Related Art

Respiratory masks are used to provide respiratory therapy to the airways of a person suffering from any of a number of respiratory illnesses or conditions. Such therapies may include but are not limited to continuous positive airway pressure (CPAP) therapy and non-invasive ventilation (NIV) therapy.

CPAP therapy can be used to treat obstructive sleep apnea (OSA), a condition in which a patient's airway intermittently collapses, during sleep, preventing the patient from breathing for a period of time. The cessation of breathing, or apnea, results in the patient awakening. Repetitive and frequent apneas may result in the patient rarely achieving a full and restorative night's sleep.

CPAP therapy involves the delivery of a supply of continuous positive air pressure to the airway of the patient via a respiratory mask. The continuous positive pressure acts as a splint within the patient's airway, which secures the airway in an open position such that the patient's breathing and sleep are not interrupted.

Respiratory masks typically comprise a patient interface and a headgear, wherein the patient interface is configured to deliver the supply of continuous positive air pressure to the patient's airway via a seal or cushion that forms an airtight seal in or around the patient's nose and/or mouth. Respiratory masks are available in a range of styles including full-face, nasal, direct nasal and oral masks, which create an airtight seal with the nose and/or mouth. The seal or cushion is held in place on the patient's face by the headgear. In order to maintain an airtight seal the headgear should provide support to the patient interface such that it is held in a stable position relative to the patient's face during use. Such respiratory masks may also be used to deliver NIV and other therapies.

### SUMMARY

The systems, methods and devices described herein have innovative aspects, no single one of which is indispensable or solely responsible for their desirable attributes. Without limiting the scope of the claims, some of the advantageous features will now be summarized.

In some configurations, a respiratory mask includes a frame configured to connect to headgear. The frame comprises a front wall having a vent and a gas inlet opening. The frame further comprises a collar extending away from the front wall. The collar surrounds the vent and the gas inlet opening. A cushion module comprises a housing and a cushion. The housing is made of a material more rigid than the cushion. The housing forms at least a portion of a breathing chamber of the respiratory mask. The housing defines a connection opening. A friction coupling is configured to selectively couple the connection opening of the cushion module to the collar of the frame such that a flow of gas can be delivered to the breathing chamber through the gas inlet opening of the frame and the connection opening of the cushion module. The friction coupling comprises an elastomeric friction member coupled to a portion of the housing that defines the connection opening.

In some configurations, the mask further comprises a divider wall within an interior of the collar. The divider wall having a first end and a second end, each connected to the collar. The divider wall separates the vent and the gas inlet opening.

In some configurations, a surface of the divider wall adjacent the vent extends substantially in a direction of gas flow through the vent.

In some configurations, the collar and the divider wall cooperate to define a socket configured to receive an elbow.

In some configurations, the mask further comprises an elbow received by the socket, wherein the elbow has a swivel connection with the frame.

In some configurations, the collar extends rearwardly from the front wall of the frame to a rearward edge. A rearward edge of the divider wall is positioned between the front wall of the frame and the rearward edge of the collar.

In some configurations, the frame forms at least a portion of the breathing chamber.

In some configurations, a space defined by an interior surface of the collar, an upper surface of the dividing wall and an interior surface of a vent wall portion of the front wall forms at least a portion of the breathing chamber.

In some configurations, the collar comprises an internal surface and an engagement surface, wherein the engagement surface engages with the friction member.

In some configurations, the collar further comprises a rim.

In some configurations, the friction member comprises an internal engagement surface and a peripheral surface.

In some configurations, the connection opening is defined by a support wall, wherein the support wall is disposed within the friction member and the housing extends through the peripheral surface of the friction member.

In some configurations, the cushion module further comprises a support flange that extends along a length of and supports the friction member.

In some configurations, the support flange is embedded within the friction member.

In some configurations, a portion of the housing disposed within the friction member comprises a plurality of apertures.

In some configurations, the friction member extends through each of the plurality of apertures.

In some configurations, a portion of the housing disposed within the friction member comprises a plurality of apertures and the friction member extends through each of the plurality of apertures, wherein at least a portion of the apertures are located between the support wall and the peripheral surface.

In some configurations, at least a portion of the apertures are located on the support flange.

In some configurations, the friction member defines an abutment surface that contacts the front wall of the frame when the cushion module is coupled to the frame.

In some configurations, the friction member defines a first length, the support flange defines a second length, an unsupported portion of the friction member that extends beyond the support flange defines a third length, and the collar defines a fourth length.

In some configurations, the first length is equal to the sum of the second length and the third length.

In some configurations, the first length is greater than the sum of the second length and the third length.

In some configurations, the first length is less than the sum of the second length and the third length.

In some configurations, the friction member defines a first thickness, the support flange defines a second thickness, a portion of the friction member located outside of the support flange defines a third thickness, a portion of the friction member located inside of the support flange defines a fourth thickness, and the collar defines a fifth thickness.

In some configurations, the first thickness equals the sum of the second thickness, the third thickness, and the fourth thickness.

In some configurations, the third thickness and the fourth thickness are equal.

In some configurations, the second thickness is equal to each of the third thickness and the fourth thickness.

In some configurations, the fourth thickness is greater than or equal to the third thickness.

In some configurations, the fourth thickness is less than or equal to the third thickness.

In some configurations, the second thickness and the fifth thickness are equal.

In some configurations, the second thickness is greater than the fifth thickness.

In some configurations, the second thickness is less than the fifth thickness.

In some configurations, the second thickness is less than or equal to either one of the third thickness and the fourth thickness.

In some configurations, the second thickness is greater than or equal to either one of the third thickness and the fourth thickness.

In some configurations, the first thickness is greater than or equal to the fifth thickness.

In some configurations, the first thickness is less than or equal to the fifth thickness.

In some configurations, the fourth thickness varies along the length of the friction member.

In some configurations, fourth thickness varies along the length of the friction member from a minimum near a proximal end of the friction member to a maximum near a distal end of the friction member.

In some configurations, the fourth thickness varies along the length of the friction member from a maximum near a proximal end of the friction member to a minimum near a distal end of the friction member.

In some configurations, the third thickness is zero.

In some configurations, a portion of the friction member that overlaps with the collar defines an overlap length.

In some configurations, the overlap length is greater than or equal to the third length.

In some configurations, the overlap length is greater than or equal to a sum of the second length and the third length.

In some configurations, the overlap length is less than the third length.

In some configurations, the overlap length is less than a sum of the second length and the third length.

In some configurations, the overlap length equals the fourth length.

In some configurations, the overlap length is less than the fourth length.

In some configurations, the overlap length is greater than the fourth length.

In some configurations, a portion of the friction member that does not overlap with the collar defines a non-overlap length and a portion of the front wall adjacent the support flange defines a first front wall thickness.

In some configurations, a sum of the first length and the first front wall thickness equals a sum of the overlap length and the non-overlap length.

In some configurations, a sum of the first length and the first front wall thickness is greater than a sum of the overlap length and the non-overlap length.

In some configurations, a sum of the first length and the first front wall thickness is less than a sum of the overlap length and the non-overlap length.

In some configurations, the support flange comprises a plurality of apertures and the friction member extends through each of the plurality of apertures. A distance between the portion of the front wall and a point on the plurality of apertures furthest from the portion of the front wall defines an aperture offset distance.

In some configurations, the non-overlap length is greater than or equal to a sum of the aperture offset distance and the first front wall thickness.

In some configurations, the non-overlap length is less than or equal to a sum of the aperture offset distance and the first front wall thickness.

In some configurations, a sum of the second length and the third length is greater than or equal to a sum of the overlap length and the aperture offset distance.

In some configurations, a sum of the second length and the third length is less than or equal to a sum of the overlap length and the aperture offset distance.

In some configurations, a sum of the overlap length and the aperture offset distance is less than or equal to first length.

In some configurations, a sum of the overlap length and the aperture offset distance is greater than or equal to first length.

In some configurations, a relative angle is defined between the collar and at least one of the support flange and a surface of the friction member that engages the collar in a direction of assembly of the cushion module to the frame.

In some configurations, the collar is angled relative to the direction of assembly.

In some configurations, at least one of the support flange and the surface of the friction member that engages the collar is angled relative to the direction of assembly.

In some configurations, a relative angle is defined between the collar and a surface of the friction member that engages the collar in a direction of assembly of the cushion module to the frame.

In some configurations, the collar is angled relative to the direction of assembly.

In some configurations, the surface of the friction member that engages the collar is angled relative to the direction of assembly.

In some configurations, the friction coupling is configured such that a force acting to compress the friction member is equal along a length of the collar.

In some configurations, the friction coupling is configured such that a force acting to compress the friction member is unequal along a length of the collar.

In some configurations, the force is larger at a location closer to the front wall relative to a location further from the front wall.

In some configurations, the force is smaller at a location closer to the front wall relative to a location further from the front wall.

In some configurations, a rearward-most extent of the friction member is at or forward of the front wall.

In some configurations, a rearward-most extent of the friction member is rearward of the front wall such that a portion of the friction member is located adjacent each of a forward surface and a rearward surface of the front wall.

In some configurations, the portion of the friction member adjacent the forward surface of the front wall has a surface that is aligned with a surface of the portion of the friction member adjacent the rearward surface of the front wall.

In some configurations, the connection opening is defined by a support wall. The support wall is disposed within the friction member such that the friction member is coupled to the cushion module.

In some configurations, the collar defines a collar length, the support wall defines a support wall thickness, and the friction member defines a total friction member length, a forward length forward of the support wall and a rearward length rearward of the support wall.

In some configurations, the total friction member length is equal to a sum of the forward length, the rearward length and the support wall thickness.

In some configurations, the total friction member length is greater than a sum of the forward length, the rearward length and the support wall thickness.

In some configurations, the total friction member length is less than a sum of the forward length, the rearward length and the support wall thickness.

In some configurations, the collar defines a collar thickness and the friction member defines a total thickness, a supported thickness along the support wall and an unsupported thickness inwardly of the support wall.

In some configurations, the total thickness equals a sum of the supported thickness and the unsupported thickness.

In some configurations, the total thickness equals twice the supported thickness.

In some configurations, the total thickness equals twice the unsupported thickness.

In some configurations, the supported thickness is equal to the unsupported thickness.

In some configurations, the supported thickness is greater than or equal to the unsupported thickness.

In some configurations, the supported thickness is less than or equal to the unsupported thickness.

In some configurations, the total thickness is greater than or equal to the collar thickness.

In some configurations, the total thickness is less than or equal to the collar thickness.

In some configurations, a portion of the friction member that overlaps with the collar defines an overlap length, a portion of the friction member that does not overlap with the collar defines a non-overlap length and a distance between a rearward end of the collar and the support wall along the direction of assembly defines a support wall offset distance.

In some configurations, the overlap length is greater than or equal to the support wall offset distance.

In some configurations, the overlap length is less than the support wall offset distance.

In some configurations, the overlap length is greater than or equal to the non-overlap length.

In some configurations, the overlap length is less than the non-overlap length.

In some configurations, the non-overlap length is greater than or equal to a sum of the support wall offset distance and the support wall thickness.

In some configurations, the non-overlap length is less than or equal to a sum of the support wall offset distance and the support wall thickness.

In some configurations, the overlap length equals the collar length.

In some configurations, the overlap length is less than the collar length.

In some configurations, the overlap length is greater than the collar length.

In some configurations, the total friction member length equals a sum of the overlap length and the non-overlap length.

In some configurations, the overlap length is less than the forward length.

In some configurations, the overlap length is greater than or equal to the forward length.

In some configurations, the overlap length is greater than or equal to a sum of the forward length, the rearward length and the support wall thickness.

In some configurations, the overlap length is less than or equal to a sum of the forward length, the rearward length and the support wall thickness.

In some configurations, the support wall thickness is less than or equal to the rearward length.

In some configurations, the support wall thickness is greater than the rearward length.

In some configurations, the housing comprises a wall portion external of the friction member and extending between the friction member and the cushion.

In some configurations, at least a surface of the friction coupling that engages the collar has a frosted or textured surface finish.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask comprises a frame configured to connect to headgear. The frame comprises a front wall at least partially defining a gas inlet opening. The frame further comprises a collar extending away from the front wall. The collar at least partially surrounds the gas inlet opening. A cushion module comprises a housing, a cushion and an elastomeric friction member. The cushion defines a face-contacting surface. The housing defines a connection opening. A portion of the housing defining the connection opening is embedded within the elastomeric friction member to couple the friction member to the housing. The housing is made of a material more rigid than the cushion and forms at least a portion of a breathing chamber of the respiratory mask. The friction member is configured to selectively connect the cushion module to the collar of the frame with a friction fit so that the flow of gas can be delivered to the breathing chamber through the gas inlet opening of the frame and the connection opening of the cushion module. The friction member engages an outer surface of the collar. An outer surface of the friction member is exposed when the cushion module is coupled to the frame.

In some configurations, the housing comprises a wall portion external of the friction member and extending between the friction member and the cushion.

In some configurations, the front wall of the frame further comprises a vent, and wherein the collar surrounds the vent in addition to the gas inlet.

In some configurations, a divider wall is located within an interior of the collar. The divider wall has a first end and a second end, each connected to the collar. The divider wall separates the vent and the gas inlet opening.

In some configurations, a surface of the divider wall adjacent the vent extends substantially in a direction of gas flow through the vent.

In some configurations, an elbow is supported by the frame and configured to deliver a flow of breathing gas through the gas inlet opening.

In some configurations, the collar and the divider wall cooperate to define a socket that receives the elbow.

In some configurations, the elbow has a swivel connection with the frame.

In some configurations, the collar extends rearwardly from the front wall of the frame to a rearward edge, wherein a rearward edge of the divider wall is positioned between the front wall of the frame and the rearward edge of the collar.

In some configurations, the housing of the cushion module further comprises a support flange, wherein the support flange defines the connection opening and extends within the friction member substantially in a direction of assembly of the cushion module to the frame.

In some configurations, the support flange comprises a plurality of apertures and wherein the friction member extends through each of the plurality of apertures.

In some configurations, the friction member defines an abutment surface that contacts the front wall of the frame when the cushion module is coupled to the frame.

In some configurations, the friction member defines a first length, the support flange defines a second length, an unsupported portion of the friction member that extends beyond the support flange defines a third length, and the collar defines a fourth length.

In some configurations, the first length is equal to the sum of the second length and the third length.

In some configurations, the first length is greater than the sum of the second length and the third length.

In some configurations, the first length is less than the sum of the second length and the third length.

In some configurations, the friction member defines a first thickness, the support flange defines a second thickness, a portion of the friction member located outside of the support flange defines a third thickness, a portion of the friction member located inside of the support flange defines a fourth thickness, and the collar defines a fifth thickness.

In some configurations, the first thickness equals the sum of the second thickness, the third thickness, and the fourth thickness.

In some configurations, the third thickness and the fourth thickness are equal.

In some configurations, the second thickness is equal to each of the third thickness and the fourth thickness.

In some configurations, the fourth thickness is greater than or equal to the third thickness.

In some configurations, the fourth thickness is less than or equal to the third thickness.

In some configurations, the second thickness and the fifth thickness are equal.

In some configurations, the second thickness is greater than the fifth thickness.

In some configurations, the second thickness is less than the fifth thickness.

In some configurations, the second thickness is less than or equal to either one of the third thickness and the fourth thickness.

In some configurations, the second thickness is greater than or equal to either one of the third thickness and the fourth thickness.

In some configurations, the first thickness is greater than or equal to the fifth thickness.

In some configurations, the first thickness is less than or equal to the fifth thickness.

In some configurations, the fourth thickness varies along the length of the friction member.

In some configurations, the fourth thickness varies along the length of the friction member from a minimum near a proximal end of the friction member to a maximum near a distal end of the friction member.

In some configurations, the fourth thickness varies along the length of the friction member from a maximum near a proximal end of the friction member to a minimum near a distal end of the friction member.

In some configurations, the third thickness is zero.

In some configurations, a portion of the friction member that overlaps with the collar defines an overlap length.

In some configurations, the overlap length is greater than or equal to the third length.

In some configurations, the overlap length is greater than or equal to a sum of the second length and the third length.

In some configurations, the overlap length is less than the third length.

In some configurations, the overlap length is less than a sum of the second length and the third length.

In some configurations, the overlap length equals the fourth length.

In some configurations, the overlap length is less than the fourth length.

In some configurations, the overlap length is greater than the fourth length.

In some configurations, a portion of the friction member that does not overlap with the collar defines a non-overlap length and wherein a portion of the front wall adjacent the support flange defines a first front wall thickness.

In some configurations, a sum of the first length and the first front wall thickness equals a sum of the overlap length and the non-overlap length.

In some configurations, a sum of the first length and the first front wall thickness is greater than a sum of the overlap length and the non-overlap length.

In some configurations, a sum of the first length and the first front wall thickness is less than a sum of the overlap length and the non-overlap length.

In some configurations, the support flange comprises a plurality of apertures and the friction member extends through each of the plurality of apertures. A distance between the portion of the front wall and a point on the plurality of apertures furthest from the portion of the front wall defines an aperture offset distance.

In some configurations, the non-overlap length is greater than or equal to a sum of the aperture offset distance and the first front wall thickness.

In some configurations, the non-overlap length is less than or equal to a sum of the aperture offset distance and the first front wall thickness.

In some configurations, a sum of the second length and the third length is greater than or equal to a sum of the overlap length and the aperture offset distance.

In some configurations, a sum of the second length and the third length is less than or equal to a sum of the overlap length and the aperture offset distance.

In some configurations, a sum of the overlap length and the aperture offset distance is less than or equal to first length.

In some configurations, a sum of the overlap length and the aperture offset distance is greater than or equal to first length.

In some configurations, a relative angle is defined between the collar and at least one of the support flange and a surface of the friction member that engages the collar in a direction of assembly of the cushion module to the frame.

In some configurations, the collar is angled relative to the direction of assembly.

In some configurations, at least one of the support flange and the surface of the friction member that engages the collar is angled relative to the direction of assembly.

In some configurations, a relative angle is defined between the collar and a surface of the friction member that engages the collar in a direction of assembly of the cushion module to the frame.

In some configurations, the collar is angled relative to the direction of assembly.

In some configurations, the surface of the friction member that engages the collar is angled relative to the direction of assembly.

In some configurations, the friction coupling is configured such that a force acting to compress the friction member is equal along a length of the collar.

In some configurations, the friction coupling is configured such that a force acting to compress the friction member is unequal along a length of the collar.

In some configurations, the force is larger at a location closer to the front wall relative to a location further from the front wall.

In some configurations, the force is smaller at a location closer to the front wall relative to a location further from the front wall.

In some configurations, a rearward-most extent of the friction member is at or forward of the front wall.

In some configurations, a rearward-most extent of the friction member is rearward of the front wall such that a portion of the friction member is located adjacent each of a forward surface and a rearward surface of the front wall.

In some configurations, the portion of the friction member adjacent the forward surface of the front wall has a surface that is aligned with a surface of the portion of the friction member adjacent the rearward surface of the front wall.

In some configurations, the connection opening is defined by a support wall. The support wall is disposed within the friction member such that the friction member is coupled to the cushion module. The support wall extends in a direction substantially perpendicular to a direction of assembly of the cushion module to the frame.

In some configurations, the collar defines a collar length, the support wall defines a support wall thickness, and the friction member defines a total friction member length, a forward length forward of the support wall and a rearward length rearward of the support wall.

In some configurations, the total friction member length is equal to a sum of the forward length, the rearward length and the support wall thickness.

In some configurations, the total friction member length is greater than a sum of the forward length, the rearward length and the support wall thickness.

In some configurations, the total friction member length is less than a sum of the forward length, the rearward length and the support wall thickness.

In some configurations, the collar defines a collar thickness and the friction member defines a total thickness, a supported thickness along the support wall and an unsupported thickness inwardly of the support wall.

In some configurations, the total thickness equals a sum of the supported thickness and the unsupported thickness.

In some configurations, the total thickness equals twice the supported thickness.

In some configurations, the total thickness equals twice the unsupported thickness.

In some configurations, the supported thickness is equal to the unsupported thickness.

In some configurations, the supported thickness is greater than or equal to the unsupported thickness.

In some configurations, the supported thickness is less than or equal to the unsupported thickness.

In some configurations, the total thickness is greater than or equal to the collar thickness.

In some configurations, the total thickness is less than or equal to the collar thickness.

In some configurations, a portion of the friction member that overlaps with the collar defines an overlap length, a portion of the friction member that does not overlap with the collar defines a non-overlap length and a distance between a rearward end of the collar and the support wall along the direction of assembly defines a support wall offset distance.

In some configurations, the overlap length is greater than or equal to the support wall offset distance.

In some configurations, the overlap length is less than the support wall offset distance.

In some configurations, the overlap length is greater than or equal to the non-overlap length.

In some configurations, the overlap length is less than the non-overlap length.

In some configurations, the non-overlap length is greater than or equal to a sum of the support wall offset distance and the support wall thickness.

In some configurations, the non-overlap length is less than or equal to a sum of the support wall offset distance and the support wall thickness.

In some configurations, the overlap length equals the collar length.

In some configurations, the overlap length is less than the collar length.

In some configurations, the overlap length is greater than the collar length.

In some configurations, the total friction member length equals a sum of the overlap length and the non-overlap length.

In some configurations, the overlap length is less than the forward length.

In some configurations, the overlap length is greater than or equal to the forward length.

In some configurations, the overlap length is greater than or equal to a sum of the forward length, the rearward length and the support wall thickness.

In some configurations, the overlap length is less than or equal to a sum of the forward length, the rearward length and the support wall thickness.

In some configurations, the support wall thickness is less than or equal to the rearward length.

In some configurations, the support wall thickness is greater than the rearward length.

In some configurations, at least a surface of the friction coupling that engages the collar has a frosted or textured surface finish.

In some configurations, the frame further comprises a conduit connector that is unitarily formed with one or both of the front wall and the collar.

In some configurations, the housing of the cushion module defines an inlet recess positioned below the connection opening, and wherein the inlet recess is configured to accommodate a portion of the conduit connector.

In some configurations, the inlet recess is configured to accommodate a rearward-facing portion of the conduit connector.

In some configurations, the housing of the cushion module comprises a recess that surrounds the connection opening.

In some configurations, the embedded portion is displaced rearward from a main wall of the housing along an axis of the connection opening by a connecting portion.

In some configurations, the housing of the cushion module comprises a recess that extends around only a portion of the connection opening.

In some configurations, the housing of the cushion module comprises a recess that extends around only an upper portion of the connection opening.

In some configurations, the housing of the cushion module comprises a recess that extends around only a lower portion of the connection opening.

In some configurations, at least a portion of the friction member is located within the recess. In some configurations, the portion of the friction member defines a portion of the recess.

In some configurations, at least a portion of the friction member protrudes in front of the recess.

In some configurations, the recess defines a depth that is less than a total length of the friction member or a length of the friction member forward of the embedded portion of the housing.

In some configurations, the recess defines a depth that is greater than or equal to a total length of the friction member or a length of the friction member forward of the embedded portion of the housing.

In some configurations, the housing further comprises a support wall that supports the friction member and defines a portion or an entirety of the embedded portion, and the support wall is connected to the front wall by a connecting portion.

In some configurations, the housing further comprises a support wall that supports the friction member and defines a portion or an entirety of the embedded portion, and a radially-inward end of the support wall is connected to a support flange having a support flange length that is greater than a thickness of the support wall.

In some configurations, the support flange defines a longitudinal axis that is offset from an axis defined by a surface of the friction member that engages the collar.

In some configurations, an opening of the friction member defines a non-circular shape.

In some configurations, the opening of the friction member comprises a first axis and a second axis, the first axis extends in a lateral direction across a widest part of the opening, and the second axis extends in a vertical direction.

In some configurations, the friction member is symmetric about the second axis.

In some configurations, the friction member is asymmetric about the first axis.

In some configurations, the first axis is a major axis and the second axis is a minor axis. In some such configurations, the seal is configured to seal below the bridge of the nose of the user.

In some configurations, the first axis is a minor axis and the second axis is a major axis. In some such configurations, the seal is configured to seal on the bridge of the nose of the user.

In some configurations, a ratio of the first axis to the second axis is between about 1.2-1.4:1, is about 1.3:1, or is about 1.27:1.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask comprises a frame configured to connect to headgear. The frame comprises a front wall at least partially defining a gas inlet opening. The frame further comprises a collar extending away from the front wall. The collar at least partially surrounds the gas inlet opening. A cushion module comprises a housing, a cushion and an elastomeric friction member. The cushion defines a face-contacting surface. The housing defines a connection opening. The housing is made of a material more rigid than the cushion and forms at least a portion of a breathing chamber of the respiratory mask. The elastomeric friction member is configured to selectively connect the cushion module to the collar of the frame with a friction fit so that the flow of gas can be delivered to the breathing chamber through the gas inlet opening of the frame and the connection opening of the cushion module. The elastomeric friction member comprises an internal engagement surface and a peripheral surface. The internal engagement surface is configured to engage an outer surface of the collar when the cushion module is coupled to the frame. An embedded portion of the housing is disposed within the elastomeric friction member and the housing extends through the peripheral surface.

In some configurations, the embedded portion comprises a forward or rearward-extending flange.

In some configurations, the housing comprises a wall portion external of the friction member and extending between the friction member and the cushion.

In some configurations, the front wall of the frame further comprises a vent, and wherein the collar surrounds the vent in addition to the gas inlet.

In some configurations, the respiratory mask further comprises a divider wall within an interior of the collar. The divider wall has a first end and a second end, each connected to the collar. The divider wall separates the vent and the gas inlet opening.

In some configurations, a surface of the divider wall adjacent the vent extends substantially in a direction of gas flow through the vent.

In some configurations, the collar and the divider wall cooperate to define a socket configured to receive an elbow.

In some configurations, the respiratory mask further comprises an elbow received in the socket. The elbow has a swivel connection with the frame.

In some configurations, the collar extends rearwardly from the front wall of the frame to a rearward edge, wherein a rearward edge of the divider wall is positioned between the front wall of the frame and the rearward edge of the collar.

In some configurations, the housing of the cushion module further comprises a support flange, wherein the support flange defines the connection opening and extends within the friction member substantially in a direction of assembly of the cushion module to the frame.

In some configurations, the support flange comprises a plurality of apertures and wherein the friction member extends through each of the plurality of apertures.

In some configurations, the friction member defines an abutment surface that contacts the front wall of the frame when the cushion module is coupled to the frame.

In some configurations, the friction member defines a first length, the support flange defines a second length, an unsupported portion of the friction member that extends beyond the support flange defines a third length, and the collar defines a fourth length.

In some configurations, the first thickness equals the sum of the second thickness, the third thickness, and the fourth thickness.

In some configurations, the third thickness and the fourth thickness are equal.

In some configurations, the second thickness is equal to each of the third thickness and the fourth thickness.

In some configurations, the fourth thickness is greater than or equal to the third thickness.

In some configurations, the fourth thickness is less than or equal to the third thickness.

In some configurations, the second thickness and the fifth thickness are equal.

In some configurations, the second thickness is greater than the fifth thickness.

In some configurations, the second thickness is less than the fifth thickness.

In some configurations, the second thickness is less than or equal to either one of the third thickness and the fourth thickness.

In some configurations, the second thickness is greater than or equal to either one of the third thickness and the fourth thickness.

In some configurations, the first thickness is greater than or equal to the fifth thickness.

In some configurations, the first thickness is less than or equal to the fifth thickness.

In some configurations, the fourth thickness varies along the length of the friction member.

In some configurations, the fourth thickness varies along the length of the friction member from a minimum near a proximal end of the friction member to a maximum near a distal end of the friction member.

In some configurations, the fourth thickness varies along the length of the friction member from a maximum near a proximal end of the friction member to a minimum near a distal end of the friction member.

In some configurations, the third thickness is zero.

In some configurations, a portion of the friction member that overlaps with the collar defines an overlap length.

In some configurations, the overlap length is greater than or equal to the third length.

In some configurations, the overlap length is greater than or equal to a sum of the second length and the third length.

In some configurations, the overlap length is less than the third length.

In some configurations, the overlap length is less than a sum of the second length and the third length.

In some configurations, the overlap length equals the fourth length.

In some configurations, the overlap length is less than the fourth length.

In some configurations, the overlap length is greater than the fourth length.

In some configurations, a portion of the friction member that does not overlap with the collar defines a non-overlap length and a portion of the front wall adjacent the support flange defines a first front wall thickness.

In some configurations, a sum of the first length and the first front wall thickness equals a sum of the overlap length and the non-overlap length.

In some configurations, a sum of the first length and the first front wall thickness is greater than a sum of the overlap length and the non-overlap length.

In some configurations, a sum of the first length and the first front wall thickness is less than a sum of the overlap length and the non-overlap length.

In some configurations, the support flange comprises a plurality of apertures and the friction member extends through each of the plurality of apertures. A distance between the portion of the front wall and a point on the plurality of apertures furthest from the portion of the front wall defines an aperture offset distance.

In some configurations, the non-overlap length is greater than or equal to a sum of the aperture offset distance and the first front wall thickness.

In some configurations, the non-overlap length is less than or equal to a sum of the aperture offset distance and the first front wall thickness.

In some configurations, a sum of the second length and the third length is greater than or equal to a sum of the overlap length and the aperture offset distance.

In some configurations, a sum of the second length and the third length is less than or equal to a sum of the overlap length and the aperture offset distance.

In some configurations, a sum of the overlap length and the aperture offset distance is less than or equal to first length.

In some configurations, a sum of the overlap length and the aperture offset distance is greater than or equal to first length.

In some configurations, a relative angle is defined between the collar and at least one of the support flange and a surface of the friction member that engages the collar in a direction of assembly of the cushion module to the frame.

In some configurations, the collar is angled relative to the direction of assembly.

In some configurations, at least one of the support flange and the surface of the friction member that engages the collar is angled relative to the direction of assembly.

In some configurations, a relative angle is defined between the collar and a surface of the friction member that engages the collar in a direction of assembly of the cushion module to the frame.

In some configurations, the collar is angled relative to the direction of assembly.

In some configurations, the surface of the friction member that engages the collar is angled relative to the direction of assembly.

In some configurations, the friction coupling is configured such that a force acting to compress the friction member is equal along a length of the collar.

In some configurations, the friction coupling is configured such that a force acting to compress the friction member is unequal along a length of the collar.

In some configurations, the force is larger at a location closer to the front wall relative to a location further from the front wall.

In some configurations, the force is smaller at a location closer to the front wall relative to a location further from the front wall.

In some configurations, a rearward-most extent of the friction member is at or forward of the front wall.

In some configurations, a rearward-most extent of the friction member is rearward of the front wall such that a portion of the friction member is located adjacent each of a forward surface and a rearward surface of the front wall.

In some configurations, the portion of the friction member adjacent the forward surface of the front wall has a surface that is aligned with a surface of the portion of the friction member adjacent the rearward surface of the front wall.

In some configurations, the connection opening is defined by a support wall, wherein the support wall is disposed within the friction member such that the friction member is coupled to the cushion module.

In some configurations, the collar defines a collar length, the support wall defines a support wall thickness, and the friction member defines a total friction member length, a forward length forward of the support wall and a rearward length rearward of the support wall.

In some configurations, the total friction member length is equal to a sum of the forward length, the rearward length and the support wall thickness.

In some configurations, the total friction member length is greater than a sum of the forward length, the rearward length and the support wall thickness.

In some configurations, the total friction member length is less than a sum of the forward length, the rearward length and the support wall thickness.

In some configurations, the collar defines a collar thickness and the friction member defines a total thickness, a supported thickness along the support wall and an unsupported thickness inwardly of the support wall.

In some configurations, the total thickness equals a sum of the supported thickness and the unsupported thickness.

In some configurations, the total thickness equals twice the supported thickness.

In some configurations, the total thickness equals twice the unsupported thickness.

In some configurations, the supported thickness is equal to the unsupported thickness.

In some configurations, the supported thickness is greater than or equal to the unsupported thickness.

In some configurations, the supported thickness is less than or equal to the unsupported thickness.

In some configurations, the total thickness is greater than or equal to the collar thickness.

In some configurations, the total thickness is less than or equal to the collar thickness.

In some configurations, a portion of the friction member that overlaps with the collar defines an overlap length, a portion of the friction member that does not overlap with the collar defines a non-overlap length and a distance between a rearward end of the collar and the support wall along the direction of assembly defines a support wall offset distance.

In some configurations, the overlap length is greater than or equal to the support wall offset distance.

In some configurations, the overlap length is less than the support wall offset distance.

In some configurations, the overlap length is greater than or equal to the non-overlap length.

In some configurations, the overlap length is less than the non-overlap length.

In some configurations, the non-overlap length is greater than or equal to a sum of the support wall offset distance and the support wall thickness.

In some configurations, the non-overlap length is less than or equal to a sum of the support wall offset distance and the support wall thickness.

In some configurations, the overlap length equals the collar length.

In some configurations, the overlap length is less than the collar length.

In some configurations, the overlap length is greater than the collar length.

In some configurations, the total friction member length equals a sum of the overlap length and the non-overlap length.

In some configurations, the overlap length is less than the forward length.

In some configurations, the overlap length is greater than or equal to the forward length.

In some configurations, the overlap length is greater than or equal to a sum of the forward length, the rearward length and the support wall thickness.

In some configurations, the overlap length is less than or equal to a sum of the forward length, the rearward length and the support wall thickness.

In some configurations, the support wall thickness is less than or equal to the rearward length.

In some configurations, the support wall thickness is greater than the rearward length.

In some configurations, at least a surface of the friction coupling that engages the collar has a frosted or textured surface finish.

In some configurations, the internal engagement surface comprises a first engagement surface portion and a second engagement surface portion. A retention force between the internal engagement surface and the outer surface of the collar varies between the first engagement surface portion and the second engagement surface portion.

In some configurations, the first engagement surface portion and the second engagement surface portion define an angle relative to another.

In some configurations, at least one of the first engagement surface portion and the second engagement surface portion define an angle relative to the outer surface of the collar.

In some configurations, the second engagement surface portion does not contact the outer surface of the collar.

In some configurations, the retention force varies over a length of the first engagement surface.

In some configurations, the elastomeric friction member extends in a rearward direction from the housing and into the breathing chamber.

In some configurations, an entirety of the second engagement surface portion is located within the breathing chamber.

In some configurations, the elastomeric friction member also extends in a forward direction from the housing away from the breathing chamber.

In some configurations, the elastomeric friction member extends in the rearward direction further than it extends in the forward direction.

In some configurations, the outer surface of the collar has a first perimeter portion and a second perimeter portion.

In some configurations, the first perimeter portion has a first perimeter, the second perimeter portion has a second perimeter, and the first perimeter is greater than the second perimeter.

In some configurations, the first perimeter portion is more proximal to the front wall than the second perimeter portion.

In some configurations, the embedded portion is adjacent the first perimeter portion when the cushion module is coupled to the frame.

In some configurations, the embedded portion is adjacent the second perimeter portion when the cushion module is coupled to the frame.

In some configurations, one or both of the elastomeric friction member and the outer surface of the collar define a circular shape and the respiratory mask further comprises structures to indicate a correct assembly orientation, inhibit or prevent incorrect assembly, or guide the cushion module and the frame towards or into the correct assembly orientation.

In some configurations, the structures inhibit or prevent relative rotational movement of the cushion module and the frame when the cushion module is properly assembled to the frame.

In some configurations, the structures comprise one or more sets of cooperating protrusions and recesses.

In some configurations, the collar comprises at least one of the recesses and the elastomeric friction member comprises at least one of the protrusions.

In some configurations, the protrusion is configured to occupy an entirety of a corresponding recess.

In some configurations, the recess comprises an angled surface relative to a direction of assembly of the cushion module to the frame.

In some configurations, the recess comprises a straight surface that is aligned with the direction of assembly.

In some configurations, the angled surface and the straight surface cooperate to form a generally triangular shape.

In some configurations, the angled surface is configured to contact the corresponding protrusion to guide the cushion module towards or to the correct assembly orientation.

In some configurations, both of a first set and a second set of the cooperating protrusions and recesses are located within an upper half of the elastomeric friction member and the collar, respectively, and the angled surfaces are located further from a vertical centerline of the frame than the straight surfaces.

In some configurations, the embedded portion provides radial support to the elastomeric friction member.

In some configurations, the frame further comprises a conduit connector that is unitarily formed with one or both of the front wall and the collar.

In some configurations, the housing of the cushion module defines an inlet recess positioned below the connection opening, and wherein the inlet recess is configured to accommodate a portion of the conduit connector.

In some configurations, the inlet recess is configured to accommodate a rearward-facing portion of the conduit connector.

In some configurations, the housing of the cushion module comprises a recess that surrounds the connection opening.

In some configurations, the embedded portion is displaced rearward from a main wall of the housing along an axis of the connection opening by a connecting portion.

In some configurations, the housing of the cushion module comprises a recess that extends around only a portion of the connection opening.

In some configurations, the housing of the cushion module comprises a recess that extends around only an upper portion of the connection opening.

In some configurations, the housing of the cushion module comprises a recess that extends around only a lower portion of the connection opening.

In some configurations, at least a portion of the friction member is located within the recess. In some configurations, the portion of the friction member defines a portion of the recess.

In some configurations, at least a portion of the friction member protrudes in front of the recess.

In some configurations, the recess defines a depth that is less than a total length of the friction member or a length of the friction member forward of the embedded portion of the housing.

In some configurations, the recess defines a depth that is greater than or equal to a total length of the friction member or a length of the friction member forward of the embedded portion of the housing.

In some configurations, the housing further comprises a support wall that supports the friction member and defines a portion or an entirety of the embedded portion, and the support wall is connected to the front wall by a connecting portion.

In some configurations, the housing further comprises a support wall that supports the friction member and defines a portion or an entirety of the embedded portion, and a radially-inward end of the support wall is connected to a support flange having a support flange length that is greater than a thickness of the support wall.

In some configurations, the support flange defines a longitudinal axis that is offset from an axis defined by a surface of the friction member that engages the collar.

In some configurations, an opening of the friction member defines a non-circular shape.

In some configurations, the opening of the friction member comprises a first axis and a second axis, the first axis extends in a lateral direction across a widest part of the opening, and the second axis extends in a vertical direction.

In some configurations, the friction member is symmetric about the second axis.

In some configurations, the friction member is asymmetric about the first axis.

In some configurations, the first axis is a major axis and the second axis is a minor axis. In some such configurations, the seal is configured to seal below the bridge of the nose of the user.

In some configurations, the first axis is a minor axis and the second axis is a major axis. In some such configurations, the seal is configured to seal on the bridge of the nose of the user.

In some configurations, a ratio of the first axis to the second axis is between about 1.2-1.4:1, is about 1.3:1, or is about 1.27:1.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes a frame configured to connect to headgear. The frame comprises a front wall at least partially defining a gas inlet opening. The frame further comprises a collar extending away from the front wall. The collar at least partially surrounds the gas inlet opening. A cushion module comprises a housing, a cushion and an elastomeric friction member. The cushion defines a face-contacting surface. The housing defines a connection opening. A portion of the housing that defines the connection opening is embedded within the elastomeric friction member to couple the friction member to the housing. The friction member engages the collar when the cushion module is coupled to the frame.

In some configurations, the housing comprises a wall portion external of the friction member and extending between the friction member and the cushion.

In some configurations, the front wall of the frame further comprises a vent, and the collar surrounds the vent in addition to the gas inlet.

In some configurations, a divider wall is located within an interior of the collar. The divider wall has a first end and a second end, each connected to the collar. The divider wall separates the vent and the gas inlet opening.

In some configurations, a surface of the divider wall adjacent the vent extends substantially in a direction of gas flow through the vent.

In some configurations, an elbow is supported by the frame and configured to deliver a flow of breathing gas through the gas inlet opening.

In some configurations, the collar and the divider wall cooperate to define a socket that receives the elbow.

In some configurations, the elbow has a swivel connection with the frame.

In some configurations, the collar extends rearwardly from the front wall of the frame to a rearward edge, and a rearward edge of the divider wall is positioned between the front wall of the frame and the rearward edge of the collar.

In some configurations, the housing of the cushion module further comprises a support flange, and the support flange defines the connection opening and extends within the friction member substantially in a direction of assembly of the cushion module to the frame.

In some configurations, the support flange comprises a plurality of apertures and the friction member extends through each of the plurality of apertures.

In some configurations, the friction member defines an abutment surface that contacts the front wall of the frame when the cushion module is coupled to the frame.

In some configurations, the friction member defines a first length, the support flange defines a second length, an unsupported portion of the friction member that extends beyond the support flange defines a third length, and the collar defines a fourth length.

In some configurations, the first length is equal to the sum of the second length and the third length.

In some configurations, the first length is greater than the sum of the second length and the third length.

In some configurations, the first length is less than the sum of the second length and the third length.

In some configurations, the friction member defines a first thickness, the support flange defines a second thickness, a portion of the friction member located outside of the support flange defines a third thickness, a portion of the friction member located inside of the support flange defines a fourth thickness, and the collar defines a fifth thickness.

In some configurations, the first thickness equals the sum of the second thickness, the third thickness, and the fourth thickness.

In some configurations, the third thickness and the fourth thickness are equal.

In some configurations, the second thickness is equal to each of the third thickness and the fourth thickness.

In some configurations, the fourth thickness is greater than or equal to the third thickness.

In some configurations, the fourth thickness is less than or equal to the third thickness.

In some configurations, the second thickness and the fifth thickness are equal.

In some configurations, the second thickness is greater than the fifth thickness.

In some configurations, the second thickness is less than the fifth thickness.

In some configurations, the second thickness is less than or equal to either one of the third thickness and the fourth thickness.

In some configurations, the second thickness is greater than or equal to either one of the third thickness and the fourth thickness.

In some configurations, the first thickness is greater than or equal to the fifth thickness.

In some configurations, the first thickness is less than or equal to the fifth thickness.

In some configurations, the fourth thickness varies along the length of the friction member.

In some configurations, the fourth thickness varies along the length of the friction member from a minimum near a proximal end of the friction member to a maximum near a distal end of the friction member.

In some configurations, the fourth thickness varies along the length of the friction member from a maximum near a proximal end of the friction member to a minimum near a distal end of the friction member.

In some configurations, the third thickness is zero.

In some configurations, a portion of the friction member that overlaps with the collar defines an overlap length.

In some configurations, the overlap length is greater than or equal to the third length.

In some configurations, the overlap length is greater than or equal to a sum of the second length and the third length.

In some configurations, the overlap length is less than the third length.

In some configurations, the overlap length is less than a sum of the second length and the third length.

In some configurations, the overlap length equals the fourth length.

In some configurations, the overlap length is less than the fourth length.

In some configurations, the overlap length is greater than the fourth length.

In some configurations, a portion of the friction member that does not overlap with the collar defines a non-overlap length and a portion of the front wall adjacent the support flange defines a first front wall thickness.

In some configurations, a sum of the first length and the first front wall thickness equals a sum of the overlap length and the non-overlap length.

In some configurations, a sum of the first length and the first front wall thickness is greater than a sum of the overlap length and the non-overlap length.

In some configurations, a sum of the first length and the first front wall thickness is less than a sum of the overlap length and the non-overlap length.

In some configurations, the support flange comprises a plurality of apertures and the friction member extends through each of the plurality of apertures. A distance between the portion of the front wall and a point on the plurality of apertures furthest from the portion of the front wall defines an aperture offset distance.

In some configurations, the non-overlap length is greater than or equal to a sum of the aperture offset distance and the first front wall thickness.

In some configurations, the non-overlap length is less than or equal to a sum of the aperture offset distance and the first front wall thickness.

In some configurations, a sum of the second length and the third length is greater than or equal to a sum of the overlap length and the aperture offset distance.

In some configurations, a sum of the second length and the third length is less than or equal to a sum of the overlap length and the aperture offset distance.

In some configurations, a sum of the overlap length and the aperture offset distance is less than or equal to first length.

In some configurations, a sum of the overlap length and the aperture offset distance is greater than or equal to first length.

In some configurations, a relative angle is defined between the collar and at least one of the support flange and a surface of the friction member that engages the collar in a direction of assembly of the cushion module to the frame.

In some configurations, the collar is angled relative to the direction of assembly.

In some configurations, at least one of the support flange and the surface of the friction member that engages the collar is angled relative to the direction of assembly.

In some configurations, a relative angle is defined between the collar and a surface of the friction member that engages the collar in a direction of assembly of the cushion module to the frame.

In some configurations, the collar is angled relative to the direction of assembly.

In some configurations, the surface of the friction member that engages the collar is angled relative to the direction of assembly.

In some configurations, the friction coupling is configured such that a force acting to compress the friction member is equal along a length of the collar.

In some configurations, the friction coupling is configured such that a force acting to compress the friction member is unequal along a length of the collar.

In some configurations, the force is larger at a location closer to the front wall relative to a location further from the front wall.

In some configurations, the force is smaller at a location closer to the front wall relative to a location further from the front wall.

In some configurations, a rearward-most extent of the friction member is at or forward of the front wall.

In some configurations, a rearward-most extent of the friction member is rearward of the front wall such that a portion of the friction member is located adjacent each of a forward surface and a rearward surface of the front wall.

In some configurations, the portion of the friction member adjacent the forward surface of the front wall has a surface that is aligned with a surface of the portion of the friction member adjacent the rearward surface of the front wall.

In some configurations, the connection opening is defined by a support wall, and the support wall is disposed within the friction member such that the friction member is coupled to the cushion module.

In some configurations, the collar defines a collar length, the support wall defines a support wall thickness, and the friction member defines a total friction member length, a forward length forward of the support wall and a rearward length rearward of the support wall.

In some configurations, the total friction member length is equal to a sum of the forward length, the rearward length and the support wall thickness.

In some configurations, the total friction member length is greater than a sum of the forward length, the rearward length and the support wall thickness.

In some configurations, the total friction member length is less than a sum of the forward length, the rearward length and the support wall thickness.

In some configurations, the collar defines a collar thickness and the friction member defines a total thickness, a supported thickness along the support wall and an unsupported thickness inwardly of the support wall.

In some configurations, the total thickness equals a sum of the supported thickness and the unsupported thickness.

In some configurations, the total thickness equals twice the supported thickness.

In some configurations, the total thickness equals twice the unsupported thickness.

In some configurations, the supported thickness is equal to the unsupported thickness.

In some configurations, the supported thickness is greater than or equal to the unsupported thickness.

In some configurations, the supported thickness is less than or equal to the unsupported thickness.

In some configurations, the total thickness is greater than or equal to the collar thickness.

In some configurations, the total thickness is less than or equal to the collar thickness.

In some configurations, a portion of the friction member that overlaps with the collar defines an overlap length, a portion of the friction member that does not overlap with the collar defines a non-overlap length and a distance between a rearward end of the collar and the support wall along the direction of assembly defines a support wall offset distance.

In some configurations, the overlap length is greater than or equal to the support wall offset distance.

In some configurations, the overlap length is less than the support wall offset distance.

In some configurations, the overlap length is greater than or equal to the non-overlap length.

In some configurations, the overlap length is less than the non-overlap length.

In some configurations, the non-overlap length is greater than or equal to a sum of the support wall offset distance and the support wall thickness.

In some configurations, the non-overlap length is less than or equal to a sum of the support wall offset distance and the support wall thickness.

In some configurations, the overlap length equals the collar length.

In some configurations, the overlap length is less than the collar length.

In some configurations, the overlap length is greater than the collar length.

In some configurations, the total friction member length equals a sum of the overlap length and the non-overlap length.

In some configurations, the overlap length is less than the forward length.

In some configurations, the overlap length is greater than or equal to the forward length.

In some configurations, the overlap length is greater than or equal to a sum of the forward length, the rearward length and the support wall thickness.

In some configurations, the overlap length is less than or equal to a sum of the forward length, the rearward length and the support wall thickness.

In some configurations, the support wall thickness is less than or equal to the rearward length.

In some configurations, the support wall thickness is greater than the rearward length.

In some configurations, the embedded portion provides radial support to the elastomeric friction member.

In some configurations, the collar is inboard of an outer perimeter of the frame.

In some configurations, the frame further comprises a conduit connector that is unitarily formed with one or both of the front wall and the collar.

In some configurations, the housing of the cushion module defines an inlet recess positioned below the connection opening, and wherein the inlet recess is configured to accommodate a portion of the conduit connector.

In some configurations, the inlet recess is configured to accommodate a rearward-facing portion of the conduit connector.

In some configurations, the housing of the cushion module comprises a recess that surrounds the connection opening.

In some configurations, the embedded portion is displaced rearward from a main wall of the housing along an axis of the connection opening by a connecting portion.

In some configurations, the housing of the cushion module comprises a recess that extends around only a portion of the connection opening.

In some configurations, the housing of the cushion module comprises a recess that extends around only an upper portion of the connection opening.

In some configurations, the housing of the cushion module comprises a recess that extends around only a lower portion of the connection opening.

In some configurations, at least a portion of the friction member is located within the recess. In some configurations, the portion of the friction member defines a portion of the recess.

In some configurations, at least a portion of the friction member protrudes in front of the recess.

In some configurations, the recess defines a depth that is less than a total length of the friction member or a length of the friction member forward of the embedded portion of the housing.

In some configurations, the recess defines a depth that is greater than or equal to a total length of the friction member or a length of the friction member forward of the embedded portion of the housing.

In some configurations, the housing further comprises a support wall that supports the friction member and defines a portion or an entirety of the embedded portion, and the support wall is connected to the front wall by a connecting portion.

In some configurations, the housing further comprises a support wall that supports the friction member and defines a portion or an entirety of the embedded portion, and a radially-inward end of the support wall is connected to a support flange having a support flange length that is greater than a thickness of the support wall.

In some configurations, the support flange defines a longitudinal axis that is offset from an axis defined by a surface of the friction member that engages the collar.

In some configurations, an opening of the friction member defines a non-circular shape.

In some configurations, the opening of the friction member comprises a first axis and a second axis, the first axis extends in a lateral direction across a widest part of the opening, and the second axis extends in a vertical direction.

In some configurations, the friction member is symmetric about the second axis.

In some configurations, the friction member is asymmetric about the first axis.

In some configurations, the first axis is a major axis and the second axis is a minor axis. In some such configurations, the seal is configured to seal below the bridge of the nose of the user.

In some configurations, the first axis is a minor axis and the second axis is a major axis. In some such configurations, the seal is configured to seal on the bridge of the nose of the user.

In some configurations, a ratio of the first axis to the second axis is between about 1.2-1.4:1, is about 1.3:1, or is about 1.27:1.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a cushion module for a respiratory mask is configured for attachment to a cooperating structure, such as a frame and/or a conduit connector (e.g., elbow). The frame can be configured to connect to headgear. The frame can include a front wall at least partially defining a gas inlet opening and a collar extending away from the front wall, which can at least partially surround the gas inlet opening. The cushion module comprises a housing, a cushion and an elastomeric friction member. The cushion defines a face-contacting surface. The housing defines a connection opening. A portion of the housing defining the connection opening is embedded within the elastomeric friction member to couple the friction member to the housing. The friction member is configured to engage a corresponding portion of the frame, such as the collar, when the cushion module is coupled to the frame.

In some configurations, the housing comprises a wall portion external of the friction member and extending between the friction member and the cushion.

In some configurations, the housing of the cushion module further comprises a support flange. The support flange defines the connection opening and extends within the friction member substantially in a direction of assembly of the cushion module to the frame.

In some configurations, the support flange comprises a plurality of apertures and the friction member extends through each of the plurality of apertures.

In some configurations, the friction member defines an abutment surface that is configured to contact the front wall of the frame when the cushion module is coupled to the frame.

In some configurations, the friction member defines a first length, the support flange defines a second length, an unsupported portion of the friction member that extends beyond the support flange defines a third length, and the collar defines a fourth length.

In some configurations, the first length is equal to the sum of the second length and the third length.

In some configurations, the first length is greater than the sum of the second length and the third length.

In some configurations, the first length is less than the sum of the second length and the third length.

In some configurations, the friction member defines a first thickness, the support flange defines a second thickness, a portion of the friction member located outside of the support flange defines a third thickness, a portion of the friction member located inside of the support flange defines a fourth thickness, and the collar defines a fifth thickness.

In some configurations, the first thickness equals the sum of the second thickness, the third thickness, and the fourth thickness.

In some configurations, the third thickness and the fourth thickness are equal.

In some configurations, the second thickness is equal to each of the third thickness and the fourth thickness.

In some configurations, the fourth thickness is greater than or equal to the third thickness.

In some configurations, the fourth thickness is less than or equal to the third thickness.

In some configurations, the second thickness and the fifth thickness are equal.

In some configurations, the second thickness is greater than the fifth thickness.

In some configurations, the second thickness is less than the fifth thickness.

In some configurations, the second thickness is less than or equal to either one of the third thickness and the fourth thickness.

In some configurations, the second thickness is greater than or equal to either one of the third thickness and the fourth thickness.

In some configurations, the first thickness is greater than or equal to the fifth thickness.

In some configurations, the first thickness is less than or equal to the fifth thickness.

In some configurations, the fourth thickness varies along the length of the friction member.

In some configurations, the fourth thickness varies along the length of the friction member from a minimum near a proximal end of the friction member to a maximum near a distal end of the friction member.

In some configurations, the fourth thickness varies along the length of the friction member from a maximum near a proximal end of the friction member to a minimum near a distal end of the friction member.

In some configurations, the third thickness is zero.

In some configurations, a portion of the friction member that overlaps with the collar defines an overlap length.

In some configurations, the overlap length is greater than or equal to the third length.

In some configurations, the overlap length is greater than or equal to a sum of the second length and the third length.

In some configurations, the overlap length is less than the third length.

In some configurations, the overlap length is less than a sum of the second length and the third length.

In some configurations, the overlap length equals the fourth length.

In some configurations, the overlap length is less than the fourth length.

In some configurations, the overlap length is greater than the fourth length.

In some configurations, a portion of the friction member that does not overlap with the collar defines a non-overlap length and a portion of the front wall adjacent the support flange defines a first front wall thickness.

In some configurations, a sum of the first length and the first front wall thickness equals a sum of the overlap length and the non-overlap length.

In some configurations, a sum of the first length and the first front wall thickness is greater than a sum of the overlap length and the non-overlap length.

In some configurations, a sum of the first length and the first front wall thickness is less than a sum of the overlap length and the non-overlap length.

In some configurations, the support flange comprises a plurality of apertures and the friction member extends through each of the plurality of apertures. A distance between the portion of the front wall and a point on the plurality of apertures furthest from the portion of the front wall defines an aperture offset distance.

In some configurations, the non-overlap length is greater than or equal to a sum of the aperture offset distance and the first front wall thickness.

In some configurations, the non-overlap length is less than or equal to a sum of the aperture offset distance and the first front wall thickness.

In some configurations, a sum of the second length and the third length is greater than or equal to a sum of the overlap length and the aperture offset distance.

In some configurations, a sum of the second length and the third length is less than or equal to a sum of the overlap length and the aperture offset distance.

In some configurations, a sum of the overlap length and the aperture offset distance is less than or equal to first length.

In some configurations, a sum of the overlap length and the aperture offset distance is greater than or equal to the first length.

In some configurations, a relative angle is defined between the collar and at least one of the support flange and a surface of the friction member that engages the collar in a direction of assembly of the cushion module to the frame.

In some configurations, at least one of the support flange and the surface of the friction member that engages the collar is angled relative to the direction of assembly.

In some configurations, a relative angle is defined between the collar and a surface of the friction member that engages the collar in a direction of assembly of the cushion module to the frame.

In some configurations, the surface of the friction member that engages the collar is angled relative to the direction of assembly.

In some configurations, the friction coupling is configured such that a force acting to compress the friction member is equal along a length of the collar.

In some configurations, the friction coupling is configured such that a force acting to compress the friction member is unequal along a length of the collar.

In some configurations, the force is larger at a location closer to the front wall relative to a location further from the front wall.

In some configurations, the force is smaller at a location closer to the front wall relative to a location further from the front wall.

In some configurations, a rearward-most extent of the friction member is at or forward of the front wall.

In some configurations, a rearward-most extent of the friction member is rearward of the front wall such that a portion of the friction member is located adjacent each of a forward surface and a rearward surface of the front wall.

In some configurations, the portion of the friction member adjacent the forward surface of the front wall has a surface that is aligned with a surface of the portion of the friction member adjacent the rearward surface of the front wall.

In some configurations, the connection opening is defined by a support wall, and the support wall is disposed within the friction member such that the friction member is coupled to the cushion module.

In some configurations, the collar defines a collar length, the support wall defines a support wall thickness, and the friction member defines a total friction member length, a forward length forward of the support wall and a rearward length rearward of the support wall.

In some configurations, the total friction member length is equal to a sum of the forward length, the rearward length and the support wall thickness.

In some configurations, the total friction member length is greater than a sum of the forward length, the rearward length and the support wall thickness.

In some configurations, the total friction member length is less than a sum of the forward length, the rearward length and the support wall thickness.

In some configurations, the collar defines a collar thickness and the friction member defines a total thickness, a supported thickness along the support wall and an unsupported thickness inwardly of the support wall.

In some configurations, the total thickness equals a sum of the supported thickness and the unsupported thickness.

In some configurations, the total thickness equals twice the supported thickness.

In some configurations, the total thickness equals twice the unsupported thickness.

In some configurations, the supported thickness is equal to the unsupported thickness.

In some configurations, the supported thickness is greater than or equal to the unsupported thickness.

In some configurations, the supported thickness is less than or equal to the unsupported thickness.

In some configurations, the total thickness is greater than or equal to the collar thickness.

In some configurations, the total thickness is less than or equal to the collar thickness.

In some configurations, a portion of the friction member that overlaps with the collar defines an overlap length, a portion of the friction member that does not overlap with the collar defines a non-overlap length and a distance between a rearward end of the collar and the support wall along the direction of assembly defines a support wall offset distance.

In some configurations, the overlap length is greater than or equal to the support wall offset distance.

In some configurations, the overlap length is less than the support wall offset distance.

In some configurations, the overlap length is greater than or equal to the non-overlap length.

In some configurations, the overlap length is less than the non-overlap length.

In some configurations, the non-overlap length is greater than or equal to a sum of the support wall offset distance and the support wall thickness.

In some configurations, the non-overlap length is less than or equal to a sum of the support wall offset distance and the support wall thickness.

In some configurations, the overlap length equals the collar length.

In some configurations, the overlap length is less than the collar length.

In some configurations, the overlap length is greater than the collar length.

In some configurations, the total friction member length equals a sum of the overlap length and the non-overlap length.

In some configurations, the overlap length is less than the forward length.

In some configurations, the overlap length is greater than or equal to the forward length.

In some configurations, the overlap length is greater than or equal to a sum of the forward length, the rearward length and the support wall thickness.

In some configurations, the overlap length is less than or equal to a sum of the forward length, the rearward length and the support wall thickness.

In some configurations, the support wall thickness is less than or equal to the rearward length.

In some configurations, the support wall thickness is greater than the rearward length.

In some configurations, the embedded portion provides radial support to the elastomeric friction member.

In some configurations, the frame further comprises a conduit connector that is unitarily formed with one or both of the front wall and the collar.

In some configurations, the housing of the cushion module defines an inlet recess positioned below the connection opening, and wherein the inlet recess is configured to accommodate a portion of the conduit connector.

In some configurations, the inlet recess is configured to accommodate a rearward-facing portion of the conduit connector.

In some configurations, the housing of the cushion module comprises a recess that surrounds the connection opening.

In some configurations, the embedded portion is displaced rearward from a main wall of the housing along an axis of the connection opening by a connecting portion.

In some configurations, the housing of the cushion module comprises a recess that extends around only a portion of the connection opening.

In some configurations, the housing of the cushion module comprises a recess that extends around only an upper portion of the connection opening.

In some configurations, the housing of the cushion module comprises a recess that extends around only a lower portion of the connection opening.

In some configurations, at least a portion of the friction member is located within the recess. In some configurations, the portion of the friction member defines a portion of the recess.

In some configurations, at least a portion of the friction member protrudes in front of the recess.

In some configurations, the recess defines a depth that is less than a total length of the friction member or a length of the friction member forward of the embedded portion of the housing.

In some configurations, the recess defines a depth that is greater than or equal to a total length of the friction member or a length of the friction member forward of the embedded portion of the housing.

In some configurations, the housing further comprises a support wall that supports the friction member and defines a portion or an entirety of the embedded portion, and the support wall is connected to the front wall by a connecting portion.

In some configurations, the housing further comprises a support wall that supports the friction member and defines a portion or an entirety of the embedded portion, and a radially-inward end of the support wall is connected to a support flange having a support flange length that is greater than a thickness of the support wall.

In some configurations, the support flange defines a longitudinal axis that is offset from an axis defined by a surface of the friction member that engages the collar.

In some configurations, an opening of the friction member defines a non-circular shape.

In some configurations, the opening of the friction member comprises a first axis and a second axis, the first axis extends in a lateral direction across a widest part of the opening, and the second axis extends in a vertical direction.

In some configurations, the friction member is symmetric about the second axis.

In some configurations, the friction member is asymmetric about the first axis.

In some configurations, the first axis is a major axis and the second axis is a minor axis. In some such configurations, the seal is configured to seal below the bridge of the nose of the user.

In some configurations, the first axis is a minor axis and the second axis is a major axis. In some such configurations, the seal is configured to seal on the bridge of the nose of the user.

In some configurations, a ratio of the first axis to the second axis is between about 1.2-1.4:1, is about 1.3:1, or is about 1.27:1.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes a frame configured to connect to headgear. The frame includes a front wall at least partially defining a gas inlet opening. The frame further includes a collar extending away from the front wall. The collar at least partially surrounds the gas inlet opening. A cushion module includes a housing, a cushion and an elastomeric friction member. The cushion defines a face-contacting surface. The housing includes a main wall that supports the cushion. The housing further includes a support wall that defines a connection opening. At least a portion of the support wall is embedded within the elastomeric friction member to couple the elastomeric friction member to the housing. The support wall is offset from the main wall to define a recess of the housing. The elastomeric friction member is configured to selectively connect the cushion module to the collar of the frame with a friction fit so that a flow of gas can be delivered to the breathing chamber through the gas inlet opening of the frame and the connection opening of the cushion module.

In some configurations, the support flange is offset rearward from the main wall along an axis of the connection opening to define the recess.

In some configurations, the support flange is connected to the main wall by a connecting portion, and a surface of the connecting portion and a surface of the support flange define at least a portion of the recess.

In some configurations, the recess surrounds the connection opening.

In some configurations, the recess extends around only a portion of the connection opening.

In some configurations, the recess extends around only an upper portion of the connection opening.

In some configurations, the recess extends around only a lower portion of the connection opening.

In some configurations, at least a portion of the friction member is located within the recess. In some configurations, the portion of the friction member defines a portion of the recess.

In some configurations, at least a portion of the friction member protrudes in front of the recess.

In some configurations, the recess defines a depth that is less than a total length of the friction member or a length of the friction member forward of the support wall.

In some configurations, the recess defines a depth that is greater than or equal to a total length of the friction member or a length of the friction member forward of the embedded portion of the housing.

In some configurations, a radially-inward end of the support wall is connected to a support flange having a support flange length that is greater than a thickness of the support wall.

In some configurations, the support flange defines a longitudinal axis that is offset from an axis defined by a surface of the friction member that engages the collar.

In some configurations, an opening of the friction member defines a non-circular shape.

In some configurations, the opening of the friction member comprises a first axis and a second axis, wherein the first axis extends in a lateral direction across a widest part of the opening and the second axis extends in a vertical direction.

In some configurations, the friction member is symmetric about the second axis.

In some configurations, the friction member is asymmetric about the first axis.

In some configurations, the first axis is a major axis and the second axis is a minor axis. In some such configurations, the seal is configured to seal below the bridge of the nose of the user.

In some configurations, the first axis is a minor axis and the second axis is a major axis. In some such configurations, the seal is configured to seal on the bridge of the nose of the user.

In some configurations, a ratio of the first axis to the second axis is between about 1.2-1.4:1, is about 1.3:1, or is about 1.27:1.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes a frame configured to connect to headgear. The frame includes a front wall at least partially defining a gas inlet opening. The frame further includes a collar extending away from the front wall. The collar at least partially surrounds the gas inlet opening. A cushion module includes a housing, a cushion and an elastomeric friction member. The cushion defines a face-contacting surface. The housing includes a main wall that supports the cushion and a support wall that defines a connection opening. The support wall is offset from the main wall. The elastomeric friction member is configured to selectively connect the cushion module to the collar of the frame with a friction fit so that a flow of gas can be delivered to the breathing chamber through the gas inlet opening of the frame and the connection opening of the cushion module.

In some configurations, the support wall is connected to the main wall by a connecting portion.

In some configurations, the support wall is offset from the main wall by the connecting portion to define a recess of the housing.

In some configurations, at least a portion of the support wall is embedded within the elastomeric friction member to couple the elastomeric friction member to the housing.

In some configurations, the support wall is offset rearward from the main wall along an axis of the connection opening.

In some configurations, the support wall is offset from the main wall around an entirety of the connection opening.

In some configurations, the support wall is offset from the main wall around only a portion of the connection opening.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes a frame configured to connect to headgear. The frame includes a front wall at least partially defining a gas inlet opening. The frame further includes a collar extending away from the front wall. The collar at least partially surrounds the gas inlet opening. A cushion module includes a housing, a cushion and an elastomeric friction member. The cushion defines a face-contacting surface. The housing includes a main wall that supports the cushion and a support wall connected to the main wall by a connecting portion. The support wall defines a connection opening. The connecting portion and the support wall at least partially define a recess of the housing. The elastomeric friction member is configured to selectively connect the cushion module to the collar of the frame with a friction fit so that a flow of gas can be delivered to the breathing chamber through the gas inlet opening of the frame and the connection opening of the cushion module.

In some configurations, at least a portion of the support wall is embedded within the elastomeric friction member to couple the elastomeric friction member to the housing.

In some configurations, the recess extends rearward from the main wall along an axis of the connection opening.

In some configurations, the recess extends around an entirety of the connection opening.

In some configurations, the recess extends around only a portion of the connection opening.

In some configurations, at least a portion of the recess is defined by a peripheral surface of the elastomeric friction member.

In some configurations, at least a portion of the elastomeric friction member is disposed in the recess.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a cushion module for a respiratory mask, the cushion module configured for attachment to a frame configured to connect to a headgear, includes a housing, a cushion, and an elastomeric friction member. The cushion defines a face-contacting surface and a cushion outlet opening. The elastomeric friction member is configured to form a friction coupling with the frame. The housing includes a main wall and a support flange extending from the main wall. At least a portion of the support flange is embedded within the elastomeric friction member to couple the elastomeric friction member to the housing. The cushion module defines a connection opening. The support flange includes a first flange portion surrounding a first perimeter portion of the connection opening and defining a first front edge portion of the support flange, and a second flange portion surrounding a second perimeter portion of the connection opening and defining a second front edge portion of the support flange. The second flange portion is offset from the first flange portion.

In some configurations, the second front edge portion of the support flange is offset from the first front edge portion of the support flange.

In some configurations, the second front edge portion of the support flange is rearwardly offset from the first front edge portion of the support flange.

In some configurations, the second flange portion includes lateral portions of the support flange.

In some configurations, the first flange portion includes top and bottom portions of the support flange.

In some configurations, a length of the support flange is constant around an entire perimeter of the connection opening.

In some configurations, a respiratory mask includes the cushion module, the frame, and headgear. In some configurations, the frame includes a front wall and a collar extending away from the front wall. In some configurations, the collar extends rearwardly relative to the front wall.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a cushion module for a respiratory mask, the cushion module configured to couple to a frame configured to connect to headgear, includes a cushion, an elastomeric friction member, and a housing. The cushion defines a face-contacting surface and a cushion outlet opening. The elastomeric friction member is configured to form a friction coupling with the frame and defines a cushion module opening. The housing includes a main wall and a support flange. The main wall includes a support wall that is embedded within the elastomeric friction member and that includes a front surface. The support flange extends from the support wall and surrounds the cushion module opening. The support flange includes an inward facing surface. The support flange is at least partially embedded within the elastomeric friction member. The support flange is configured to support the elastomeric friction member. The housing includes a curved transition region between the front surface of the support wall and the inward facing surface of the support flange. The front surface is forward of the support flange.

In some configurations, the housing is disposed between the cushion and the elastomeric friction member.

In some configurations, the support wall comprises a plurality of apertures, and the elastomeric friction member extends through the plurality of apertures.

In some configurations, a compressive force on the elastomeric friction member during assembly with the frame compresses the elastomeric friction member against the support wall and the transition region.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes the cushion module, the frame and headgear. In some configurations, the frame is configured to couple to the cushion module by movement of the frame relative to the cushion module. In some configurations, the frame is configured to couple to the cushion module by movement of the frame relative to the cushion module in a coupling direction.

In some configurations, a cushion module for a respiratory mask, the cushion module configured for coupling to a frame configured to connect to headgear, includes a cushion, a housing, and an elastomeric friction member. The cushion is configured to seal against a patient in use. The housing has a first end and a second end, the first end connected to the cushion. The elastomeric friction member is connected to the second end of the housing. The elastomeric friction member comprises an inner perimeter that defines a cushion module opening. The elastomeric friction member is configured to form a friction coupling with the frame. The inner perimeter comprises a major dimension along a major axis and a minor dimension along a minor axis.

In some configurations, the major axis is perpendicular to the minor axis. In some configurations, the major axis and the minor axis intersect in a lower portion of the cushion module opening. In some configurations, the major axis and the minor axis intersect within a lower third of the cushion module opening. In some configurations, the major axis and the minor axis intersect above a lower third of the cushion module opening. In some configurations, the major axis and the minor axis intersect below the midpoint of a vertical dimension of the cushion module opening. In some configurations, the major axis and the minor axis intersect below the midpoint of a vertical dimension of the cushion module opening, and above a lower quartile of the vertical dimension.

In some configurations, the cushion module opening is symmetric about the major axis.

In some configurations, the cushion module opening is asymmetric about the minor axis.

In some configurations, the major axis corresponds with a vertical axis of the cushion module.

In some configurations, the minor axis corresponds with a horizontal axis of the cushion module.

In some configurations, the housing comprises an inner surface exposed to therapeutic pressure, and an outer surface exposed to ambient pressure.

In some configurations, the housing comprises a support flange, the support flange being disposed at or adjacent the second end. In some configurations, the elastomeric friction member is connected to the support flange. In some configurations, the housing comprises a support wall, and the support flange extends away from the support wall. In some configurations, the support flange extends away from the second end of the housing towards the first end. In some configurations, the support flange extends away from the first end of the housing. In some configurations, the support wall and the support flange are embedded within the elastomeric friction member.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes the cushion module, the frame and headgear.

In some configurations, a cushion module for a respiratory mask, the cushion module configured for coupling to a frame configured to connect to headgear, includes a housing, a cushion, and an elastomeric friction member. The housing has a first end and a second end. The cushion is connected to the first end of the housing and configured to seal against a patient in use. The elastomeric friction member is connected to the second end of the housing and configured to form a friction coupling with the frame. The first end of the housing has a first end perimeter defining a first opening, the first opening having a first opening major dimension along a first opening major axis and a first opening minor dimension along a first opening minor axis, the first opening having a generally triangular shape when the cushion module is viewed from the rear. The second end of the housing has a second end perimeter defining a second opening, the second opening having a second opening major dimension along a second opening major axis and a second opening minor dimension along a second opening minor axis, the second opening having a generally triangular shape when the cushion module is viewed from the front. At least one of the first opening major dimension and the first opening minor dimension is greater than at least one of the second opening major dimension and the second opening minor dimension.

In some configurations, the second opening has a generally triangular shape when viewed through the first opening when the cushion module is viewed from the rear.

In some configurations, the housing separates the cushion from the elastomeric friction member.

In some configurations, the first opening major dimension is greater than the second opening major dimension.

In some configurations, the first opening minor dimension is greater than the second opening minor dimension.

In some configurations, the first opening major dimension is greater than twice the second opening major dimension.

In some configurations, the first opening major dimension is greater than the second opening major dimension, and less than twice the second opening major dimension.

In some configurations, the first opening minor dimension is greater than twice the second opening minor dimension.

In some configurations, the first opening minor dimension is greater than the second opening minor dimension, and less than twice the second opening minor dimension.

In some configurations, the first end perimeter is greater than the second end perimeter.

In some configurations, the first opening major axis and the second opening major axis and/or the first opening minor axis and the second opening minor axis are parallel.

In some configurations, the housing comprises a main wall and a support flange extending from the main wall, the support flange at least partially surrounding the second opening. In some configurations, the main wall comprises a support wall adjacent the support flange. In some configurations, the support wall and the support flange are embedded within the elastomeric friction member. In some configurations, the support wall comprises a plurality of apertures, the elastomeric friction member extending through the plurality of apertures.

In some configurations, the housing comprises a first material, and the elastomeric friction member comprises a second material, the first material being harder than the second material. In some configurations, the cushion comprises the second material.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes the cushion module, the frame and headgear.

In some configurations, a cushion module for a respiratory mask, the cushion module configured for coupling to a frame configured to connect to headgear, includes a housing, a cushion, and an elastomeric friction member. The housing has a first end and a second end. The cushion is connected to the first end of the housing and configured to seal against a patient in use. The elastomeric friction member is connected to the second end of the housing and configured to form a friction coupling with the frame. The first end of the housing has a first end perimeter defining a first opening. The second end of the housing has a second end perimeter defining a second opening, the second opening having a second opening major dimension along a second opening major axis and a second opening minor dimension along a second opening minor axis. The second opening minor dimension is offset from a midpoint of the second opening major dimension.

In some configurations, the first opening has a first opening vertical dimension along a first opening vertical axis and a first opening horizontal dimension along a first opening horizontal axis.

In some configurations, the first opening has a generally triangular shape when the cushion module is viewed from the rear.

In some configurations, the second opening minor dimension is offset below the midpoint of the second opening major dimension.

In some configurations, the second opening has a generally triangular shape when the cushion module is viewed from the front.

In some configurations, the second opening has a generally triangular shape when viewed through the first opening when the cushion module is viewed from the rear.

In some configurations, the housing separates the cushion from the elastomeric friction member.

In some configurations, the first opening vertical dimension is greater than the second opening major dimension.

In some configurations, the first opening horizontal dimension is greater than the second opening minor dimension.

In some configurations, the first opening vertical dimension is greater than twice the second opening major dimension.

In some configurations, the first opening vertical dimension is greater than the second opening major dimension, and less than twice the second opening major dimension.

In some configurations, the first opening horizontal dimension is greater than twice the second opening minor dimension.

In some configurations, the first opening horizontal dimension is greater than the second opening minor dimension, and less than twice the second opening minor dimension.

In some configurations, the first end perimeter is greater than the second end perimeter.

In some configurations, the first opening major axis and the second opening major axis and/or the first opening minor axis and the second opening minor axis are parallel.

In some configurations, the housing comprises a main wall and a support flange extending from the main wall, the support flange at least partially surrounding the second opening. In some configurations, the main wall comprises a support wall adjacent the support flange. In some configurations, the support wall and the support flange are embedded within the elastomeric friction member. In some configurations, the support wall comprises a plurality of apertures, the elastomeric friction member extending through the plurality of apertures.

In some configurations, the housing comprises a first material, and the elastomeric friction member comprises a second material, the first material being harder than the second material. In some configurations, the cushion comprises the second material.

In some configurations, the housing separates the cushion and the elastomeric friction member.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes the cushion module, the frame and headgear.

In some configurations, a cushion module for a respiratory mask, the cushion module configured to couple to a frame configured to connect to headgear, includes a housing, a cushion, and an elastomeric friction member. The housing has a first end, a second end and a main wall. The cushion has a face-contacting surface and a cushion outlet opening, the cushion being connected to the first end of the housing. The elastomeric friction member is configured to form a friction coupling with the frame, the elastomeric friction member being connected to the second end of the housing and defining a cushion module opening, the elastomeric friction member having a lower portion and an upper portion. The elastomeric friction member extends away from the main wall to define an elastomeric friction member length configured to contact the frame to form the friction coupling. The elastomeric friction member length is greater at the lower portion than at the upper portion. The elastomeric friction member is configured to couple to the frame such that a frame retention force is greater at the lower portion of the elastomeric friction member than at the upper portion of the elastomeric friction member.

In some configurations, the second end of the housing comprises a support flange extending from the main wall and surrounding the cushion module opening, the support flange being at least partially embedded within the elastomeric friction member and configured to support the elastomeric friction member. In some configurations, the support flange extends away from the main wall to define a support flange length. In some configurations, the support flange comprises an upper support flange portion corresponding to the upper portion of the elastomeric friction member, and a lower support flange portion corresponding to the lower portion of the elastomeric friction member, the support flange length being greater at the lower support flange portion than at the upper support flange portion.

In some configurations, the housing comprises a first material, and the elastomeric friction member comprises a second material, the first material being harder than the second material. In some configurations, the cushion comprises the second material.

In some configurations, the housing separates the cushion and the elastomeric friction member.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes the cushion module, the frame and headgear.

In some configurations, a cushion module for a respiratory mask, the cushion module configured to couple to a frame configured to connect to headgear, includes a housing, a cushion, and an elastomeric friction member. The housing has a first end, a second end and a main wall. The cushion has a face-contacting surface and a cushion outlet opening, the cushion being connected to the first end of the housing. The elastomeric friction member is configured to form a friction coupling with the frame, the elastomeric friction member being connected to the second end of the housing and defining a cushion module opening, the elastomeric friction member having a lower portion and an upper portion. The elastomeric friction member extends away from the main wall to define an elastomeric friction member length configured to contact the frame. The elastomeric friction member length is greater at the lower portion than at the upper portion.

In some configurations, the second end of the housing comprises a support flange extending from the main wall and surrounding the cushion module opening, the support flange being at least partially embedded within the elastomeric friction member and configured to support the elastomeric friction member. In some configurations, the support flange extends away from the main wall to define a support flange length. In some configurations, the support flange comprises an upper support flange portion corresponding to the upper portion of the elastomeric friction member, and a lower support flange portion corresponding to the lower portion of the elastomeric friction member, the support flange length being greater at the lower support flange portion than at the upper support flange portion.

In some configurations, the housing comprises a first material, and the elastomeric friction member comprises a second material, the first material being harder than the second material. In some configurations, the cushion comprises the second material.

In some configurations, the housing separates the cushion and the elastomeric friction member.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a respiratory mask includes the cushion module, the frame and headgear.

In some configurations, a respiratory mask includes a cushion module and a frame. The cushion module includes a housing, a cushion and an elastomeric friction member. The frame includes a main body configured to connect to headgear and a frame insert. The frame insert includes a vent arrangement, a vent wall and an insert wall. The insert wall at least partially surrounds a gas inlet opening. One or both of the vent wall and the insert wall form at least a portion of an insert collar. The frame insert is configured to couple to the main body. The elastomeric friction member is configured to selectively connect the cushion module to the insert collar with a friction fit.

In some configurations, the elastomeric friction member engages an outer surface of the insert collar.

In some configurations, the vent wall at least partially surrounds the vent arrangement.

In some configurations, the frame insert is configured to couple to a diffusing element such that the diffusing element diffuses gas flow through the vent arrangement.

In some configurations, each of the vent wall and the insert wall forms at least a portion of the insert collar.

In some configurations, the vent wall and the insert wall form an entirety of the insert collar.

In some configurations, the insert wall completely surrounds the gas inlet opening.

In some configurations, the insert wall comprises a recess.

In some configurations, the insert wall defines a socket configured to receive a conduit connector.

In some configurations, the vent arrangement is disposed in a projecting surface relative to a remainder of the frame insert.

In some configurations, the elastomeric friction member comprises a lip configured to contact the collar to enhance a seal between the collar and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the collar to provide feedback to a user indicating proper connection of the frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, a frame for a respiratory mask includes a main body configured to connect to headgear, a frame insert comprising a vent wall and an insert wall. The insert wall at least partially surrounds and/or defines an elbow socket. One or both of the vent wall and the insert wall form at least a portion of an insert collar. An elbow configured to engage the elbow socket such that the elbow has at least two rotational degrees of freedom with respect to the elbow socket. The frame insert further comprises a projecting surface that projects forward relative to the elbow socket. The projecting surface comprises a vent arrangement. The projecting surface is configured to restrict rotation of the elbow such that a minimum distance is maintained between the vent arrangement and the elbow.

In some configurations, contact between the elbow and the projecting surface maintains the minimum distance between the vent arrangement and the elbow.

In some configurations, the elbow engages the elbow socket such that the elbow has three rotational degrees of freedom with respect to the elbow socket.

In some configurations, a bottom edge of the projecting surface contacts the elbow when the elbow is positioned to route an associated breathing tube over a user's head.

In some configurations, the elastomeric friction member is configured to engage an outer surface of the insert collar.

In some configurations, the vent wall at least partially surrounds the vent arrangement.

In some configurations, each of the vent wall and the insert wall forms at least a portion of the insert collar.

In some configurations, the vent wall and the insert wall form an entirety of the insert collar.

In some configurations, the insert wall completely surrounds the gas inlet opening.

In some configurations, a respiratory mask frame configured to support a cushion includes a main body and a frame insert. The main body is configured to connect to headgear. The main body includes an opening and a main body flange. At least a portion of the opening is bound by the main body flange. The main body flange comprising a first sealing engagement surface. A frame insert is configured to be inserted into the opening of the main body. The frame insert comprises an insert flange. The insert flange comprises a second sealing engagement surface. The first sealing engagement surface and the second sealing engagement surface cooperate to form a cushion sealing engagement surface.

In some configurations, the frame insert comprises a vent arrangement.

In some configurations, the main body comprises a vent arrangement.

In some configurations, the frame insert comprises an elbow socket.

In some configurations, the elbow socket is a ball joint.

In some configurations, the frame insert comprises a vent arrangement and an elbow socket.

In some configurations, the insert flange at least partially defines the elbow socket.

In some configurations, the main body flange spans an apex of the opening.

In some configurations, the main body flange at least partially defines an elbow socket.

In some configurations, the frame insert is a vent insert.

In some configurations, the vent arrangement is removably connected to the frame insert.

In some configurations, the frame insert is inserted into the main body from the front of the main body.

In some configurations, the cushion is a portion of a cushion module comprising a housing and an elastomeric friction member, wherein the cushion sealing engagement surface is configured to directly engage the elastomeric friction member.

In some configurations, a respiratory mask frame configured to connect to a cushion module includes a main body, a frame insert and a vent arrangement. The main body is configured to connect to headgear. The main body comprises an opening and a main body flange. The frame insert is configured to be inserted into the opening of the main body. The frame insert comprises an insert opening and an insert flange. The insert opening defines a socket configured to receive a conduit. The main body flange at least partially surrounds the vent arrangement and comprises a cushion engagement surface configured to engage with the cushion module.

In some configurations, the main body comprises the vent arrangement.

In some configurations, the frame insert comprises the vent arrangement.

In some configurations, the main body flange entirely surrounds the vent arrangement.

In some configurations, the main body flange entirely surrounds the insert opening.

In some configurations, the frame insert is permanently connected to the main body.

In some configurations, the socket is a ball joint.

In some configurations, the insert flange at least partially defines the socket.

In some configurations, the main body flange spans an apex of the opening.

In some configurations, the vent arrangement is removably connected to the frame insert.

In some configurations, the frame insert is inserted into the main body from the front of the main body.

In some configurations, the cushion module further comprises a housing and an elastomeric friction member. The cushion sealing engagement surface is configured to directly engage the elastomeric friction member.

In some configurations, a respiratory mask frame configured to connect to a cushion module includes a main body and a frame insert. The main body is configured to connect to headgear. The main body comprises an opening and a vent arrangement. The frame insert is configured to be inserted into the opening of the main body. The frame insert comprises an insert opening and an insert flange. The insert opening defines a socket configured to receive a conduit. The insert flange at least partially defines the socket. The insert flange further comprises a cushion engagement surface configured to engage with the cushion module.

In some configurations, the frame insert is permanently connected to the main body.

In some configurations, the socket is a ball joint.

In some configurations, the frame insert is inserted into the main body from the front of the main body.

In some configurations, the frame insert is inserted into the main body from the rear of the main body.

In some configurations, the respiratory mask includes a cushion module comprising a cushion, a housing and an elastomeric friction member. The elastomeric friction member directly engages the cushion sealing engagement surface when the cushion module is connected to the respiratory mask frame.

In some configurations, the housing comprises a vent portion that cooperates with the vent arrangement of the respiratory mask frame.

In some configurations, the vent portion of the housing comprises an opening and the vent arrangement of the respiratory mask frame comprises a plurality of vent openings.

In some configurations, the vent arrangement of the respiratory mask frame further comprises a vent flange surrounding the plurality of openings and that engages the opening of the vent portion of the housing when the cushion module is connected to the respiratory mask frame.

In some configurations, the vent portion of the housing comprises a vent flange surrounding the opening, wherein the vent flange contacts a surface of the respiratory mask frame and surrounds the plurality of openings.

In some configurations, the vent flange and the elastomeric friction member are formed as an integrated or unitary structure.

In some configurations, the vent portion of the housing comprises a plurality of vent openings and the vent arrangement of the respiratory mask frame comprises an opening that is aligned with the plurality of vent openings of the housing.

In some configurations, the insert flange surrounds the socket and the vent arrangement.

In some configurations, a respiratory mask frame configured to connect to a cushion module includes a main body comprising one or more headgear mounts configured to connect to headgear. The respiratory mask frame includes a gas inlet opening and an elastomeric friction member configured to engage the cushion module.

In some configurations, a rim surrounds the gas inlet opening, wherein the rim is embedded in the elastomeric friction member.

In some configurations, a support flange is embedded within the elastomeric friction member, a free end of the support flange defining the rim.

In some configurations, the elastomeric friction member is configured to engage an outer surface of a collar of the cushion module.

In some configurations, an outer surface of the elastomeric friction member is exposed when the respiratory mask frame is connected to the cushion module.

In some configurations, the friction member is configured to engage an inner surface of a collar of the cushion module.

In some configurations, an inner surface of the elastomeric friction member is exposed when the respiratory mask frame is connected to the cushion module.

In some configurations, the elastomeric friction member encircles a vent on the respiratory mask frame.

In some configurations, the elastomeric friction member extends rearwardly from the main body toward the cushion module.

In some configurations, the main body has a support wall extending from the support flange. At least a portion of the support wall is embedded within the elastomeric friction member. A curved transition region is between the support wall and the support flange.

In some configurations, the support flange surrounds the gas inlet opening and a vent on the respiratory mask frame, wherein the elastomeric friction member covers an entirety of the support flange along a top portion adjacent the vent, and wherein the elastomeric friction member is located only on an outer surface of the support flange on a bottom portion adjacent the gas inlet opening.

In some configurations, the bottom portion of the support flange at least partially defines a socket configured to receive a conduit.

In some configurations, a vent arrangement is formed as a unitary or integral structure with the elastomeric friction member.

In some configurations, the respiratory mask includes a cushion module comprising a cushion and a housing, the housing comprising a collar, wherein the collar engages the elastomeric friction member when the cushion module is connected to the respiratory mask frame.

In some configurations, the collar extends rearwardly from an adjacent portion of the housing.

In some configurations, the cushion is configured to surround a nose and a mouth of a user.

In some configurations, the elastomeric friction member is located only on an outside of the support flange.

In some configurations, the support flange defines a socket configured to receive a conduit.

In some configurations, the respiratory mask includes a cushion module comprising a cushion and a housing. The housing is configured to engage the elastomeric friction member when the cushion module is connected to the respiratory mask frame.

In some configurations, the housing further comprises a collar that engages the elastomeric friction member when the cushion module is connected to the respiratory mask frame.

In some configurations, the collar extends rearwardly from an adjacent portion of the housing.

In some configurations, the cushion is configured to surround a nose and a mouth of a user.

In some configurations, the housing includes a recess surrounding the collar.

In some configurations, the elastomeric friction member engages an outside surface of the collar and an outer surface of the elastomeric friction member is exposed within the recess.

In some configurations, the cushion is configured to surround a nose and a mouth of a user.

In some configurations, the cushion is configured to engage an underside of the nose of the user.

In some configurations, the respiratory mask includes a cushion module comprising a cushion and a housing, the cushion configured to surround the nares of user.

In some configurations, the elastomeric friction member comprises at least one protrusion configured to engage the respiratory mask frame to inhibit relative rotational movement between the elastomeric friction member and the respiratory mask frame.

In some configurations, the elastomeric friction member extends in a forward direction from the main body.

In some configurations, the elastomeric friction member extends in a rearward direction from the main body.

In some configurations, the elastomeric friction member comprises a lip configured to contact a cooperating portion of the cushion module to enhance a seal between the cushion module and the elastomeric friction member.

In some configurations, the lip is oriented at an oblique rearward angle relative to a longitudinal axis of the elastomeric friction member.

In some configurations, the elastomeric friction member comprises a projection configured to engage a cooperating recess in the cushion module to provide feedback to a user of proper connection of the respiratory mask frame and the cushion module.

In some configurations, the projection is a discrete structure extending around only a portion of a closed loop of the elastomeric friction member.

In some configurations, the projection extends around an entirety of a closed loop of the elastomeric friction member.

In some configurations, the projection is located on a rearward portion of the elastomeric friction member relative to a direction of assembly of the respiratory mask frame and the cushion module.

In some configurations, the respiratory mask includes a cushion module having a support flange configured to engage the elastomeric friction member.

In some configurations, the support flange extends in a forward direction away from a breathing chamber of the cushion module.

In some configurations, the support flange extends in a rearward direction toward or into a breathing chamber of the cushion module.

### BRIEF DESCRIPTION OF THE DRAWINGS

Throughout the drawings, reference numbers can be reused to indicate general correspondence between reference elements. The drawings are provided to illustrate example embodiments described herein and are not intended to limit the scope of the disclosure.

FIG. 1 is a side view of a patient interface comprising a frame and a cushion module shown separated from one another.

FIG. 2 is a side view of the patient interface of FIG. 1 with the frame and the cushion module assembled.

FIG. 3 is an enlarged view of a portion of the patient interface of FIG. 1 showing a friction coupling of the interface with the frame and the cushion module separated from one another and illustrating several length dimensions.

FIG. 4 is an enlarged view of the portion of the patient interface of FIG. 3 illustrating several thickness dimensions.

FIG. 5 is an enlarged of the portion of the patient interface of FIG. 3 with the frame and the cushion module assembled and illustrating several length dimensions.

FIG. 6 illustrates an alternative friction coupling in which a relative angle is illustrated between a portion of the frame and a portion of the cushion module.

FIG. 7 is an enlarged view of the friction coupling illustrating a retention force at several axial locations.

FIG. 8 is a side view of another patient interface comprising a frame and a cushion module shown separated from one another.

FIG. 9 is a side view of the patient interface of FIG. 8 with the frame and the cushion module assembled.

FIG. 10 is an enlarged view of a portion of the patient interface of FIG. 8 showing a friction coupling of the interface with the frame and the cushion module separated from one another and illustrating several length dimensions.

FIG. 11 is an enlarged view of the portion of the patient interface of FIG. 10 illustrating several thickness dimensions.

FIG. 12 is an enlarged of the portion of the patient interface of FIG. 10 with the frame and the cushion module assembled and illustrating several length dimensions.

FIG. 13 is a partial view of the friction coupling of the interface of FIGS. 1-7 with mold shut-off portions illustrated.

FIG. 14 is a partial view of the friction coupling of the interface of FIGS. 8-12 with mold shut-off portions illustrated.

FIG. 15 is a front perspective view of a patient interface comprising a frame and a cushion module coupled to one another by a friction coupling.

FIG. 16 is a front perspective view of the cushion module of the patient interface of FIG. 1 separate from the frame.

FIG. 17 is a central, vertical cross-sectional view of the cushion module of FIG. 16.

FIG. 18 is a rear perspective view of the frame of the patient interface of FIG. 1.

FIG. 19 is a central, vertical cross-sectional view of the patient interface of FIG. 1.

FIG. 20 is an enlarged view of a portion of the cross-sectional view of FIG. 19.

FIG. 21 is a front perspective view of a patient interface including a frame, a cushion module and a conduit connector elbow, wherein the cushion module is removably coupled to the frame by a friction coupling.

FIG. 22 is a front perspective view of the cushion module of FIG. 21 separate from the frame and conduit connector elbow.

FIG. 23 is a front view of the cushion module of FIG. 22 with a friction coupling portion of the cushion module removed to expose an underlying support structure.

FIG. 24 is a side view of the cushion module of FIG. 23.

FIG. 25 is a front view of the cushion module of FIG. 22.

FIG. 26 is a side view of the cushion module of FIG. 22.

FIG. 27 is a sectional view of the cushion module of FIG. 22 taken along a vertical, central plane.

FIG. 28 is a top view of the cushion module of FIG. 22.

FIG. 29 is a bottom view of the cushion module of FIG. 22.

FIG. 30 is an enlarged view of a portion of the cushion module indicated by the perimeter 30 in FIG. 27 illustrating the friction coupling portion.

FIG. 31 is an enlarged view of a portion of the cushion module indicated by the perimeter 31 in FIG. 30 illustrating an upper portion of the friction coupling portion.

FIG. 32 is an enlarged view of a portion of the cushion module indicated by the perimeter 32 in FIG. 30 illustrating a lower portion of the friction coupling portion.

FIG. 33 is a front perspective view of the frame of the patient interface of FIG. 21 separate from the cushion module and the conduit connector elbow.

FIG. 34 is a front view of the frame of FIG. 33.

FIG. 35 is a sectional view of the frame of FIG. 33 taken along the line 35-35 in FIG. 34.

FIG. 36 is an enlarged view of a portion of the frame indicated by the perimeter 36 in FIG. 35 illustrating a collar of the frame.

FIG. 37 is a side view of the frame of FIG. 33.

FIG. 38 is a rear view of the frame of FIG. 33.

FIG. 39 is a rear perspective view of the frame of FIG. 33.

FIG. 40 is a sectional view of the frame similar to FIG. 35 but including the conduit connector elbow.

FIG. 41 is a front view of the patient interface of FIG. 21.

FIG. 42 is a sectional view of the patient interface taken along the line 42-42 in FIG. 41.

FIG. 43 is an enlarged view of a portion of the patient interface indicated by the perimeter 43 in FIG. 42 illustrating an upper portion of the friction coupling.

FIG. 44 is an enlarged view of a portion of the patient interface indicated by the perimeter 44 in FIG. 43 illustrating the friction coupling.

FIG. 45 is an enlarged view of a portion of the patient interface indicated by the perimeter 45 in FIG. 42 illustrating a lower portion of the friction coupling.

FIG. 46 is a front perspective view of a patient interface including a cushion module and a frame having an integrated a conduit connector, wherein the cushion module is removably coupled to the frame by a friction coupling.

FIG. 47 is a front perspective view of the cushion module separate from the frame.

FIG. 48 is a front view of the cushion module of FIG. 47.

FIG. 49 is a side view of the cushion module of FIG. 47.

FIG. 50 is a front view of the cushion module of FIG. 47 with the friction coupling portion of the cushion module removed to expose underlying support structure.

FIG. 51 is a front perspective view of the cushion module of FIG. 50.

FIG. 52 is a sectional view of the cushion module of FIG. 47 taken along a vertical, central plane passing through the cushion module in a forward-rearward direction.

FIG. 53 is a top view of the cushion module of FIG. 47.

FIG. 54 is a bottom view of the cushion module of FIG. 47.

FIG. 55 is a rear view of the cushion module of FIG. 47.

FIG. 56 is a front view of the cushion module of FIG. 47, wherein the viewing axis is aligned with an axis of an inlet opening and/or the friction coupling portion of the cushion module.

FIG. 57 is an enlarged view of the cushion module indicated by the perimeter 57 in FIG. 52 illustrating an upper portion of the friction coupling portion.

FIG. 58 is an enlarged view of the cushion module indicated by the perimeter 58 in FIG. 52 illustrating a lower portion of the friction coupling portion.

FIG. 59 is a front perspective of the frame separate from the cushion module.

FIG. 60 is a front view of the frame of FIG. 59.

FIG. 61 is a side view of the frame of FIG. 59.

FIG. 62 is a sectional view of the frame of FIG. 59 taken along a vertical, central plane passing through the frame in a forward-rearward direction.

FIG. 63 is a rear view of the frame of FIG. 59.

FIG. 64 is a front view of the patient interface of FIG. 46.

FIG. 65 is a side view of the patient interface of FIG. 46.

FIG. 66 is a rear view of the patient interface of FIG. 46.

FIG. 67 is a sectional view of the patient interface of FIG. 46 taken along a vertical, central plane passing through the patient interface in a forward-rearward direction.

FIG. 68 is an enlarged view of a portion of the patient interface indicated by the perimeter 68 in FIG. 67 illustrating an upper portion of the friction coupling portion.

FIG. 69 is an enlarged view of a portion of the patient interface indicated by the perimeter 69 in FIG. 67 illustrating a lower portion of the friction coupling portion.

FIG. 70 is a schematic representation of a respiratory system configured to supply pressurized and humidified breathing gases to a user through a patient interface.

FIG. 71 is a front perspective view of a patient interface comprising a frame and a cushion module coupled to one another by a friction coupling, with an elbow coupled to the interface.

FIG. 72 is a front perspective view of the cushion module of FIG. 71.

FIG. 73 is a front perspective view of the cushion module of FIG. 72 with a friction member deleted to show underlying structure.

FIG. 74 is a front view of the cushion module of FIG. 73.

FIG. 75A is a side view of the cushion module of FIG. 73.

FIG. 75B is a side view of the cushion module of FIG. 73, highlighting certain dimensions.

FIG. 76 is a front view of the cushion module of FIG. 72.

FIG. 77 is a side view of the cushion module of FIG. 72.

FIG. 78 is a section view of the cushion module of FIG. 72, taken along line 78 in FIG. 76.

FIG. 79 is a top view of the cushion module of FIG. 72.

FIG. 80 is a bottom view of the cushion module of FIG. 72.

FIG. 81 is a magnified section view of the region identified by box 81 in

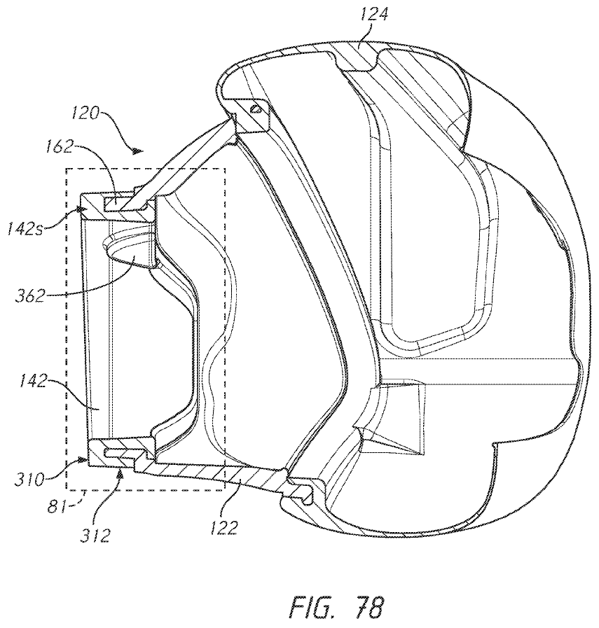

FIG. 78.

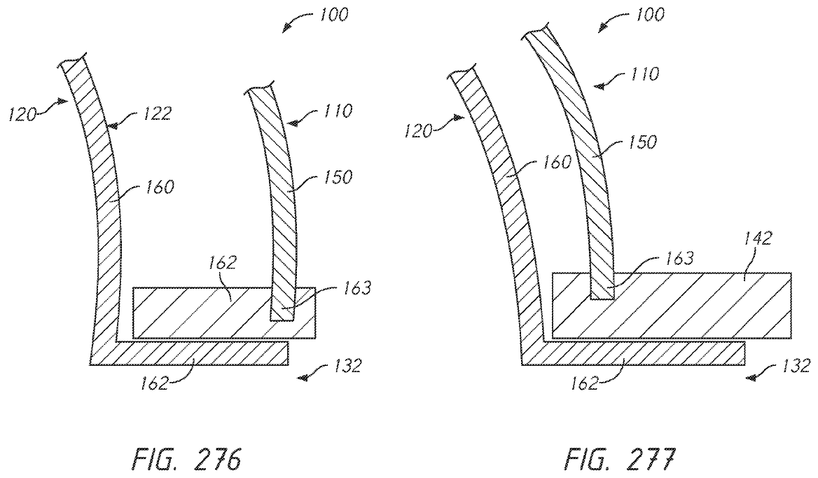

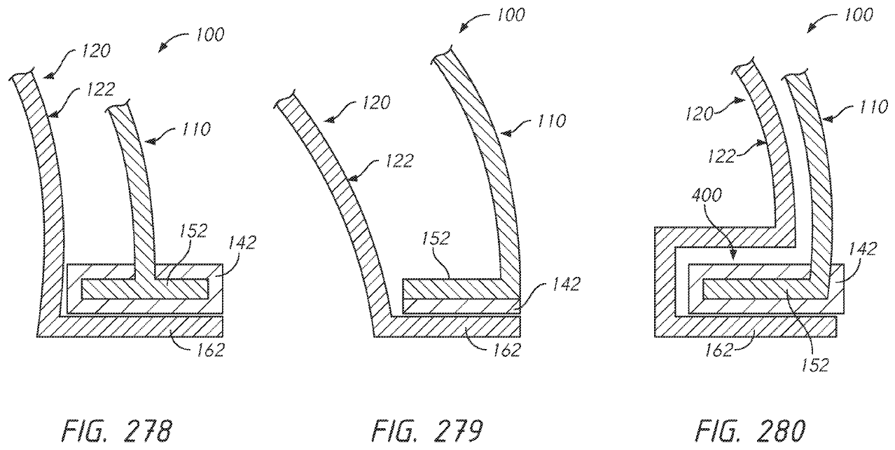

FIG. 82 is a magnified section view of the region identified by box 82 in FIG. 81.

FIG. 83 is a front view of the cushion module of FIG. 72.

FIG. 84 is a section view of the cushion module of FIG. 72, taken along line 84 in FIG. 83.

FIG. 85 is a magnified section view of the region identified by box 85 in FIG. 84.

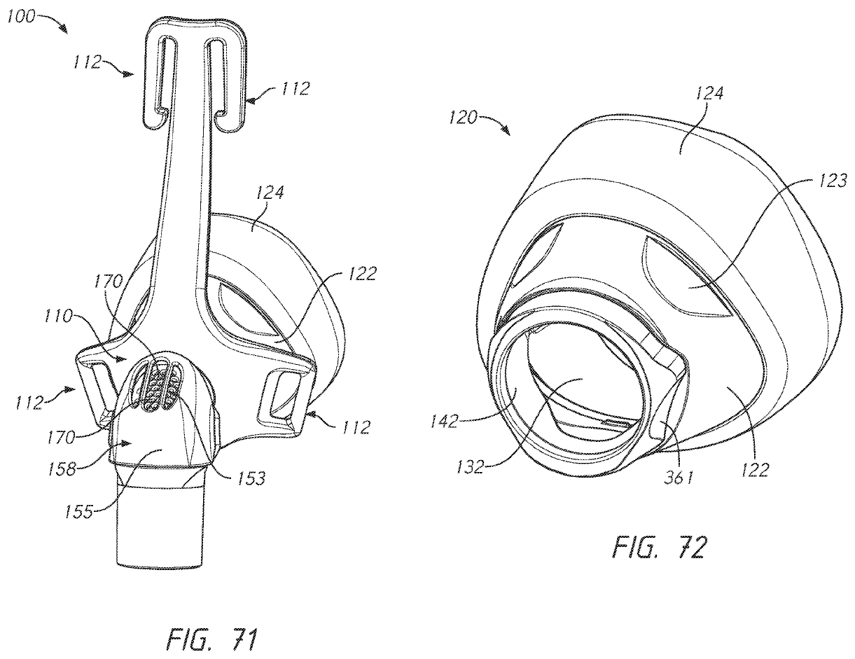

FIG. 86 is a front perspective view of the frame of FIG. 71.

FIG. 87 is a front view of the frame of FIG. 86.

FIG. 88 is a side view of the frame of FIG. 86.

FIG. 89 is a rear view of the frame of FIG. 86.

FIG. 90 is a section view of the frame of FIG. 86, taken along line 90 in FIG. 87.

FIG. 91 is a magnified section view of the region identified by box 91 in FIG. 90.

FIG. 92 is a rear perspective view of the frame of FIG. 71.

FIG. 93 is a section view of the elbow and frame of FIG. 71.

FIG. 94 is a front view of the assembly of FIG. 71.

FIG. 95 is a section view of the assembly of FIG. 71, taken along line 95 in FIG. 94.

FIG. 96 is a magnified section view of the region indicated by box 96 in FIG. 95 with the elbow removed.

FIG. 97 is a magnified section view of the region indicated by box 97 in FIG. 95 with the elbow removed.

FIG. 98 is a front view of the assembly of FIG. 71.

FIG. 99 is a section view of the assembly of FIG. 71, taken along line 99 in FIG. 98.

FIG. 100 is a magnified section view of the region indicated by box 100 in FIG. 99.

FIG. 101 is a front perspective view of a patient interface comprising a frame and a cushion module coupled to one another by a friction coupling, with an elbow coupled to the interface.

FIG. 102 is a front perspective view of the cushion module of FIG. 101 with a friction member removed to show underlying structure.

FIG. 103 is a front view of the cushion module of FIG. 102.

FIG. 104 is a side view of the cushion module of FIG. 102.

FIG. 105 is a front perspective view of the cushion module of FIG. 101.

FIG. 106 is a front view of the cushion module of FIG. 101.

FIG. 107 is a side view of the cushion module of FIG. 101.

FIG. 108 is a section view of the cushion module taken along line 108 in

FIG. 106.

FIG. 109 is a top view of the cushion module of FIG. 101.

FIG. 110 is a bottom view of the cushion module of FIG. 101.

FIG. 111 is a magnified section view of the region indicated by box 111 in FIG. 108.

FIG. 112 is a magnified section view of the region indicated by box 112 in FIG. 108.

FIG. 113 is the magnified section view of FIG. 112, indicating certain dimensions.

FIG. 114 is the magnified section view of FIG. 112, indicating certain dimensions.

FIG. 115 is a front perspective view of the frame of FIG. 101.

FIG. 116 is a front view of the frame of FIG. 115.

FIG. 117 is a side view of the frame of FIG. 115.

FIG. 118 is a rear view of the frame of FIG. 115.

FIG. 119 is a section view of the frame of FIG. 115, taken along line 119 in FIG. 116.

FIG. 120 is a magnified section view of the region indicated by box 120 in FIG. 119.

FIG. 121 is a front perspective view of a frame insert of the frame of FIG. 115.

FIG. 122 is a front view of the frame insert of FIG. 121.

FIG. 123 is a top view of the frame insert of FIG. 121.

FIG. 124 is a bottom view of the frame insert of FIG. 121.

FIG. 125 is a side view of the frame insert of FIG. 121.

FIG. 126 is a rear view of the frame insert of FIG. 121.

FIG. 127 is a rear perspective view of the frame insert of FIG. 121.

FIG. 128 is a section view of the frame insert of FIG. 121, taken along line 128 in FIG. 122.

FIG. 129 is a front perspective view of the frame of the frame insert of FIG. 121 assembled with the frame of FIG. 115.

FIG. 130 is a rear perspective view of the assembled frame of FIG. 129.

FIG. 131 is a front perspective view of the assembly of FIG. 101.

FIG. 132 is a front view of the assembly of FIG. 131 with the elbow removed.

FIG. 133 is a section view of the assembly of FIG. 131, taken along line 133 in FIG. 132.

FIG. 134 is a magnified section view of the region indicated by box 134 in FIG. 133.

FIG. 135 is a magnified section view of the region indicated by box 135 in FIG. 133.

FIG. 136 is a front view of the assembly of FIG. 132.

FIG. 137 is a section view of the assembly of FIG. 132, taken along line 137 in FIG. 136.

FIG. 138 is a magnified section view of the region indicated by box 138 in FIG. 137.

FIG. 139 is a front perspective view of a patient interface comprising a frame and a cushion module coupled to one another by a friction coupling, with an elbow coupled to the interface.

FIG. 140 is a perspective view of the cushion module of FIG. 139.

FIG. 141 is a front view of the cushion module of FIG. 140.

FIG. 142 is a side view of the cushion module of FIG. 140.

FIG. 143 is a section view of the cushion module of FIG. 140, taken along line 143 in FIG. 141.

FIG. 144 is a front perspective view of the cushion module of FIG. 140, with a friction member removed to show underlying structure.

FIG. 145 is a front view of the cushion module of FIG. 144.

FIG. 146 is a section view of the cushion module of FIG. 144, taken along line 143 in FIG. 141.

FIG. 147 is a top view of the cushion module of FIG. 140.

FIG. 148 is a bottom view of the cushion module of FIG. 140.

FIG. 149 is a front view of the cushion module of FIG. 140 showing certain dimensions.

FIG. 150 is a front view of the cushion module of FIG. 140 showing certain dimensions.

FIG. 151 is a magnified section view of the region indicated by box 151 in FIG. 143.

FIG. 152 is a magnified section view of the region indicated by box 152 in FIG. 143.

FIG. 153 is a front perspective view of the frame of FIG. 139 including a diffuser.

FIG. 154 is a front perspective view of the frame of FIG. 153 with the diffuser removed.

FIG. 155 is a front view of the frame of FIG. 153.

FIG. 156 is a rear perspective view of the frame of FIG. 153.

FIG. 157 is a bottom section view of the frame of FIG. 153, taken along line 157 in FIG. 155.

FIG. 158 is a side view of the frame of FIG. 153.

FIG. 159 is a section view of the frame of FIG. 153, taken along line 153 in FIG. 155.

FIG. 160 is a rear view of the frame of FIG. 153.

FIG. 161 is a front view of the patient interface of FIG. 139.

FIG. 162 is a side view of the patient interface of FIG. 139.

FIG. 163 is a section view of the patient interface of FIG. 139, taken along line 163 in FIG. 161.

FIG. 164 is a magnified section view of the region indicated by box 164 in FIG. 163.

FIG. 165 is a magnified section view of the region indicated by box 165 in FIG. 163.

FIG. 166 is a front view of a variation of the cushion module of FIG. 140.

FIG. 167 is a section view showing coupling of the frame of FIG. 153 to the cushion module of FIG. 140.

FIG. 168 is a front perspective view of a patient interface comprising a frame and a cushion module coupled to one another by a friction coupling, with an elbow coupled to the interface.

FIG. 169 is a perspective view of the cushion module of FIG. 168, with a friction member removed to show underlying structure.

FIG. 170 is a front view of the cushion module of FIG. 169.

FIG. 171 is a side view of the cushion module of FIG. 169.

FIG. 172 is a perspective view of the cushion module of FIG. 168.

FIG. 173 is a front view of the cushion module of FIG. 168.

FIG. 174 is a side view of the cushion module of FIG. 168.

FIG. 175 is a section view of the cushion module of FIG. 168, taken along line 175 in FIG. 173.

FIG. 176 is a top view of the cushion module of FIG. 168.

FIG. 177 is a bottom view of the cushion module of FIG. 168.

FIG. 178 is a front view of the cushion module of FIG. 168, showing certain dimensions.

FIG. 179 is a side view of the cushion module of FIG. 168, showing certain dimensions.

FIG. 180 is a front view of the cushion module of FIG. 168, showing certain dimensions.

FIG. 181 is a side view of the cushion module of FIG. 168, showing certain dimensions.

FIG. 182 is a magnified section view of the region indicated by box 182 in FIG. 175.

FIG. 183 is a schematic view of the housing of the cushion module of FIG. 168 positioned in a mold tool.

FIG. 184 is a magnified view of the housing and mold tool of FIG. 183.

FIG. 185 is a magnified section view of the region indicated by box 182 in FIG. 175, showing certain dimensions.

FIG. 186 is a magnified section view of the region indicated by box 182 in FIG. 175, showing certain dimensions.

FIG. 187 is a front perspective view of the frame of FIG. 168.

FIG. 188 is a front view of the frame of FIG. 187.

FIG. 189 is a section view of the frame of FIG. 187 taken along line 189 in FIG. 188.

FIG. 190 is a magnified section view of the region indicated by box 190 in FIG. 189.

FIG. 191 is a rear view of the frame of FIG. 187.

FIG. 192 is a front perspective view of the frame insert of FIG. 168.

FIG. 193 is a front view of the frame insert of FIG. 192.

FIG. 194 is a top view of the frame insert of FIG. 192.

FIG. 195 is a bottom view of the frame insert of FIG. 192.

FIG. 196 is a side view of the frame insert of FIG. 192.

FIG. 197 is a rear view of the frame insert of FIG. 192.

FIG. 198 is a rear perspective view of the frame insert of FIG. 192.

FIG. 199 is a section view of the frame insert of FIG. 192, taken along line 199 in FIG. 193.

FIG. 200 is a section view of the frame insert and elbow of FIG. 168.

FIG. 201 is a partial section view of the frame insert and elbow of FIG. 168 showing two positions of the elbow.

FIG. 202 is an exploded perspective view showing assembly of a variation of the frame insert of FIG. 192 with the frame of FIG. 187.

FIG. 203 is a partial section view of the frame insert of FIG. 202 and elbow of FIG. 168 showing two positions of the elbow.

FIG. 204 is a perspective view of a variation of the frame insert.

FIG. 205 is a front perspective view of the frame insert of FIG. 192 coupled with the frame of FIG. 187.

FIG. 206 is a rear perspective view of the frame insert and frame assembly of FIG. 205.

FIG. 207 is a front perspective view of the patient interface of FIG. 168.

FIG. 208 is a front view of the patient interface of FIG. 207 with the elbow hidden.

FIG. 209 is a section view of the patient interface of FIG. 208, taken along line 209 in FIG. 208.

FIG. 210 is a magnified section view of the region indicated by box 210 in FIG. 209.

FIG. 211 is a magnified section view of the region indicated by box 211 in FIG. 209.

FIG. 212 is a magnified section view of the region indicated by box 212 in FIG. 209.

FIG. 213 is a magnified section view of the region indicated by box 213 in FIG. 209.

FIG. 214 is a magnified section view of the region indicated by box 214 in FIG. 213.

FIG. 215 is a front view of a variation of the cushion module with the seal removed.

FIG. 216 is a side view of the cushion module of FIG. 215.

FIG. 217 is a front view of the cushion module of FIG. 215 showing certain dimensions.

FIG. 218 is a front view of the cushion module of FIG. 215 showing certain dimensions.

FIG. 219 is a magnified section view of the region of FIG. 185 for the cushion module of FIG. 215.

FIG. 220 shows the region of FIG. 219 showing certain dimensions.

FIG. 221 shows the region of FIG. 211 for the cushion module of FIG. 215.

FIG. 222 is a section view of a portion of the cushion module of FIG. 215 taken at a bottom of the friction member.

FIG. 223 shows the region of FIG. 223 showing certain dimensions.

FIG. 224 shows the region of FIG. 223 showing certain dimensions.

FIG. 225 shows the region of FIG. 213 for the cushion module of FIG. 215.

FIG. 226 is a side view of the cushion module of FIG. 215 during coupling of the frame insert.

FIG. 227 is a front view of a variation of the cushion module of FIG. 215.

FIG. 228 is a perspective view of the patient interface of FIG. 168 with certain components including a color indicator.

FIG. 229 is an exploded front perspective view of the patient interface of FIG. 228.

FIG. 230 is an exploded rear perspective view of the patient interface of FIG. 228.

FIG. 231 is a front perspective view of a variation of the frame insert of FIG. 192 coupled to the frame of FIG. 187 and including a diffuser.

FIG. 232 is a front perspective view of the frame insert and frame of FIG. 231 with the diffuser removed.

FIG. 233 is a front perspective view of the frame insert of FIG. 231.

FIG. 234 is a front view of the frame insert of FIG. 233.

FIG. 235 is a top view of the frame insert of FIG. 233.

FIG. 236 is a bottom view of the frame insert of FIG. 233.

FIG. 237 is a left view of the frame insert of FIG. 233.

FIG. 238 is a rear view of the frame insert of FIG. 233.

FIG. 239 is a rear perspective view of the frame insert of FIG. 233.

FIG. 240 is a section view of the frame insert of FIG. 233 taken along line 240 in FIG. 234.

FIG. 241 is the section view of FIG. 240 including the diffuser.

FIG. 242 is a magnified section view of the region indicated by box 242 in FIG. 241.

FIG. 243 is a front perspective view of the diffuser of FIG. 231.

FIG. 244 is a front view of the diffuser of FIG. 243.

FIG. 245 is a top view of the diffuser of FIG. 243.

FIG. 246 is a bottom view of the diffuser of FIG. 243.

FIG. 247 is a side view of the diffuser of FIG. 243.

FIG. 248 is a rear view of the diffuser of FIG. 243.

FIG. 249 is a front view of the assembly of FIG. 231.

FIG. 250 is a section view taken along line 250 in FIG. 249.

FIG. 251 is a schematic section view of a variation of the housing, frame, and frame insert.

FIG. 252 is a schematic section view of a variation of the housing, frame, and frame insert.

FIG. 253 is a schematic section view of a variation of the housing, frame, and frame insert.

FIG. 254 is a schematic section view of a variation of the housing, frame, and frame insert.

FIG. 255 is a schematic section view of a variation of the housing, frame, and frame insert.

FIG. 256 is a schematic section view of a variation of the housing, frame, and frame insert.

FIG. 257 is a schematic section view of a variation of the housing, frame, and frame insert.

FIG. 258 is a schematic section view of a variation of the housing, frame, and frame insert.

FIG. 259 is a schematic section view of a variation of the housing, frame, and frame insert.

FIG. 260 is a schematic section view of a variation of the housing, frame, and frame insert.

FIG. 261 is a schematic section view of a variation of the housing, frame, and frame insert.

FIG. 262 is a schematic section view of a variation of the housing, frame, and frame insert.

FIG. 263 is a schematic section view of a variation of the housing, frame, and frame insert.

FIG. 264 is a schematic section view of a variation of the housing, frame, and frame insert.

FIG. 265 is a schematic section view of a variation of the housing, frame, and frame insert.

FIG. 266 is a schematic section view of a variation of the housing, frame, and frame insert.

FIG. 267 is a schematic section view of a variation of the housing, frame, and frame insert.

FIG. 268 is a section view of a portion of a patient interface having a frame and a cushion module, and a user feedback arrangement configured to provide an indication of connection to a user.

FIG. 269 is a section view of a portion of a modification of the patient interface of FIG. 268.

FIG. 270 is a section view of a portion of a patient interface having a frame and a cushion module, wherein an elastomeric friction member comprises a seal enhancement feature.

FIG. 271 is a section view of a portion of a modification of the patient interface of FIG. 270.

FIG. 272 is a section view of a portion of a patient interface having a frame and a cushion module, wherein an elastomeric friction member is carried by the frame and contacts an outer surface of a collar of the cushion module.

FIG. 273 is a section view of a portion of a modification of the patient interface of FIG. 272.

FIG. 274 is a section view of a portion of a patient interface having a frame and a cushion module, wherein an elastomeric friction member is carried by the frame and contacts an inner surface of a collar of the cushion module.

FIG. 275 is a section view of a portion of a modification of the patient interface of FIG. 274.

FIG. 276 is a section view of a portion of a patient interface having a frame and a cushion module, wherein an elastomeric friction member is carried by and extends in a rearward direction from the frame and has a substantial unsupported length.

FIG. 277 is a section view of a portion of a modification of the patient interface of FIG. 276 in which the elastomeric friction member extends in a forward direction from the frame.

FIG. 278 is a section view of a portion of a patient interface having a frame and a cushion module, wherein an elastomeric friction member is carried by and extends in both a forward and rearward direction from the frame.

FIG. 279 is a section view of a portion of a patient interface having a frame and a cushion module, wherein an elastomeric friction member is provided on only one surface of a support flange of the frame.

FIG. 280 is a section view of a portion of a patient interface having a frame and a cushion module, wherein an elastomeric friction member is carried by the frame and is received within a recess of the cushion module.

### DETAILED DESCRIPTION

Embodiments of systems, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. In addition, the orientation and/or location of the assembly or portions thereof (e.g., components or elements) can be described with reference to the planes of the human body, in which the sagittal plane or median plane (longitudinal, anteroposterior) divides the body into left and right, the coronal plane or frontal plane (vertical) divides the body into dorsal and ventral (back and front, posterior and anterior) portions, and the transverse plane or axial plane (lateral, horizontal) divides the body into cranial and caudal (head and tail, upper and lower) portions. Such references to these planes assume the assembly is properly positioned for the intended use on a user. Moreover, terms such as “first,” “second,” “third,” and so on may be used to describe separate components. Such terminology may include the words specifically mentioned above, derivatives thereof, and words of similar import.

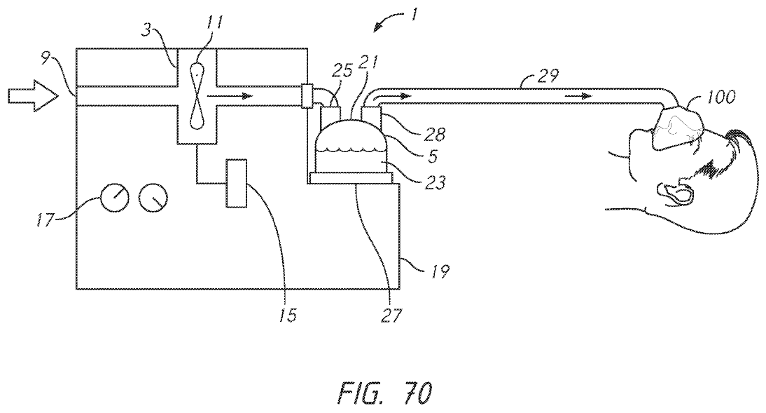

An example respiratory therapy system suitable for supplying breathing gases to a user for positive airway pressure (PAP) therapy (e.g., continuous positive airway pressure (CPAP) therapy) or non-invasive ventilation (NIV) therapy is illustrated in FIG. 70. The example respiratory therapy system 1 may include a gas source 3, a humidifier 5, a patient interface 100 and a breathing gas circuit 29 that connects the humidifier (or gas source) to the patient interface 100. The gas source 3 can provide a supply of breathing gas to the humidifier 5. The gas source 3 may comprise a blower in which breathing gas, e.g., ambient air, is drawn into the gas source 3 through an inlet 9 in the gas source casing by an impeller 11. The rotational speed of the impeller 11 may be modulated to regulate the quantity of air drawn into the gas source 3 and the supply of breathing gas delivered to the respiratory therapy system 1.

Breathing gas may include any single gas or multiple gases that are breathable by a user of the system 1.

The pressure and/or flow rate of breathing gas exiting the gas source 3 may be regulated by a controller 15. The controller 15 may modulate the rotational speed of the impeller 11 according to one or more predetermined algorithms and in accordance with one or more user inputs that may be provided via a user input 17.

The gas source 3 represents an actively controlled flow generator. Other gas sources, such as a compressed air cylinder with suitable pressure or flow regulation, may also be used to supply breathing gas. The outlet of the gas source 3 may be coupled to a separate humidifier 5. The humidifier 5 may be configured to heat and/or humidify the breathing gas prior to delivery, e.g., delivery to the user. In some embodiments, the humidifier is integrated with the gas supply. The humidifier 5 may include a base 19 and a humidifier chamber 21. The chamber 21 may be configured to hold humidification fluid 23, such as water, and may be disengaged, e.g., temporarily disengaged or permanently disengaged, from the humidifier base 19 to allow it to be filled or replaced. The humidifier 5 receives gases from the gas source 3 through chamber inlet 25. The humidifier base 19 can include a heater such as a heater plate 27. The chamber 21 rests on the heater plate 27 when engaged with the humidifier base 19. The heater plate 27 dissipates heat, e.g., heat generated by electrical resistance, to the chamber 21. The chamber 21 preferably has a heat conductive base to enable the heat generated by the heater plate 27 to pass efficiently to the humidification fluid 23. Controller 15 can also control the humidifier 5, and in particular the supply of electrical energy to the heater plate 27, to regulate any function of the humidifier 5, e.g., the temperature and humidity of the breathing gas supplied to the user.

The breathing gas can be supplied to the user via a chamber outlet 28 and the breathing gas circuit 29 in the form of a conduit which may incorporate a heating or warming element, e.g., a heater wire, to heat or warm (e.g., keep hot or warm) the breathing gases during transportation to the patient interface 7. The electrical energy supplied to the heater wire may be controlled by the controller 15. The controller 15 may receive feedback from one or more sensors incorporated in a control network throughout the respiratory therapy system to monitor properties of the breathing gas, such as, but not limited to, pressure, flow, temperature, and/or humidity.

The patient interface 100 couples the user with the respiratory therapy system 1, such that gases, e.g., heated and humidified gases from the humidifier 5, may be delivered to the user's respiratory system. Breathing gases can be delivered to the user at, or near, optimal temperature and humidity (e.g., warmed and fully saturated with water vapor at temperatures of between 27 and 37° C.) as the gases are delivered to the user's nares and or mouth. Emulating the conditions within healthy adult lungs (37° C., 44 mg/L humidity) can help maintain healthy mucocilliary function in users with respiratory disorders affecting secretion and for all patients humidifying the gas helps maintain comfort and compliance. A number of different styles of patient interface 100, such as those disclosed herein, may be used in the example system 1 or a similar system.

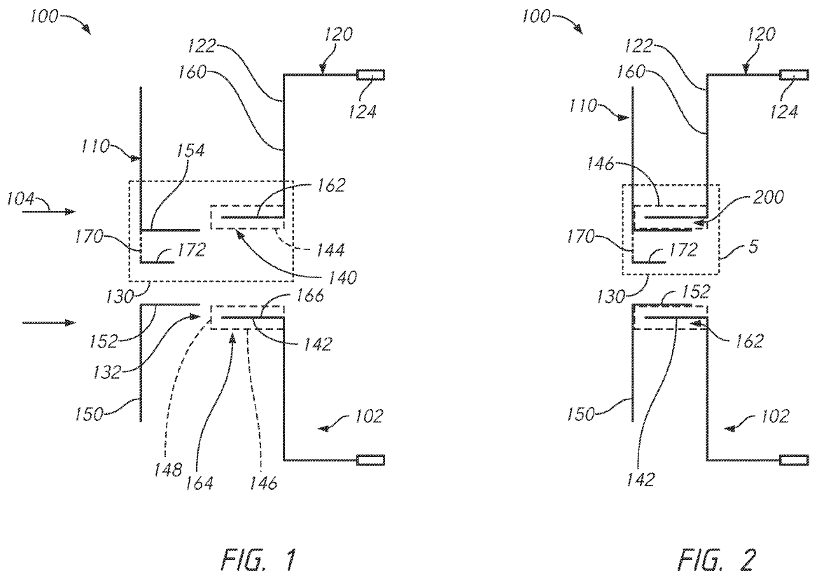

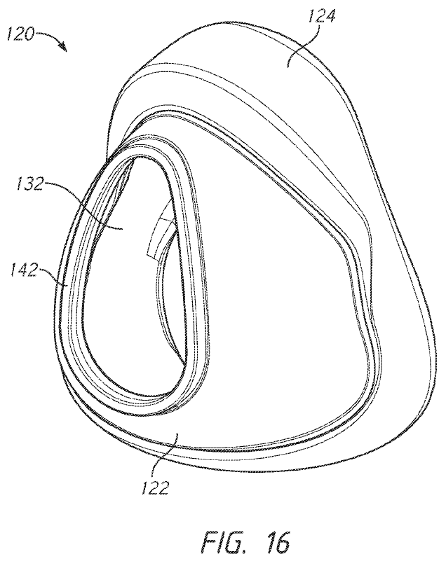

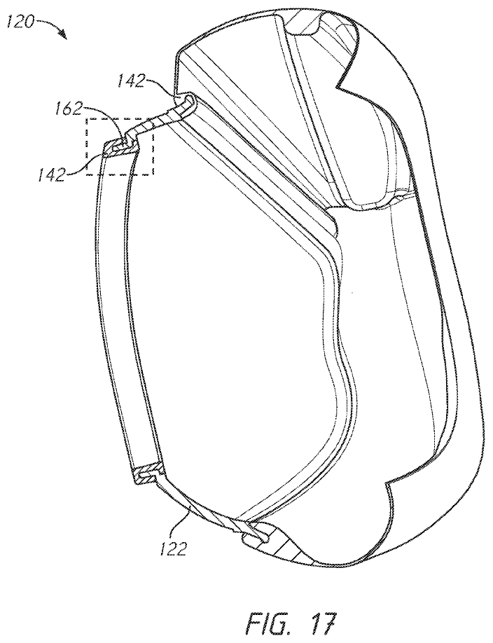

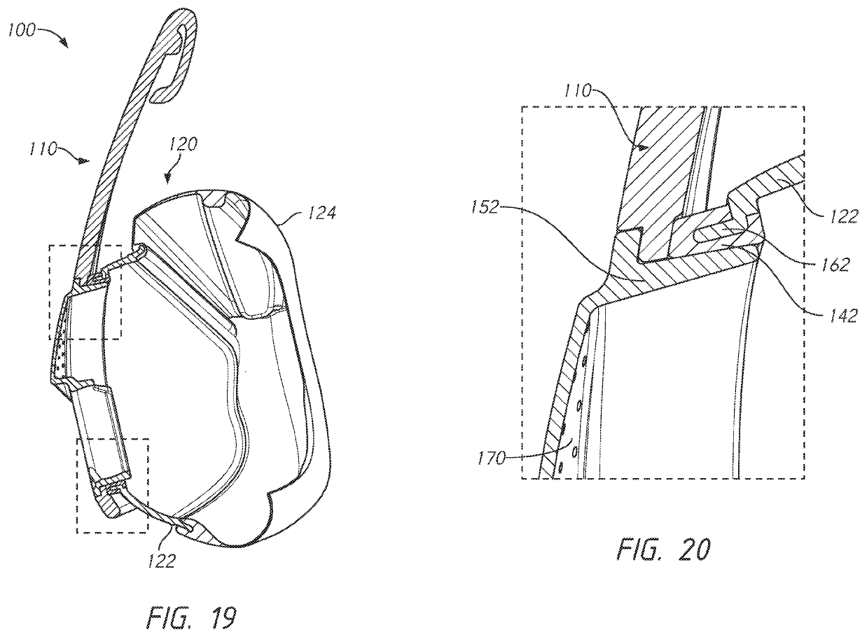

FIGS. 1, 2 and 15-20 illustrate a patient interface 100 configured for the delivery of respiratory therapy to a patient. The illustrated patient interface 100 is in the form of a mask, which is secured against the face of the user to define a substantially sealed breathing chamber 102. The respiratory therapy often involves the delivery of a flow of pressurized breathing gas (e.g., air) to the breathing chamber 102 of the patient interface 100 and, eventually, to the user's airways.

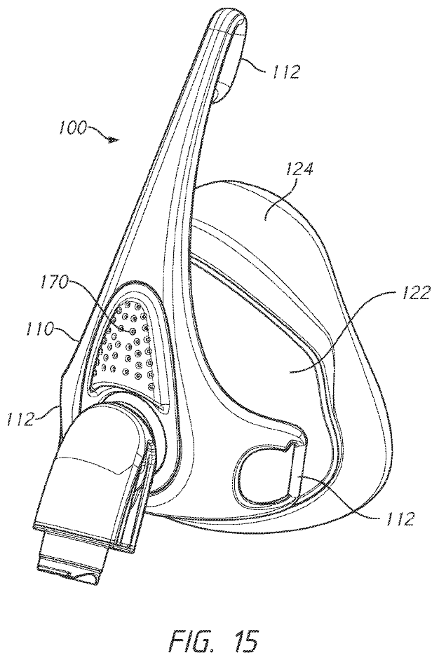

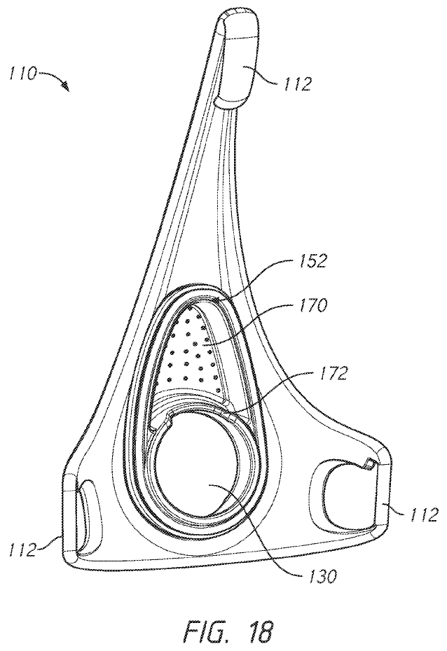

The illustrated patient interface 100 includes a frame 110 and a cushion module 120. In the illustrated arrangement, the cushion module 120 can be removably coupled to the frame 110. The frame 110 is configured to be connected to headgear (not shown), which holds the patient interface 100 in a sealed relation to the user's face. The frame 110 can include any suitable number or type of headgear mounts. In some configurations, the frame 110 includes two, three, four, five or more headgear mounts configured to receive two, three, four, five or more straps of a headgear or headgear assembly. In some configurations, a single headgear mount can be configured to receive more than one headgear strap. The mounts can be configured to directly or indirectly receive the headgear strap. For example, in some configurations, the headgear mount comprises an opening and/or a mounting post through which or around which the headgear strap is looped. In other configurations, the headgear strap is secured to the headgear mount by an intermediate structure, such as a headgear clip. The frame 110 can include a single type of mount (e.g., direct or indirect) or multiple types of mounts (e.g., both direct and indirect). For example, as illustrated in FIGS. 15 and 18, the frame 110 includes a single upper headgear mount 112, which is configured to receive a headgear clip that connects to two upper headgear straps. The frame 110 also includes two lower headgear mounts 112, with one mount located on each side of the frame 110.

The cushion module 120 defines at least a portion of the breathing chamber 102 of the patient interface 100. In the illustrated arrangement, the cushion module 120 includes a housing 122 and a cushion or seal 124. Preferably, the housing 122 includes a wall that extends in both a radial and axial direction relative to an axis 104 along which the frame 110 and the cushion module 120 overlap or are coupled. In some configurations, the axis 104 along which the frame 110 and the cushion module 120 overlap is generally or substantially aligned with a direction along which the frame 110 and the cushion module 120 are assembled. That is, in some arrangements, the frame 110 and the cushion module 120 are assembled by relative linear movement along the axis 104. However, in other arrangements, the frame 110 and/or the cushion module 120 can experience relative movement in directions other than along the axis 104. For example, assembly of the cushion module 120 to the frame 110 can employ a rocking motion, such that engagement between the frame 110 and the cushion module 120 occurs generally along the axis 104, but with relative movement in additional directions. For convenience, the axis 104 is referred to as the assembly axis or direction of assembly, which includes movement solely along the axis or movement along the axis with relative movement in additional direction, unless indicated otherwise. In some configurations, the assembly direction 104 or assembly axis can generally or substantially lie within the sagittal plane. Thus, the housing 122 defines a depth in a front-back or anteroposterior direction. As used herein, the terms “radial” and “axial” refer to directions relative to the assembly axis 104, which can be parallel to or coincident with an axis of a gas inlet opening of the frame 110 and/or the cushion module 120. The gas inlet opening axis is often referred to as the z-axis. Unless indicated otherwise, the terms “radial” and “axial” are not intended to refer to exact directions, but are used to distinguish between the two directions or to describe relative locations of two or more different locations.

The seal 124 is coupled to a rear or posterior portion of the housing 122.

The seal 124 defines a face-contacting surface configured to contact the face of a user of the interface 100. Preferably, the face-contacting surface of the seal 124 creates a sufficient scal with the user's face such that the desired positive pressure can be maintained throughout the vast majority of the therapy session when properly fitted and absent extenuating circumstances.

The seal 124 can be configured such that it surrounds either or both of the nose and mouth of the user. The seal 124 can be an inflation-type seal having an interior-concave curved wall that inflates when the interior of the interface 100 is pressurized to provide a good seal and better conform to the face of the particular user. However, other types of seals can be used, if desired. Moreover, certain features, aspects and advantages of the present disclosure can be utilized with other types of patient interfaces, such as direct nasal interfaces, among others.

In some configurations, the housing 122 is harder and/or more rigid than the seal 124. In any of the embodiments described herein, the housing 122 can be constructed from a material having a higher modulus of elasticity than the material of the seal 124. The material utilized to construct a portion or an entirety of the housing 122 can exhibit a greater rigidity or stiffness for a solid body of a given size and shape than a solid body of the same size and shape constructed from the material of the seal 124. Thus, a solid body constructed from the material of the seal 124 can have less rigidity or stiffness (or greater flexibility or compliance) than a solid body of the same size and shape constructed from the material of the housing 122. Unless indicated otherwise, descriptions of relative rigidity or stiffness of materials herein refer to differences in the modulus of elasticity between the materials or differences in rigidity or stiffness of solid bodies of the same size and shape constructed from the materials being compared. Descriptions of relative rigidity or stiffness of particular structures can refer to resistance to deformation of those structures, taking into account both material properties and physical characteristics of the structures (e.g., size, shape, wall thickness).

In some configurations, the housing 122 can be constructed from a relatively stiff or rigid plastic (e.g., polycarbonate). The seal 124 can be constructed from a relatively soft, flexible or pliable material, such as an elastomer (e.g., silicone). In some configurations, the housing 122 and the seal 124 are molded (e.g., injection molded). Thus, the housing 122 and the seal 124 can be constructed from moldable materials. In some configurations, the seal 124 is coupled to the housing 122 during a molding process, such as an over-molding process. In addition, or in the alternative, the seal 124 may be mechanically coupled to the housing 122. For example, the housing 122 can include a plurality of apertures through which the material of the seal 124 passes to create a mechanical interlock between the seal 124 and the housing 122.

As described above, the frame 110 defines or otherwise includes a gas inlet opening or gas inlet 130 that permits a flow of breathing gas to pass through the frame 110. In at least one configuration, the gas inlet 130 can be in the form of a gas inlet opening. The cushion module 120 defines or otherwise includes a connection opening 132 that permits a flow of breathing gas to pass through the cushion module 120. In at least one configuration, the connection opening 132 can be in the form of a gas inlet opening 132. In at least one configuration, the connection opening 132 can be in the form of a cushion module opening 132. When the cushion module 120 is assembled to the frame 110, the connection opening 132 of the cushion module 120 is aligned with the gas inlet 130 of the frame 110 such that a flow of breathing gas is permitted to pass through the gas inlet 130 of the frame 110 and the connection opening 132 of the cushion module 120 into the breathing chamber 102.

In some configurations, the patient interface 100 includes a friction coupling 140 that couples the cushion module 120 and the frame 110. In some arrangements, the friction coupling 140 is the only coupling between the frame 110 and the cushion module 120. In some configurations, the friction force provided by the friction coupling 140 is the only retention force that holds the cushion module 120 and the frame 110 together. That is, the friction coupling 140 can be the only direct coupling between the frame 110 and the cushion module 120. However, in other arrangements, the patient interface 100 can include additional couplings between the frame 110 and the cushion module 120.

In the illustrated arrangement, the friction coupling 140 is or comprises an elastomeric friction member 142 that in addition to coupling the cushion module 120 to the frame 110 also creates an airtight or substantially airtight seal between the frame 110 and the cushion module 120. With such an arrangement, leakage of breathing gas (e.g., air) between the frame 110 and the cushion module 120 can be prevented or sufficiently inhibited such that the desired positive pressure can be maintained within the breathing chamber 102. The elastomeric friction member 142 can have a greater hardness than the seal 124. For example, the friction member 142 can have a 60-80 shore A hardness and the seal 124 can have a lower shore A hardness, such as about a 40 shore A hardness, for example. In some configurations, the elastomeric friction member 142 can have the same hardness as the seal 124 and/or can be constructed from the same material. For example, the elastomeric friction member 142 and the seal 124 can each have a 40 shore A hardness. In some configurations, the seal 124 can have a greater hardness than the elastomeric friction member 142. At least a surface of the friction member 142 that engages the frame 110 can have a textured surface finish, which can provide a desirable balance between retention and allowing sliding movement for assembly and disassembly. A very smooth finish of the elastomeric friction member 142 can inhibit sliding. Thus, a frosted or textured surface finish—or another finish that provides some reasonable degree of roughness—can provide a desirable compromise between retention and sliding.

The illustrated frame 110 includes at least a main wall or a front wall 150 and a collar 152, which can be in the form of a tubular wall that extends rearwardly (posteriorly or toward the user in an assembled and fitted in-use orientation) from the front wall 150 to a rearward or free end, edge or rim and surrounds the gas inlet 130. In at least one embodiment, the collar 152 can be in the form of an inlet collar 152. The perimeter defined by the collar 152 can be circular, ovate, oval, triangular or other polygonal shapes, or other combinations thereof, for example. For example, the perimeter defined by the collar 152 can be substantially triangular in shape with rounded corners. Further, the sides of the triangular shapes could be curved or straight. Non-circular shapes can advantageously rotationally fix the frame 110 and cushion module 120 about the assembly axis 104. Moreover, asymmetric shapes can inhibit or prevent the cushion module 120 from being assembled to the frame 110 in an upside-down or otherwise improper orientation. Advantageously, a tapered ovate or triangular shape, such as that illustrated in the interface 100 of FIGS. 15-20, provides for both non-rotation and proper alignment. The front wall 150 and the collar 152 can be unitarily formed of a material with sufficient strength and rigidity to maintain a breathing chamber 102 during use, such as a relatively rigid polymeric material (e.g., polycarbonate), by a process such as molding (e.g., injection molding).

The housing 122 of the illustrated cushion module 120 includes a main wall 160 and a support flange 162 that extends forwardly (anteriorly or away from the user in an assembled and fitted in-use orientation). The main wall 160 and/or the support flange 162 surrounds and/or defines the connection opening 132. The housing 122 also includes a support wall 163 that surrounds and is adjacent the connection opening 132 and/or the support flange 162. In the arrangement of FIGS. 1-7, the support wall 163 is a portion of the main wall 160 and connects the support flange 162 to a remainder of the main wall 160. However, the support wall 163 could be separate from the main wall 160, such as spaced forwardly or rearwardly from the main wall 160. The support flange 162 defines a perimeter shape that preferably corresponds to the perimeter shape of the collar 152, but preferably is somewhat larger to accommodate the portion of the friction member 142 therebetween. The support flange 162 of the housing 122 could extend in a rearward direction (toward the user) in addition or in the alternative of the forward-extending support flange 162.

In the illustrated arrangement, the elastomeric friction member 142 is carried by or coupled to the cushion module 120. More specifically, in the illustrated arrangement, the elastomeric friction member 142 is carried by or coupled to the housing 122. In particular, the friction member 142 is coupled to the support flange 162. At least a portion of the main wall 160 is located external to the friction member 142 and extends rearwardly and outwardly from the friction member 142 toward or to the seal 124. Preferably, the main wall 160 provides sufficient strength and rigidity to maintain a breathing chamber 102 during use. The illustrated friction member 142 includes a peripheral surface 146. In at least one configuration, the peripheral surface 146 can be an outward-facing surface 146. In at least one configuration, the peripheral surface 146 can be an exterior surface 146. In the illustrated arrangement, the housing 122 of the cushion module 120 extends through the friction member 142 such that a portion of the housing 122 is embedded within the friction member 142. This embedded portion forms a mechanical anchor to secure the friction member 142 to the housing 122. In addition, the embedded portion can provide hoop strength to the friction member 142 or limit outward expansion of the friction member 142 to enhance the friction fit with the collar 152 of the frame 110.

In the illustrated arrangement, the support flange 162 is disposed within the friction member 142 and the housing 122 extends through the peripheral surface 146 of the friction member 142. With such an arrangement, the support flange 162 reinforces the friction member 142 such that the retention force between the frame 110 and the cushion module 120 is determined by compression of the friction member 142 to a greater extent than similar arrangements without the support flange 162. In addition, such an arrangement can facilitate or allow for the friction coupling 140 to have a single overlap engagement, in which a surface of the friction member 142 on an opposite side of the engaging surface is exposed—in contrast to an arrangement in which the friction member 142 is received within a channel and engaged on both sides. A single overlap engagement can be easier to assemble than arrangements in which an elastomer seal is received within a channel, which can be difficult or impossible to engage the entire seal at once and typically require sections of the seal to be engaged sequentially.

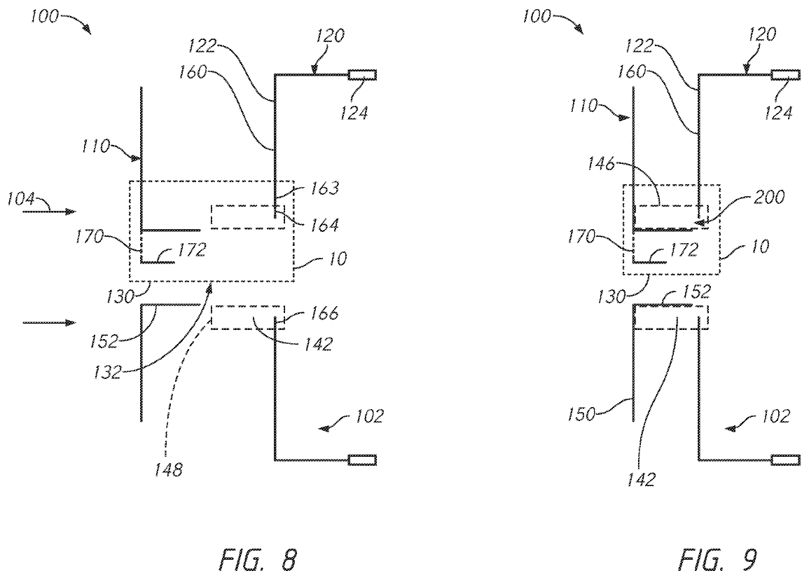

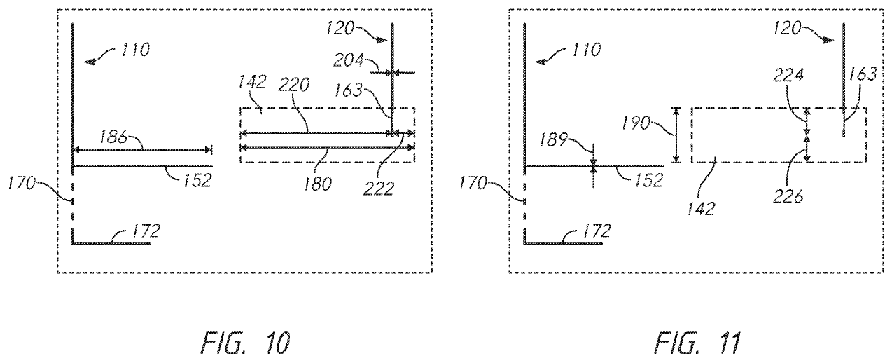

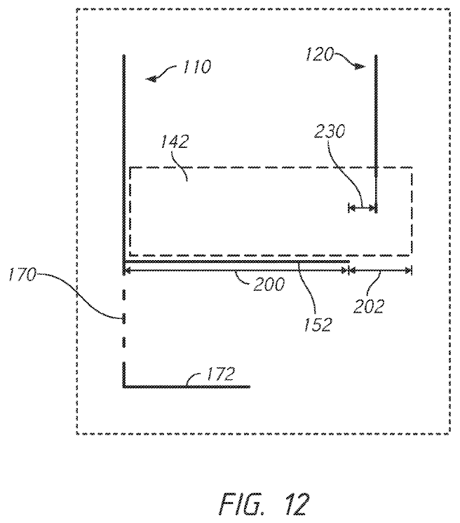

In the illustrated arrangement, the housing 122 extends into the friction member 142 in a direction defining a non-zero angle relative to the peripheral surface 146 of the friction member 142 and/or to an axis of the friction member 142, which can be coincident with the assembly axis 104. In the illustrated arrangement, a rearward end of the friction member 142 is flush or substantially flush with a rearward surface of the housing 122 adjacent the friction member 142. Thus, the housing 122 extends through a rearward end portion of the friction member 142. However, in other arrangements, the friction member 142 can extend rearwardly of the rearward surface of the housing 122 adjacent the friction member 142, as illustrated and described in connection with FIGS. 8-12. In such an arrangement, the housing 122 extends through a portion of the friction member 142 spaced from the rearward end, as well as the forward end.

The friction member 142 engages the insert collar 152 to couple the cushion module 120 to the frame 110 in a sealed or substantially sealed manner. In the illustrated arrangement, the friction member 142 comprises an engagement surface 144 that engages an engagement surface 154 of the collar 152. In at least one configuration, the engagement surface 144 is an interior or radially inward-facing surface of the friction member 142. In at least one configuration, the engagement surface 154 is an exterior or radially outward-facing surface of the collar 152. However, in other arrangements, the friction member 142 could engage an interior surface of the collar 152. Preferably, with either arrangement the frame 110 defines at least a portion of the breathing chamber 102. For example, in the illustrated configuration, an interior space defined by the collar 152 defines a portion of the breathing chamber 102. As described above, in the illustrated arrangement, the peripheral surface 146 is exposed or is not directly covered when the cushion module 120 is assembled to the frame 110. With such an arrangement, the friction member 142 is compressed from a single surface (e.g., the engagement surface 144). Accordingly, dimensional variations as a result of the manufacturing process will not impact the assembly and removal force to as great an extent as a design in which both the interior and exterior surfaces of the friction member 142 engage a surface of a cooperating structure. Moreover, the single collar 152 arrangement is easier to clean than an arrangement having a channel, which can be difficult to access by the user.

In the illustrated arrangement, the friction member 142 defines an abutment surface 148 that abuts or contacts the front wall 150 of the frame 110 when the cushion module 120 is coupled to the frame 110. The abutment surface 148 is a forward-facing surface of the friction member 142 and is configured to contact a portion of the rearward-facing surface of the front wall 150 of the frame 110 surrounding and/or adjacent the collar 152. Contact of the abutment surface 148 with the frame 110 can provide feedback to the user that the assembly of the cushion module 120 to the frame 110 is sufficient or complete. Although contact of the engagement surfaces 144 and 154 in most cases will be sufficient to create a reliable seal, in some cases or in some designs, contact of the abutment surface 148 with the frame 110 can also contribute to creating or maintaining a seal between the cushion module 120 and the frame 110. Contact between the friction member 142 and the frame 110 can be advantageous for at least the reasons described above; however, such a feature is not essential and can be omitted if desired. If desired, other feedback arrangements can be provided to give user tactile, auditory, visual or other feedback of complete engagement between the cushion module 120 and the frame 110.

In some configurations, the friction member 142 is permanently coupled to the housing 122. The friction member 142 can be permanently coupled to the main wall 160 and/or the support flange 162. For example, the friction member 142 can be coupled to the support flange 162 by an over-molding process. In some configurations, the housing 122 comprises one or more apertures 166 through which the material of the friction member 142 extends as a result of the over-molding process. In the illustrated arrangement, at least a portion of the apertures 166 are located in the support flange 162. In other arrangements, a portion or an entirety of the apertures 166 are located in a portion of the main wall 160 surrounding and relatively adjacent to or at the connection opening 132. Such an arrangement provides a mechanical interlock between the friction member 142 and the support flange 162 and/or the main wall 160 of the housing 122. In the illustrated arrangement, the support flange 162 includes the plurality of apertures 166, each of which passes radially through the support flange 162. In some configurations, the apertures 166 are located closer to the main wall 160 of the housing 122 than a free end of the support flange 162. In some configurations, the apertures 166 are evenly distributed around the perimeter of the support flange 162. In other configurations, the apertures 166 are unevenly distributed around the perimeter of the support flange 162. Although the apertures 166 are shown solely on the support flange 162, in some configurations, apertures 166 could be provided on the main wall 160 as well.

In the illustrated arrangement, the patient interface 100 comprises a vent 170 in fluid communication with the breathing chamber 102. The vent 170 is configured to allow a flow of breathing gases and/or gases exhaled by the user to pass from the breathing chamber 102 to the atmosphere external of the patient interface 100. In some configurations, the vent 170 is a bias flow vent configured to maintain a desired or sufficient positive pressure with the breathing chamber 102 in use. The vent 170 can comprise a plurality of small openings.

In some configurations, the vent 170 is carried by or located in the frame 110. However, in other configurations, the vent can be located on the cushion module 120 (e.g., the housing 122) or another component of the patient interface 100. In the illustrated arrangement, the vent 170 is located within the perimeter of the collar 152. In particular, the vent 170 is located in the front wall 150 of the frame 110.

In some configurations, the frame 110 includes a divider wall 172 between the vent 170 and the gas inlet 130. The divider wall 172 can be positioned between and/or separate the vent 170 from the gas inlet 130. In some configurations, the divider wall 172 extends from one side to the other side of the collar 152. In such an arrangement, the ends of the divider wall 172 can be connected to the interior surface of the collar 152. However, in other arrangements, the divider wall 172 is interrupted or spans only a portion of the distance from one side to the other side of the collar 152. In some configurations, a forward edge of the divider wall 172 can be connected to the front wall 150 of the frame 110. In the illustrated arrangement, a portion or an entirety of a rearward edge of the divider wall 172 is located forward of or closer to the front wall 150 of the frame 110 relative to a rearward edge or rim of a portion or an entirety of the collar 152. An upper surface or the surface of the divider wall 172 adjacent the vent 170 can extend generally in an anteroposterior direction or in a direction of gas flow through the vent 170. In some configurations, a portion or an entirety of the upper surface of the divider wall 172 can be angled relative to the anteroposterior direction or the direction of gas flow through the vent 170, but preferably does not substantially or completely overlap the vent 170 in the direction of gas flow through the vent 170. In some configurations, a space defined by an interior surface of the collar 152, an upper surface of the dividing wall 172 and a rearwardly-facing surface of a vent wall of the vent 170 forms at least a portion of the breathing chamber 102.

The vent 170, the collar 152 and the divider wall 172 can be constructed in any suitable manner. For example, any one or more of the vent 170, the collar 152 and the divider wall 172 can be constructed in a unitary manner with another portion of the frame 110 (e.g., the front wall 150). In other arrangements, any one or more of the vent 170, the collar 152 and the divider wall 172 can be formed as a separate structure and coupled to another portion of the frame 110 (e.g., the front wall 150). For example, the vent 170 can be formed as a separate structure and coupled to a portion or the remainder of the frame 110. In some configurations, the vent 170, divider wall 172 and collar 152 are formed as a unitary structure and coupled to a portion or the remainder of the frame 110. Such separate structures can be coupled to a portion or the remainder of the frame 110 by any suitable method or arrangement, such as by adhesives, over-molding, welding (e.g., ultrasonic welding), mechanical fasteners or snap-fits.

In the illustrated arrangement, the frame 110 defines a socket configured to receive a conduit connector, such as an elbow, configured to connect a breathing circuit to the patient interface 100. However, in other arrangements, the conduit connector can be connected to other portions or components of the patient interface 100, such as a portion of the cushion module 120 (e.g., the housing 122). In some configurations, the collar 152 defines at least a portion of the elbow socket. In the illustrated arrangement, the divider wall 172 and the collar 152 cooperate to define a portion or an entirety of the elbow socket. In some configurations, the conduit connector or elbow is capable of pivoting about one or more axes relative to the frame 110. For example, the conduit connector or elbow can have a swivel connection with the frame 110.

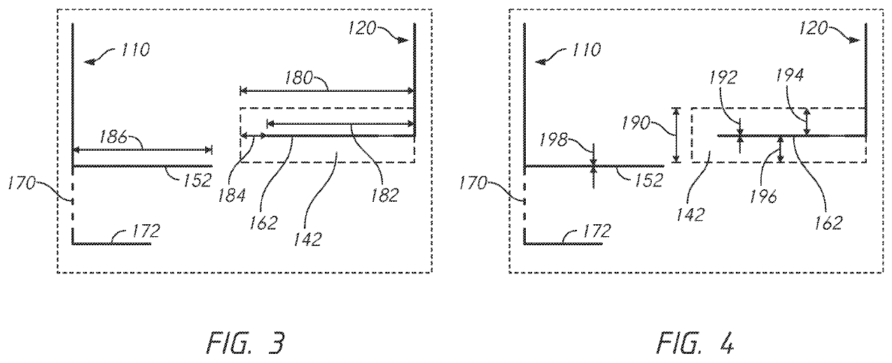

The friction coupling 140 is configured to provide any one or number of desirable characteristics related to the assembly and disassembly of the cushion module 120 to the frame 100 or use of the assembly. Such characteristics can include, for example and without limitation: a desired magnitude or range of assembly force, a desired magnitude or range of disassembly force, relatively consistent assembly or disassembly forces amongst different components (e.g., so that the forces will not vary drastically from what the user is accustomed to when using a replacement cushion module 120), relatively smooth assembly or disassembly, feedback upon complete assembly, reliable seal between the cushion module 120 and the frame 110. Any one or combination of these characteristics, or other advantages, can be provided or facilitated by the selection of suitable dimensions or proportions of the different structures of the friction coupling 140. Examples of such dimensions or proportions, and related advantages, are provided below with reference to FIGS. 3-5.

In the illustrated arrangement, the friction member 142 defines a first length 180 that extends in or substantially in an axial direction or the direction of assembly 104. The support flange 162 defines a second length 182 that extends in or substantially in an axial direction or the direction of assembly 104. An unsupported portion of the friction member 142 that extends beyond a free end of the support flange 162 defines a third length 184 that extends in or substantially in an axial direction or the direction of assembly 104. The collar 152 defines a fourth length 186 that extends in or substantially in an axial direction or the direction of assembly 104.

In some configurations, the first length 180 is equal to the sum of the second length 182 and the third length 184. With such an arrangement, the elastomeric friction member 142 is compact since the entire support flange 162 is embedded in the elastomeric friction member 142. Accordingly, the arrangement makes efficient use of available space in at least an axial direction. As used herein, the term “equal” or “substantially equal” means that the identified dimensions are equivalent within measurement error using common or reasonable measurement techniques and/or within normal or reasonable manufacturing tolerances.

However, in other configurations, the first length 180 is greater than the sum of the second length 182 and the third length 184 or the first length 180 is less than the sum of the second length 182 and the third length 184. These situations can occur if the end or the abutment surface 148 of the elastomeric friction member 142 is angled or oriented at a non-perpendicular angle relative to the direction of assembly 104 and depending on the radial location at which the lengths are measured. These situations can also occur if the end of the support flange 162 nearest the main wall 160 terminates within or beyond the corresponding end of the elastomeric friction member 142. Advantages of these configurations can include allowing different geometries of the cushion module 120 and/or frame 110. For example, by having a longer support flange 162 than what is necessary or desirable strictly to support the friction member 142, more space can be created for a user's nose within the breathing chamber 102. In other configurations, the first length 180 is less than the second length 182. In such configurations, the elastomeric friction member 142 spans only a portion of the support flange 162. As such, a portion of the support flange 162 is embedded within the elastomeric friction member 142, and a portion of the support flange 162 is exposed outside of the elastomeric friction member 142 (i.e. not embedded within the elastomeric friction member 142).

In the illustrated arrangement, the friction member 142 defines a first thickness 190 that extends in or substantially in a radial direction or perpendicular to the direction of assembly 104. The support flange 162 defines a second thickness 192 that extends in or substantially in a radial direction or perpendicular to the direction of assembly 104. A portion of the friction member 142 located radially outside of the support flange 162 defines a third thickness 194 that extends in or substantially in a radial direction or perpendicular to the direction of assembly 104. A portion of the friction member 142 located radially inside of the support flange 162 defines a fourth thickness 196 that extends in or substantially in a radial direction or perpendicular to the direction of assembly 104. The collar 152 defines a fifth thickness 198 that extends in or substantially in a radial direction or perpendicular to the direction of assembly 104.

In some configurations, the first thickness 190 equals the sum of the second thickness 192, the third thickness 194, and the fourth thickness 196. In such an arrangement, there are no hollow spaces within the friction member 142 and no additional layers of material. Such an arrangement is compact in an axial direction.

In some configurations, the third thickness 194 and the fourth thickness 196 are equal. Such an arrangement provides uniform compression behavior on each side of the friction member 142 in configurations in which each of the engagement surface 144 and the peripheral surface 146 are engaged by a cooperating component of the patient interface 100.

In some configurations, the second thickness 192 is equal to one or both of the third thickness 194 and the fourth thickness 196. When accomplished by a relatively thicker support flange 162, such an arrangement can provide improved strength of the support flange 162 in comparison to thinner support flanges 162. Advantageously, in comparison to thinner support flanges 162, such an arrangement can reduce flex of the support flange 162 such that the application force of the friction fit is determined solely or to a greater extent by compression of the friction member 142 rather than flex of the support flange 162.

In some configurations, the fourth thickness 196 is greater than or equal to the third thickness 194. Advantageously, a larger fourth thickness 196 improves consistency of the removal force between the frame 110 and cushion module 120 for different components in comparison to smaller thicknesses, wherein differences between different components can be the result of normal manufacturing tolerances. With a larger fourth thickness 196, the compression distance as a proportion of the total thickness for a given retention force is reduced. Accordingly, normal variations due to manufacturing tolerances will cause less variation of the retention, removal or application force in comparison to smaller thicknesses. In other configurations, the fourth thickness 196 is less than or equal to the third thickness 194. Such an arrangement can reduce overall material use for the friction member 142 in comparison to greater thicknesses.

In some configurations, the second thickness 192 and the fifth thickness 198 are equal. With such an arrangement, a relatively similar support strength is provided by a similarly thick collar 152 and support flange 162. However, in other configurations, the second thickness 192 is greater than the fifth thickness 198. With such an arrangement, deformation of the support flange 162 is minimized. In yet other configurations, the second thickness 192 is less than the fifth thickness 198. With such an arrangement, deformation of the collar 152 is minimized or at least reduced relative to a thinner collar 152. This minimizes or reduces the effect of the deformation of the collar 152 on the rotation of the elbow in the elbow socket, which in some arrangements is at least partially formed by the collar 152.

In some configurations, the second thickness 192 is less than or equal to either one of the third thickness 194 and the fourth thickness 196. Such an arrangement allows for reduced material of the support flange 162 and, thus, the housing 122 of the cushion module 120 relative to arrangements having a thicker support flange 162. However, in other configurations, the second thickness 192 is greater than or equal to either one of the third thickness 194 and the fourth thickness 196. Such an arrangement provides for increased strength of the support flange 162 in comparison to arrangements having a thinner support flange 162.

In some configurations, the first thickness 190 is greater than or equal to the fifth thickness 198. Such an arrangement allows for reduced material on the frame 110 in comparison to configurations having a thicker collar 152. Such an arrangement also provides a sufficiently thick friction member 142 to allow for a sufficient deformation of the friction member 142. However, in other configurations, the first thickness 190 is less than or equal to the fifth thickness 198. Such an arrangement allows for reduced material on the friction member 142 in comparison to configurations having a thicker friction member 142.

In some configurations, the fourth thickness 196 varies along the length of the friction member 142 or in the assembly direction 104. Such an arrangement provides for an angled or non-parallel orientation of the engagement surface 144 of the friction member 142 and the engagement surface 154 of the collar 152 and a variation in the retention force along the length of the friction coupling 140. In some configurations, the fourth thickness 196 varies along the length of the friction member 142 from a minimum near a forward or free end of the friction member 142 to a maximum near a rearward end of the friction member 142 nearest the main wall 160 of the cushion module 120. In some configurations, the angled or non-parallel orientation can provide a lead-in or tapered connection for the assembly of the friction member 142 and the collar 152. Such an arrangement can facilitate assembly by casing the initial alignment of the collar 152 and the friction member 142 and increasing the assembly or retention force as the overlap increases between the collar 152 and the friction member 142. In other configurations, the fourth thickness 196 varies along the length of the friction member 142 from a maximum near the forward or free end of the friction member 142 to a minimum near the rearward end of the friction member 142 nearest the main wall 160. The engagement surface 144 of the friction member 142 can be non-linear, such that the fourth thickness 196 increases and decreases along the length of the friction member 142. In some configurations, the fourth thickness 196 can be larger at one or more locations (e.g., regions or sections) intermediate the forward and rearward ends relative to a thickness at one or both of the forward and rearward ends of the friction member 142 or relative to a thickness between those locations. In some configurations, the fourth thickness 196 can be smaller at one or more locations (e.g., regions or sections) intermediate the forward and rearward ends relative to a thickness at one or both of the forward and rearward ends of the friction member 142 or relative to a thickness between those locations. For example, in some configurations, the engagement surface 144 can define one or more annular or part-annular retention beads or protrusions and/or one or more annular or part-annular relief channels or notches along the length of the friction member 142, which can result in variations in the fourth thickness 196.

In some configurations, no portion of the friction member 142 is located radially outward of the support flange 162 such that there is no third thickness 194 (i.e., the third thickness 194 is zero). Such an arrangement provides for reduced material on the friction member 142 relative to configurations in which a portion of the friction member 142 is located radially outward of the support flange 162. In some such arrangements, the friction member 142 may be chemically bonded by use of a self-adhesive elastomer, an adhesive or bonding agent, or pre-treatment process to enhance the bond with the support flange 162.

In some configurations, a portion of the friction member 142 that overlaps with the collar 152 defines an overlap length 200. In some configurations, the overlap length 200 is greater than or equal to the third length 184. Such an arrangement allows the support flange 162 to support the friction member 142 as it deforms to enhance the grip of the friction member 142 on the collar 152. In some configurations, the overlap length 200 is greater than or equal to a sum of the second length 182 and the third length 184. Such an arrangement allows for increased or maximum grip of the friction member 142 on the collar 152.

In other configurations, the overlap length 200 is less than the third length 184. In such an arrangement, since the thickness of the friction member 142 that can deform in the portion defining the third length 184 is greater than in the portion defining the fourth thickness 196, more reliable grip or retention forces are able to be maintained between different cushion modules 120 and frames 110, which may vary due to normal manufacturing tolerances. In some configurations, the overlap length 200 is less than a sum of the second length 182 and the third length 184. Such an arrangement allows for efficient use of space.

In some configurations, the overlap length 200 equals the fourth length 186. In such an arrangement, the entire collar 152 is utilized for engagement with the friction member 142. Such an arrangement provides for efficient use of space and material. In other configurations, the overlap length 200 is less than the fourth length 186. In yet other configurations, the overlap length 200 is greater than the fourth length 186. Such an arrangement is possible where the collar 152 has, for example, different lengths around the perimeter and the fourth length 186 is taken as a shorter length that is less than the first length 180 of the friction member 142. Such an arrangement provides for varying amounts of overlap around the perimeter of the collar 152.

In some configurations, a portion of the friction member 142 that does not overlap with the collar 152 defines a non-overlap length 202 and wherein a portion (e.g., the support wall 163) of the main wall 160 adjacent the support flange 162 defines a support wall thickness 204, which can also be referred to as a first main wall thickness 204. In some configurations, a sum of the first length 180 and the first main wall thickness 204 equals a sum of the overlap length 200 and the non-overlap length 202. With such an arrangement, the full length of the friction member 142 is utilized for gripping the collar 152 without excess material or space being used. However, in other configurations, a sum of the first length 180 and the first main wall thickness 204 is greater than a sum of the overlap length 200 and the non-overlap length 202. With such an arrangement, the frame 110 is spaced further away from the face of the user. In yet other configurations, a sum of the first length 180 and the first main wall thickness 204 is less than a sum of the overlap length 200 and the non-overlap length 202. Such an arrangement provides for a reduced amount of material required for the friction member 142 in comparison to configurations having a longer friction member 142.

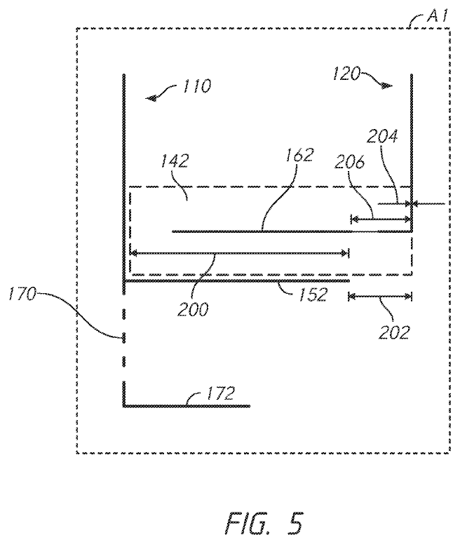

With reference to FIG. 5, in some configurations, a distance between the portion of the main wall 160 surrounding and/or adjacent the connection opening 132 and a point on the plurality of apertures 166 furthest from that portion of the main wall 160 defines an aperture offset distance 206. In some configurations, the non-overlap length 202 is greater than or equal to a sum of the aperture offset distance 206 and the first main wall thickness 204. With such an arrangement, the rearward or free end of the collar 152 is located forward of the forward or closest edge or point of the apertures 166. That is, the collar 152 is shorter than when the non-overlap length 202 is smaller or non-existent. A shorter or smaller collar 152 allows for less material and smaller parts in comparison to longer or larger collars 152. In other configurations, the non-overlap length 202 is less than or equal to a sum of the aperture offset distance 206 and the first main wall thickness 204. Such an arrangement allows for a stronger connection between the collar 152 and the friction member 142 with a longer overlap length 200 (shorter non-overlap length 202).

In some configurations, a sum of the second length 182 and the third length 184 is greater than or equal to a sum of the overlap length 200 and the aperture offset distance 206. With such an arrangement, a longitudinal gap is provided between the apertures 166 and the free end of the collar 152. The presence of such a longitudinal gap can provide for or facilitate more flex of the friction member 142 in a cantilever or cantilever-like manner, which may be beneficial in certain embodiments. In other configurations, a sum of the second length 182 and the third length 184 is less than or equal to a sum of the overlap length 200 and the aperture offset distance 166. With such an arrangement, a stronger connection can be made between the collar 152 and the friction member 142 with a relatively longer overlap length 200 in comparison to configurations having a smaller or shorter overlap length 200.

In some configurations, a sum of the overlap length 200 and the aperture offset distance 206 is less than or equal to first length 180. With such an arrangement, a longitudinal gap is provided between the apertures 166 and the free end of the collar 152. The presence of such a longitudinal gap can provide for or facilitate more flex of the friction member 142, as described above. However, in other configurations, a sum of the overlap length 200 and the aperture offset distance 206 is greater than or equal to the first length 180. Such an arrangement can be accomplished by providing a collar 152 having a sufficient length to overlap in a longitudinal direction with the apertures 166. Such an arrangement provides for more secure engagement between the collar 152 and the friction member 142 and, in some embodiments, may facilitate or provide for better direction of a flow of breathing gases airflow into the breathing chamber 102.

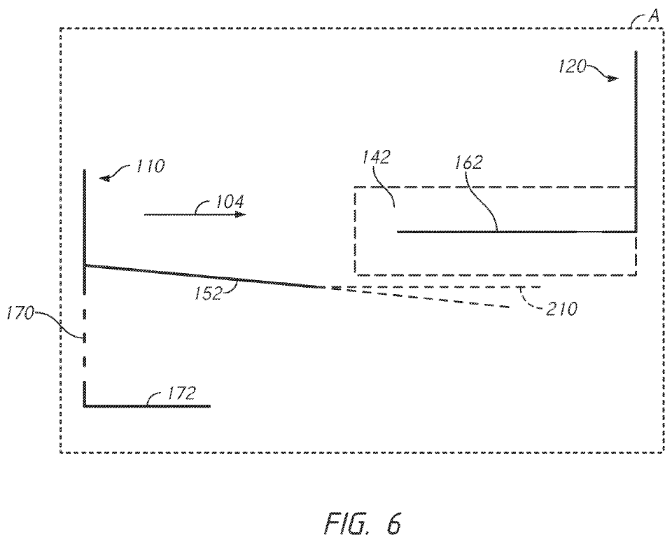

With reference to FIG. 6, in some configurations, one or both of the friction member 142 and the collar 152 are configured such that a relative angle 210 is defined between the engagement surface 144 of the friction member 142 and the engagement surface 154 of the collar 152. Such an arrangement can be created by, for example, providing a tapered collar 152 and/or a tapered friction member 142 or support flange 162 in a longitudinal direction or along the assembly direction 104. In some configurations, the collar 152 is angled relative to the direction of assembly 104. In some configurations, at least one of the support flange 162 and the engagement surface 144 of the friction member 142 is angled relative to the direction of assembly 104. Such arrangements allow for a variable assembly, removal or retention force along the length of the collar 152 to be controlled between the collar 152 and the friction member 142. Such an arrangement can also or alternatively allow for simpler tooling by providing a draft angle in the mold tool to facilitate removal of the molded part.

With the configurations described above, the friction coupling 140 can be selectively configured to provide desired assembly, removal or retention force characteristics. For example, the friction coupling 140 can be configured such that a force acting to compress the friction member 142 is equal along a length of the collar 152. Such arrangements can provide for a more predictable deformation of the friction member 142 (or support flange 162 and/or collar 152). In other configurations, the friction coupling 140 can be configured such that a force acting to compress the friction member 142 is unequal along a length of the collar 152. Such arrangements can provide for a variable force along the length of the collar 152 to be controlled between the collar 152 and the friction member 142.

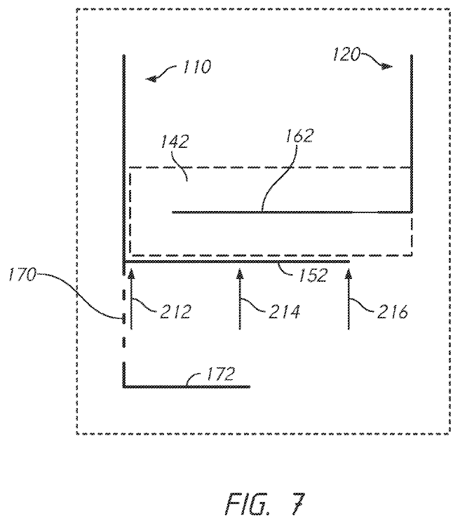

For the sake of comparison, FIG. 7 illustrates a retention force or force acting to compress the friction member 142 at three different locations on the collar 152. A first retention force 212 is located at or near an end of the collar 152 closest to the front wall 150 of the frame 110. A second retention force 214 is located at an intermediate position on the collar 152. A third retention force 216 is located at or near a free end of the collar 152. In some configurations, more ‘grip’ or a larger retention force is provided in certain areas of the friction coupling 140, such as close to the front wall 150 of the frame 110. That is, in such configurations, the first retention force 212 can be greater than either or both of the second retention force 214 and the third retention force 216. Such an arrangement can reduce deformation of the collar 152 by having a relatively higher retention force at or nearer the front wall 150 where the collar 152 is supported and a relatively lower retention force at or nearer the free end where the collar 152 is unsupported by another structure. In other configurations, the retention force is configured to be smaller at a location closer to the front wall 150 relative to a location further from the front wall 150. That is, in such arrangements, the first retention force 212 can be less than either or both of the second retention force 214 and the third retention force 216. Such an arrangement can result, for example, from having a chamfer or other shaping of the forward end of the friction member 142 to avoid interference with the transition between the collar 152 and the front wall 150.

In some configurations, a rearward-most extent of the friction member 142 is at or forward of a portion of the main wall 160 surrounding and/or adjacent to the connection opening 132. Such an arrangement avoids the friction member 142 extending into the breathing chamber 102 thereby providing more room and/or avoiding interference with the user's nose.

In other configurations, a rearward-most extent of the friction member 142 is rearward of the main wall 160 such that a portion of the friction member 142 is located along the main wall 160 in a longitudinal direction. That is, a portion of the friction member 142 is located adjacent each of a forward surface and a rearward surface of the main wall 160. Such configurations can include a friction member 142 that projects rearwardly from the main wall 160 and into the breathing chamber 102. With such an arrangement, the frame 110 can be brought closer to the face of the user while provide for the same length of overlap of the friction member 142 and the collar 152, thereby reducing the profile of the patient interface 100. In such arrangements, the friction member 142 may occupy a portion of the breathing chamber 102. Accordingly, such arrangements may be preferable in circumstances in which the location of the connection opening 132 is positioned below the user's nose or in another location in which contact with the user's face will be avoided.

FIGS. 8-12 illustrate another patient interface 100 that is similar to the interface 100 of FIGS. 1-7 with the notable exception that the friction member 142 is unsupported along a substantial portion of its length. In other respects, the patient interface 100 of FIGS. 8-12 is substantially similar to the patient interface 100 of FIGS. 1-7. Accordingly, the same reference numbers are used to refer to the same or similar features or components between the two interfaces 100. Any features or components of the interface 100 of FIGS. 8-12 that are not described in detail can be the same as or similar to the corresponding feature or component of the interface 100 of FIGS. 1-7, or can be of another suitable arrangement.

With reference to FIGS. 8-12, in some configurations, an embedded portion of the cushion module 120 is defined by a portion of the housing 122 located within the friction member 142. In the illustrated arrangement, the embedded portion is defined by a portion of the main wall 160, or a support wall 163, located radially outward of an opening rim 164, which surrounds and defines the connection opening 132. In some configurations, the embedded portion 163 extends substantially in a radial direction or a direction substantially perpendicular to the assembly direction 104 of the cushion module 120 to the frame 110. In other configurations, the embedded portion can extend in both an axial and a radial direction (or have both an axial and radial component). In some configurations, the embedded portion comprises forward and/or rearward-extending support flange portions, which can provide a mechanical interlock between the housing 122 and the friction member 142. However, preferably, the embedded portion does not extend along a substantial length of the friction member 142 in contrast to the arrangement of FIGS. 1-7.

In some configurations, the friction member 142 extends in both forward and rearward directions from the portion of the housing 122 to which the friction member 142 is coupled. In the illustrated arrangement, the friction member 142 has a portion that extends in a forward direction from a forward-facing surface of the main wall 160 and a portion that extends in a rearward direction from a rearward-facing surface of the main wall 160. In some configurations, the portion of the friction member 142 adjacent the forward surface of the main wall 160 has a surface (e.g., a radially-outward-facing surface) that is aligned in a radial direction with a surface (e.g., a radially-outward-facing surface) of the portion of the friction member 142 adjacent the rearward surface of the main wall 160.

In the illustrated arrangement, the collar 152 defines a collar length 186, which corresponds to the fourth length 186 of the interface 100 of FIGS. 1-7. A portion of the housing 122 embedded within the friction member 142 (e.g., the support wall 163 and/or the opening rim 164) defines a thickness 204, which is referred to herein for convenience as a support wall thickness 204. The support wall thickness 204 corresponds to the first main wall thickness 204 of the interface of FIGS. 1-7. The friction member 142 defines a total friction member length 180, which corresponds to the first length 180 of the interface 100 of FIGS. 1-7. The friction member 142 also defines a forward length 220 forward of the opening rim 164/support wall 163 and a rearward length 222 rearward of the opening rim 164/support wall 163.

In some configurations, the total friction member length 180 is equal to a sum of the forward length 220, the rearward length 222 and the support wall thickness 204. However, in other configurations, the total friction member length 180 is greater than or less than a sum of the forward length 220, the rearward length 222 and the support wall thickness 204. Such situations can result from, for example, the friction member 142 having angled front and/or rear end surfaces or front and/or rear end surfaces that are irregular in shape or otherwise not normal to the length direction and the length measurements are taken at a portion of the friction member 142 that has a greater or lesser length than the average length.

In the illustrated arrangement, the collar 152 defines a collar thickness 198, which corresponds to the fifth thickness 198 of the interface 100 of FIGS. 1-7. The friction member 142 defines a total thickness 190, which corresponds to the first thickness 190 of the interface 100 of FIGS. 1-7. A portion of the friction member 142 that extends along the embedded portion of the housing 122 (the opening rim 164 and/or the support wall 163) defines a supported thickness 224 and a portion of the friction member 142 that extends inwardly of the embedded portion of the housing 122 (the opening rim 164 and/or the support wall 163) defines an unsupported thickness 226.

In some configurations, the total thickness 190 equals a sum of the supported thickness 224 and the unsupported thickness 226. In some configurations, the total thickness 190 equals twice the supported thickness 224. Such an arrangement provides or allows for space for the apertures 166 on the embedded portion of the housing 122 through which the friction member 142 can project while also providing a sufficiently large unsupported thickness 226 to allow for a desired amount of deformation of the friction member 142 when the friction member 142 is connected to the collar 152. In some configurations, the total thickness 190 equals twice the unsupported thickness 226. Such an arrangement allows for a sufficiently large unsupported thickness 226 to allow for a desired amount of deformation of the friction member 142 when the friction member 142 is connected to the collar 152.

In some configurations, the supported thickness 224 is equal to the unsupported thickness 226. Such an arrangement can provide a balance between support or engagement between the housing 122 and the friction member 142 and allowing for deformation of the friction member 142 when the cushion module 120 is assembled to the frame 110. In some configurations, these characteristics are equally desirable and an equal balance between the two can be desirable. However, in other configurations, the supported thickness 224 is greater than or equal to the unsupported thickness 226. Such an arrangement can allow for or facilitate a stronger connection of the friction member 142 to the housing 122 as a result of an increased area of engagement and/or using larger apertures 166 to provide for a mechanical interlock between the friction member 142 and the housing 122. In yet other configurations, the supported thickness 224 is less than or equal to the unsupported thickness 226. Such an arrangement can allow for reduced material use and/or a smaller profile of the friction coupling 140 or entire interface 100 for a given size of gas inlet 130.

In some configurations, the total thickness 190 is greater than or equal to the collar thickness 198. Such an arrangement allows for more substantial compression of the friction member 142 and/or a more flex of the collar 152 to provide an increased retention force and/or to facilitate assembly. However, in other configurations, the total thickness 190 is less than or equal to the collar thickness 198. Such an arrangement allows for reduced material use on the friction member 142, greater expansion of the friction member 142 for case of assembly or reduced flex of the collar 152.

As described with respect to the interface 100 of FIGS. 1-7, a portion of the friction member 142 that overlaps with the collar 152 defines an overlap length 200. A portion of the friction member 142 that does not overlap with the collar 152 defines a non-overlap length 202. A distance between a rearward end of the collar 152 and the embedded portion of the housing 122 (the opening rim 164 and/or the support wall 163) along the direction of assembly 104 defines a support wall offset distance 230. In some configurations, the overlap length 200 is greater than or equal to the support wall offset distance 230. Such an arrangement provides more surface area for engagement between the collar 152 and the friction member 142. However, in other configurations, the overlap length 200 is less than the support wall offset distance 230. Such an arrangement allows for deformation of a larger length of the friction member 142 in comparison to configurations with a larger overlap length 200 and/or a smaller support wall offset distance 230.

In some configurations, the overlap length 200 is greater than or equal to the non-overlap length 202. Such an arrangement provides more surface area for engagement between the collar 152 and the friction member 142. However, in other configurations, the overlap length 200 is less than the non-overlap length 202. Such an arrangement allows for deformation of a larger length of the friction member 142 in comparison to configurations with a larger overlap length 200 and/or a smaller support wall offset distance 230.

In some configurations, the non-overlap length 202 is greater than or equal to a sum of the support wall offset distance 230 and the support wall thickness 204. Such an arrangement can result from the friction member 142 extending rearwardly of the embedded portion of the housing 122, which can provide for a stronger and more durable connection between the friction member 142 and the housing 122. However, in other configurations, the non-overlap length 202 is less than or equal to a sum of the support wall offset distance 230 and the support wall thickness 204. Such an arrangement can allow for the use of a reduced amount of material for the friction member 142.

In some configurations, the overlap length 200 equals the collar length 186. Such an arrangement allows for an efficient use of the length of the collar 152. However, in other configurations, the overlap length 200 is less than the collar length 152. Such an arrangement allows for the possibility of reduced material for the friction member 142 assuming the friction member 142 is made shorter than the collar 152. In yet other configurations, the overlap length 200 is greater than the collar length 186. Such an arrangement is possible where the collar 152 has, for example, different lengths around the perimeter and the collar length 186 is taken as a shorter length than the maximum length of the collar 152 that overlaps with the friction member 142. Such an arrangement provides for varying amounts of overlap around the perimeter of the collar 152.

In some configurations, the total friction member length 180 equals a sum of the overlap length 200 and the non-overlap length 202. In some configurations, the overlap length 200 is less than the forward length 220. Such an arrangement can allow for more cantilever flex of the friction member 142. In other configurations, the overlap length 200 is greater than or equal to the forward length 220. Such an arrangement can allow for a stronger grip between the friction member 142 and the collar 152. In some configurations, the overlap length 200 is greater than or equal to a sum of the forward length 220, the rearward length 222 and the support wall thickness 204. Such an arrangement can allow for a stronger grip between the friction member 142 and the collar 152. In other configurations, the overlap length 200 is less than or equal to a sum of the forward length 220, the rearward length 222 and the support wall thickness 204. Such an arrangement can allow for more cantilever flex of the friction member 142.

In some configurations, the support wall thickness 204 is less than or equal to the rearward length 222. Such an arrangement can allow for more flex or deformation of the friction member 142. In other configurations, the support wall thickness 204 is greater than the rearward length 222. Such an arrangement can allow for a more rigid friction member 142, if desired.

The housing 122 of the interface 100 of FIGS. 8-12 can include apertures 166 through which the material of the friction member 142 extends. Such an arrangement provides for a mechanical interlock between the friction member 142 and the housing 122. In some configurations, at least a portion of the apertures 166 are located between the opening rim 164 and the peripheral surface 146.

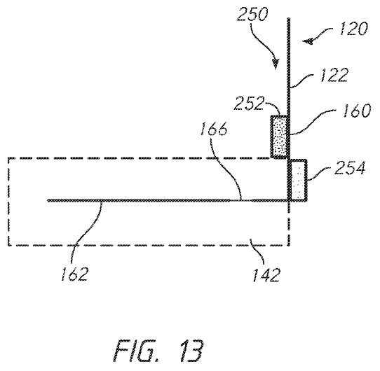

FIG. 13 illustrates a portion of the cushion module 120 of FIGS. 1-7 that includes the friction member 142 and a shut-off arrangement 250 of a molding tool for molding the friction member 142 by an over-molding process. In the illustrated arrangement, a first shut-off portion 252 of a first mold tool portion is located radially outward of and adjacent to the friction member 142. A second shut-off portion 254 of a second mold tool portion is located radially outward of and adjacent to the connection opening 132 or support wall 163. The first shut-off portion 252 and the second shut-off portion 254 are configured to receive the main wall 160 of the housing 122 of the cushion module 120 between them. The first shut-off portion 252 and the second shut-off portion 254 are configured to pinch against the main wall 160 of the housing 122 to provide a physical barrier that retains the injection molded silicone within the mold tool at the surface transitions between the friction member 142 and the housing 122.

As illustrated, a rearward end of the friction member 142 is flush or substantially flush with an interior surface of the main wall 160. To achieve this arrangement, the first shut-off portion 252 and the second shut-off portion 254 are offset from one another in a radial direction. As a result, mold closing forces acting on the shut-off portions 252, 254 are not directly opposed to each other, but are also offset in a radial direction.

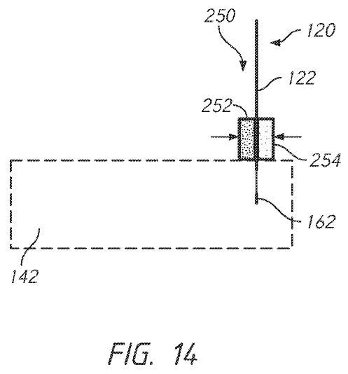

FIG. 14 illustrates a portion of the cushion module 120 of FIGS. 8-12 that includes the friction member 142 and a shut-off arrangement 250 of a molding tool for molding the friction member 142 by an over-molding process. The shut-off arrangement 250 of FIG. 14 can be the same as or similar to the shut-off arrangement 250 of FIG. 13 except as described below. In the illustrated arrangement, a first shut-off portion 252 of a first mold tool portion is located radially outward of and adjacent to the friction member 142 on an exterior side of the main wall 160. A second shut-off portion 254 of a second mold tool portion is located radially outward of and adjacent to the friction member 142 on an interior side of the main wall 160.

As illustrated, a rearward end of the friction member 142 extends rearwardly of an interior surface of the main wall 160 toward the seal 124 (the seal 124 is not shown in FIG. 14—see, for example, FIG. 8) of the cushion module 120. In the illustrated arrangement, a radially outer surface of the friction member 142 on an exterior side of the main wall 160 is aligned in a radial direction with a radially outer surface of the friction member 142 on an interior side of the main wall 160. With such an arrangement, the first shut-off portion 252 and the second shut-off portion 254 are aligned with or oppose one another in a radial direction. As a result, mold closing forces acting on the shut-off portions 252, 254 are directly opposed to each other in a radial direction. Such an arrangement may reduce flexing of the housing 122 when the mold is closed relative to the arrangement of FIG. 13, which could improve sealing of the mold portions against the housing 122 and reduce flashing of the friction member 142 material.

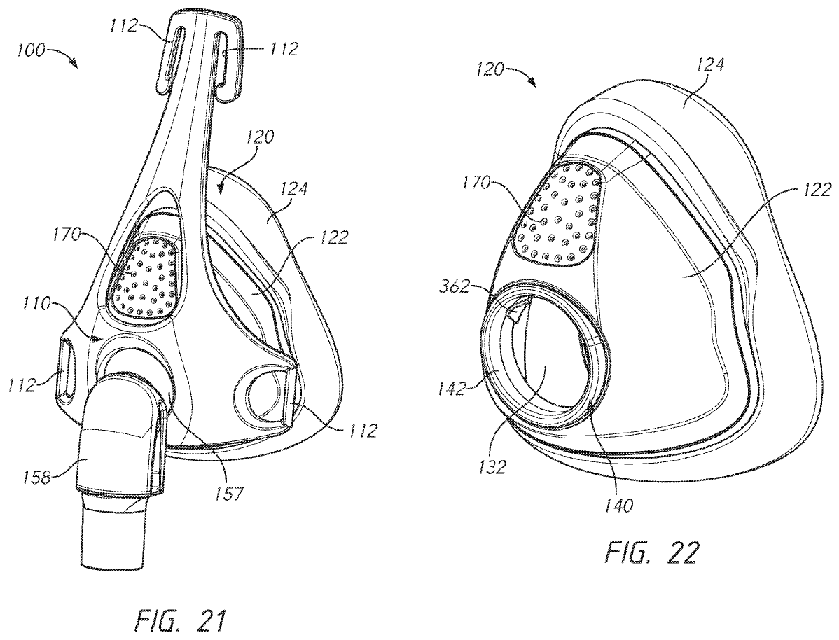

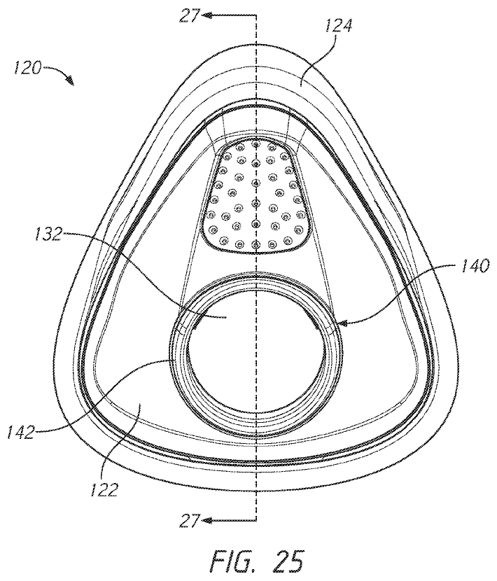

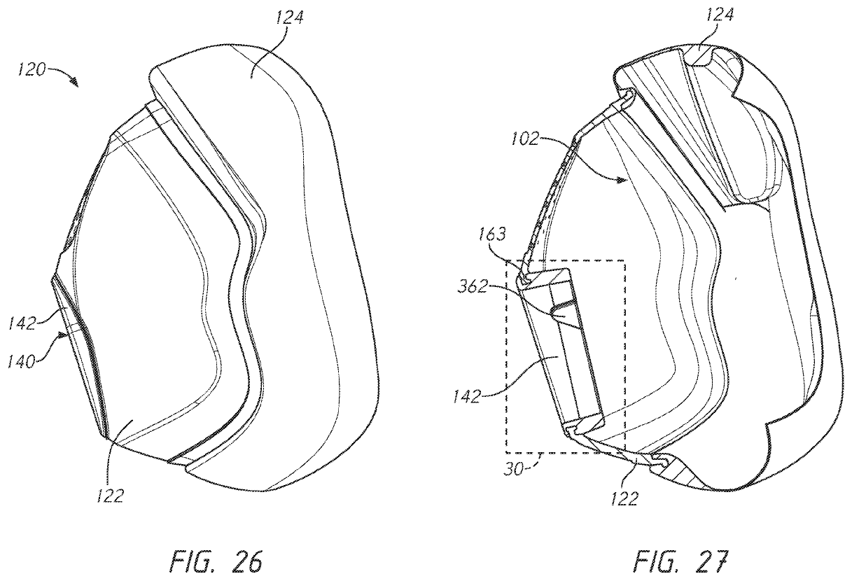

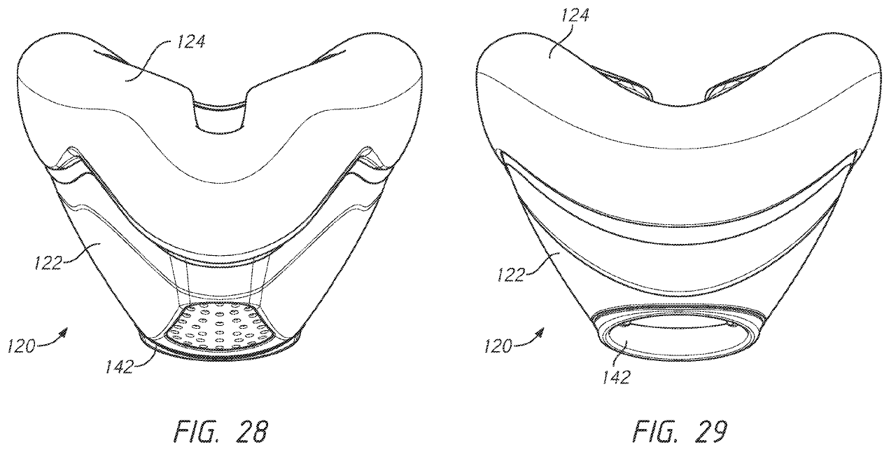

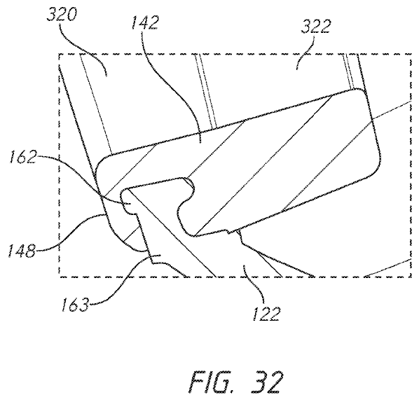

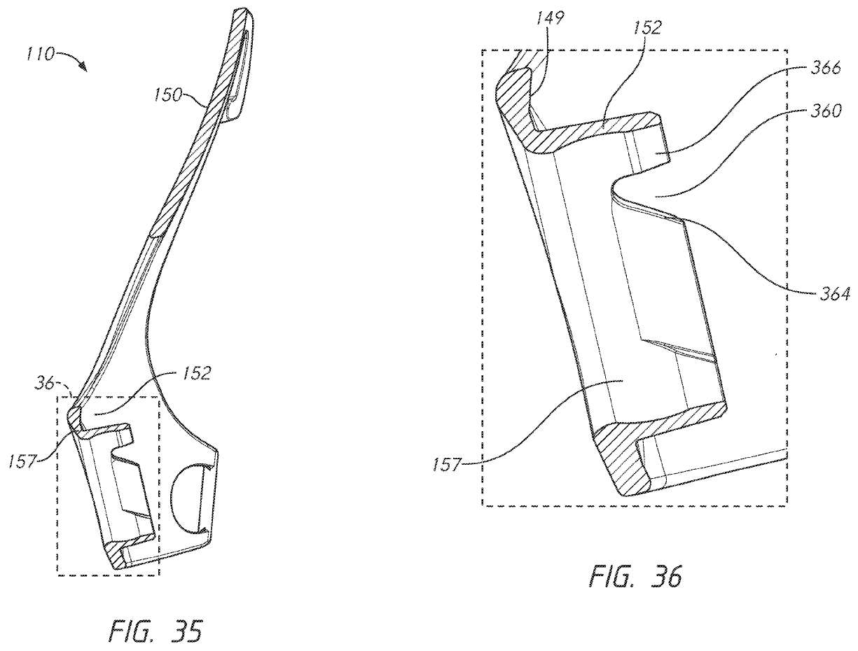

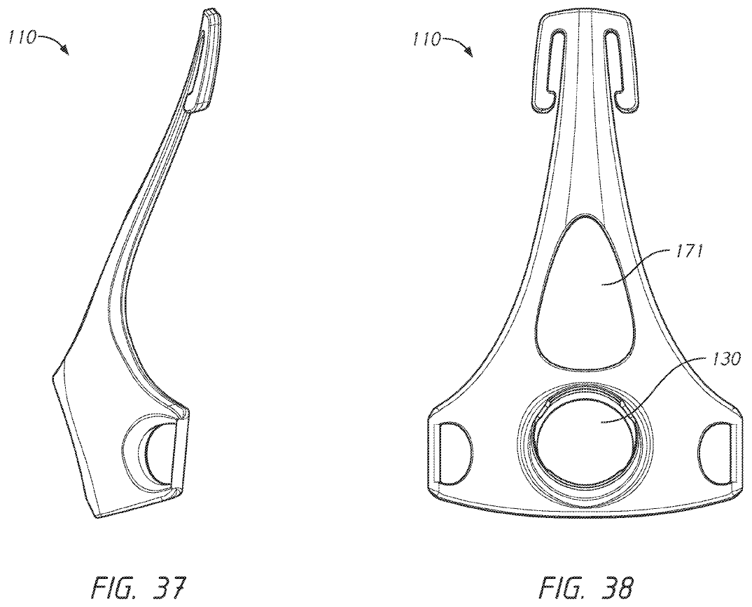

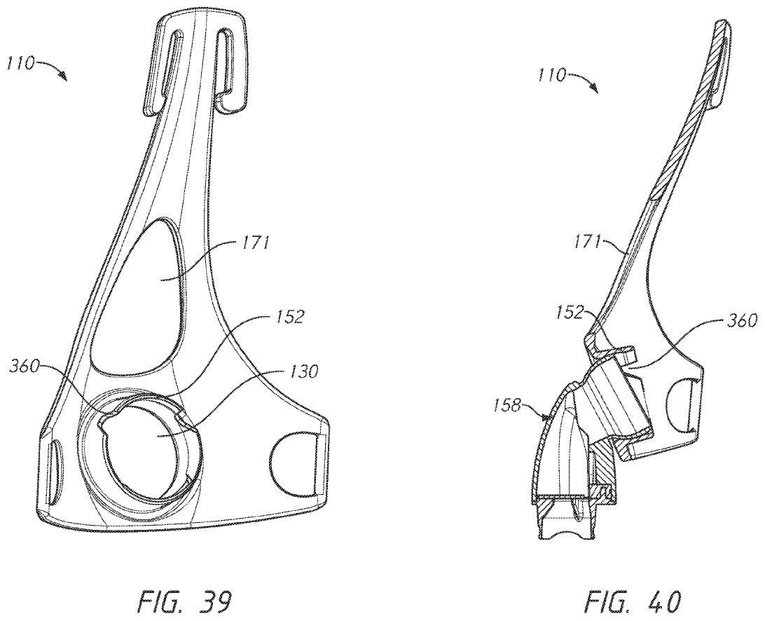

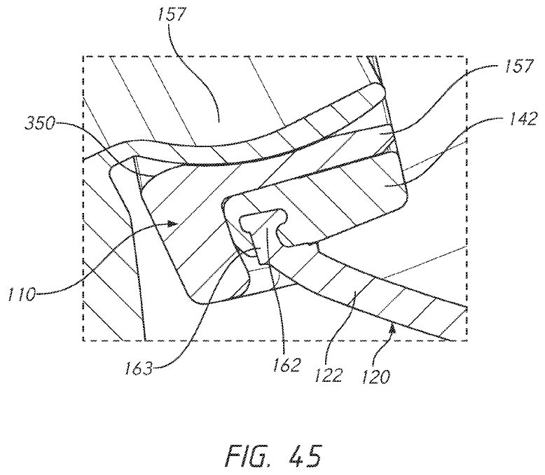

The patient interface 100 and portions thereof shown in FIGS. 21-45 is similar in many respects to the other interfaces disclosed herein. Accordingly, the same reference characters can be used to refer to the same or similar components or features and the following description is focused on the unique features of the interface 100 relative to the other interfaces. Accordingly, the interface 100 can incorporate features of the other interfaces disclosed herein. In addition, features that are not disclosed in detail can be the same as or similar to other interfaces disclosed herein, or can be of another suitable arrangement.

The patient interface 100 includes a frame 110 and a cushion module 120 removably coupled to the frame 110. The frame 110 is configured to be connected to headgear (not shown), which holds the patient interface 100 in a sealed relation to the user's face. The illustrated frame 110 includes a pair of upper headgear mounts 112 and a pair of lower headgear mounts 112. However, the frame 110 could include other desirable number of headgear mounts depending on the type of headgear desired to be used with the patient interface 100. The mounts 112 can be configured to directly or indirectly receive an associated headgear strap(s).

The cushion module 120 defines at least a portion of the breathing chamber 102 (FIG. 27) of the patient interface 100. The cushion module 120 includes a housing 122 and a cushion or seal 124. Preferably, the housing 122 includes a wall that extends in both a radial and axial direction relative to an axis 104 along which the frame 110 and the cushion module 120 overlap or are coupled. Thus, the housing 122 defines a depth in an anteroposterior direction. In some configurations, the axis along which the frame 110 and the cushion module 120 overlap is generally or substantially aligned with the assembly axis or direction of assembly between the frame 110 and the cushion module 120. An axis of a gas inlet 130, or z-axis 104, of the frame 110 and/or the cushion module 120 can be aligned with the assembly axis.

The seal 124 is coupled to a rear or posterior portion of the housing 122.

The seal 124 defines a face-contacting surface configured to contact the face of a user of the interface 100 and create a seal or substantial seal with the user's face. The seal 124 can be configured such that it surrounds either or both of the nose and mouth of the user. The scal 124 can be an inflation-type seal having an interior-concave curved wall that inflates when the interior of the interface 100 is pressurized to provide a good seal and better conform to the face of the particular user. However, other types of seals can be used, if desired. Moreover, certain features, aspects and advantages of the present disclosure can be utilized with other types of patient interfaces, such as direct nasal interfaces, among others.

With reference to FIG. 27, the wall of the housing 122 surrounds, defines or includes a first opening (connection opening 132) at a forward end and a second opening at a rearward end. As discussed above, the seal 124 is connected to the second opening at the rearward end of the housing 122. Accordingly, the wall of the housing 122 extends from the first opening in a rearward direction towards the second opening. Preferably, the first opening is smaller than the second opening. That is, the second opening can have a larger vertical and/or lateral dimension than the first opening. In some configurations, a perimeter of the second opening is larger than a perimeter of the first opening. In some configurations, an area of the second opening is larger than an area of the first opening. In some configurations, because it is configured for connection to the frame 110, which provides for connection to headgear, the cushion module 120 does not include integrated headgear connector structures.

In some configurations, the housing 122 has a higher hardness and/or is more rigid than the seal 124. The housing 122 can be constructed from a more rigid material than the material of the seal 124. For example, the housing 122 can be constructed from a relatively rigid plastic (e.g., polycarbonate). The seal 124 can be constructed partially or completely from a less rigid material than the material of the housing 122, which can be a relatively soft, flexible, pliable, resilient or elastic material, such as an elastomer (e.g., silicone), a foam or a textile. In some configurations, the seal 124 is coupled to the housing 122 during a molding process, such as an over-molding process. In addition, or in the alternative, the seal 124 may be mechanically coupled to the housing 122. For example, the housing 122 can include a plurality of apertures through which the material of the seal 124 passes to create a mechanical interlock between the seal 124 and the housing 122. Preferably, the housing 122 provides sufficient strength and rigidity to maintain the breathing chamber 102 during use.

In the illustrated arrangement, the housing 122 of the cushion module 120 includes a vent 170 in fluid communication with the breathing chamber 102. The vent 170 is configured to allow a flow of breathing gases and/or gases exhaled by the user to pass from the breathing chamber 102 to the atmosphere external of the patient interface 100. As previously described, the vent 170 can be a bias flow vent comprising a plurality of small holes. The vent 170 can be located in a nasal region of the cushion module 120. The frame 110 can include a vent opening 171 that is aligned with and accommodates the vent 170 when the cushion module 120 is connected to the frame 110.

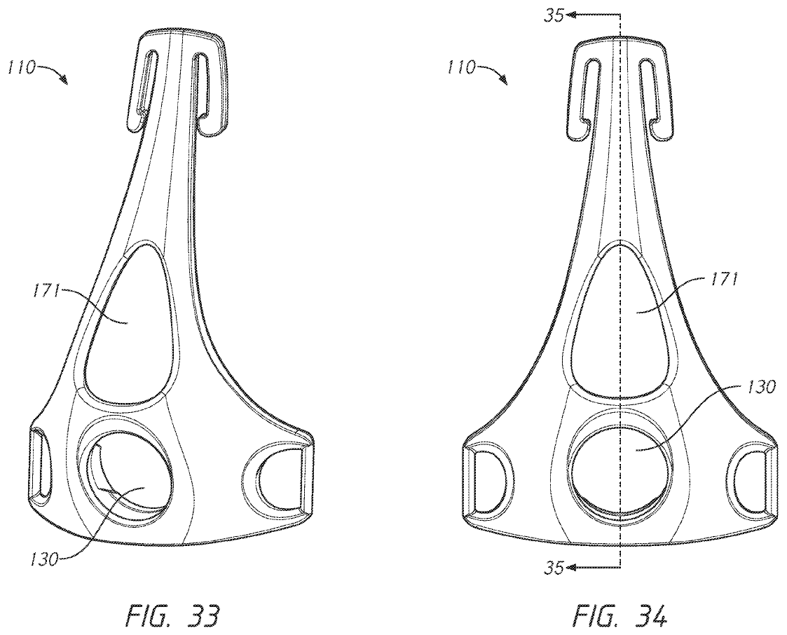

As described above, the frame 110 defines or otherwise includes a frame opening or gas inlet 130 (FIG. 33) that permits a flow of breathing gas to pass through the frame 110. The cushion module 120 includes a gas inlet, cushion module opening or connection opening 132 (FIG. 22) that permits a flow of breathing gas to pass through the cushion module 120. When the cushion module 120 is assembled to the frame 110, the connection opening 132 of the cushion module 120 is aligned with the gas inlet 130 of the frame 110 such that a flow of breathing gas is permitted to pass through the gas inlet 130 of the frame 110 and the connection opening 132 of the cushion module 120 into the breathing chamber 102.

A friction coupling 140 selectively couples the cushion module 120 and the frame 110. The friction coupling 140 can be designed in accordance with the criteria or methodology described above with reference to the embodiments of FIGS. 1-20. In some arrangements, the friction coupling 140 is the only coupling between the frame 110 and the cushion module 120. In some configurations, the friction force provided by the friction coupling 140 is the only retention force that holds the cushion module 120 and the frame 110 together when the interface 100 is not positioned on a user. That is, the friction coupling 140 can be the only direct coupling between the frame 110 and the cushion module 120. However, in other arrangements, the patient interface 100 can include additional couplings between the frame 110 and the cushion module 120.

In the illustrated arrangement, the friction coupling 140 is or comprises an elastomeric friction member 142 that creates an airtight or substantially airtight seal between the frame 110 and the cushion module 120. With such an arrangement, leakage of breathing gas (e.g., air) between the frame 110 and the cushion module 120 can be prevented or sufficiently inhibited such that the desired positive pressure can be maintained within the breathing chamber 102. The elastomeric friction member 142 can have a higher hardness than the seal 124. For example, the friction member 142 can have a 60-80 shore hardness and the seal 124 can have a lower shore A hardness, such as about a 40 shore A hardness, for example. In some configurations, the elastomeric friction member 142 can have the same hardness as the seal 124 and/or can be constructed from the same material. For example, the elastomeric friction member 142 and the seal 124 can each have a 40 shore A hardness. At least a surface of the friction member 142 that engages the frame 110 can have a textured surface finish, which as described previously herein can provide a desirable balance between retention and allowing sliding movement for assembly and disassembly.

The illustrated frame 110 includes at least a main wall or a front wall 150 and a collar 152, which extends rearwardly (e.g., posteriorly or toward the user) from the front wall 150 to a rearward or free end, edge or rim and surrounds the gas inlet 130. In the illustrated arrangement, the perimeter defined by the collar 152 is circular, or generally or substantially circular. However, non-circular shapes could also be used, as described herein. The front wall 150 and the collar 152 can be unitarily formed of a material with sufficient strength and rigidity to transfer headgear forces from headgear to the cushion module 120 and/or maintain a shape of the collar 152 or any portion of the breathing chamber 102 defined by the frame 110 during use. For example, the frame 110 can be formed partially or entirely from a relatively rigid polymeric material (e.g., polycarbonate), by a process such as molding (e.g., injection molding).

In the arrangement of FIGS. 21-45, and any of the arrangements described elsewhere herein, a property can differ between any one or more of the components of the patient interface 100 and any other of the components. For example, one or more properties can differ between any one of the frame 110, housing 122, friction member 142 and the seal 124. Such properties can include, for example, material, modulus of elasticity, hardness, stiffness, rigidity or any combination thereof. In some configurations, one or both of the frame 110 and the housing 122 is more rigid or harder than one or both of the friction member 142 and the seal 124. In at least one configuration, the housing 122 is more rigid or harder than the friction member 142. In at least one configuration, the housing 122 is more rigid or harder than the seal 124. In at least one configuration, the frame 110 is more rigid or harder than the friction member 142. In at least one configuration, the frame 110 is more rigid or harder than the seal 124. Such arrangements can permit the frame 110 and/or the housing 122 to maintain a desired shape and transfer loads and permit the friction member 142 and/or the seal 124 to better conform to and/or seal against the frame 110 and the user, respectively.

In some configurations, one or both of the frame 110 and the housing 122 is less rigid, less hard or softer than one or both of the friction member 142 and the seal 124. In at least one configuration, the housing 122 is less rigid, less hard or softer than the friction member 142. In at least one configuration, the housing 122 is less rigid, less hard or softer than the seal 124. In at least one configuration, the frame 110 is less rigid, less hard or softer than the friction member 142. In at least one configuration, the frame 110 is less rigid, less hard or softer than the seal 124. Such arrangements can permit the frame 110 and/or the housing 122 to deform in response to loads when desirable, such as when other structures are provided to absorb the applied loads.

In some configurations, one or both of the frame 110 and the housing 122 is of the same rigidity or hardness as one or both of the friction member 142 and the seal 124. Such arrangements can include the housing 122 being formed as a unitary structure with one or both of the friction member 142 and the seal 124. The housing 122 can be the same rigidity or hardness as the frame 110, can be more rigid or harder than the frame 110, or can be less rigid, less hard or softer than the frame 110. Such arrangements allow the relative rigidity or hardness of the frame 110 and the housing 122 to be selected to facilitate deformation in one of the frame 110 or the housing 122 and/or to reduce or prevent deformation in one of the frame 110 or the housing 122, as desired.

As described above, the friction member 142 can have a different rigidity and/or hardness than the seal 124. In at least one configuration, the friction member 142 can be more rigid or harder than the seal 124, or the seal 124 less rigid or softer than the friction member 142. Such an arrangement can provide for improved durability of the friction member 142 and increased comfort of the seal 124. Also as described above, the friction member 142 can be of the same rigidity or hardness as the seal 124. Such an arrangement can provide for improved sealing and/or retention of the friction member 142. In other configurations, the friction member 142 could be less rigid, less hard or softer than the seal 124. Such an arrangement could provide for enhanced conformance of the friction member 142 relative to the frame 110 and/or sealing of the friction member 142 with the frame 110, while the seal 124 is configured for the desired level of support and comfort for the user.

In at least one configuration, the friction member 142 and the seal 124 can comprise a common material, for example a silicone. The friction member 142 and scal 124 of said configuration can have the same or substantially similar rigidity and/or hardness. Alternatively, the friction member 142 and scal 124 of said configuration can have different rigidities. Alternatively, each of the friction member 142 and scal 124 of said configuration can have a different hardness. For example, the friction member 142 and the seal 124 can comprise silicone. The silicone friction member 142 can have a 70 shore A hardness, and the silicone seal 124 can have a 40 shore A hardness. In said configuration, the friction member 142 and the seal 124 comprise a common material, and are of differing hardness, such that the seal 124 has a lower hardness than the friction member 142.

In at least one configuration, the friction member 142 and the seal 124 can comprise an uncommon material. In other words, the friction member 142 can comprise a material that the seal 124 does not comprise, or the seal 124 can comprise a material that the friction member 142 does not comprise. The friction member 142 and scal 124 of said configuration can have the same or substantially similar rigidity and/or hardness. Alternatively, the friction member and scal 124 of said configuration can have different rigidities. Alternatively, each of the friction member 142 and scal 124 of said configuration can have a different hardness. In at least one configuration, the uncommon material can be an additive. In at least one configuration, the friction member 142 and the seal 124 comprise no common materials such that the friction member 142 and scal 124 are formed from entirely different materials, or materials with different atomic structures.

In at least one configuration, the frame 110 and the housing 122 can comprise a common material, for example a polycarbonate. The frame 110 and housing 122 of said configuration can have the same or substantially similar rigidity and/or hardness.

Alternatively, the frame 110 and the housing 122 of said configuration can have different rigidities and/or each can have a different hardness.

In at least one configuration, the frame 110 and the housing 122 can comprise an uncommon material. In other words, the frame 110 can comprise a material that the housing 122 does not comprise, or the housing 122 can comprise a material that the frame 110 does not comprise. The frame 110 and the housing 122 of said configuration can have the same or substantially similar rigidity and/or hardness. Alternatively, the frame 110 and the housing 122 of said configuration can have different rigidities. Alternatively, each of the frame 110 and the housing 122 of said configuration can have a different hardness. In at least one configuration, the uncommon material can be an additive. In at least one configuration, the frame 110 and the housing 122 comprise no common materials such that the frame 110 and the housing 122 are formed from entirely different materials, or materials with different atomic structures.

The collar 152 of the frame 110 defines a socket 157 configured to receive a conduit connector, such as a conduit connector portion 158, configured to connect a breathing circuit to the patient interface 100. In at least one embodiment, the conduit connector portion 158 can be in the form of an elbow 158. However, in other arrangements, the conduit connector can be connected to other portions or components of the patient interface 100, such as a portion of the cushion module 120 (e.g., the housing 122). In some configurations, the collar 152 defines at least a portion of the elbow socket 157. The conduit connector or elbow can be capable of pivoting about one or more axes relative to the frame 110. For example, the conduit connector or elbow can have a swivel connection with the frame 110.

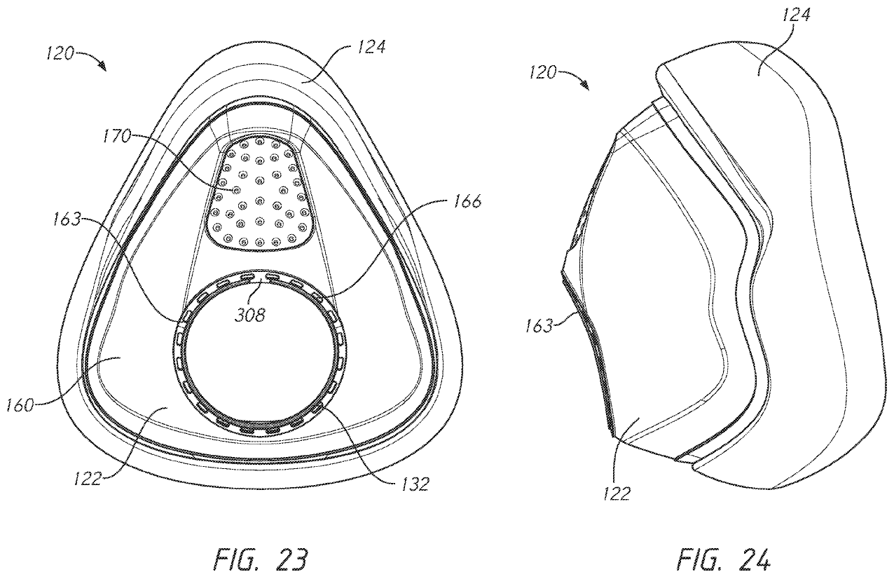

The housing 122 of the cushion module 120 includes a main wall 160 and a support flange 162. The support flange 162 and/or a portion of the main wall 160 surrounds and/or defines the connection opening 132. The housing 122 also includes a support wall 163 that surrounds and is located between the main wall 160 and the support flange 162. The support flange 162 defines a circular, generally circular or substantially circular perimeter shape from a front view that preferably corresponds to the perimeter shape of the collar 152, but preferably is somewhat larger to accommodate the portion of the friction member 142 therebetween. In the illustrated arrangement, the support flange 162 and the support wall 163 are not planar, but are curved from a side view with a slightly forward-facing concave shape, as illustrated in FIG. 24. The illustrated support wall 163 extends radially inward from the main wall 160; however, the support wall 163 could also or alternatively extend in a somewhat forward or rearward direction.

The elastomeric friction member 142 is carried by or coupled to the cushion module 120. In particular, the friction member 142 is coupled to the support flange 162 and/or the support wall 163. In the illustrated arrangement, a portion or an entirety of the support wall 163 and the support flange 162 are embedded within the friction member 142 to form a mechanical anchor to secure the friction member 142 to the housing 122. The support flange 162 and/or the support wall 163 can also provide hoop strength to the friction member 142 or limit outward expansion of the friction member 142 to enhance the friction fit with the collar 152 of the frame 110.

The friction member 142 can be permanently coupled to the support flange 162 and/or the support wall 163, such as by an over-molding process. The support flange 162 and/or the support wall 163 can include one or more apertures 166 through which the material of the friction member 142 extends as a result of the over-molding process. Such an arrangement provides a mechanical interlock between the friction member 142 and the housing 122. In the illustrated arrangement, the support wall 163 includes the plurality of apertures 166, each of which extends axially through the support wall 163. In some configurations, the apertures 166 are evenly distributed around the perimeter of the support wall 163. In other configurations, the apertures 166 can be unevenly distributed around the perimeter of the support wall 163.

The apertures 166 preferably are sized such that the portions of the friction member 142 passing therethrough are strong enough to resist breaking in response to normal or expected forces acting on the friction member 142 taking into account the material properties of the friction member 142. If the size of the apertures 166 is too small, the portions of the friction member 142 extending through the apertures 166 can possibly tear or break in response to normal or expected forces in use. If a sufficient number of the portions of the friction member 142 extending through the apertures 166 break, the remaining portions can be insufficient to absorb the forces applied to the friction member 142, which can result in cascading breakage of additional portions of the friction member 142 extending through the apertures 166. Conversely, if the apertures 166 are too large, the support wall 163 may not be strong enough to absorb normal or expected forces applied to the cushion module 120, such as from compression or squeezing of the cushion module 120, or large forces applied to the friction member 142. In some configurations, the apertures 166 can have a dimension of about 1 mm in a radial direction and/or about 3 mm in a circumferential direction. In other words, the ratio of a radial dimension of the apertures 166 and a circumferential dimension of the apertures 166 can be about 1:3. Each aperture 166 can be circumferentially spaced apart from each adjacent aperture 166 by an angle of about 10-25°, about 15-20°, or about 18° with respect to a central point of the connection opening 132.

In addition or in the alternative of a mechanical interlock between the friction member 142 and the housing 122 (such as provided by the apertures 166), a self-adhesive elastomer, such as a self-adhesive silicone, can be used to form a chemical bond the friction member 142 and the corresponding adjacent surface(s) of the housing 122. Such an arrangement can be used alone or in combination with an interlock arrangement. For example, the chemical bond of the self-adhesive material can provide enhanced connection on surfaces in which it is difficult, impractical or impossible to form apertures 166. As described herein, it may be beneficial to form the apertures 166 in the pull direction of the mold tools. For example, with reference to a general arrangement as shown in FIGS. 1-7, if the apertures 166 are formed in a first portion (e.g., the main wall 160) of the housing 122, the chemical bond can enhance the connection with a second portion (e.g., the support wall 163) of the housing 122, which second portion does not include apertures 166 and/or is oriented perpendicular to or at an oblique angle with respect to the first portion.

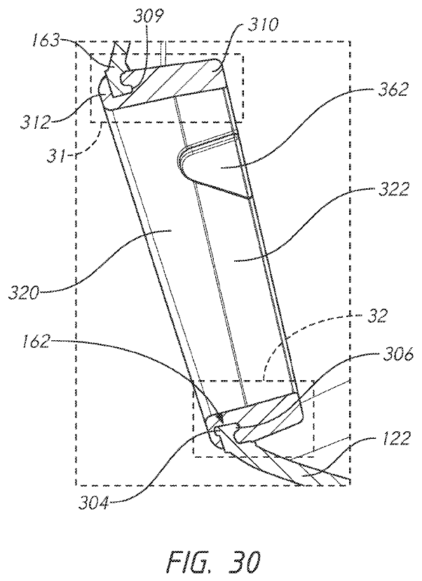

With reference to FIGS. 30-32, a radially-inward edge of the support wall 163 is coupled to the support flange 162. The support flange 162 defines a length that is larger than a thickness of a portion or an entirety of the support wall 163. In other words, an axial dimension of the support flange 162 is greater than an axial dimension of at least a portion or an entirety of the support wall 163. The support flange 162 increases the strength and reduces deformation of the support wall 163 in comparison to an arrangement without a support flange 162. The support flange 162 can also assist with the mechanical interlock of the friction member 142 to the cushion module 120 and/or can provide support to the friction member 142 such that the friction coupling 140 can exhibit desirable characteristics related to the assembly and disassembly of the cushion module 120, as described above with respect to FIGS. 1-12, for example.

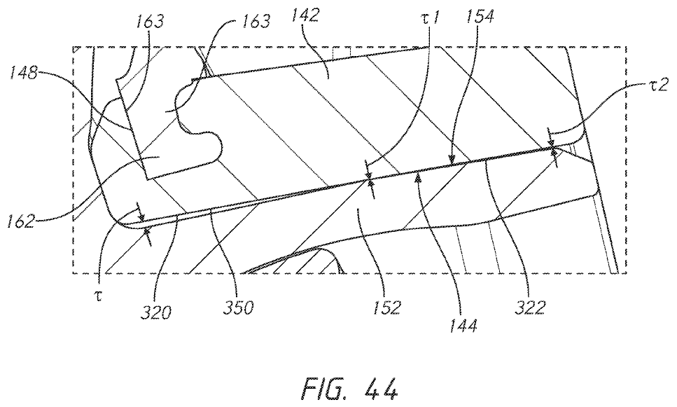

The support flange 162 can extend in either one or both axial directions relative to the support wall 163. In the illustrated arrangement, the support flange 162 includes an outer flange portion 304 (distal relative the user) and an inner flange portion 306 (proximal relative the user). The outer flange portion 304 and/or the inner flange portion 306 can completely surround the connection opening 132. In other words, the outer flange portion 304 and the inner flange portion 306 can define a closed loop. However, either or both of the outer flange portion 304 and the inner flange portion 306 can be interrupted in the circumferential direction. In the illustrated arrangement, the outer flange portion 304 includes an interruption 308 (FIG. 23) at an upper portion of the support flange 162. The interruption 308 can accommodate a gate in the molding tool used to mold the housing 122 of the cushion module 120. One or both of an axial dimension (length) or radial dimension (width) of the support flange 162, including one or both of the outer flange portion 304 and the inner flange portion 306, can be constant or variable in a circumferential direction. For example, the axial and/or radial dimension can be relatively larger at or near a location or region in which additional support is desirable, such as within the inner flange portion 306 in the illustrated arrangement in which the interference is the greatest, as described below with reference to FIG. 44.

The support flange 162 can be sized and shaped to provide a sufficient or desirable level of reinforcement to the support wall 163 and/or mechanical interlocking with the friction member 142. In the illustrated arrangement, a ratio of the axial dimension of the support flange 162 to an axial dimension of support wall 163 can be between about 1.375:1, 2:1, or 3:1 to about 8:1 (or greater), between about 4:1 to about 8:1, or about 6:1. In some configurations, the support wall 163 has an axial dimension or thickness of about 1.4-1.8 mm, or about 1.6 mm. The support flange 162 can have a total axial dimension or length of between about 2.2 mm, 3.2 mm, or 4.8 mm to about 12.8 mm, between about 6.4 mm to 12.8 mm, or about 9.6 mm or 10 mm. In some configurations, each of the outer flange portion 304 and the inner flange portion 306 can have equal axial dimensions or lengths. In other arrangements, the axial dimension or length of a portion or an entirety of the inner flange portion 306 can be selected to extend within a substantial portion of the friction member 142 in a manner similar to the embodiment of FIGS. 1-7. In some such arrangements, a length of the support flange 162 or the inner flange portion 306 is variable in the circumferential direction or around the perimeter of the support flange 162, such as relatively longer on the upper portion and relatively shorter on the lower portion. As described above, the thickness of the support wall 163 can also vary in the circumferential direction or around the perimeter of the support wall 163.

One or both of the outer flange portion 304 and the inner flange portion 306 can have rounded shapes to reduce stress concentrations in the support flange 162 and the boundaries between the support flange 162 and the friction member 142 in comparison to an arrangement with sharp corners. For example, an axial end of each of the outer flange portion 304 and the inner flange portion 306 can have a rounded edge 309. In the illustrated arrangement, the rounded edge 309 extends across the entire axial ends of the outer flange portion 304 and the inner flange portion 306. However, in other arrangements, only the corners of the axial ends are rounded. In some configurations, a transition between the support wall 163 and one or both of the outer flange portion 304 and the inner flange portion 306 can be rounded.

As described, the support flange 162 and/or the support wall 163 are embedded within the friction member 142. A portion of the friction member 142 is located on a forward side (distal the user) of the support wall 163. In the illustrated arrangement, the portion of the friction member 142 located on the forward side of the support wall 163 is relatively small compared to a portion of the friction member 142 located on a rearward side (proximal the user) of the support wall 163. In some configurations, the portion of the friction member 142 located on the forward side of the support wall 163 is sufficient to provide a suitable or desirable level of engagement or interlocking with the support flange 162 and/or the support wall 163 and/or to cover the support flange 162 and/or the support wall 163 to provide the cushion module 120 with an attractive appearance. As described above, in some configurations the support wall 163 is non-planar. In some such configurations, an axial dimension or length of the portion of the friction member 142 located on the forward side of the support wall 163 varies around the circumference of the friction member 142. In some configurations, a length of the portion of the friction member 142 located on the forward side of the support wall 163 is greater at the sides than at the top and bottom. In some configurations, the support wall 163 defines a planar forward surface.

The portion of the friction member 142 located on the rearward side of the support wall 163 is significantly greater than the portion located on the forward side of the support wall 163. In the illustrated arrangement, the connection opening 132 is positioned below the nasal region of the cushion module 120 and can be within an oral region of the cushion module 120 and/or approximately aligned with a mouth of the user. Accordingly, the primarily rearward projection of the illustrated friction member 142 takes advantage of the available space within the oral region of the breathing chamber 102 located below the user's nose. Such an arrangement allows the frame 110 and the conduit connector portion 158 to be located closer to the user's face, which reduces the effect of hose pull forces on the interface 100.

The portion of the friction member 142 located on a rearward side of the support wall 163 defines an unsupported portion 310 of the friction member 142, which can define an unsupported length dimension in the axial direction. In other words, the unsupported portion 310 is not directly supported in a radial direction by the support wall 163 or the support flange 162. A portion of the friction member 142 radially inward from the support wall 163 and/or the support flange 162 defines a supported portion 312 of the friction member 142, which can define a supported length in the axial direction. The supported portion 312 is directly supported in a radial direction by the support wall 163 and/or the support flange 162. Thus, the unsupported portion 310 can deform radially outward or increase in diameter or circumference with less force than the supported portion 312. In some configurations, as described further herein, the unsupported portion 310 applies less radial force to the collar 152 than the supported portion 312.

In some configurations, the interior or radially inward-facing surface, or engagement surface 144 of the friction member 142 includes a first engagement surface portion 320 and a second engagement surface portion 322, each of which is referred to as an “engagement surface” for convenience. The first engagement surface 320 is located forward (distal relative to the user) of the second engagement surface 322. In other words, the second engagement surface 322 is disposed at a generally greater distance away from the support wall 163 relative to the first engagement surface 320. A portion of the first engagement surface 320 and/or a portion of the second engagement surface 322 can be located within the breathing chamber 102 of the cushion module 120. In some configurations, an entirety of the second engagement surface 322 is located within the breathing chamber 102 of the cushion module 120.

In some configurations, the first engagement surface 320 and the second engagement surface 322 are the same size (e.g., length or area) and/or define about one-half of a length or surface area of the engagement surface 144. In the illustrated arrangement, the supported portion 312 defines a portion of the first engagement surface 320. The unsupported portion 310 also defines a portion of the first engagement surface 320. In addition, the unsupported portion 310 defines at least a portion or an entirety of the second engagement surface 322. The engagement surface 144, including the first engagement surface 320 and the second engagement surface 322, faces the collar 152 in use when the cushion module 120 is connected to the frame 110. At least a portion of the engagement surface 144 can be in contact with the collar 152 when the cushion module 120 is connected to the frame 110. In some configurations, as described further below, a portion of the engagement surface 144 can be spaced from the collar 152 when the cushion module 120 is connected to the frame 110. Thus, the use of the term “engagement surface” is used for convenience and does not necessarily require contact with another surface unless otherwise indicated.

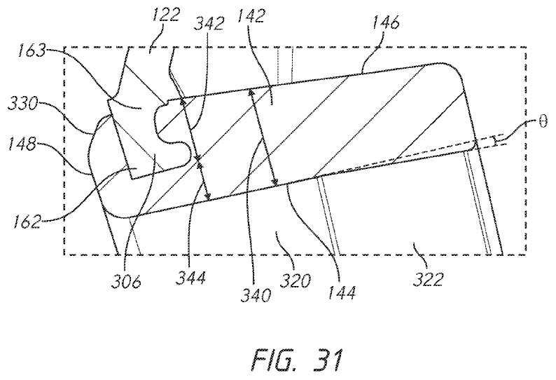

The first engagement surface 320 and the second engagement surface 322 can provide or contribute to the friction coupling 140 having different characteristics between a proximal or rearward portion and a distal or forward portion. In other words, the respective perimeters defined by the first and second engagement surfaces 320, 322 can be different in size (e.g., in perimeter length or perimeter area). In the illustrated arrangement, the first engagement surface 320 and the second engagement surface 322 can be angled (i.e., define an angle θ other than 0 or 180 degrees) relative to one another. The first engagement surface 320 and the second engagement surface 322 can define different angles relative to a longitudinal axis of the friction member 142, which can coincide with the assembly axis and/or z-axis 104. In FIG. 31, the angle θ is illustrated between the second engagement surface 322 and a projection of the first engagement surface 320. The angle θ can be adjusted depending on the material of the friction member 142 (e.g., the type of elastomer or the grade of silicone) and/or the desired force profile for connection and disconnection of the cushion module 120 to the frame 110. For example, the angle θ can be between about 1-5 degrees. In some configurations, the angle θ can be about 3 degrees when a material such as a 70 shore A hardness elastomer (e.g., Silopren® LSR 4070 silicone or a similar material) is used for the friction member 142.

In the illustrated arrangement, at least a substantial portion or an entirety of the first engagement surface 320 is tapered or angled relative to the axis of the friction member 142. At least a substantial portion or an entirety of the second engagement surface 322 is cylindrical or parallel to the axis of the friction member 142. In the illustrated arrangement, the radius or radial dimension of the first engagement surface 320 increases in a forward-to-rearward direction. In other words, an (e.g., circular) area defined within the friction member 142 nearer a forward (distal the user) end of the first engagement surface 320 is smaller than an (e.g., circular) area defined within the friction member 142 nearer a rearward (proximal the user) end of the first engagement surface 320. With such an arrangement, assuming the collar 152 is cylindrical, an engagement or retention force generated by the friction coupling 140 is larger at or near the forward end relative to the forces elsewhere along the friction member 142. The above discussion ignores the rounded forward and rearward-most portions of the interior surface of the friction member 142, which may be provided to reduce stress concentrations and/or to provide a lead-in to facilitate assembly of the cushion module 120 to the frame 110.

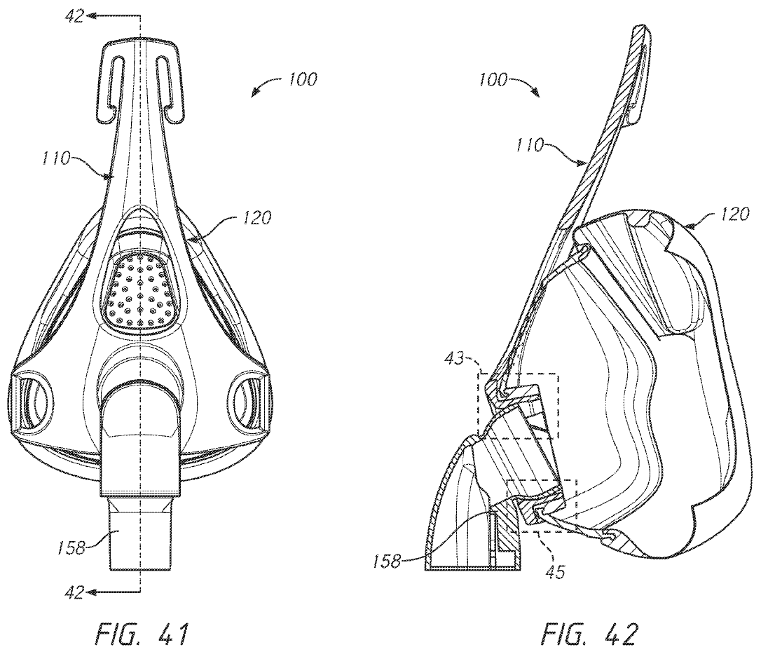

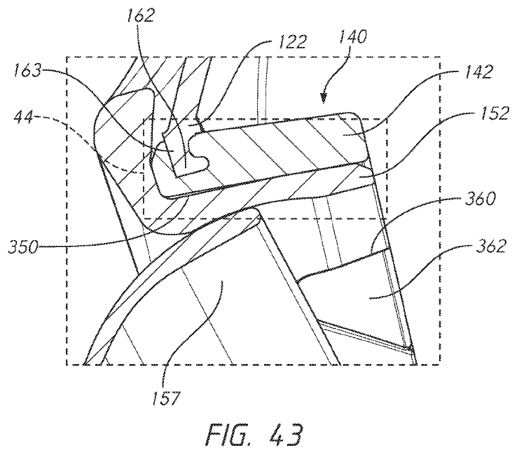

In some configurations, the friction member 142 defines an abutment surface 148 configured to contact a corresponding frame abutment surface 149 of the frame 110 when the cushion module 120 is fully assembled to or engaged with the frame 110. In the illustrated arrangement, the abutment surface 148 is defined by a forward end of the friction member 142. The abutment surface 148 is planar in the illustrated arrangement, but can have any suitable or desirable shape that corresponds to or complements the shape of a surface of the frame 110 contacted by the abutment surface 148. As illustrated in FIGS. 42, 43 and 44, the abutment surface 148 contacts a rearward surface of the front wall 150 adjacent to and/or surrounding the collar 152. The abutment surface 148 can provide feedback to a user that the assembly or engagement of the cushion module 120 to the frame 110 is complete. In some configurations, the abutment surface 148 can provide a seal or substantial seal with the frame 110 in addition to or in place of a seal or substantial seal between the friction member 142 and the collar 152.

In the illustrated arrangement, the friction member 142 includes a chamfered or curved surface 330 radially outward of the abutment surface 148. The curved surface 330 can define a gap or space along with the frame 110, which provides a void into which portions of the friction member 142 can enter as a result of deformation of the friction member 142 when the cushion module 120 is fully engaged with the frame 110. In other words, the void can accommodate expansion of the portion of the friction member 142 forward of the support wall 163 when the cushion module 120 is assembled to or fully engaged with the frame 110. Such an arrangement can improve the performance of the cushion module 120-to-frame 110 connection by allowing for or facilitating complete engagement despite deformation of the forward end of the friction member 142.

With reference to FIG. 31, the friction member 142 can define a wall thickness 340 between the engagement surface 144 and the peripheral surface 146. As described above, because one or both of the first engagement surface 320 and the second engagement surface 322 can be angled relative to the longitudinal axis of the friction member 142, the wall thickness 340 can vary along the length of the friction member 142. The wall thickness 340 can generally correspond to the first thickness or total thickness 190 described with respect to prior embodiments.

In the illustrated arrangement, an embedded portion of the housing 122 defined by the support wall 163 and/or the support flange 162 defines a radial dimension or supported thickness 342. A portion of the friction member 142 radially inward of the support wall 163 (e.g., the supported portion 312) defines a radial dimension or an inner thickness 344. The inner thickness 344 generally corresponds to the fourth thickness 196 described with respect to prior embodiments. A ratio of the supported thickness 342 to the inner thickness 344 or to the wall thickness 340 can be selected to provide or contribute to desirable attributes of the friction coupling 140, as described herein. For example, the supported thickness 342 can be selected to provide for a desirable radial dimension of the apertures 166 (e.g., 1 mm) and/or a desirable radial dimension of the support flange 162 (e.g., 1 mm). The supported thickness 342 can also be selected to provide for a desirable amount of support for the supported portion 312.

The inner thickness 344 can be selected to provide for or facilitate a desirable interference (e.g., frictional) fit with the frame 110, which can provide a sufficient retention force to maintain the cushion module 120 to the frame 110 during normal use and assembly and removal (disassembly) forces that are low enough for easy or relatively easy assembly and removal for an intended user. In some configurations, the assembly and/or removal forces are desirably between about 10-50 Newtons. For example, the inner thickness 344 can be selected such that the compression of the portion of the friction member 142 defining the inner thickness 344 after assembly of the cushion module 120 to the frame 110 provides a desirable retention force or a desirable contribution to the overall retention force. It can be desirable for the inner thickness 344 to be large enough to ensure that manufacturing variations are small enough to have a small impact on the retention force. Such an arrangement provides for a desirable amount of consistency in the assembly/disassembly force and the retention force of the cushion module 120 to the frame 110. A reasonable degree of consistency in the “feel” of assembly and disassembly can be desirable to provide the user with a level of comfort when using a new combination of a frame 110 and a cushion module 120. In some configurations, the inner thickness 344 can be about 1 mm.

FIGS. 42-45 illustrate the friction member 142 of the cushion module 120 engaged with the collar 152 of the frame 110. As described, when the cushion module 120 is fully assembled to the frame 110, the abutment surface 148 of the friction member 142 can contact the frame abutment surface 149 of the frame 110. The engagement surface 144 of the friction member 142 engages the peripheral surface 154 of the collar 152. The engagement surface 144 can engage the peripheral surface 154 with an interference fit along at least a portion of the friction member 142 or the collar 152. As a result of the interference fit, one or both of the friction member 142 and the collar 152 is deformed when the cushion module 120 is assembled to the frame 110.

In some configurations, a retention force generated by the interference fit varies along the length of the friction coupling 140. For example, the collar 152 can be tapered along a portion or an entirety of its length such that a first perimeter defined by a first perimeter portion at a first axial location or section of the collar 152 closer to the front wall 150 is different than a second perimeter defined by a second perimeter portion at a second axial location or section of the collar 152 further from the front wall 150. In the illustrated arrangement, the collar 152 is tapers in a forward-rearward direction such that the first perimeter is greater than the second perimeter. Accordingly, the embedded portion of the housing 122, such as the support wall 163 and/or support flange 162, is located adjacent the first perimeter portion or closer to the first perimeter portion than the second perimeter portion. In such an arrangement, the embedded portion can support the friction member 142 to provide a higher retention force than when the embedded portion is located further from the first perimeter portion. However, in other arrangements, the embedded portion could be adjacent to the second perimeter portion or closer to the second perimeter portion than the first perimeter portion. Such an arrangement can be desirable if a lower retention force and/or a forward-extending friction member 142 is desired.

In some configurations, an interference fit is provided along only a portion of the length of the friction coupling 140. In other words, the retention force can be zero or negligible along at least a portion of the length of the friction coupling 140. For example, as illustrated in FIG. 44, an interference fit 350 can be created along a portion or an entirety of the first engagement surface 320. In contrast, no interference fit or a relatively insignificant interference fit can be created along a portion or an entirety of the second engagement surface 322.

FIGS. 42-45 illustrate the first engagement surface 320 in an as-formed state to illustrate the positive interference fit 350 between the friction member 142 and the collar 152 when the cushion module 120 is assembled to the frame 110. The interference fit 350 represents the total amount of deformation required by the friction member 142 and/or the collar 152 for the cushion module 120 to fit onto the frame 110. In reality, one or both of the friction member 142 and the collar 152 would deform so that the first engagement surface 320 and the corresponding surface of the collar 152 are coincident.

With particular reference to FIG. 44, due to the tapered shape or angle θ of the first engagement surface 320, the amount or the radial dimension of the interference fit 350 varies along a length of the first engagement surface 320 or the friction member 142. In the illustrated arrangement, the degree of the interference fit 350 is at a maximum at or near a forward end of the friction member 142 and decreases in a direction moving toward the rearward end of the friction member 142. A maximum amount or degree of interference is designated by the reference character t. The reference character τ1 indicates the location of minimum interference or an edge of the interference fit 350. The location indicated by the reference character 11 can substantially coincide with a transition between the first engagement surface 320 and the second engagement surface 322. The reference character τ2 indicates a location or region of no interference. For example, the entire second engagement surface 322 can define a region of no interference in which the second engagement surface 322 does not contact or barely contacts a corresponding portion of the collar 152 (e.g., an inner diameter of the second engagement surface 322 is greater than an outer diameter of the corresponding portion of the collar 152).

A length of the friction member 142 and/or the collar 152 can be related to the size and/or the diameter (cross-sectional dimensions) of the opening 130 or 132. For example, a ratio of a length of the friction member 142 and/or the collar 152 to a diameter or a cross-sectional dimension (e.g., vertical or horizontal dimension) can be about 1:2 to about 1:4, or about 1:3. In at least one embodiment, the ratio can be a ratio of the thickness of the friction member 142 and/or the collar 152 to a maximum diameter or maximum cross sectional dimension of the opening 130 or 132. In at least one embodiment, the ratio can be a ratio of the thickness of the friction member 142 and/or the collar 152 to a minimum diameter or minimum cross sectional dimension of the opening 130 or 132. Increasing the relative length of the friction member 142 and/or the collar 152 provides resistance to removal as a result of rotation about a lateral or vertical axis, that is, an axis other than the z-axis. The second engagement surface 322 (or a friction member 142 surface of little or no interference) can be useful to engage the collar 152 when relative non-z-axis rotational forces are applied to the frame 110 and the cushion module 120 to resist instability and/or disconnection of the cushion module 120 from the frame 110.

In at least one configuration, a length of the support flange 162 can be related to the size and/or the diameter (cross sectional dimensions) of the opening 130 or 132. For example, a ratio of the thickness of the support flange 162 to a diameter or cross sectional dimension of the opening 130 or 132 can be about 1:2 to about 1:4, or about 1:3. In at least one embodiment, the ratio can be a ratio of the thickness of the support flange 162 to a maximum diameter or maximum cross sectional dimension of the opening 130 or 132. In at least one embodiment, the ratio can be a ratio of the thickness of the support flange 162 to a minimum diameter or minimum cross sectional dimension of the opening 130 or 132. Increasing the relative length of the support flange 162 provides resistance to removal as a result of rotation about a lateral or vertical axis. That is, an axis other than the z-axis. The second engagement surface 322 (or a friction member 142 surface of little or no interference) can be useful to engage the collar 152 when relative non-z-axis rotational forces are applied to the frame and the cushion module 120 to resist instability and/or disconnection of the cushion module 120 from the frame 110.

An arrangement in which the interference is greater near the forward end of the friction coupling 140 or is only provided within a forward portion (e.g., a portion defined by the first engagement surface 320) advantageously improves the performance of the movable conduit connector portion 158, such as when the conduit connector portion 158 is supported relative to the frame 110 by a swivel arrangement or a ball joint arrangement having one axis or two axes of rotation. Such an arrangement limits or avoids compression forces acting on the rearward portion of the collar 152, which is unsupported by the front wall 150 and more susceptible to deformation. Deformation of the rearward end of the collar 152 in some cases can reduce the size of the elbow socket 157 to such an extent that binding occurs within the ball joint or swivel joint arrangement and movement of the conduit connector portion 158 is impinged. By locating a majority of the interference fit 350 or compressive forces near the forward end (e.g., in the forward half) of the collar 152 where it is supported by the front wall 150 reduces the deformation of the collar 152 such that restriction of movement of the conduit connector portion 158 is reduced or eliminated.

In some configurations, the radial dimension at the axial location identified by the reference character t with respect to the axis of the connection opening 132 is greater than the radial dimension at the axial location identified by the reference character τ1 with respect to a corresponding point disposed along the axis aligned with the location of τ1. In some configurations, the radial dimension at the location identified by the reference character τ is greater than 0 (zero) mm (i.e., positive interference). In some configurations, the radial dimension at the location identified by the reference character τ1 is equal to 0 (zero) mm. In some configurations, the radial dimension at the location identified by the reference character τ2 is less than 0 (zero) mm (i.e., there is no interference). Presently less preferred alternatives include having a constant interference along an entire length of the overlap between the friction member 142 and the collar 152 (τ=τ1=τ2>0), having more interference at the front and rear of the overlap between the friction member 142 and the collar 152 and less in the middle (τ>τ1<τ2), having more interference at the rear of the overlap between the friction member 142 and the collar 152 (τ2≥τ1>τ), and having more interference at the center of the overlap between the friction member 142 and the collar 152 and less at the front and rear (τ<τ1>τ2).

Although a friction member 142 having two discrete engagement surface portions 320, 322 is illustrated, other arrangements that provide for a variable retention force along a length of the friction member 142 (or friction coupling 140) can also be used to vary the retention force as desired, such as in accordance with the principles discussed herein. For example, a curved (e.g., continuously curved) engagement surface 144 could be provided. Such an engagement surface 144 could define a curve between a first end and a second end or between a first axial location and a second axial location. Such an arrangement could include a single curved surface having a constant or variable radius or could comprise multiple curved portions. In other configurations, as described herein, the friction member 142 could include a conical engagement surface, such as in the form of a truncated conc.

In the illustrated arrangement, the collar 152 of the frame 110 includes a pair of recesses 360 extending in a forward direction from a rearward end of the collar 152. However, in other arrangements the collar 152 can include a single recess 360 or more than two recesses 360. In the illustrated arrangement, the recesses 360 are located in an upper portion (e.g., upper half) of the collar 152 and on opposite sides of a central, vertical plane passing in a forward-rearward direction through the frame 110. The friction member 142 of the cushion module 120 includes one or more protrusions 362 corresponding to some or all of the recesses 360. In the illustrated arrangement, the friction member 142 includes a pair of protrusions 362, each configured to be received by one of the pair of recesses 360 of the collar 152 when the cushion module 120 is assembled to the frame 110.

The recesses 360 and the protrusions 362 can have any suitable corresponding or complementary shapes. In some configurations, each protrusion 362 occupies an entirety of the corresponding recess 360 to substantially inhibit or prevent relative rotational movement of the cushion module 120 and the frame 110 about an axis of the friction coupling 140, the friction member 142 and/or the collar 152 (e.g., the z-axis). Rotation about the other axes (e.g., the x-axis and the y-axis) can be substantially inhibited or prevented as a result of the overlap of the friction member 142 and the collar 152. In other configurations, each protrusion 362 can be configured to occupy only a portion of the corresponding recess 360 such that some rotational movement is permitted. However, such an arrangement can provide an indication or guidance as to the correct assembly orientation and can inhibit or prevent incorrect assembly (e.g., the cushion module 120 being oriented upside-down relative to the frame 110).

The recesses 360 can each include at least one angled surface 364 that is angled with respect to, or non-parallel to, the assembly axis 104 or the longitudinal axis of the collar 152. The angled surface 364 can act as a lead-in for the corresponding protrusion 362 so that the cushion module 120 can be guided into the correct orientation during assembly if it is initially misaligned. In the illustrated arrangement, each of the recesses 360 also includes a straight surface 366 that is parallel to, or substantially parallel to, the assembly axis 104 or the longitudinal axis of the collar 152. However, both surfaces 364, 366 could be angled, if desired.

In the illustrated arrangement, the angled surface 364 and the straight surface 366 of each recess 360 are connected by a rounded transition, which reduces stress concentrations. The angled surface 364 and the straight surface 366 cooperate to provide the recess 360 with a generally triangular shape. In the illustrated arrangement, the angled surfaces 364 are located further from a vertical centerline of the frame 110 and the straight surfaces 366 are located closer to the vertical centerline of the frame 110. As described above, each of the protrusions 362 have a shape that corresponds to the generally triangular shape of the recesses 360. Accordingly, each of the protrusions 362 has an angled edge surface and a straight edge surface.

One type of patient interface used with PAP therapy or other respiratory therapies involving the administration of gas to a user, such as those described above, includes a seal that contacts the bridge of the user's nose. The bridge of the nose is sensitive to pressure applied by the seal of the interface assembly. More recently, interface assemblies have been developed that do not contact the bridge of the nose. Such interface assemblies can be referred to as “under-nose” interface assemblies. Such under-nose interfaces can be nasal or oro-nasal (“full face”) interfaces.

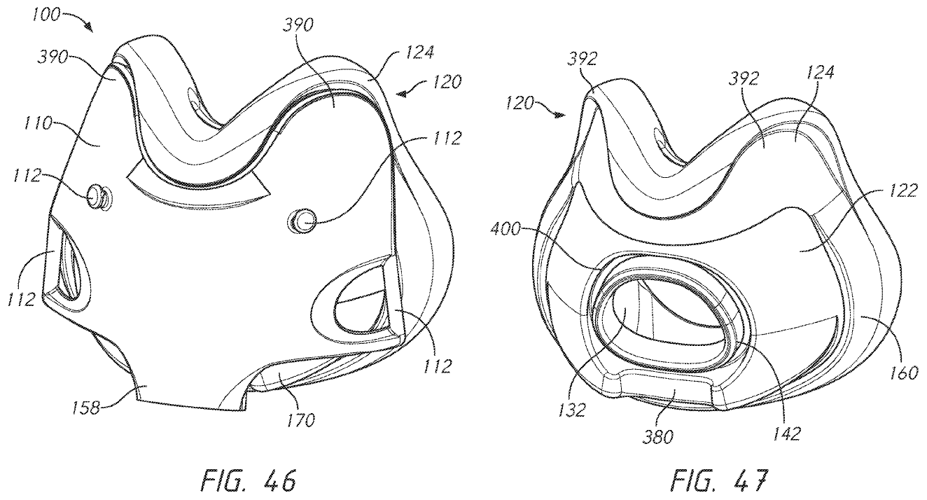

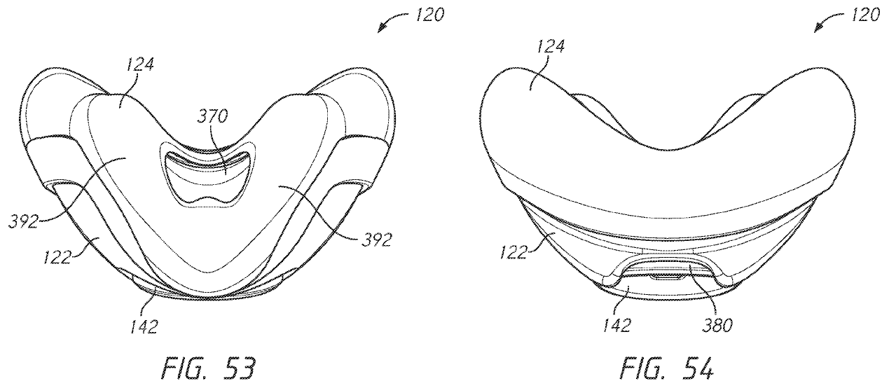

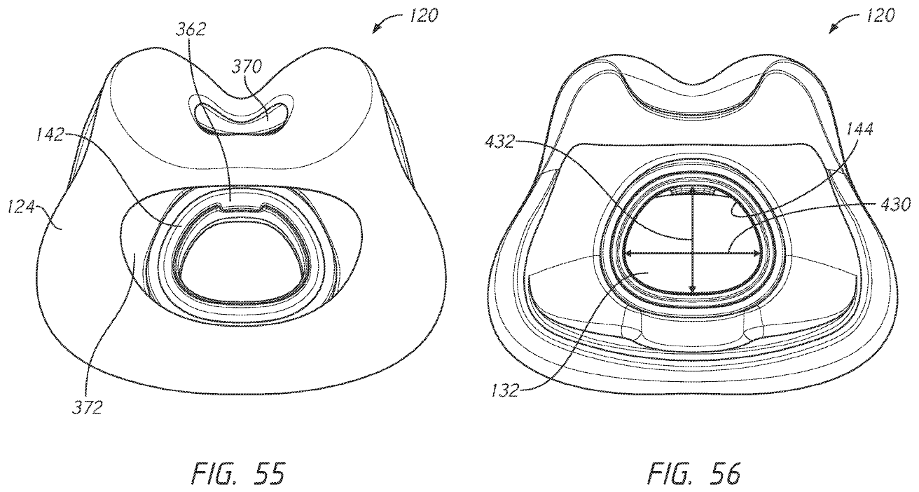

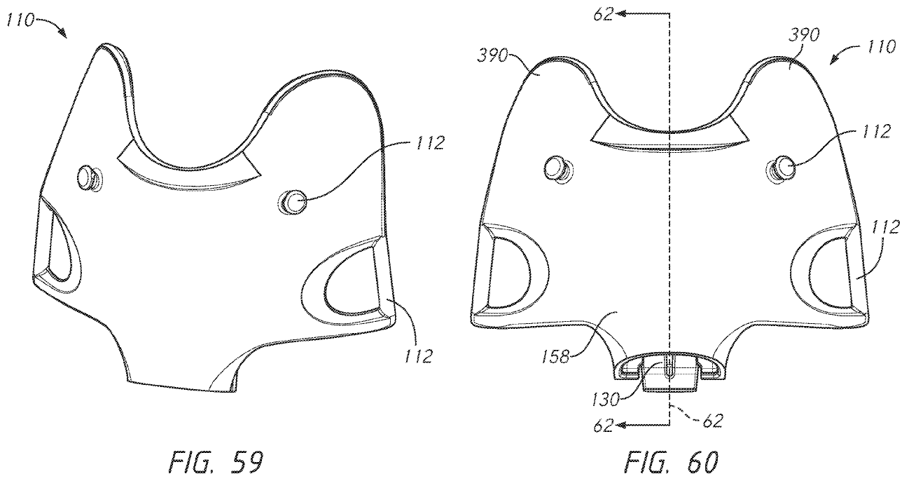

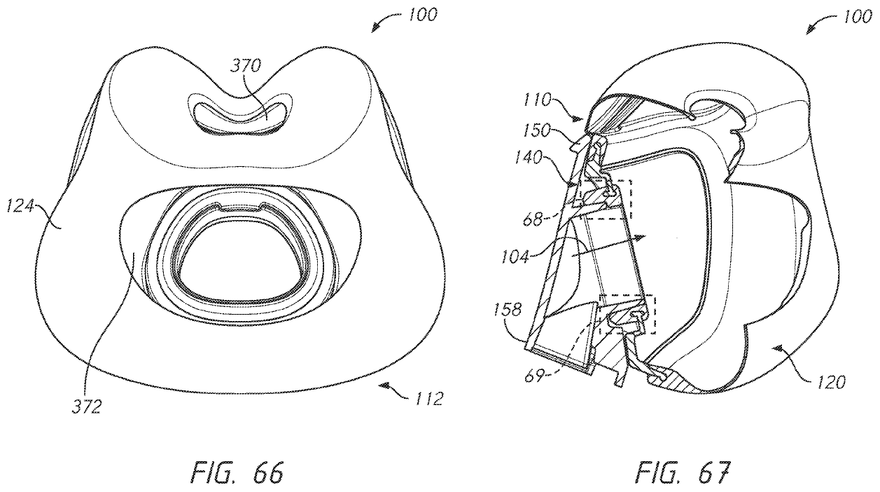

FIGS. 46-69 illustrate an embodiment of an oro-nasal or full-face under-nose interface assembly 100, which is similar in many respects to the other interfaces disclosed herein. Accordingly, the same reference characters can be used to refer to the same or similar components or features. The following description is focused on the unique features of the interface 100 relative to the other interfaces described herein. The interface 100 can incorporate compatible features of the other interfaces disclosed herein. In addition, features that are not disclosed in detail can be the same as or similar to other interfaces disclosed herein, or can be of another suitable arrangement.

Similar to other embodiments described herein, the patient interface 100 includes a frame 110 and a cushion module 120 removably coupled to the frame 110 by a friction coupling 140. The frame 110 is configured to be connected to headgear (not shown), which holds the patient interface 100 in a sealed relation to the user's face. The illustrated frame 110 includes a pair of upper headgear mounts 112 and a pair of lower headgear mounts 112. However, the frame 110 could include other desirable number of headgear mounts depending on the type of headgear desired to be used with the patient interface 100. The mounts 112 can be configured to directly or indirectly receive an associated headgear strap(s).

In the illustrated arrangement, each of the lower headgear mounts 112 is configured to receive a lower headgear connector clip of a corresponding lower headgear strap. Each of the lower headgear mounts 112 includes a post extending alongside an edge of the frame 110. The posts of the lower headgear mounts 112 can be spaced away from a portion of the frame 110 to define an aperture sized and shaped to accommodate a hook portion of the lower headgear connector clip. Each of the upper headgear mounts 112 is in the form of a mushroom-shaped post extending from a front surface of the frame 110. The posts of the upper headgear mounts 112 are configured to receive a headgear connector element. In some configurations, the headgear connector element is a single yoke, each end of which connects to an upper headgear strap.

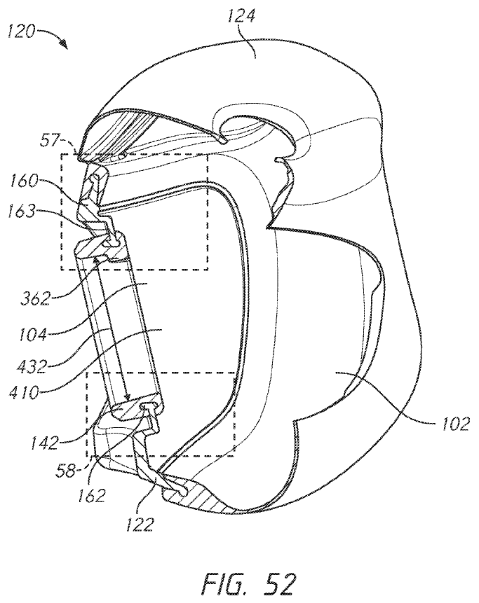

The cushion module 120 defines at least a portion of the breathing chamber 102 (FIG. 52) of the patient interface 100. The cushion module 120 includes a housing 122 and a cushion or seal 124. Preferably, the housing 122 includes a wall that extends in both a radial and axial direction relative to an axis 104 (FIG. 67) along which the frame 110 and the cushion module 120 overlap or are coupled. Thus, the housing 122 defines a depth in an anteroposterior direction. In some configurations, the axis 104 along which the frame 110 and the cushion module 120 overlap is generally or substantially aligned with the assembly axis or direction of assembly between the frame 110 and the cushion module 120.

The seal 124 is coupled to a rear or posterior portion of the housing 122 and defines a face-contacting surface configured to contact the face of a user of the interface 100 in a sealing or substantially sealing manner. The seal 124 can be an inflation-type seal configured such that it surrounds either or both of the nose and mouth of the user. In the illustrated arrangement, the seal 124 includes a nasal opening 370 and a separate oral or mouth opening 372. The portion of the seal 124 surrounding the nasal opening 370 is configured to contact an underside of the user's nose. The portion of the seal 124 surrounding the mouth opening 372 is configured to contact the upper lip, checks and lower lip/chin region of the user's face. In other configurations, the seal 124 could include a single opening or more than two openings (e.g., two nasal openings and a mouth opening). In addition, the seal 124 could include one or more superstructures (e.g., nasal pillows or prongs) for engaging the nares of the user.

The wall of the housing 122 surrounds, defines or includes a first opening (connection opening 132) at a forward end and a second opening at a rearward end. As discussed above, the seal 124 is connected to the second opening at the rearward end of the housing 122. Accordingly, the wall of the housing 122 extends from the first opening in a rearward direction towards the second opening. Preferably, the first opening is smaller than the second opening. That is, the second opening can have a larger vertical and/or lateral dimension than the first opening. In some configurations, a perimeter of the second opening is larger than a perimeter of the first opening. In some configurations, an area of the second opening is larger than an area of the first opening. In some configurations, because it is configured for connection to the frame 110, which provides for connection to headgear, the cushion module 120 does not include integrated headgear connector structures.

In some configurations, the housing 122 has a higher hardness and/or is more rigid than the seal 124. The housing 122 can be constructed from a more rigid material than the material of the seal 124. For example, the housing 122 can be constructed from a relatively rigid plastic (e.g., polycarbonate). The seal 124 can be constructed partially or completely from a less rigid material than the material of the housing 122, which can be a relatively soft, flexible, pliable, resilient or elastic material, such as an elastomer (e.g., silicone), a foam or a textile. In some configurations, the seal 124 is coupled to the housing 122 during a molding process, such as an over-molding process. In addition, or in the alternative, the seal 124 may be mechanically coupled to the housing 122. For example, the housing 122 can include a plurality of apertures through which the material of the scal 124 passes to create a mechanical interlock between the seal 124 and the housing 122. Preferably, the housing 122 provides sufficient strength and rigidity to maintain the breathing chamber 102 during use.

In some embodiments, the frame 110 can include upwardly extending support 390. In at least one configuration, the upwardly extending supports 390 can be in the form of upper lateral support portions 390. In at least one configuration, the upwardly extending supports 390 can be in the form of frame paddles 390. The upwardly extending supports 390 are configured to provide support to forward facing lateral sides of a nasal region of the mask seal 124 when the cushion module 120 is assembled to the frame 110. The lateral sides of the nasal region are referred to herein as upwardly extending portions or upward extensions 392. In at least one configuration, the upwardly extending portions 392 can be in the form of seal paddles 392. In the illustrated arrangement, the upwardly extending portions 392 are positioned above the relatively rigid housing 122. Such an arrangement allows the upwardly extending portions 392 to deflect or deform in shape to accommodate variations in the facial geometry of the individual user. For example, the upwardly extending portions 392 can deflect inwardly away from the upwardly extending supports 390 when the user's nose is pressed against the nasal portion of the seal 124. Such deflection can be advantageous in maintaining a seal with the sides of the user's nose. However, the upwardly extending supports 390 can help to minimize undesirable deflection of portions of the mask seal 124. For example, the upwardly extending supports 390 can help to maintain contact between the nasal region and the user's nose by contacting the upwardly extending portions 392 to inhibit or prevent the nasal region and/or the upwardly extending portions 392 of the seal 124 from inflating or moving outwardly or forwardly away from and/or otherwise disengaging from the user's nose. In the illustrated arrangement, the upwardly extending supports 390 are unitarily-formed with a remainder or a primary portion of the frame 110. Therefore, the upwardly extending supports 390 can have the same hardness or rigidity as the frame 110 or can be constructed of a material having the same hardness or rigidity as the material of the frame 110, including the variations of hardness or rigidity relative to other portions of the interface 100 as described in connection with the frame 110.

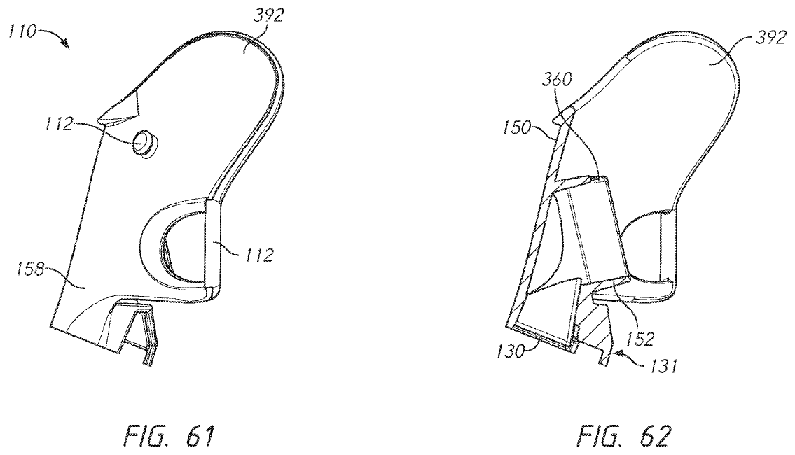

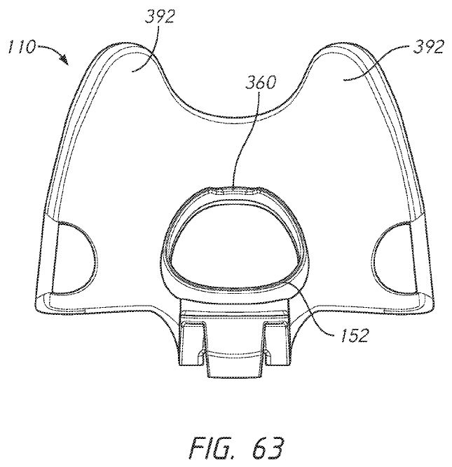

As described above, the frame 110 defines or otherwise includes a gas inlet 130 (FIGS. 60 and 62) that permits a flow of breathing gas to pass through the frame 110.

The cushion module 120 includes a connection opening 132 (FIG. 47) that permits a flow of breathing gas to pass through the cushion module 120. When the cushion module 120 is assembled to the frame 110, the gas inlet 130 of the frame 110 communicates with the connection opening 132 of the cushion module 120 such that a flow of breathing gas is permitted to pass through the frame 110 and the cushion module 120 into the breathing chamber 102.

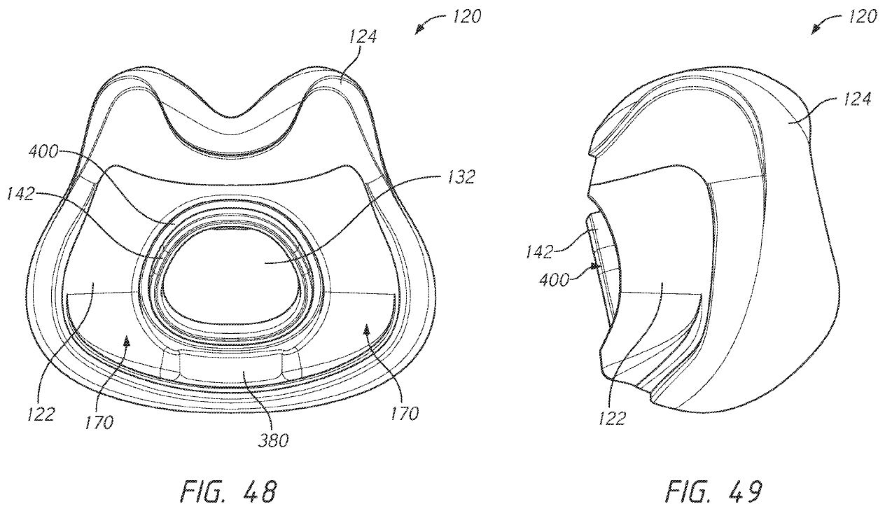

In the illustrated arrangement, the housing 122 of the cushion module 120 includes a vent 170 (FIG. 48) in fluid communication with the breathing chamber 102. The vent 170 is configured to allow a flow of breathing gases and/or gases exhaled by the user to pass from the breathing chamber 102 to the atmosphere external of the patient interface 100. As previously described, the vent 170 can be a bias flow vent comprising a plurality of small holes. A portion or an entirety of the vent 170 can be located below the connection opening 132. In some configurations, the vent 170 is provided in multiple discrete portions (e.g., two portions), which can be spaced apart from one another in a lateral direction on each side of a central, vertical plane of the cushion module 120 or interface 100. In the illustrated arrangement, the vent 170 is located below an edge of the frame 110 on each side of the gas inlet 130 when the cushion module 120 is connected to the frame 110.

A friction coupling 140 (FIGS. 67-69) selectively couples the cushion module 120 and the frame 110. The friction coupling 140 can be designed in accordance with the criteria or methodology described above with reference to the embodiments of FIGS. 1-45. As described above, the friction force provided by the friction coupling 140 can be the only retention force that holds the cushion module 120 and the frame 110 together when the interface 100 is not positioned on a user. That is, the friction coupling 140 can be the only direct coupling between the frame 110 and the cushion module 120. However, in other arrangements, the patient interface 100 can include additional couplings between the frame 110 and the cushion module 120.

In the illustrated arrangement, the friction coupling 140 is or comprises an elastomeric friction member 142 that creates at least a portion of a retention coupling between the frame 110 and the cushion module 120. In some configurations, the friction member 142 can provide or contribute to creating an airtight or substantially airtight seal between the frame 110 and the cushion module 120. With such an arrangement, leakage of breathing gas (e.g., air) between the frame 110 and the cushion module 120 can be prevented or sufficiently inhibited such that the desired positive pressure can be maintained within the breathing chamber 102.

As described above, the elastomeric friction member 142 can have a greater hardness than the seal 124. However, in other configurations the elastomeric friction member 142 can have the same hardness as the seal 124. In at least one configuration, the elastomeric friction member 142 can be constructed from the same material as the seal 124. For example, in at least one embodiment, the elastomeric friction member 142 and the seal 124 can each be formed from a silicone material and, thus, can be formed from the same material. In at least one configuration, the elastomeric friction member 140 can have a lower hardness than the seal 124. At least a surface of the friction member 142 that engages the frame 110 can have a textured surface finish, which as described previously herein can provide a desirable balance between retention and allowing sliding movement for assembly and disassembly.

The illustrated frame 110 includes at least a main wall or a front wall 150 and a collar 152, which extends rearwardly (e.g., posteriorly or toward the user) from the front wall 150 to a rearward or free end, edge or rim and surrounds the gas inlet 130. In the illustrated arrangement, the perimeter defined by the collar 152 is non-circular, which can inhibit or prevent relative rotation between the frame 110 and the cushion module 120 about an axis of the collar 152. The collar 152 can be or include a somewhat rounded ‘D’ shape, a rounded trapezoidal shape, elliptical shape and/or oval shape, among other possible shapes. In at least one embodiment, the collar 152 can be asymmetric about at least one axis. The collar 152 being a somewhat rounded ‘D’ shape, a rounded trapezoidal shape, elliptical shape, oval shape, and/or asymmetric about at least one axis can be advantageous as the correct orientation of the frame 110 with respect to the cushion module 120 is more easily determined. The aforementioned configuration can also be advantageous as said configuration can provide a self-aligning connection between the frame 110 and the cushion module 120. At least one advantage of the aforementioned configuration with the elastomeric friction member 142 is that the elastomeric friction member 142 can deform during connection of the frame 110 and the cushion module 120 to improve the performance of the self-aligning connection when the frame 110 is connected to the cushion module 120.

The illustrated frame 110 also includes an integrated conduit connector portion 158, which can serve the same or a similar function as the conduit connector portion 158 of other embodiments disclosed herein. In particular, the conduit connector portion 158 couples a gas delivery conduit or breathing circuit (not shown) to the frame 110 for delivery of a flow of breathing gases to the gas inlet 130. The conduit connector portion 158 defines a portion of the gas inlet 130 along with the collar 152 and the front wall 150. As described herein, the conduit connector portion 158 can include a swivel connector, which provides for a rotational connection to the associated gas delivery conduit or breathing circuit.

In the illustrated arrangement, the cushion module 120 includes an inlet recess 380 (FIG. 47) configured to accommodate at least a portion of the conduit connector portion 158. The inlet recess 380 can receive within it a rearward-facing portion of the conduit connector portion 158. The inlet recess 380 is positioned below the connection opening 132 and extends in a lateral direction along a substantial portion of a width of the connection opening 132 and/or the conduit connector portion 158. The inlet recess 380 allows the conduit connector portion 158 and/or the frame 110 to be positioned closer to the user's face in comparison to a design without the inlet recess 380. Such an arrangement can reduce the likelihood that hose pull forces will disrupt the seal of the cushion module 120 with the user's face. At least one advantage of the inlet recess 380 can be a reduced size, profile or depth of the interface 100, or at least an improved perception of the interface 100 by users who may be intimidated by larger masks.

The conduit connector portion 158 can include an anti-asphyxiation valve (AAV) 131, which can be positioned rearwardly of the gas inlet 130. The AAV 131 opens to allow ambient air to enter the breathing chamber 102 via the gas inlet 130 in the event that the flow of breathing gases is disrupted or ceases, or if the pressure within the breathing chamber 102 otherwise falls below the ambient pressure. The AAV 131 often includes a flap valve that selectively opens or closes an auxiliary passage between the gas inlet 130 and the ambient air. However, the AAV 131 can be of any suitable arrangement.

The front wall 150, the collar 152 and the conduit connector portion 158 can be unitarily formed of a material with sufficient strength and rigidity to transfer headgear forces from headgear to the cushion module 120 and/or maintain a shape of the collar 152, the conduit connector portion 158 or any portion of the breathing chamber 102 defined by the frame 110 during use. For example, the frame 110 can be formed partially or entirely from a relatively rigid polymeric material (e.g., polycarbonate), by a process such as molding (e.g., injection molding). The axis of the gas inlet 130 of the frame 110 and/or the connection opening 132 of the cushion module 120, or the z-axis 104, can be aligned with the assembly axis.

In some configurations, the integration of the conduit connector portion 158 with the frame 110 can allow the gas delivery conduit to extend in a generally downward direction from a lower front portion of the frame 110. Such a configuration can reduce the overall bulkiness of the patient interface. For example, in such configurations, the gas delivery conduit can be positioned closer to the user in use. In some configurations, a dimension of the gas inlet 130 in a forward-rearward direction is smaller than a dimension of the gas inlet 130 in a lateral direction. Accordingly, extra space is provided to accommodate the AAV 131 in a forward-rearward direction in comparison to a design in which the gas inlet 130 is circular without increasing the forward-rearward dimension of the frame 110 or moving the gas inlet 130 further away from the user's face.

The housing 122 of the cushion module 120 includes a main wall 160 and a support wall 163 (FIG. 52). The support wall 163 and/or a portion of the main wall 160 surrounds and/or defines the connection opening 132. The support wall 163 defines a non-circular perimeter shape from a front view that preferably corresponds to the perimeter shape of the collar 152, but preferably is somewhat larger to accommodate a portion of the friction member 142 therebetween.

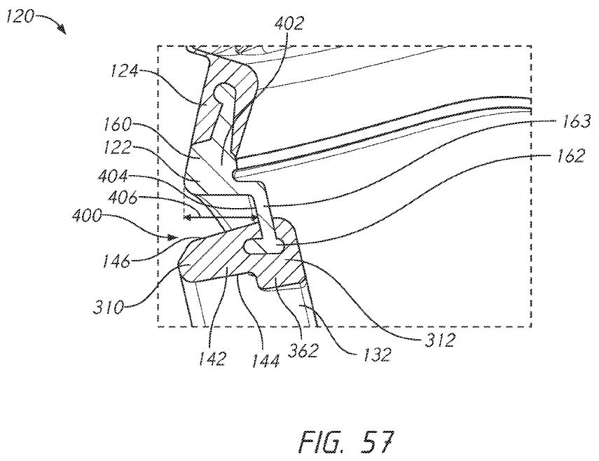

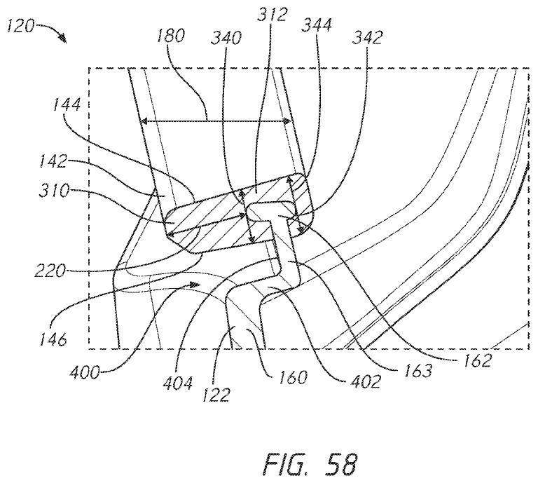

With reference to FIGS. 57 and 58, the illustrated support wall 163 extends radially inward from the main wall 160 and is offset from a portion of the main wall 160 immediately surrounding the support wall 163. In the illustrated arrangement, the support wall 163 is rearwardly offset from the main wall 160 to define a recess 400 of the housing 122 of the cushion module 120. The support wall 163 is connected to the main wall 160 by a connecting portion 402. In some forms, the recess 400 can be defined by the connecting portion 402 and the non-embedded portion of the support wall 163. In one form the recess 400 is bound by the connecting portion 402, the non-embedded portion of the support wall 163, and a portion of the peripheral surface 146 of the friction member 142. An axial dimension or length of the connecting portion 402 can be variable around its periphery and, in some cases, can have no axial dimension along a portion of the periphery of the recess 400. The support wall 163 defines a recessed surface 404 that is offset from a portion or an entirety of the main wall 160 by a recess distance 406. In other arrangements, the support wall 163 could be aligned with the main wall 160 or offset in a forward direction. The recess distance 406 can be constant around a periphery of the support wall 163. The recess distance 406 can be variable around a periphery of the support wall 163. The dimensions (e.g., lengths) of the structures defining the recess 400 (e.g., one or more of the connecting portion 402, the non-embedded portion of the support wall 163, and a portion of the peripheral surface 146 of the friction member 142) can be selected to create a recess 400 of a desirable size, shape or orientation to position the friction member 142 relative to the user when the cushion module 120 is properly positioned on the user's face, relative to other portions of the cushion module 120, relative to the frame 110 or relative to any other portions of the interface 100, in accordance with the considerations discussed herein, including but not limited to reducing the effect of hose pull forces acting on the interface 100.

A variable recess distance 406 around a periphery of the support wall 163 can advantageously angle the connection opening 132 with respect to the rest of the interface 100. In at least one configuration, providing the variable recess distance 406 angles the connection opening 132 such that the connection opening 132 is effectively rotated about a lateral axis of the interface 100. In at least one configuration, the variable recess distance 406 provides a connection opening 132 that is angled forward or downward about the lateral axis of the interface 100. In such a configuration, the variable recess distance 406 is greater towards a lower portion or bottom of the connection opening 132 than toward an upper portion or top. In such a configuration, the assembly axis 104 can be rotated about the lateral axis of the interface corresponding with, or approximating the rotation of the connection opening 132.

The aforementioned configuration can advantageously reduce a bending moment exerted on the interface 100 by hose pull. In at least one configuration, the variable recess distance 406 provides a connection opening 132 that is angled rearward or upward about the lateral axis of the interface 100. In such a configuration, the variable recess distance 406 is greater towards the upper portion or top of the connection opening 132 than towards the lower portion or bottom. The aforementioned configuration can advantageously improve the comfort of the interface 100. In at least one configuration, the variable recess distance 406 is the same, or substantially similar at or near a lower portion or bottom of the connection opening 132 and an upper portion or top of the connection opening 132. In at least one configuration, the variable recess distance 406 at or near lateral sides of the connection opening 132 is the same, or substantially similar.

In at least one configuration, the recessed surface 400 spans only a portion of the perimeter of the support wall 163. In at least one configuration, the recess distance 406 is a variable recess distance 406. For example, in such a configuration the recess distance 406 can vary from a maximum at or near an upper portion of the connection opening 132 to a minimum away from the upper portion of the connection opening 132, such as at or near each lateral side and/or the lower portion of the connection opening 132. In such a configuration, a lower portion of the interface 100 can be offset from an upper portion of the interface 100. The upper portion of the interface 100 can extend further forward than the lower portion. This can allow sufficient room for a user's nose within the upper portion, while reducing the volume of the breathing chamber because of the offset lower portion, thereby reducing the dead space of the interface 100.

The elastomeric friction member 142 is carried by or coupled to the cushion module 120. In particular, the friction member 142 is coupled to the support wall 163. At least a portion of the support wall 163 is embedded within the friction member 142 to form a mechanical anchor to secure the friction member 142 to the housing 122. In the illustrated arrangement, only a portion of the support wall 163 is embedded within the friction member 142 and the support wall 163 passes through a peripheral surface 146 of the friction member 142. A portion or an entirety of a perimeter of the support wall 163 defines a first portion that is embedded within the friction member 142. A portion or an entirety of a perimeter of the support wall 163 a second portion that is exposed or not embedded within the friction member 142. In the illustrated arrangement, the second portion is annular or extends along an entirety of a perimeter of the support wall 163 such that the peripheral surface 146 of the friction member 142 is spaced from the connecting portion 402 or an adjacent portion of the housing 122 in a radial direction. The second portion is located radially outward of the first portion. The support wall 163 can also provide hoop strength to the friction member 142 or limit outward expansion of the friction member 142 to enhance the friction fit with the collar 152 of the frame 110.

The friction member 142 can be permanently coupled to the support wall 163, such as by an over-molding process. The support wall 163 can include one or more apertures 166 through which the material of the friction member 142 extends. This can be a result of the over-molding process. Such an arrangement provides a mechanical interlock between the friction member 142 and the support wall 163 of the housing 122. In the illustrated arrangement, the support wall 163 includes the plurality of apertures 166, each of which extends axially through the support wall 163. With such an arrangement, the apertures 166 can be aligned with a pull direction of the tooling used to form the housing 122. The apertures 166 can be evenly or unevenly distributed around the perimeter of the support wall 163. The apertures 166 preferably are sized such that the portions of the friction member 142 passing therethrough are strong enough to resist breaking in response to normal or expected forces acting on the friction member 142.

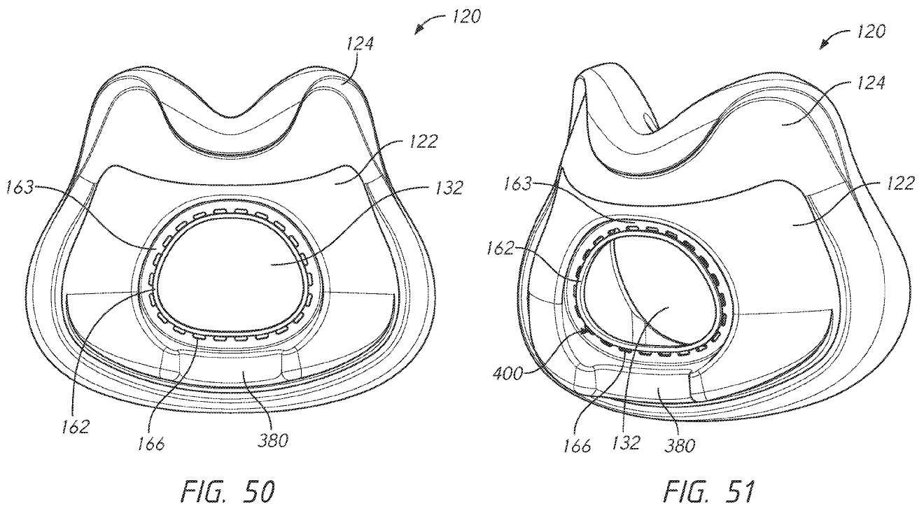

With reference to FIGS. 50-52, 57 and 58, a radially-inward edge of the support wall 163 is connected to a support flange 162. The support wall 163 defines a wall thickness and the support flange 162 defines a length in generally the same direction that is greater than the wall thickness of the support wall 163. The support flange 162 can increase the strength and reduce the deformation of the support wall 163 in comparison to an arrangement without a support flange 162. The support flange 162 can also assist with the mechanical interlock of the friction member 142 to the cushion module 120 and/or can provide support to the friction member 142 such that the friction coupling 140 can exhibit desirable characteristics related to the assembly and disassembly of the cushion module 120, as described above with respect to FIGS. 1-12, for example. In some configurations, the apertures 166 are located on the support wall 163 adjacent the support flange 162.

The support flange 162 can be sized and shaped to provide a sufficient or desirable level of reinforcement to the support wall 163 and/or mechanical interlocking with the friction member 142. In the illustrated arrangement, the support flange 162 extends in both axial directions relative to the support wall 163. In other words, the support flange 162 projects in both forward and rearward directions from the support wall 163. In the illustrated arrangement, the support flange 162 projects a greater distance in a forward direction from the support wall 163 relative to the distance of the rearward projection. However, in other arrangements this could be reversed or the portions on each side of the support flange 162 could have equal axial dimensions. The ends of the support flange 162 can have rounded corners or edges to reduce stress concentrations in the support wall 163 and the boundaries between the support wall 163 and the friction member 142 in comparison to an arrangement with sharp corners or edges.

As illustrated in FIG. 52, the support flange 162 defines a central, longitudinal axis 410 that is offset with respect to, or defines an angle relative to, the axis of the friction member 142, which in the illustrated arrangement coincides with the assembly axis 104. Such an arrangement allows the pull direction of the tooling for the housing 122 and the tooling for the friction member 142 to be different from one another. In some configurations, the pull direction of the tooling is improved or optimized for each of the housing 122 and the friction member 142. In the illustrated arrangement, the axis 410 of the support flange 162 is closer to horizontal than the assembly axis 104 when the cushion module 120 is vertically-oriented, as shown in FIG. 52. A forward portion of the assembly axis 104 is tilted downward relative to the axis 410 and a rearward portion of the assembly axis 104 is tilted upward relative to the axis 410. In other respects, the support flange 162 can be the same as or similar to the support flange 162 of the interface 100 of FIGS. 21-45.

As described, at least a portion of the support wall 163 is embedded within the friction member 142. The friction member 142 extends axially away from the support wall 163. A portion of the friction member 142 is located on a forward side (distal the user) of the support wall 163 or extends axially away from the support wall 163 in a forward direction. A portion of the friction member 142 is located a rearward side (proximal the user) of the support wall 163 or extends axially away from the support wall 163 in a rearward direction. In the illustrated arrangement, the portion of the friction member 142 located on the forward side of the support wall 163 is larger than the portion of the friction member 142 located on a rearward side of the support wall 163 in at least one dimension. For example, the portion of the friction member 142 located on the forward side of the support wall 163 can have a larger axial dimension or is longer than the portion of the friction member 142 located on a rearward side of the support wall 163, which has a smaller axial dimension or is shorter than the portion on the forward side.

In the illustrated arrangement, the connection opening 132 is positioned below the nasal region of the cushion module 120 and can be within an oral region of the cushion module 120 and/or approximately aligned with a mouth of the user in a vertical direction. Accordingly, the recessed support wall 163 in combination with the primarily forward projection of the illustrated friction member 142 takes advantage of the available space within the oral region of the breathing chamber 102 located below the user's nose. Such an arrangement allows at least the conduit connector portion 158 of the frame 110 to be located closer to the user's face, which reduces the effect of hose pull forces on the interface 100. Hose pull considerations can have greater importance in under-nose interfaces or other interfaces having a forward-leaning orientation, such as when the nasal/nose portion projects further forward than the oral/mouth portion, and/or in interfaces lacking a forehead support or other similar support arrangement (e.g., overhead strap). Such interfaces generally are less stable and more prone to displacement as a result of hose pull forces. Thus, the recessed support wall 163 of the illustrated interface 100 can improve stability as a result of allowing the conduit connector portion 158 to be located closer to the user's face.

The portion of the friction member 142 located on a forward side of the support flange 162 (or the support wall 163 if there is no support flange 162 or if the support flange 162 does not extend forward of the support wall 163) defines an unsupported portion 310 of the friction member 142, which can define an unsupported length dimension in the axial direction. In other words, the unsupported portion 310 is not directly supported in a radial direction by the support flange 162 (or the support wall 163). A portion of the friction member 142 radially inward from the support flange 162 (or the support wall 163) defines a supported portion 312 of the friction member 142, which can define a supported length in the axial direction. The supported portion 312 is directly supported in a radial direction by the support wall 163 and/or the support flange 162. Thus, the unsupported portion 310 can deform radially outward or increase in diameter or circumference with less force than the supported portion 312. In some configurations, as described above, the unsupported portion 310 applies less radial force to the collar 152 than the supported portion 312.

In the illustrated arrangement, the peripheral surface 146 of the friction member 142 has portions in front of and behind the support wall 163 that lie along a continuous path. That is, the portion of the peripheral surface 146 behind the support wall 163 lies on the same trajectory as the portion in front of the support wall 163. This is a substantially linear trajectory. In an alternate configuration, rather than the same trajectory, the portion of the peripheral surface 146 behind the support wall 163 can lie along a continuous trajectory, for example that defined by a curve or spline with respect to the portion in front of the support wall 163. In the illustrated arrangement, the peripheral surface 146 is oriented at a slightly oblique angle relative to the support wall 163. In other arrangements, the peripheral surface 146 can be perpendicular to the support wall 163. In either case, the portions of the peripheral surface 146 immediately adjacent the front and rear surfaces of the support wall 163 oppose one another or are substantially aligned or aligned with one another. That is, preferably there is no significant offset between the portions of the peripheral surface 146 immediately adjacent the front and rear surfaces of the support wall 163. As described above, preferably the friction member 142 covers only a portion of the support wall 163 such that a portion of the support wall 163 is exposed, which provides space for and/or can be contacted by the shut-off portion of the molding tool for the friction member 142. With such an arrangement, the shut-off portions of the molding tool for the friction member 142 are substantially aligned or aligned with one another, which results in a more reliable molding process that is less prone to flashing than when the shut-off portions of the molding tool are not aligned, as described above with reference to FIGS. 13 and 14.

As described above, the recess distance 406 can be selected to position the friction member 142 desirably close to the user's mouth in use to position the frame 110, or at least the conduit connector portion 158 of the frame 110, close to the user's face to reduce the instability induced by hose pull forces. The friction member 142 can define a total friction member length 180 (FIG. 58) between a forward end and a rearward end selected to provide a desirable level of retention or other attributes of the friction coupling 140, as described above. Furthermore, a forward length 220 (FIG. 58) of the unsupported portion 310 can also be selected to provide a desirable level of retention or other attributes of the friction coupling 140. In the illustrated arrangement, the recess distance 406, the total friction member length 180 and the forward length 220 of the unsupported portion 310 are selected such that at least a portion of the friction member 142 projects forwardly from the recess 400 when the cushion module 120 is viewed from the side, as illustrated in FIG. 49, for example.

In some configurations, the recess distance 406 is less than the total friction member length 180 and/or the forward length 220 of the unsupported portion 310 to balance the positioning of the friction member 142 close to the user's mouth while also providing a desirable amount of clearance. However, in other configurations, the recess distance 406 can be equal to or greater than one or both of the total friction member length 180 and the forward length 220 of the unsupported portion 310. Such an arrangement can occur by making the total friction member length 180 relatively small to reduce the retention force or material of the friction member 142 or by making the recess distance 406 relatively large to position the friction member 142 close to the user. For example, a cushion module 120 intended for user's with smaller faces (e.g., a size small or relatively smaller size) could have a greater recess distance 406 than a cushion module 120 intended for user's with larger faces (e.g., a size large or relatively larger size) assuming the remainder of the housing 122 of the cushion module 120 is the same or similar in size and/or shape.

The illustrated friction member 142 has a somewhat rounded ‘D’ shape or a rounded trapezoidal shape when viewed from the front or rear of the cushion module 120 or along the assembly axis 104. The shape of the friction member 142 corresponds with or is complementary to the shape of the collar 152. With reference to FIG. 56, the connection opening 132 of the friction member 142 defines a major axis 430 and a minor axis 432. The major axis 430 has a relatively larger dimension compared to the minor axis 432. The minor axis 432 has a relatively smaller dimension compared to the major axis 430. In the illustrated arrangement, the minor axis 432 extends between the top and the bottom of an engagement surface 144 of the friction member 142 and lies within a vertical, central plane of the cushion module 120 in the anteroposterior direction. This plane can correspond to or be parallel with a sagittal plane of the user of the patient interface. This plane can be perpendicular to a transverse plane of the user of the patient interface. The major axis 430 is perpendicular to the minor axis 430 and extends between the widest points of the left and right lateral sides of the engagement surface 144. However, this arrangement could be reversed.

The dimensions of the major axis 430 and the minor axis 432 can be selected, with consideration to the shape of the connection opening 132, to provide a suitable area through which a desirable flow rate of breathing gases into the cushion module 120 can be accommodated. The dimensions of the major axis 430 and the minor axis 432 can be selected to provide a desirable retention force or other desirable attributes of the friction coupling 140. A ratio of the major axis 430 dimension to the minor axis 432 dimension can be about 1.2-1.4:1, about 1.3:1 or about 1.27:1. The major axis 430 can have a dimension of about 25-45 mm, about 30-40 mm, or about 33 mm or 35 mm. The minor axis 432 can have a dimension of about 15-35 mm, about 20-30 mm, or about 25 mm or 26 mm.

The major axis 430 dimension can change along the friction member 142.

The major axis 430 dimension can decrease along the length of the friction member 142 from the front of the friction member 142 (the distal end of the friction member with respect to the user) to the rear of the friction member 142 (the proximal end of the friction member 142 with respect to the user). In at least one configuration, the major axis 430 dimension can increase along the length of the friction member 142 from the front of the friction member 142 (the distal end of the friction member with respect to the user) to the rear of the friction member 142 (the proximal end of the friction member 142 with respect to the user).

The minor axis 432 dimension can change along the friction member 142.

The minor axis 432 dimension can decrease along the length of the friction member 142 from the front of the friction member 142 (the distal end of the friction member with respect to the user) to the rear of the friction member 142 (the proximal end of the friction member 142 with respect to the user). In at least one configuration, the minor axis 432 dimension can increase along the length of the friction member 142 from the front of the friction member 142 (the distal end of the friction member with respect to the user) to the rear of the friction member 142 (the proximal end of the friction member 142 with respect to the user).

With reference to FIG. 58, the friction member 142 can define a wall thickness 340 between the engagement surface 144 and the peripheral surface 146. Because one or both of the engagement surface 144 and the peripheral surface 146 can be angled relative to the longitudinal axis 104 of the friction member 142, the wall thickness 340 can vary along the length of the friction member 142. The wall thickness 340 can generally correspond to the first thickness or total thickness 190 described with respect to prior embodiments.

The embedded portion of combination of the support wall 163 and support flange 162 (if present) defines a radial dimension or supported thickness 342. A portion of the friction member 142 radially inward of the support wall 163 (e.g., the supported portion 312) defines a radial dimension or an inner thickness 344. The inner thickness 344 generally corresponds to the fourth thickness 196 described with respect to prior embodiments. A ratio of the supported thickness 342 to the inner thickness 344 or to the wall thickness 340 can be selected to provide or contribute to desirable attributes of the friction coupling 140, as described herein. For example, the supported thickness 342 can be selected to provide for a desirable radial dimension of the apertures 166 (e.g., 1 mm) and/or a desirable radial dimension of the support flange 162 (e.g., 1 mm). The supported thickness 342 can also be selected to provide for a desirable amount of support for the supported portion 312.

The inner thickness 344 can be selected to provide for or facilitate a desirable interference fit with the frame 110. For example, the inner thickness 344 can be selected such that the compression of the portion of the friction member 142 defining the inner thickness 344 after assembly of the cushion module 120 to the frame 110 provides a desirable retention force or a desirable contribution to the overall retention force, as described above.

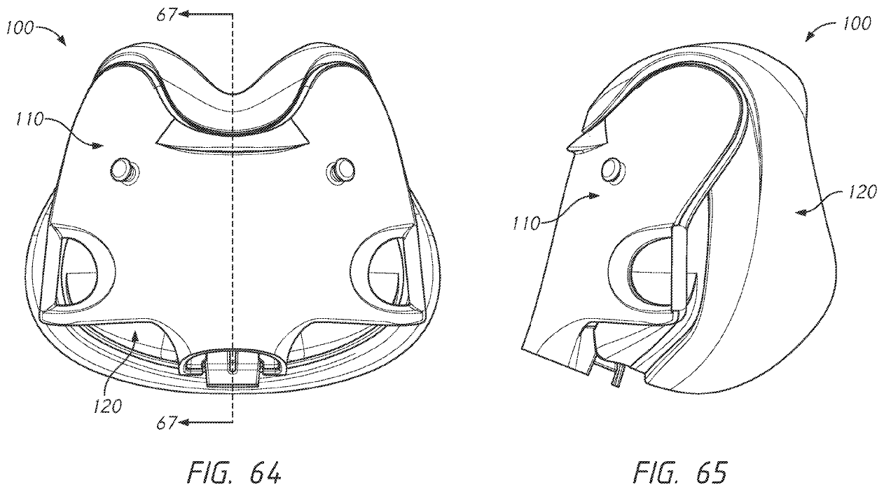

FIGS. 64-69 illustrate the friction member 142 of the cushion module 120 engaged with the collar 152 of the frame 110. As described, when the cushion module 120 is fully assembled to the frame 110, the interior or engagement surface 144 of the friction member 142 engages an exterior or engagement surface 154 of the collar 152. The engagement surface 144 can engage the exterior surface 154 with an interference (e.g., friction) fit along at least a portion of the friction member 142 or the collar 152. As a result of the interference or friction fit, one or both of the friction member 142 and the collar 152 is deformed when the cushion module 120 is assembled to the frame 110. In the illustrated arrangement, the peripheral surface 146 of the friction member 142 is exposed. That is, the peripheral surface 146 is not engaged by an outer wall or other similar structure.

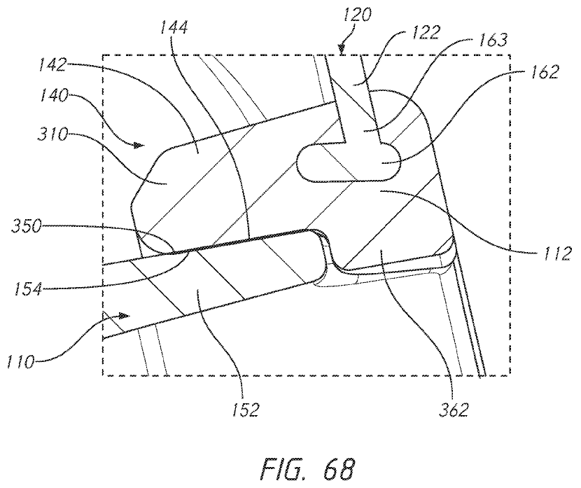

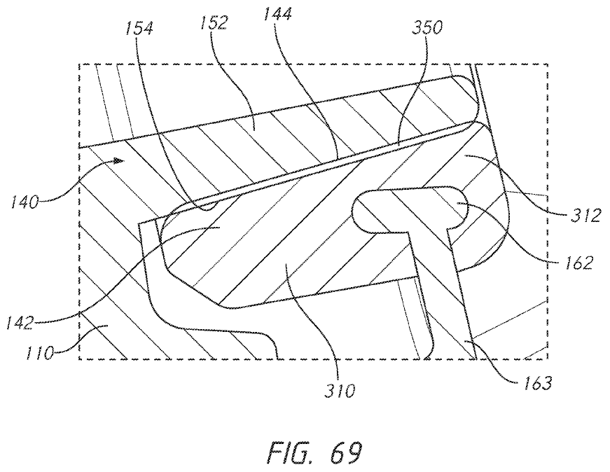

In some configurations, a retention force generated by the interference fit varies along the length of the friction coupling 140 because of, for example, variation in the wall thickness 340 along the length of the friction member 142 and/or the level of support provided to the friction member 142 (e.g., between the unsupported portion 310 and the supported portion 312). In the illustrated arrangement, an entirety or a substantial entirety of the length of the friction member 142 provides for an interference fit. For example, as illustrated in FIGS. 68 and 69, an interference fit 350 is created along an entirety or a substantial entirety of the engagement surface 144 or a length of the friction member 142 (excluding cut-outs or chamfered edges). However, in other configurations, an interference fit could be provided along only a portion of the length of the friction coupling 140.

FIGS. 68 and 69 illustrate the collar 152 of the frame 110 and the friction member 142 of the cushion module 120 in an as-formed state to illustrate the positive interference fit 350 between the friction member 142 and the collar 152 when the cushion module 120 is assembled to the frame 110. The interference fit 350 represents the total amount of deformation required by the friction member 142 and/or the collar 152 for the cushion module 120 to fit onto the frame 110. In reality, one or both of the friction member 142 and the collar 152 would deform so that the engagement surface 144 and a corresponding portion of the exterior surface 154 of the collar 152 are coincident. In the illustrated arrangement, the amount or the radial dimension of the interference fit 350 is constant along the length of the friction member 142 or friction coupling 140. However, in other arrangements, the amount or radial dimension of the interference fit 350 can vary along the length of the friction member 142 or friction coupling 140, such as due to a tapered shape or angle of the engagement surface 144 and/or exterior surface 154, as described with respect to other embodiments herein.

The cushion module 120 and the frame 110 can incorporate structures to indicate correct assembly orientation, to inhibit or prevent incorrect assembly and/or to guide the cushion module 120 and the frame 110 towards the correct assembly orientation. In some configurations, such structures can inhibit or prevent relative movement (e.g., rotational movement) after the cushion module 120 is correctly and fully assembled to the frame 110. In some configurations, the cushion module 120 and the frame 110 can incorporate one or more sets of cooperating protrusions and recesses that provide any or all of the above-described functions.

In the illustrated arrangement, the collar 152 of the frame 110 includes a recess 360 extending in a forward direction from a rearward end of the collar 152. In the illustrated arrangement, the recess 360 is located in an upper portion (e.g., upper half) of the collar 152 and aligned with a central, vertical plane passing in a forward-rearward direction through the frame 110. The friction member 142 of the cushion module 120 includes a protrusion 362 configured to be received by the recess 360 when the cushion module 120 is assembled to the frame 110. The protrusion 362 protrudes radially inwards into the connection opening 132. In at least one configuration, the protrusion can protrude away from the support wall 163.

The recess 360 and the protrusion 362 can have suitable corresponding or complementary shapes. In some configurations, the protrusion 362 occupies an entirety of the recess 360. Such an arrangement can substantially inhibit or prevent relative rotational movement of the cushion module 120 and the frame 110 about an axis of the friction coupling 140, the friction member 142 and/or the collar 152 (e.g., the z-axis) when circular shapes are employed. In some embodiments, the recess 360 has a generally trapezoidal shape oriented such that a width at rearward edge of the collar 152 is greater than a width at a position closer to the forward end of the collar 152. In other configurations, the protrusion 362 can be configured to occupy only a portion of the recess 360. Such arrangements can provide an indication or guidance as to the correct assembly orientation and can inhibit or prevent incorrect assembly (e.g., the cushion module 120 being oriented upside-down relative to the frame 110).

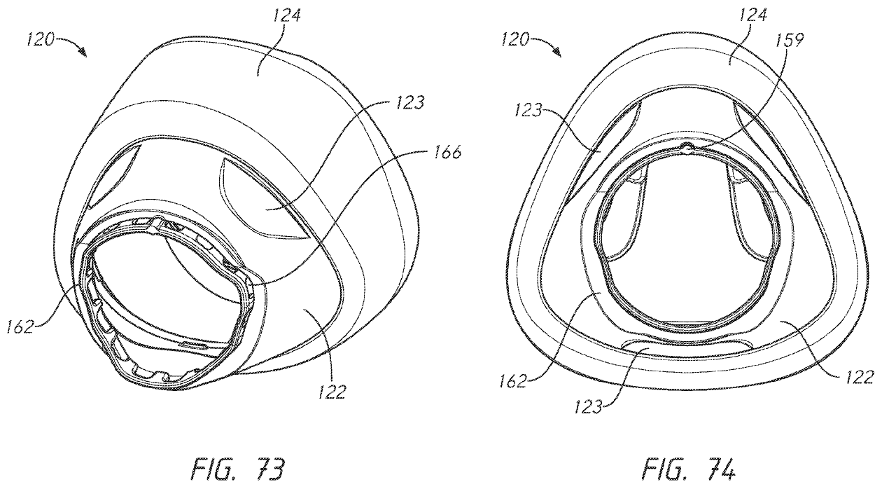

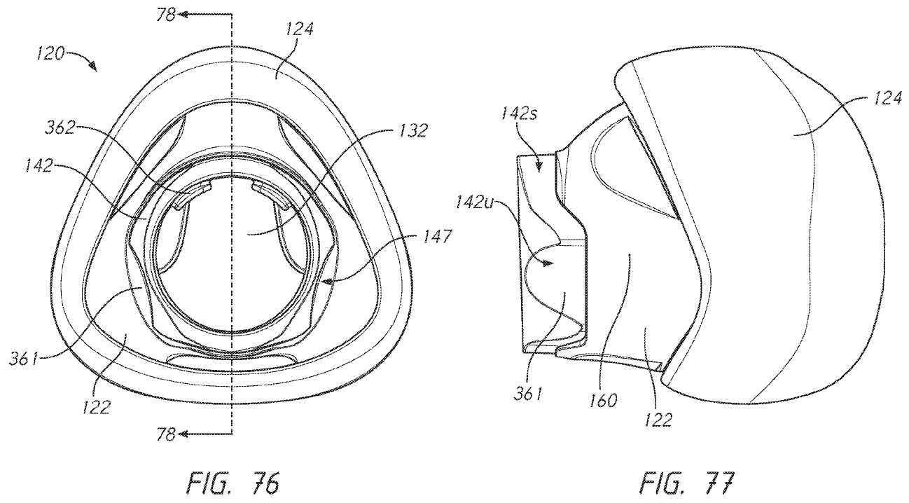

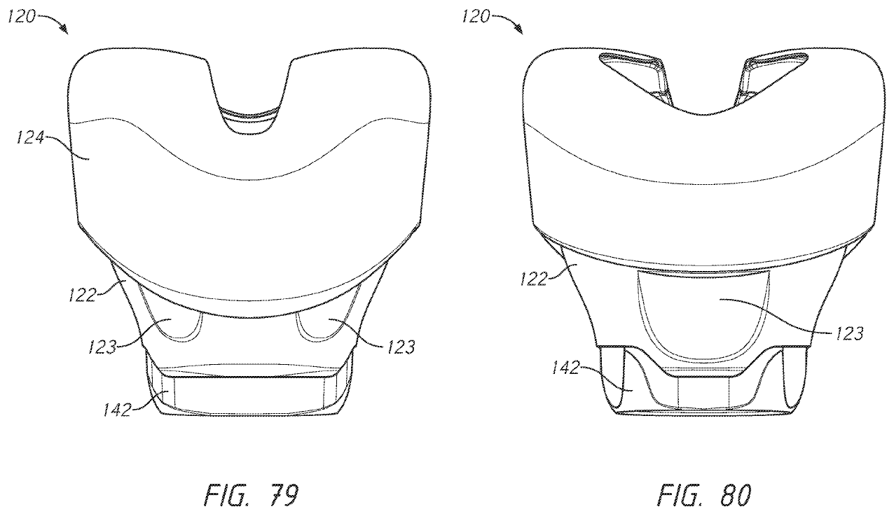

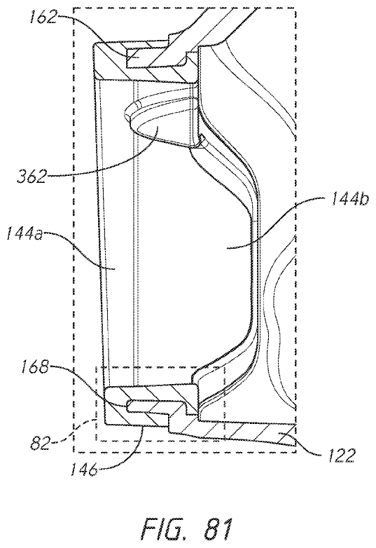

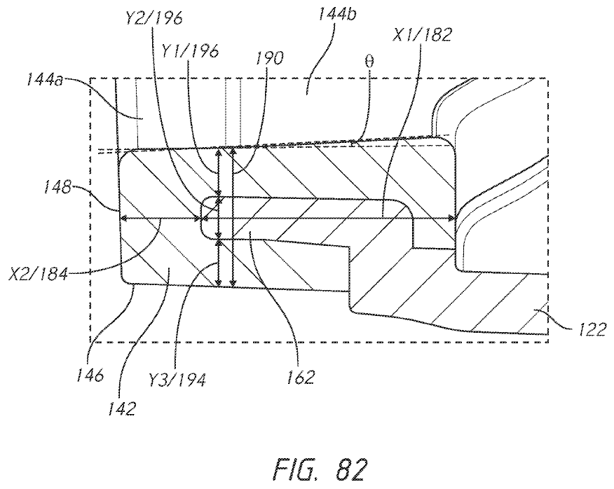

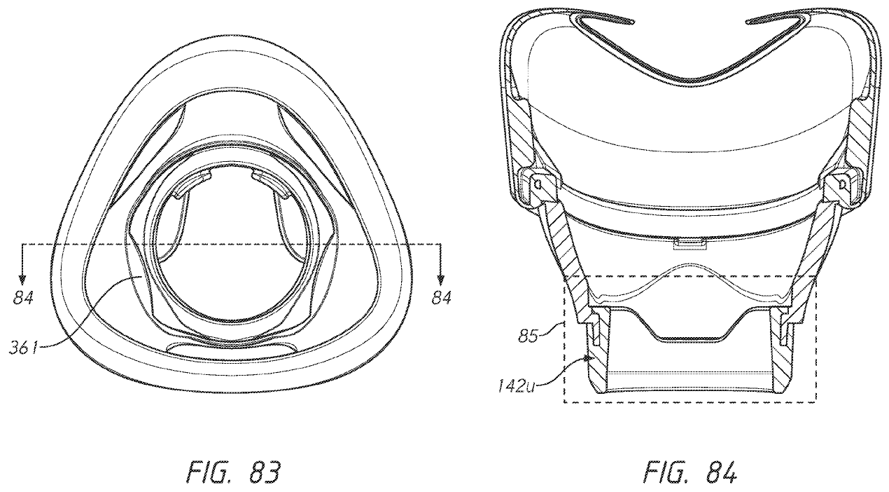

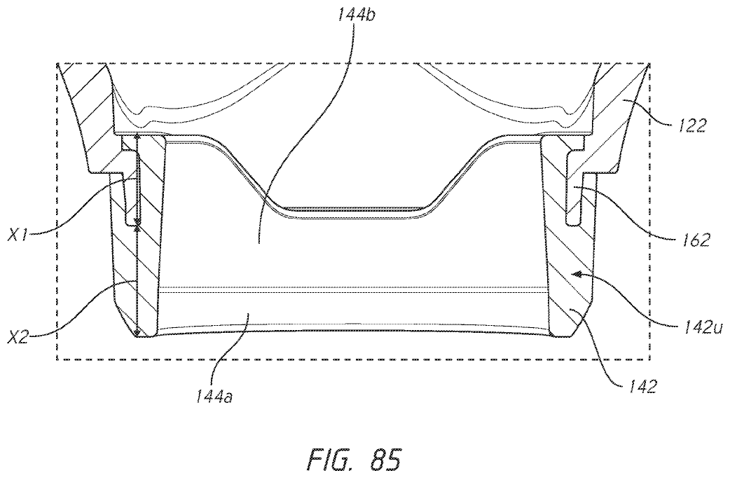

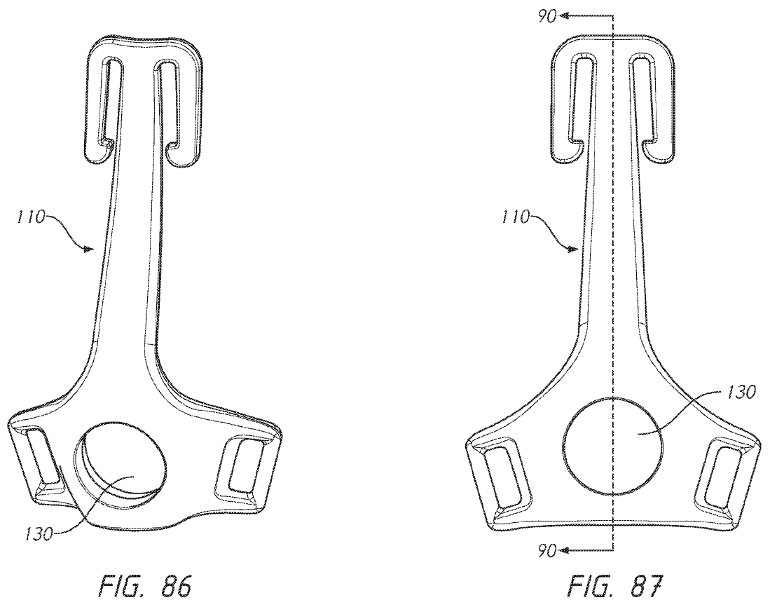

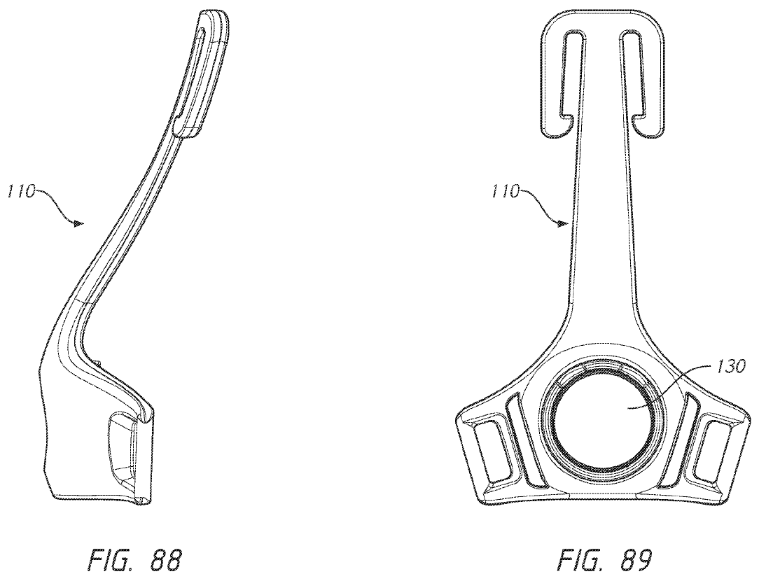

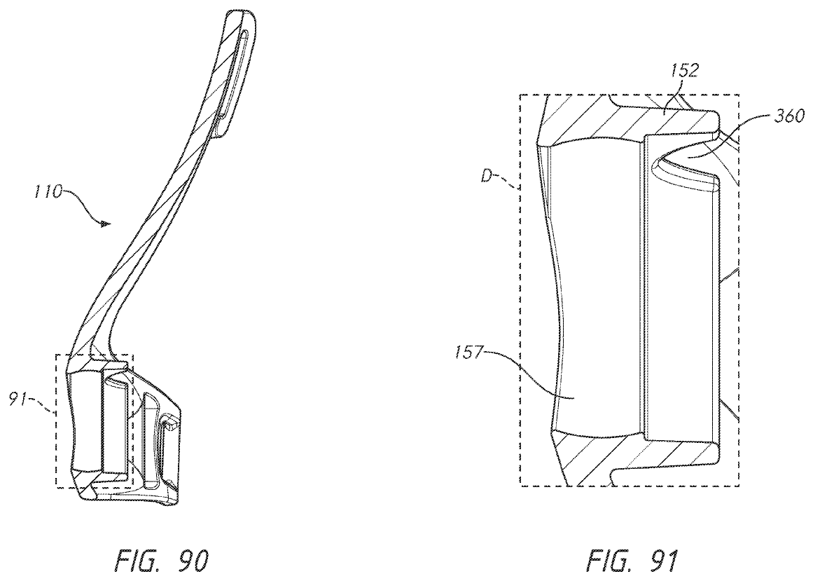

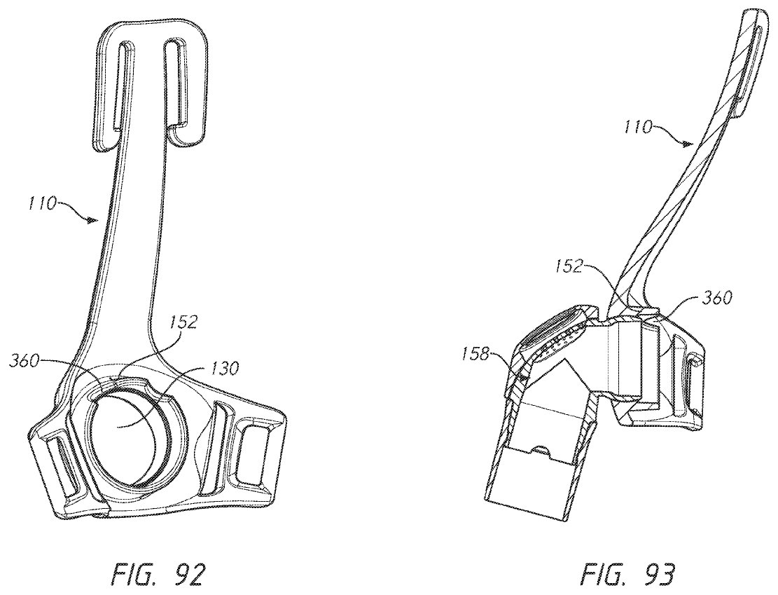

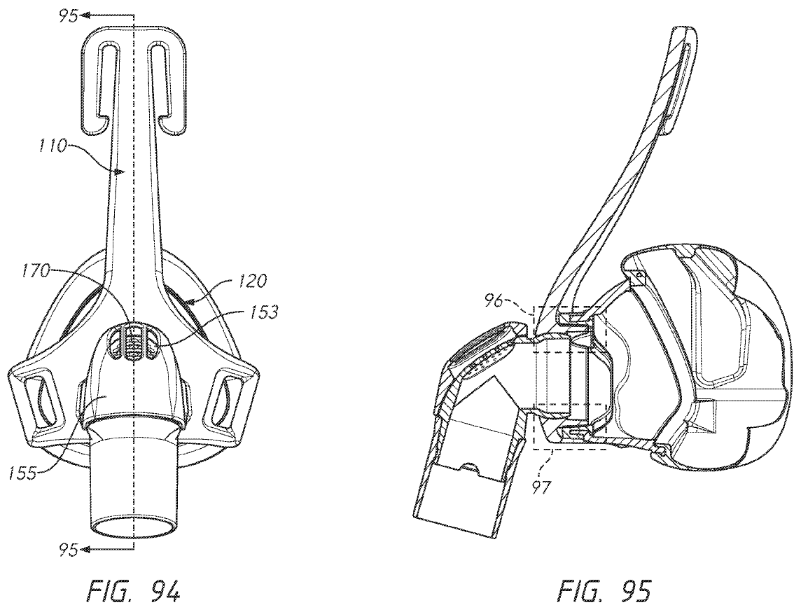

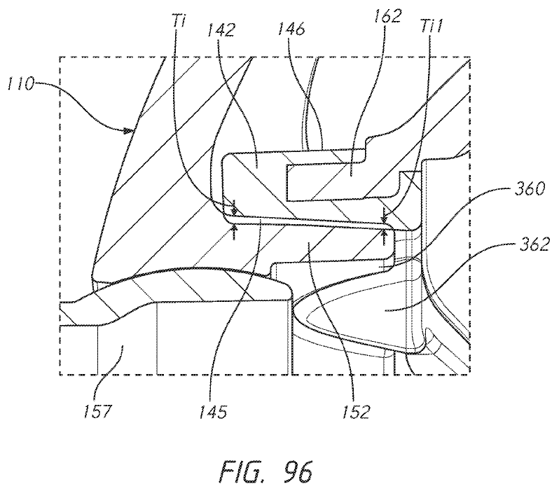

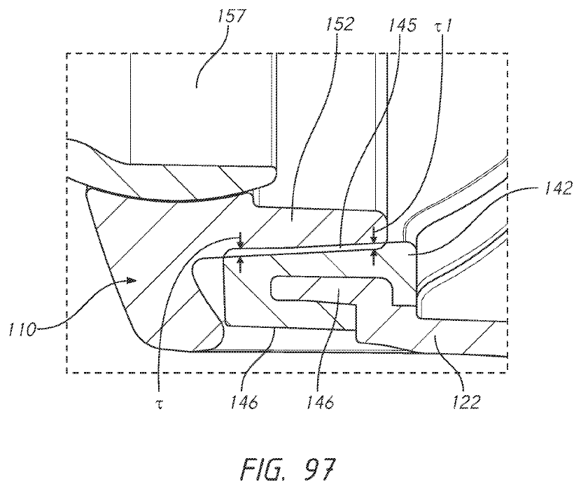

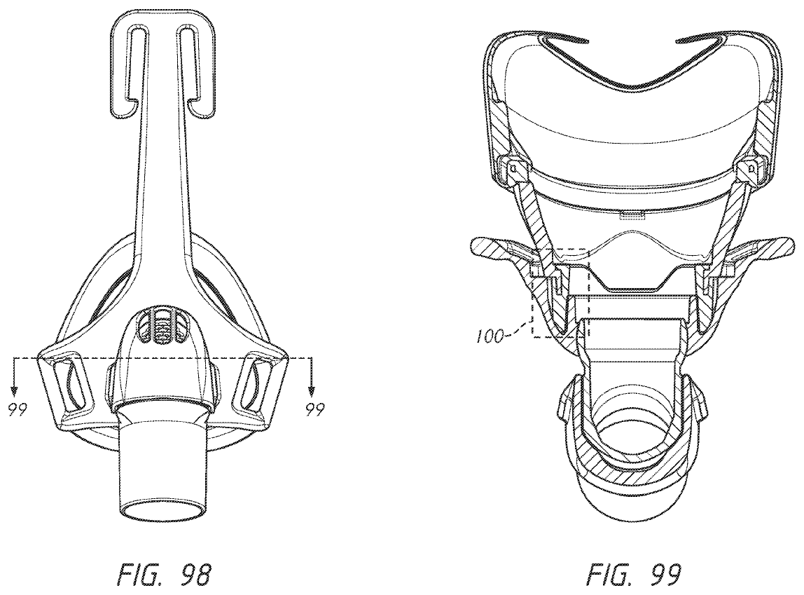

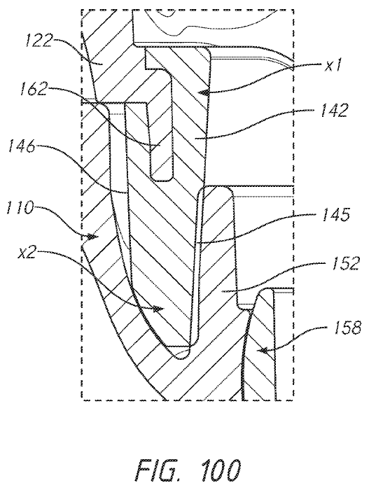

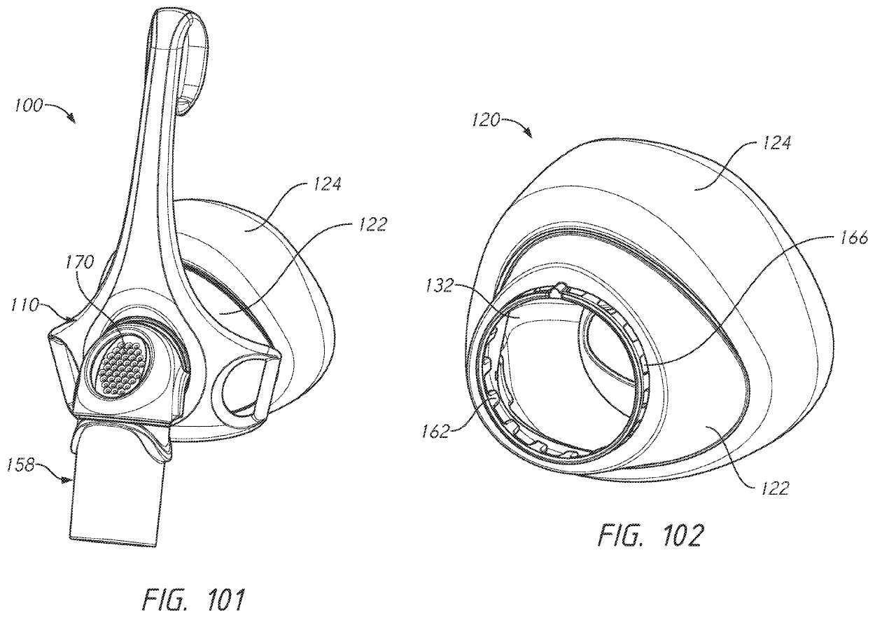

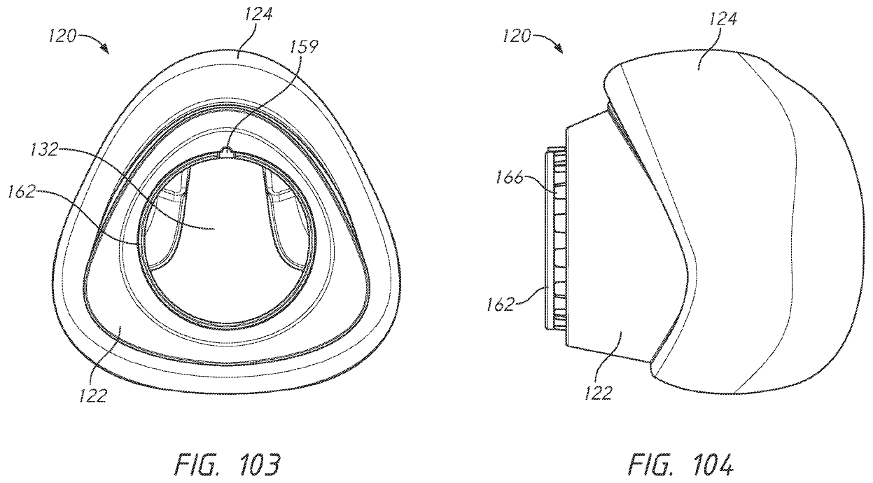

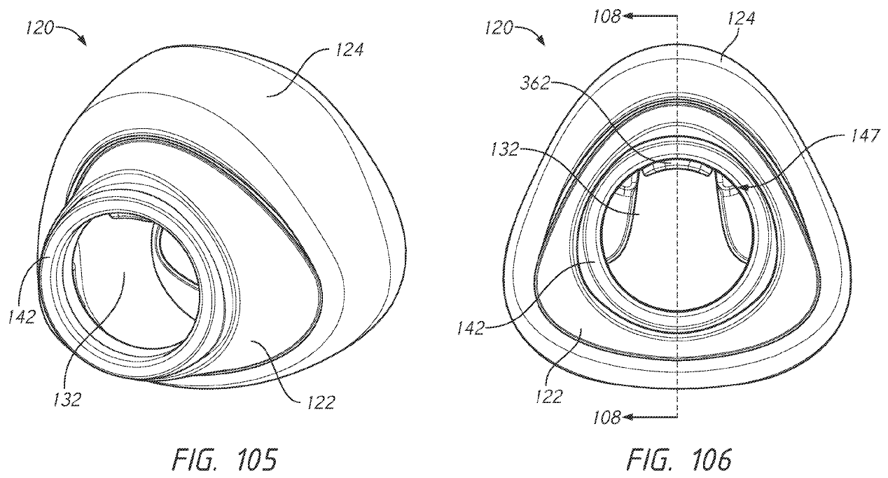

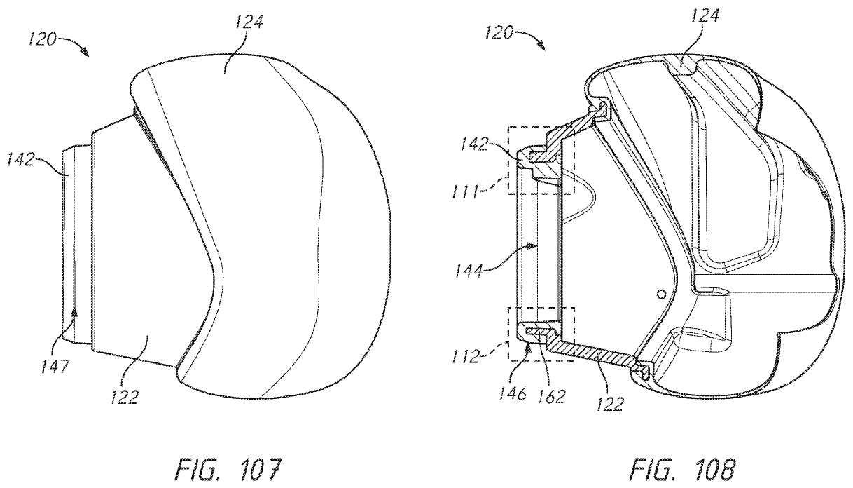

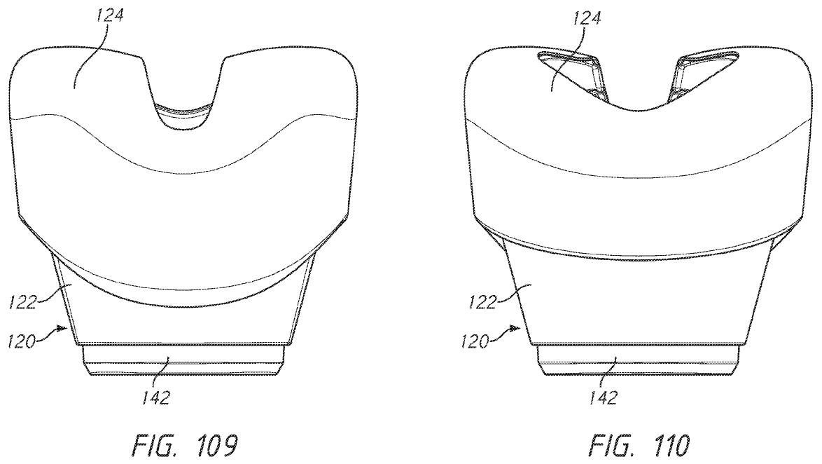

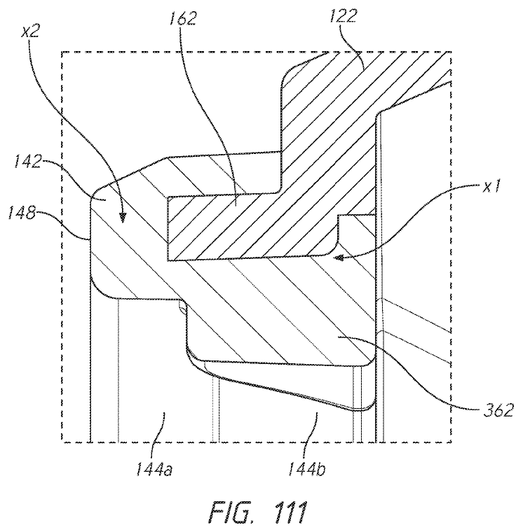

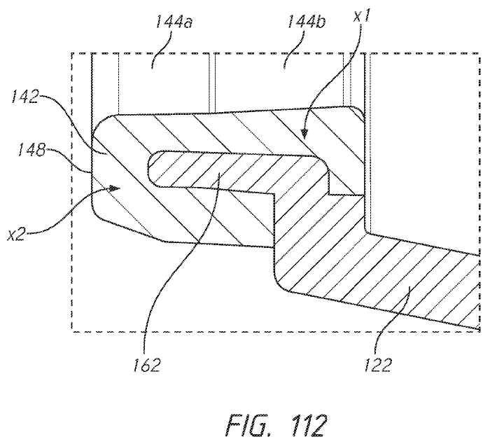

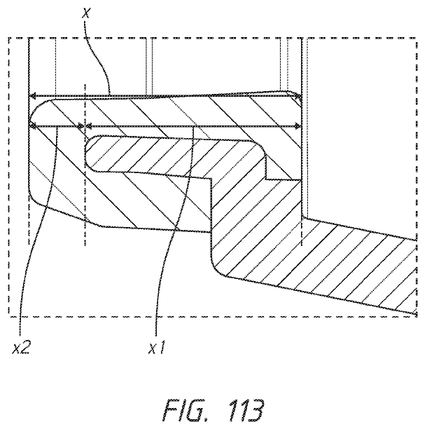

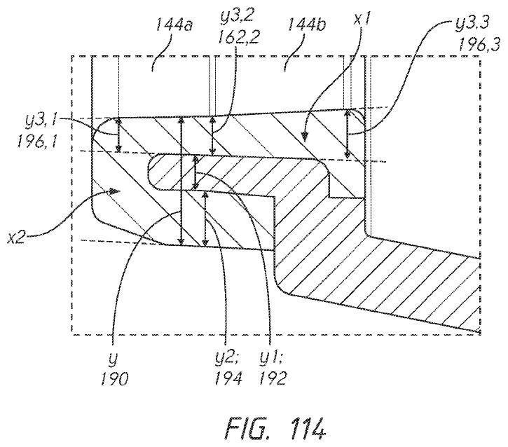

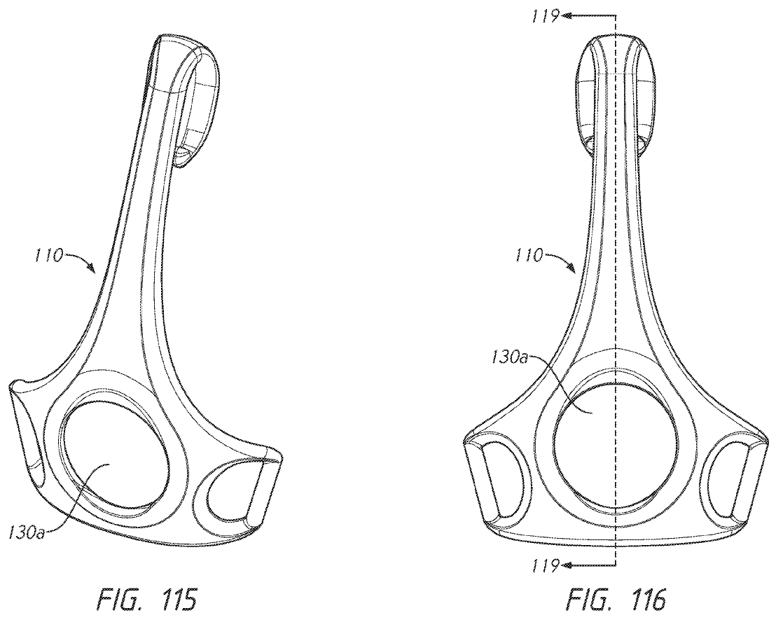

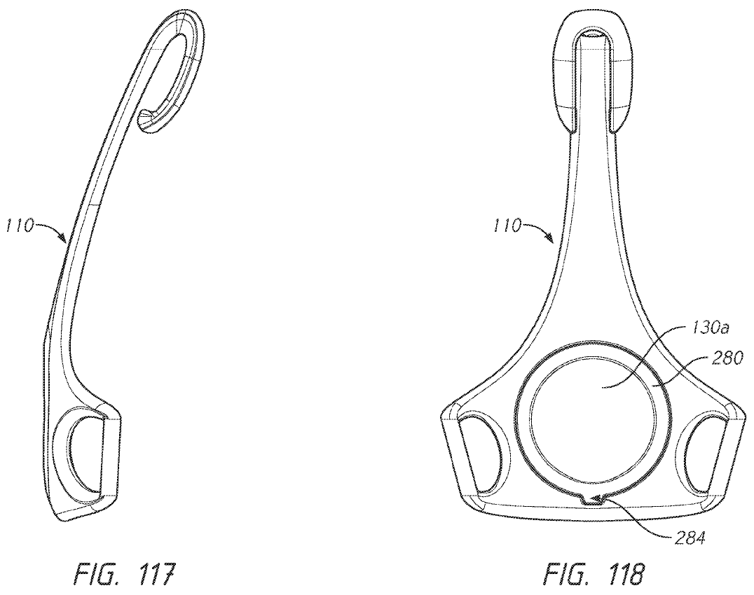

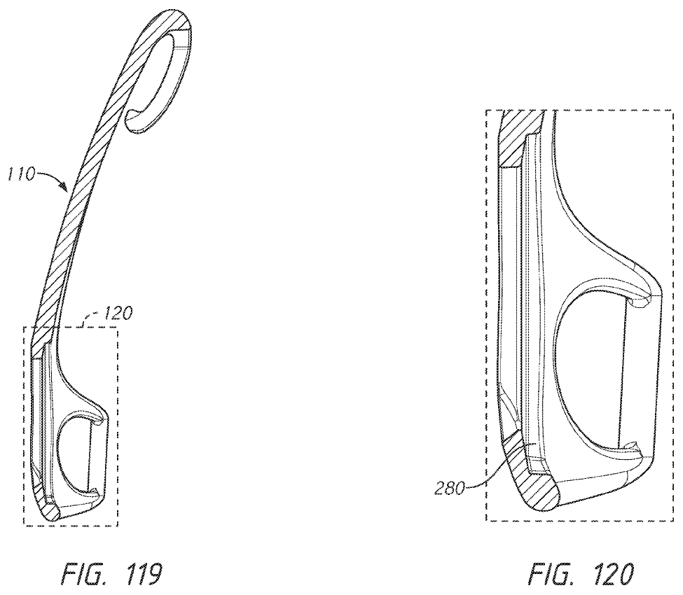

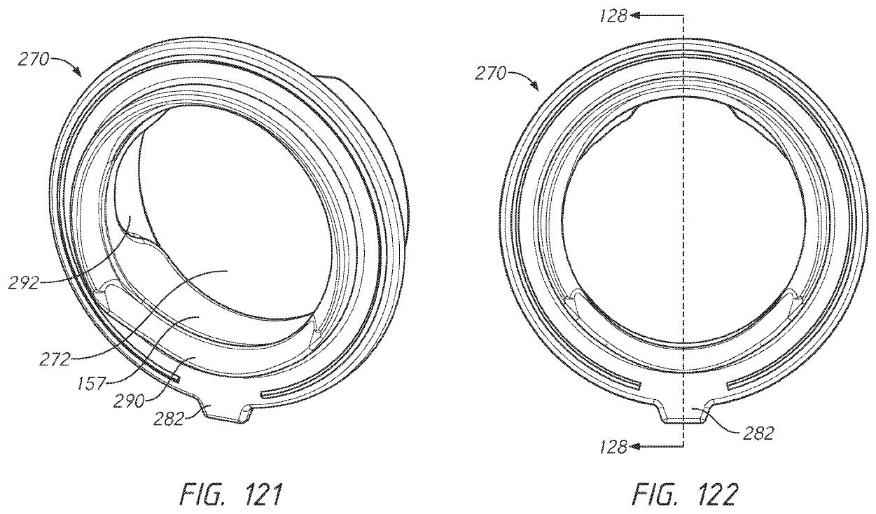

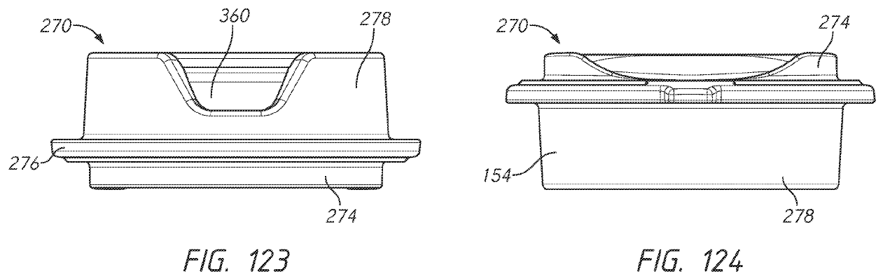

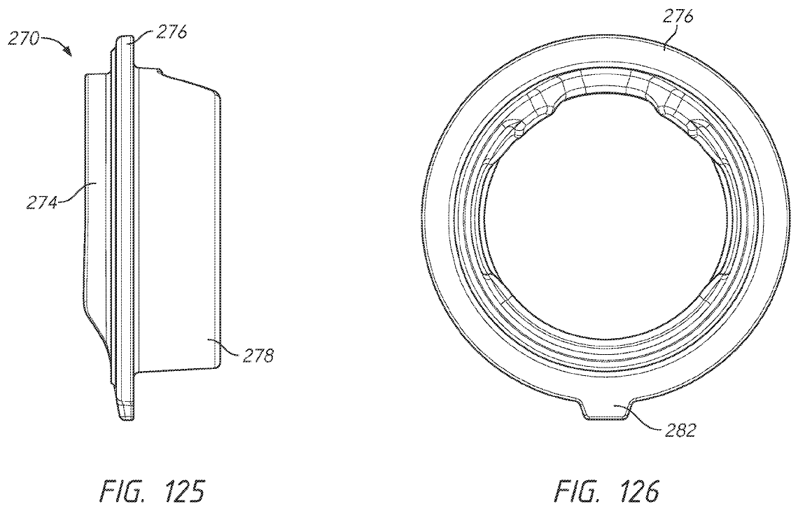

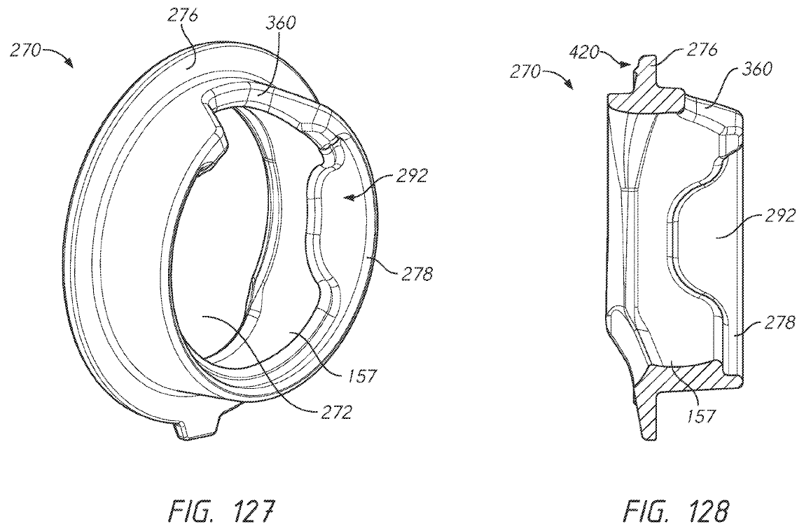

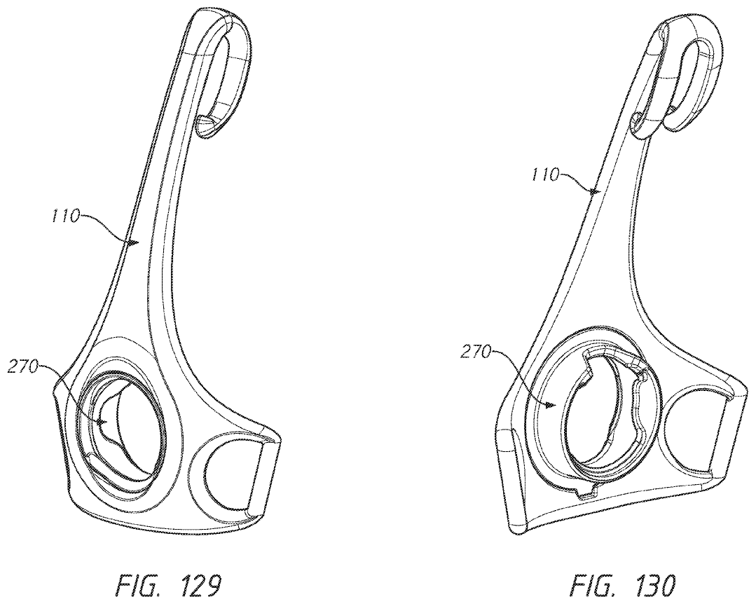

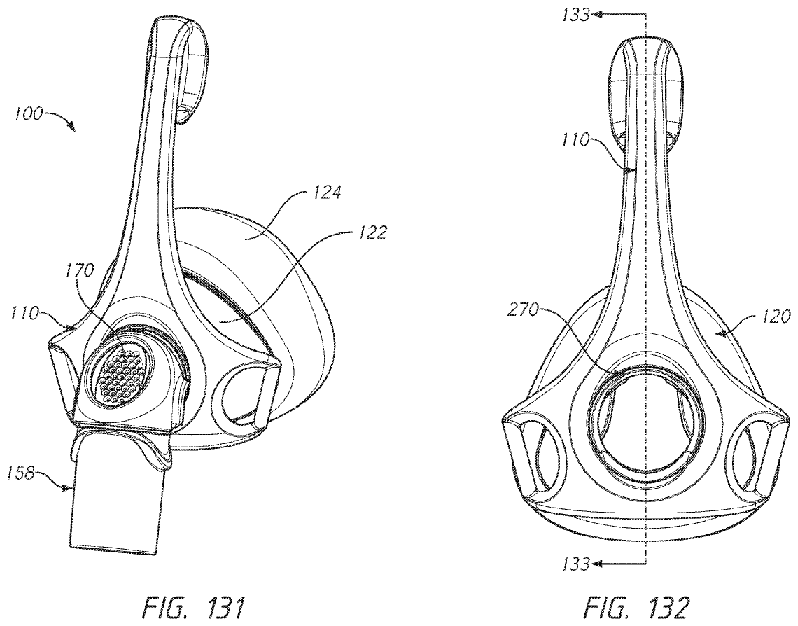

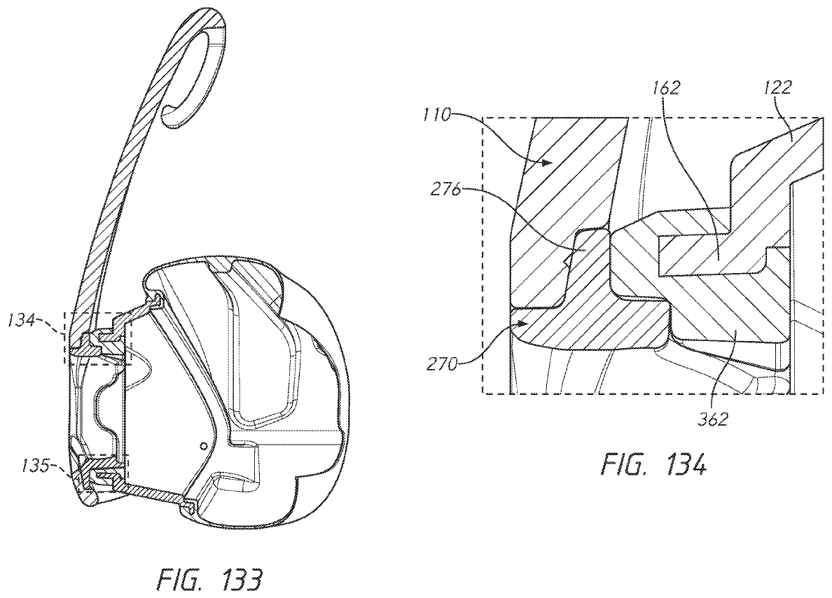

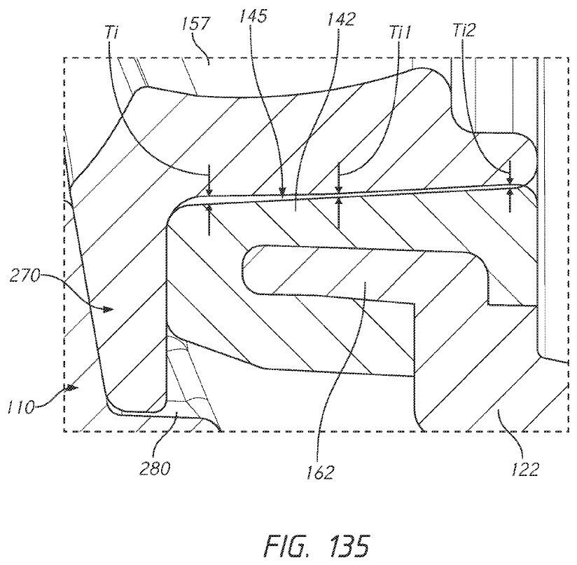

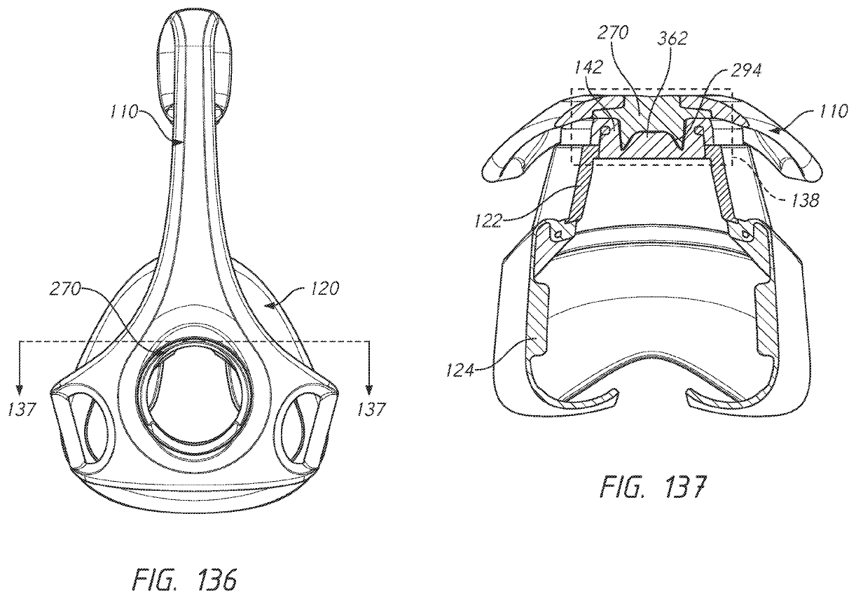

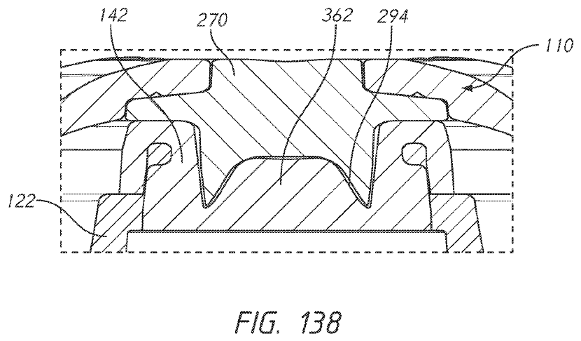

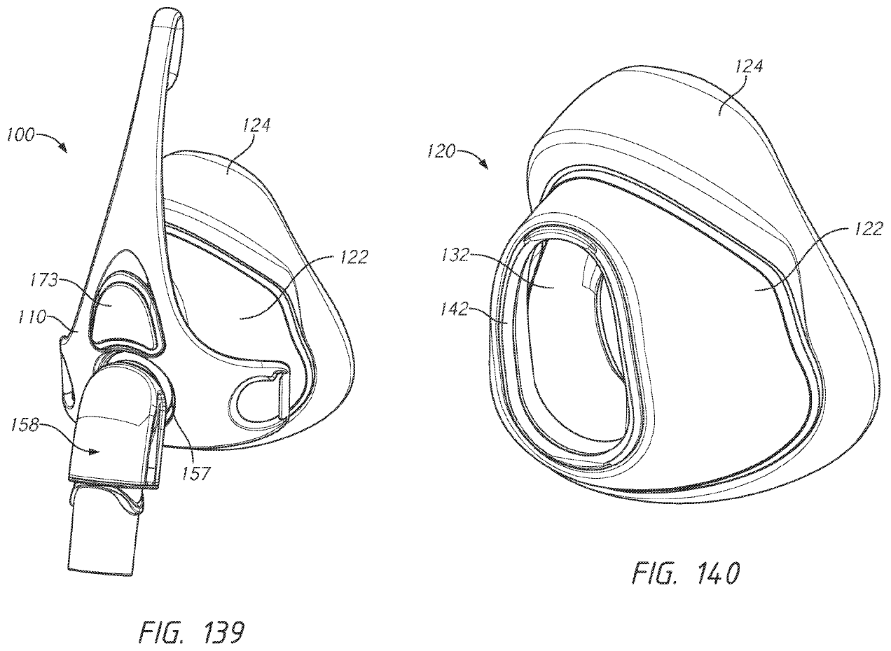

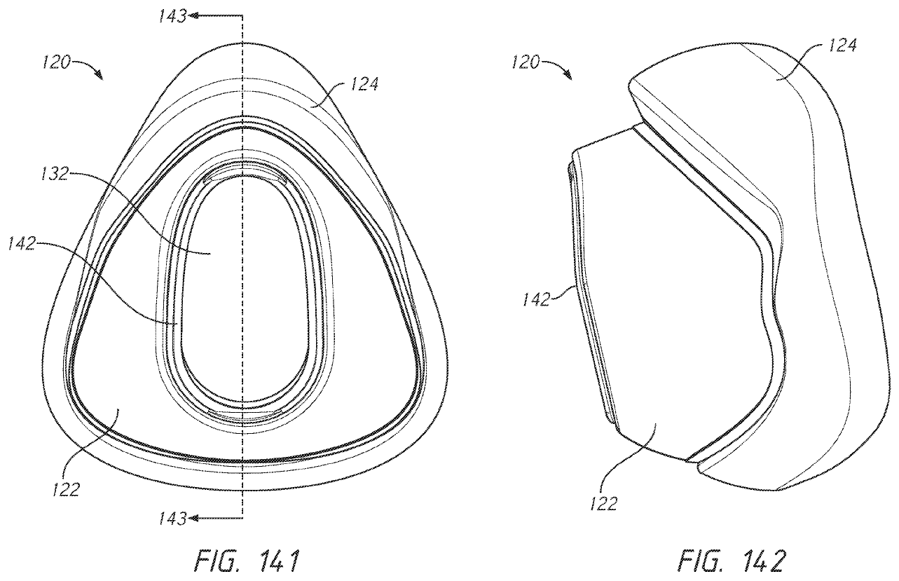

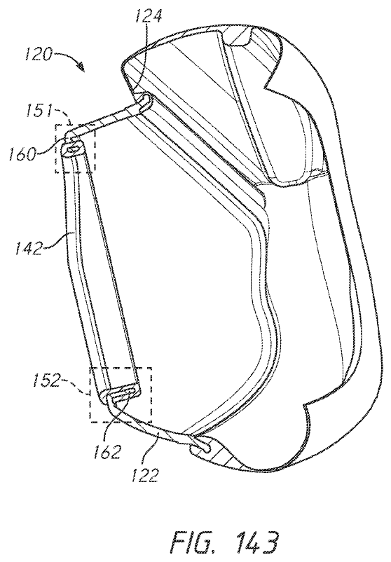

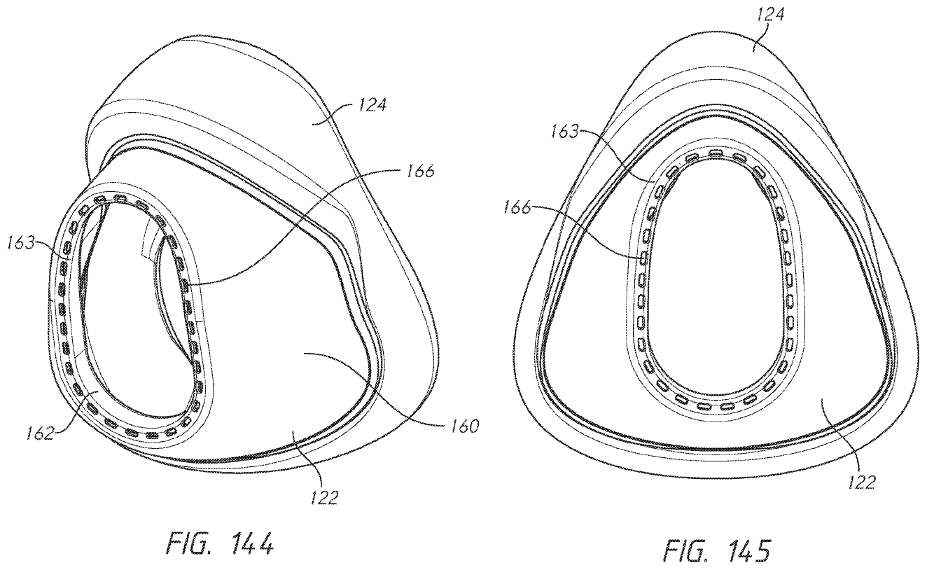

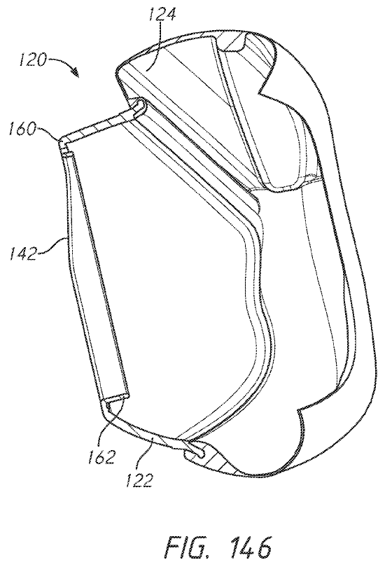

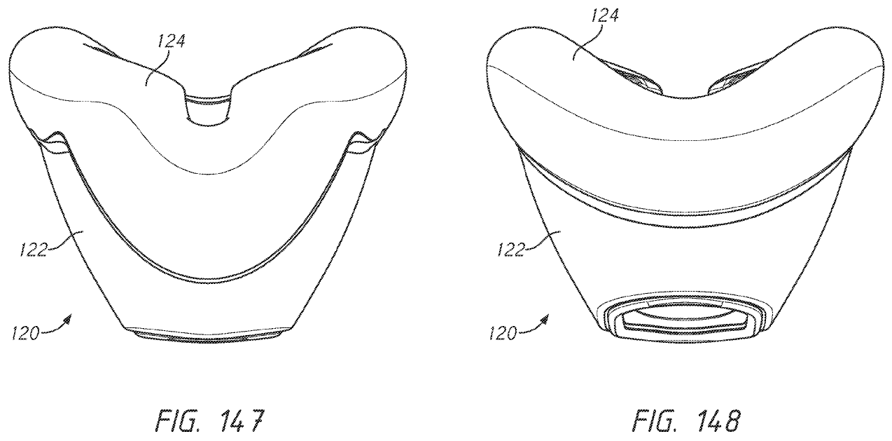

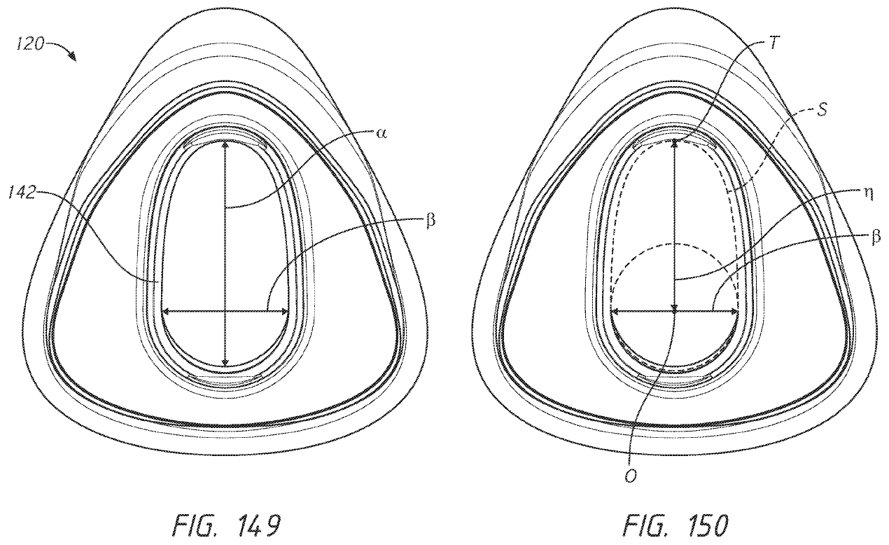

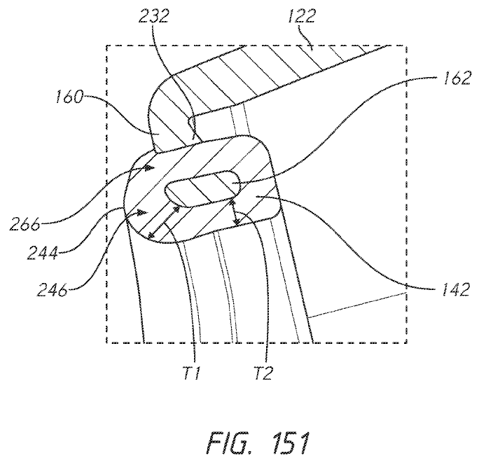

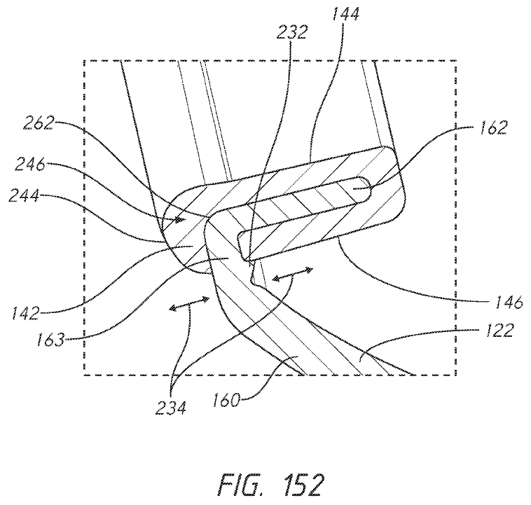

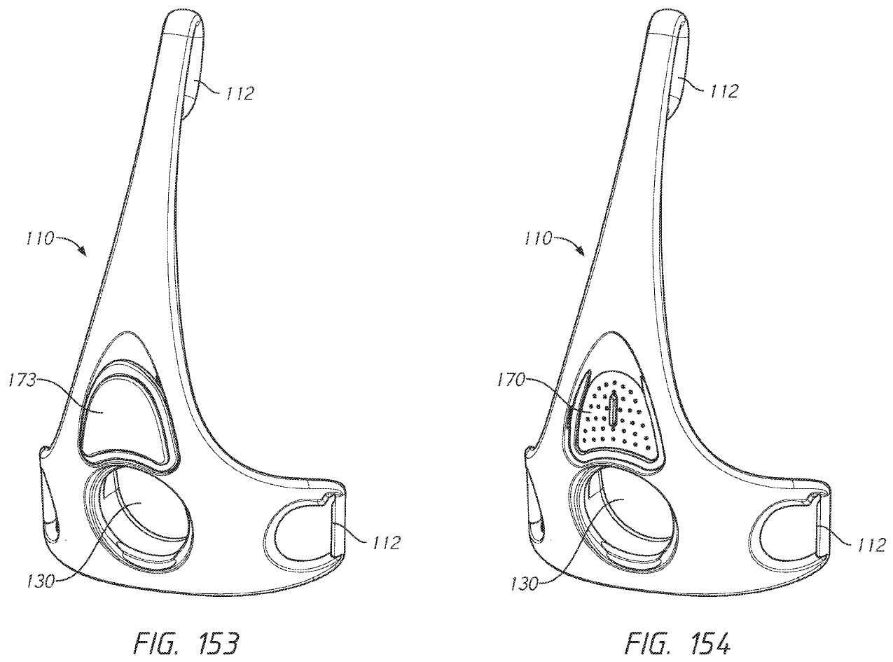

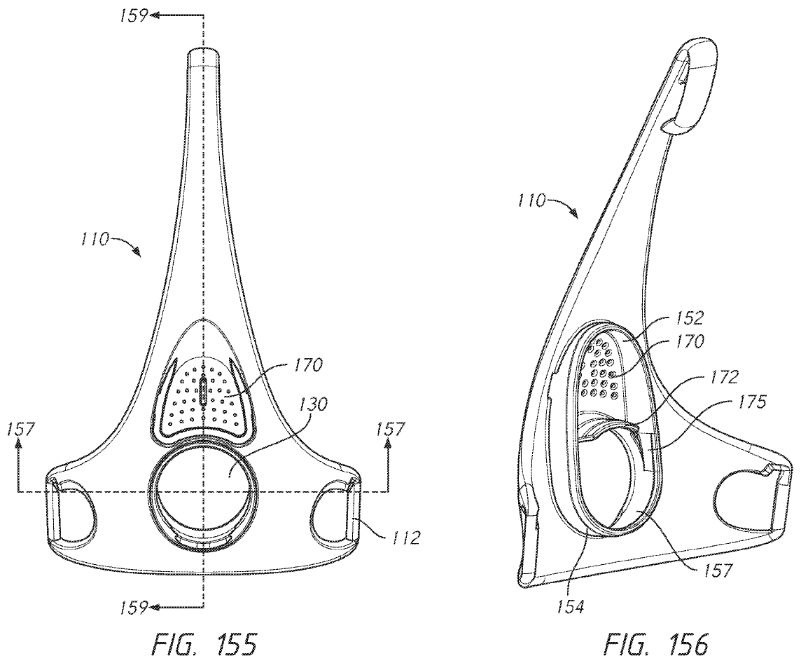

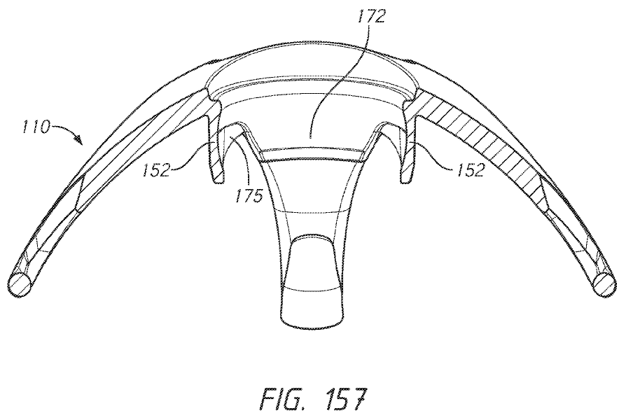

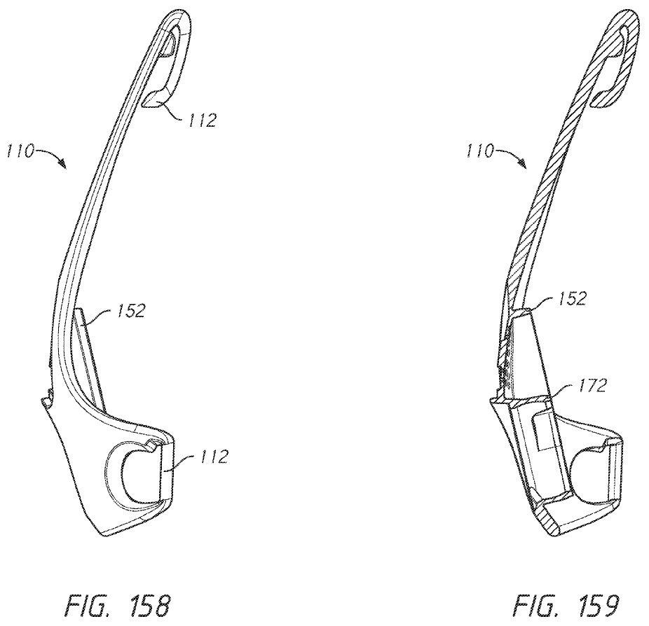

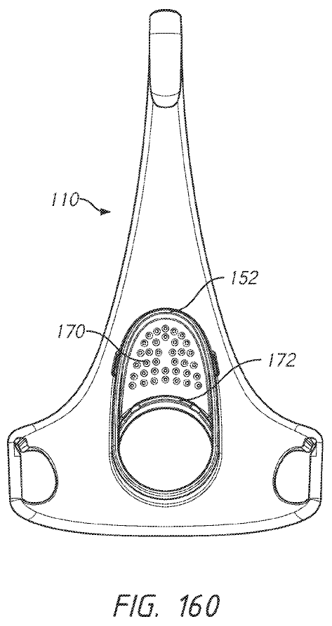

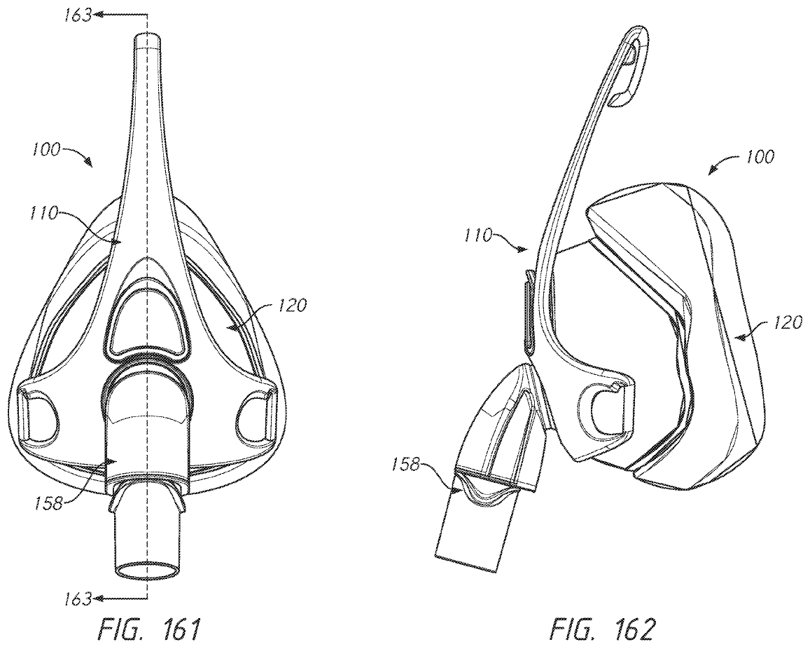

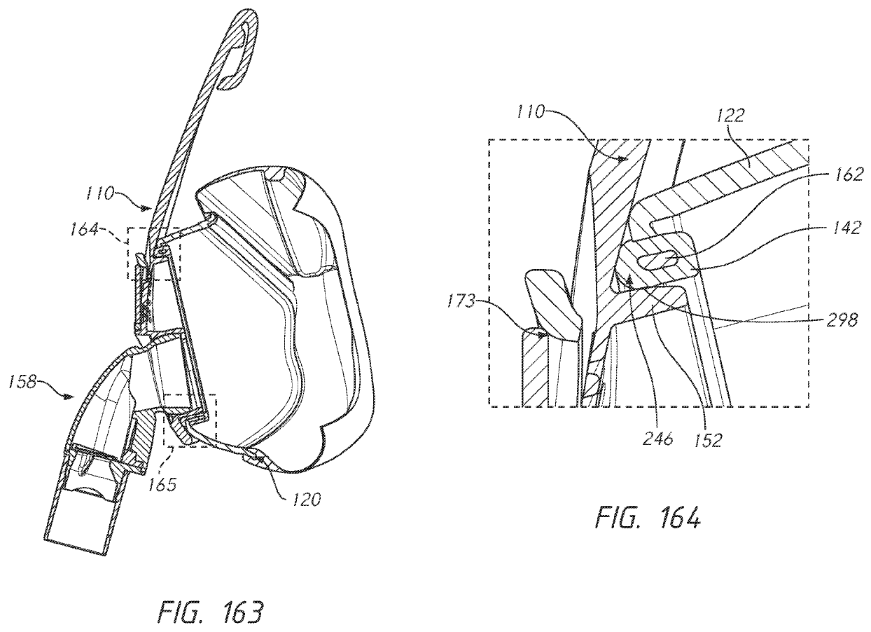

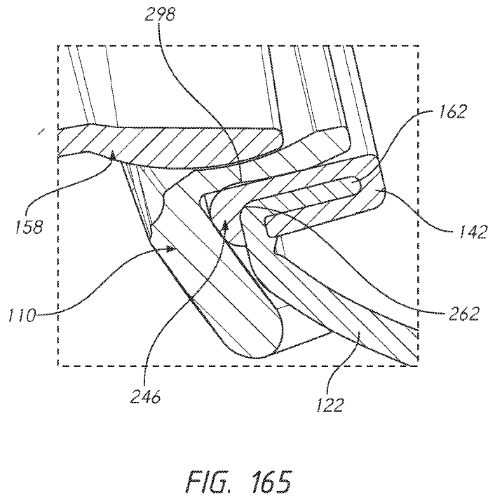

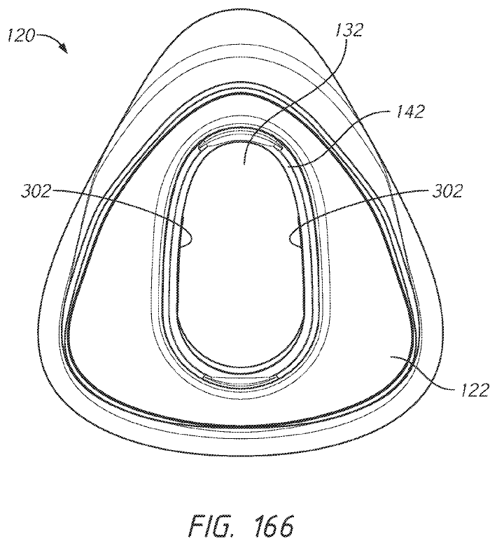

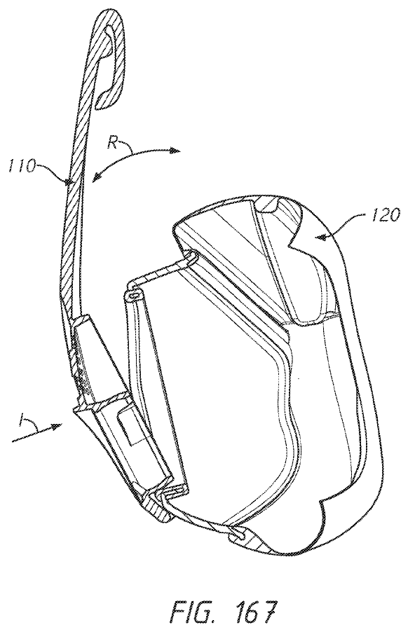

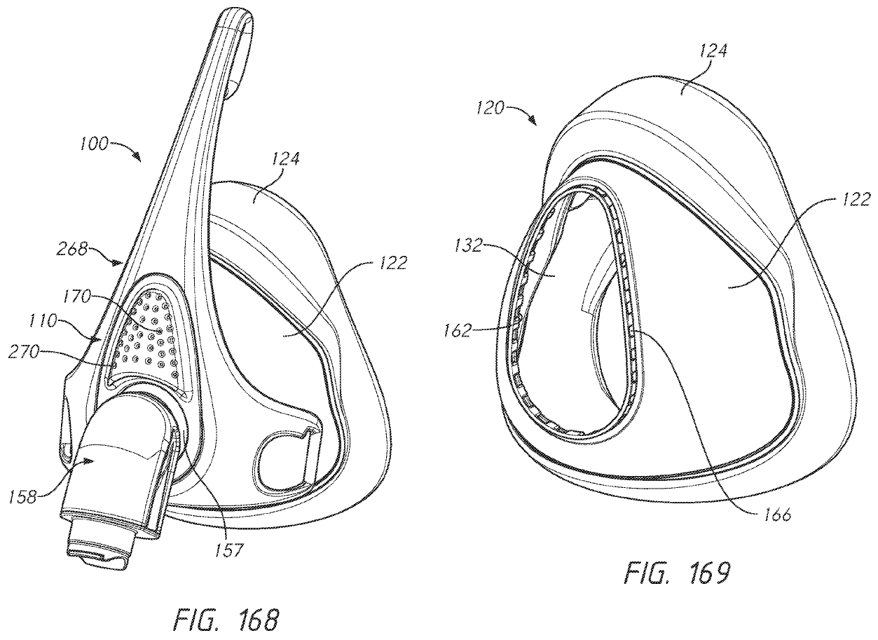

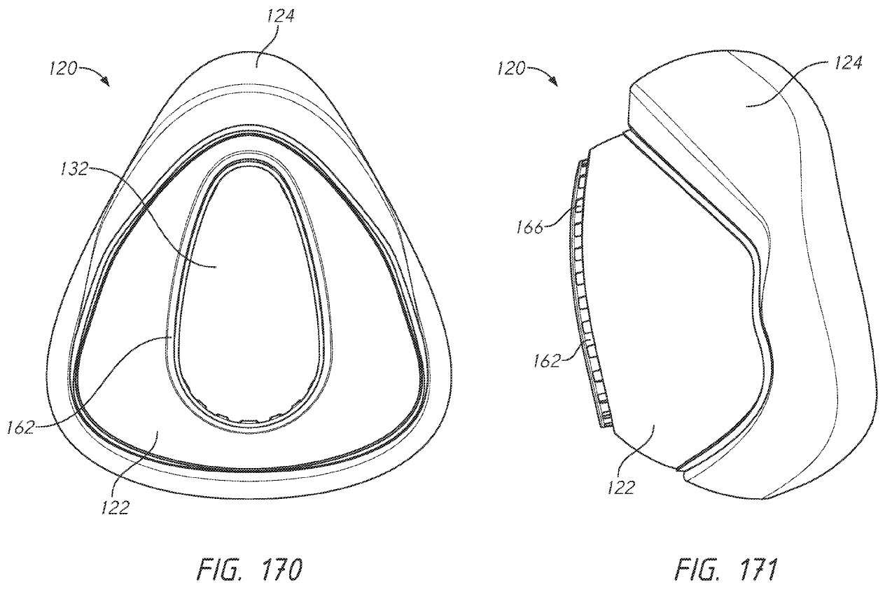

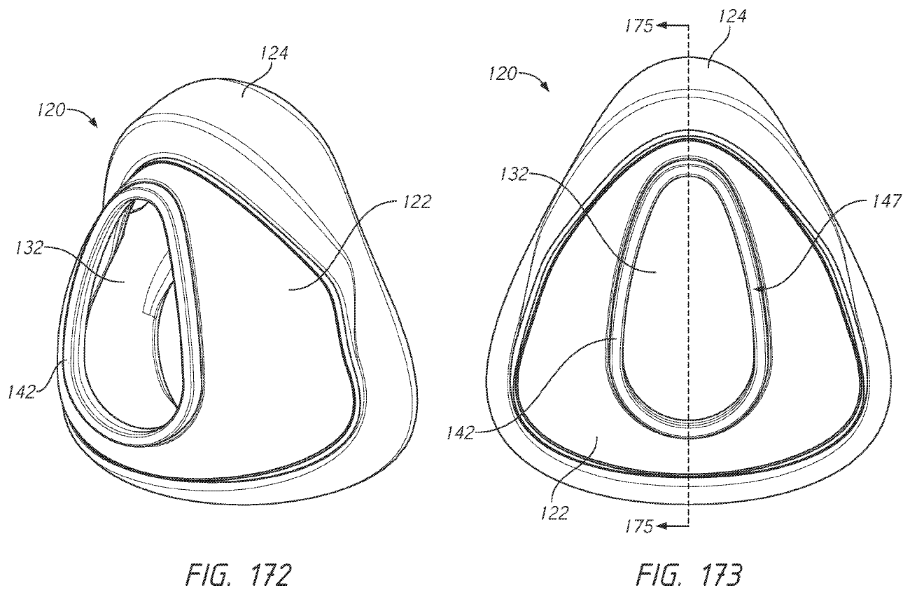

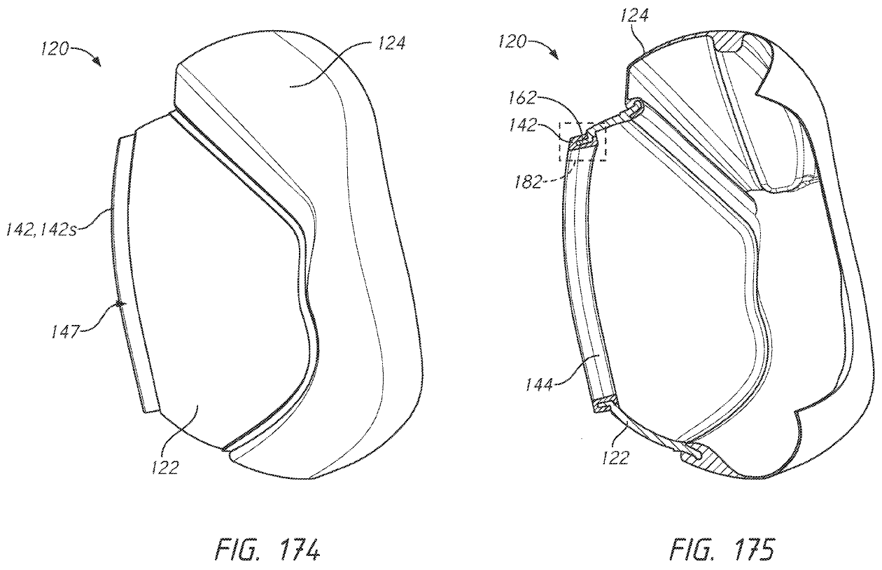

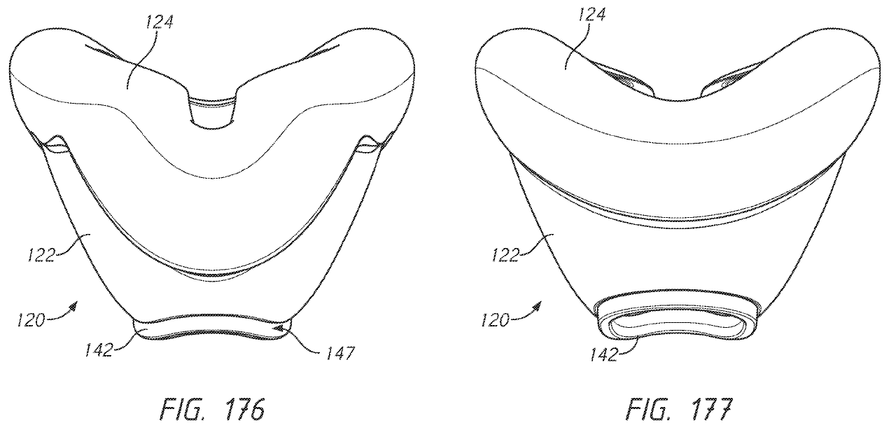

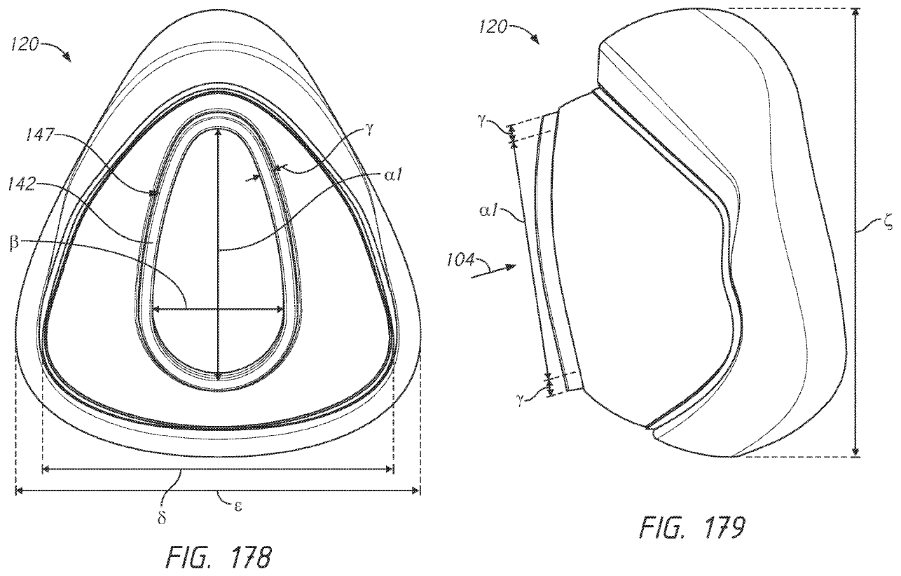

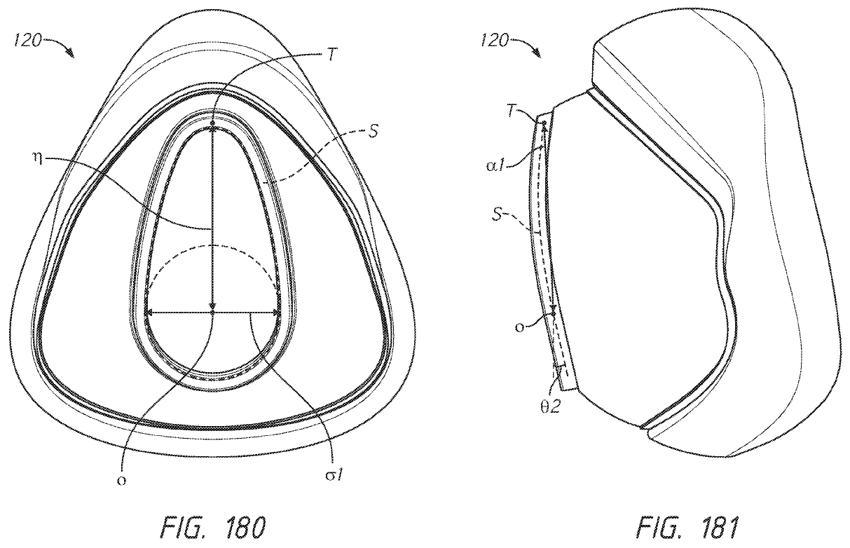

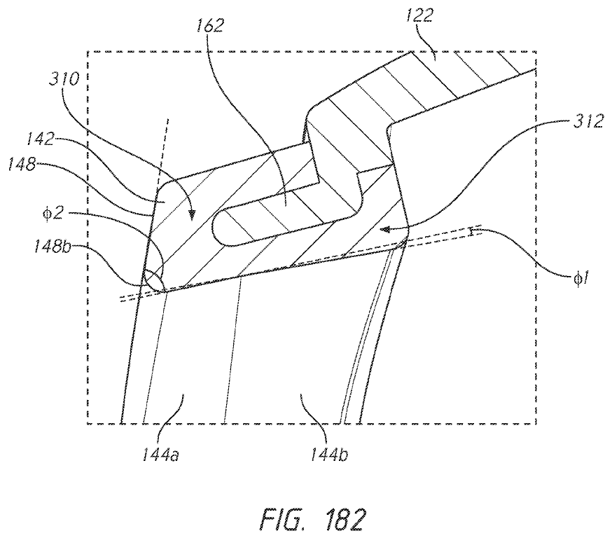

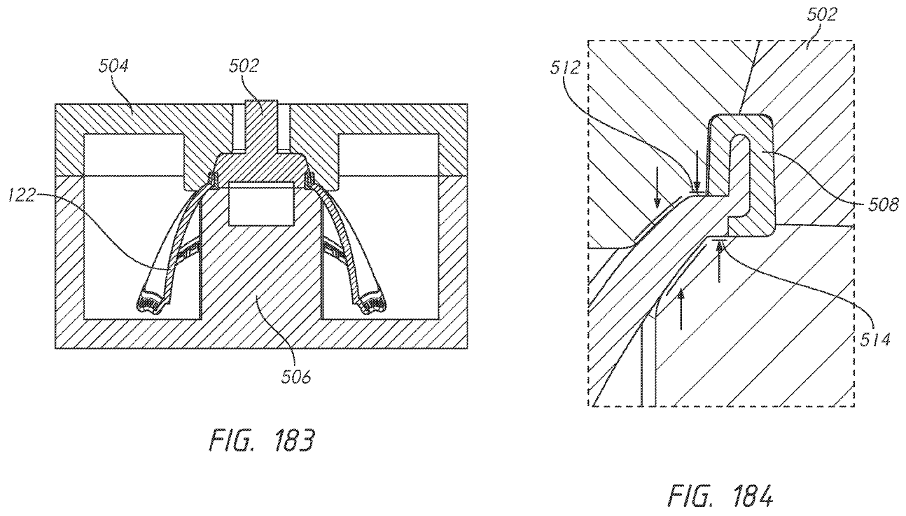

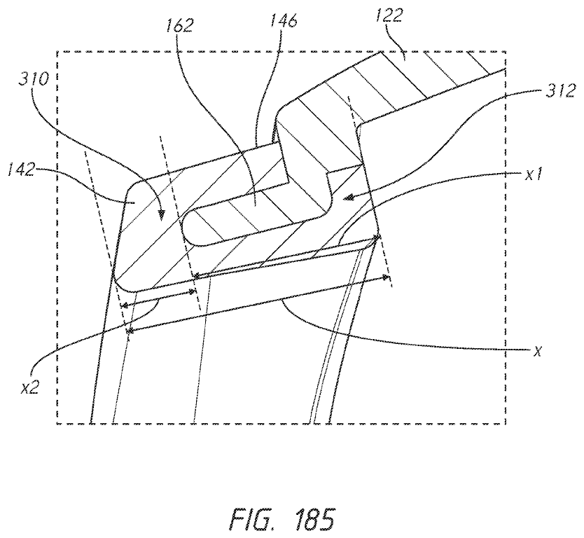

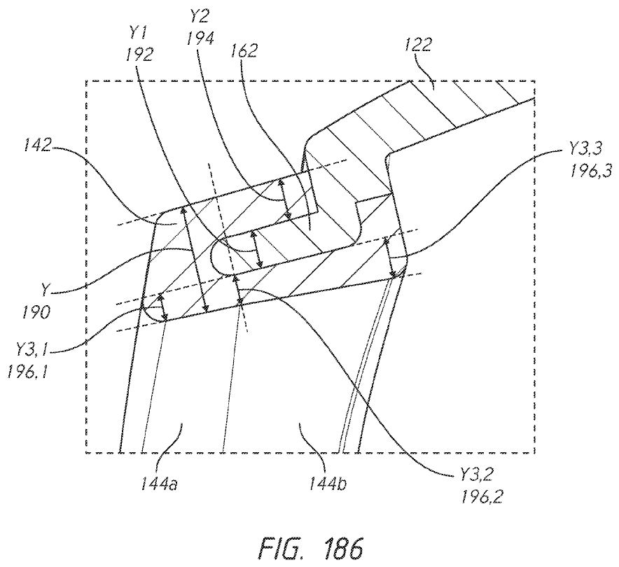

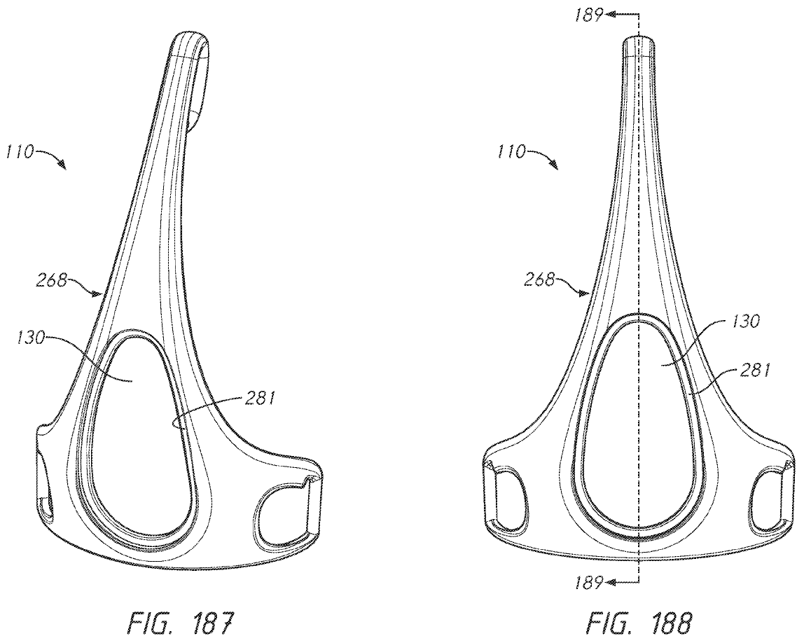

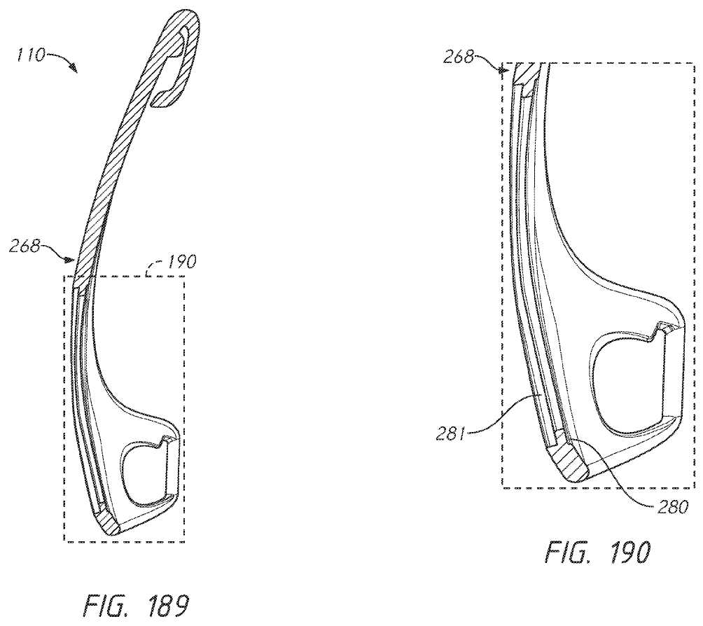

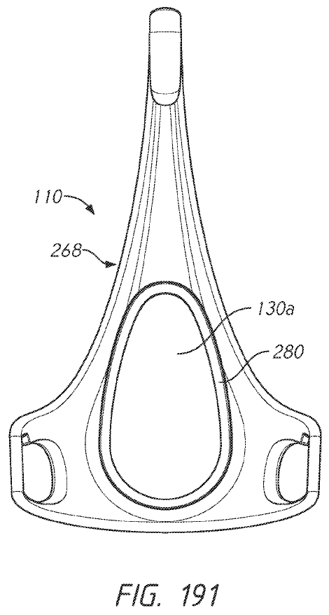

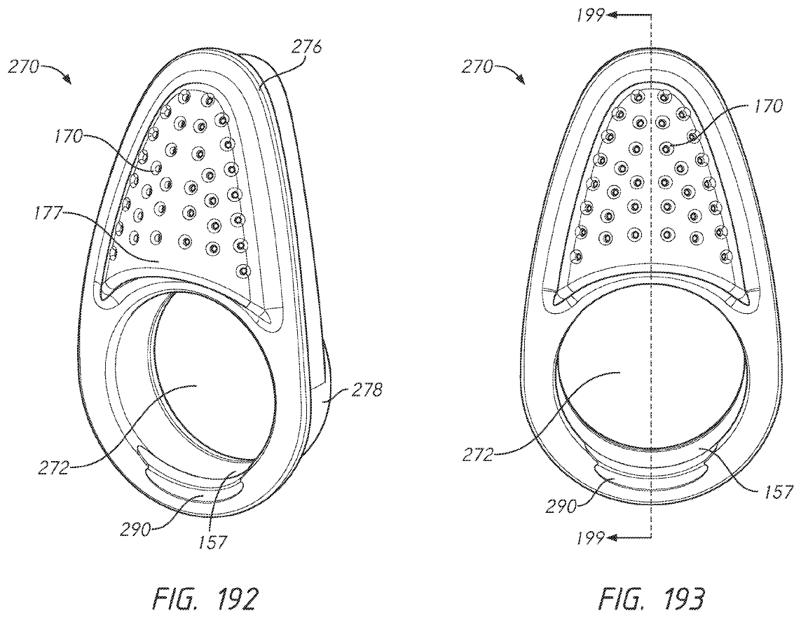

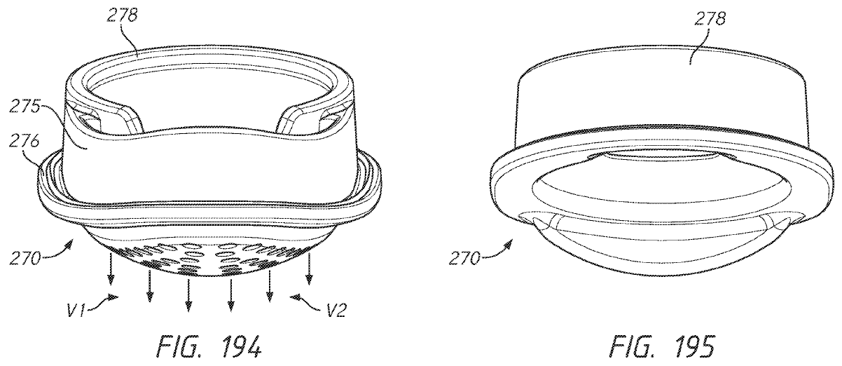

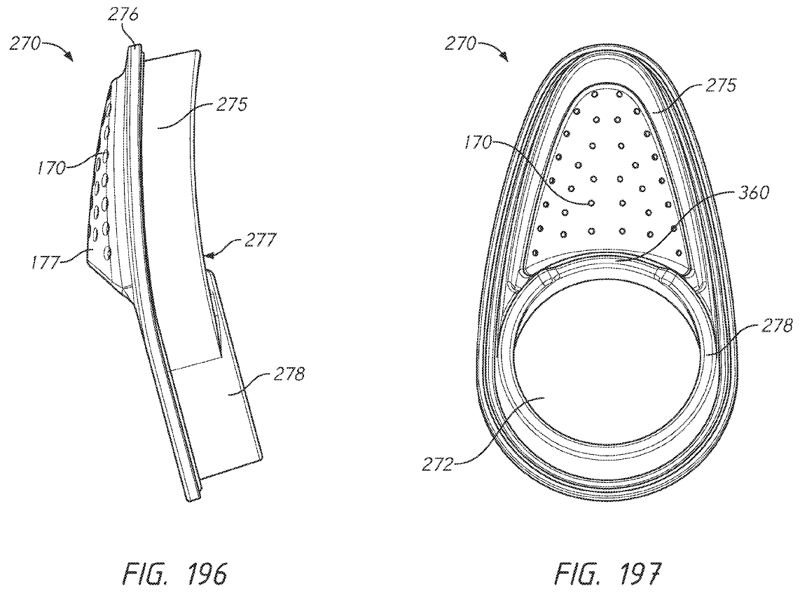

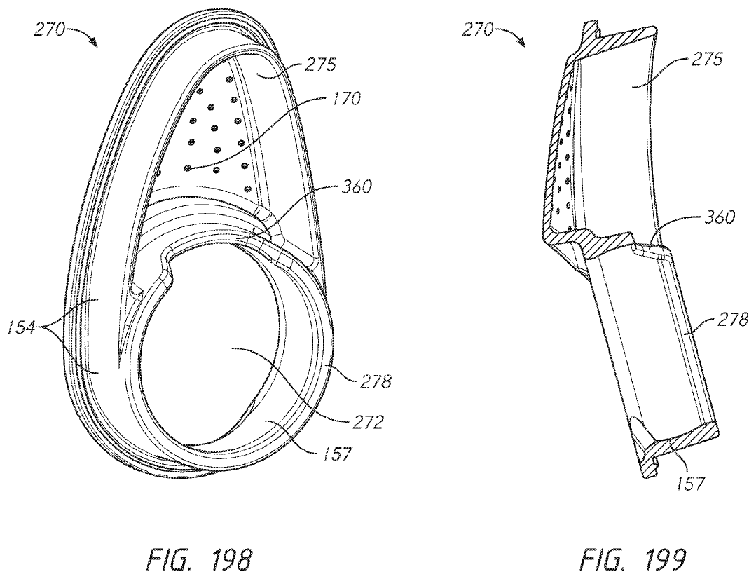

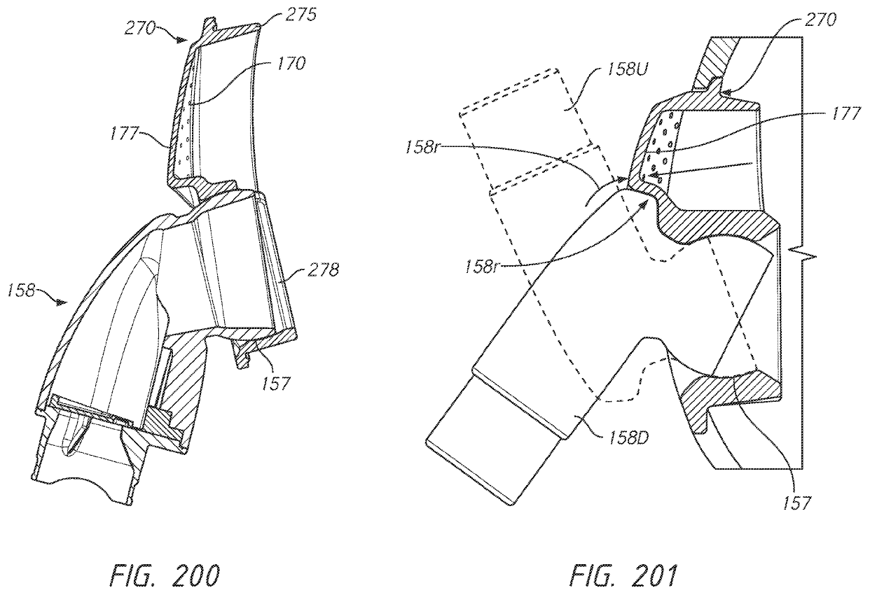

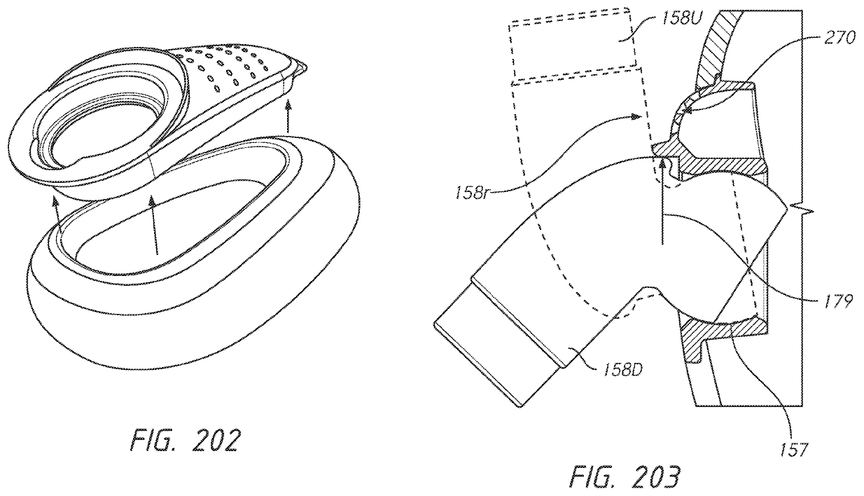

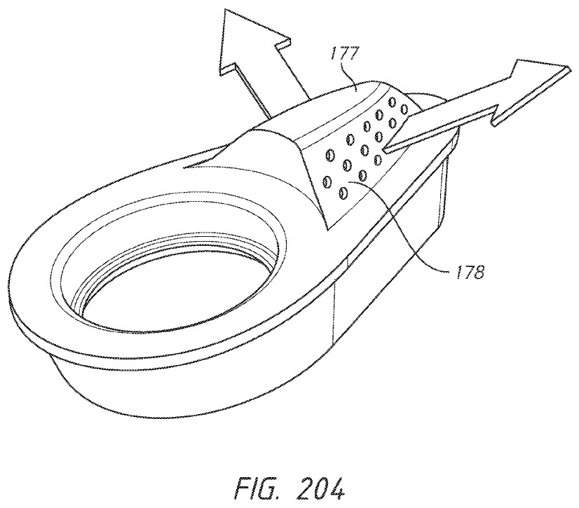

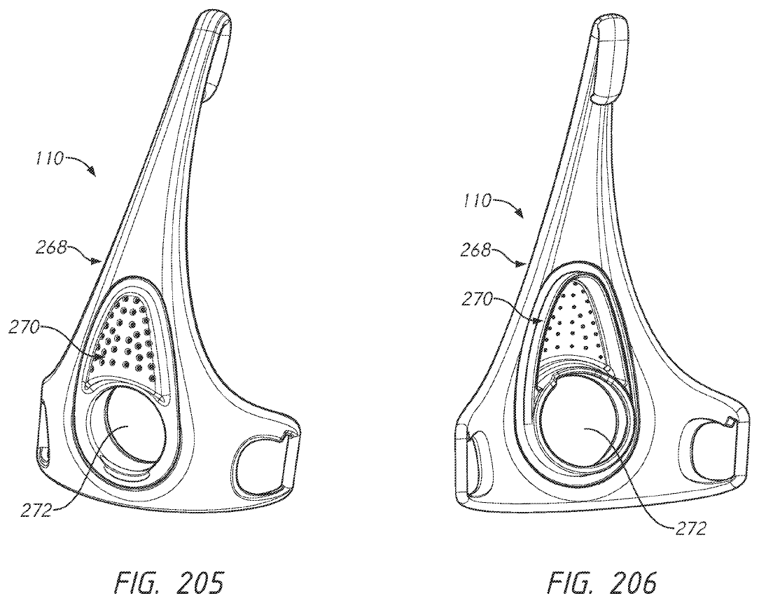

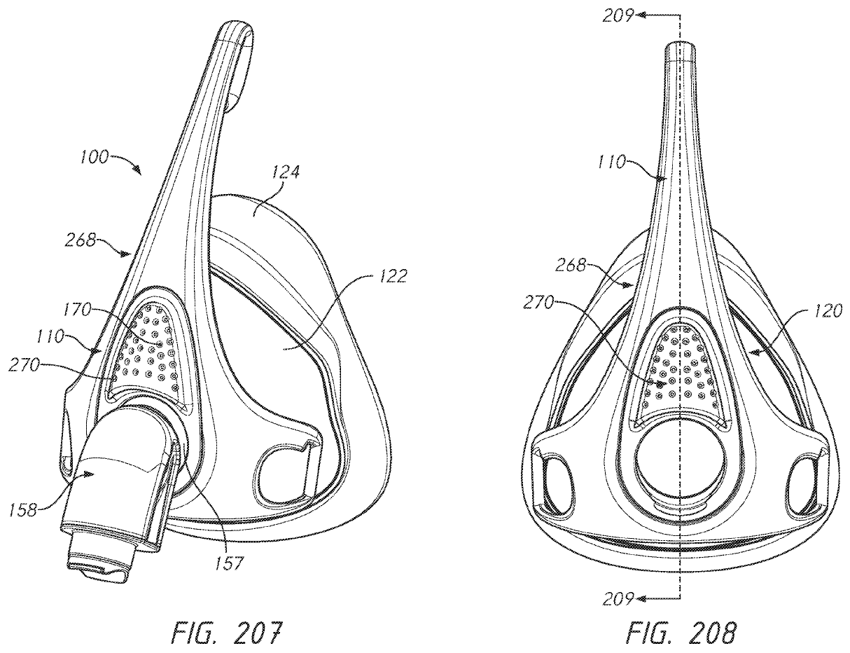

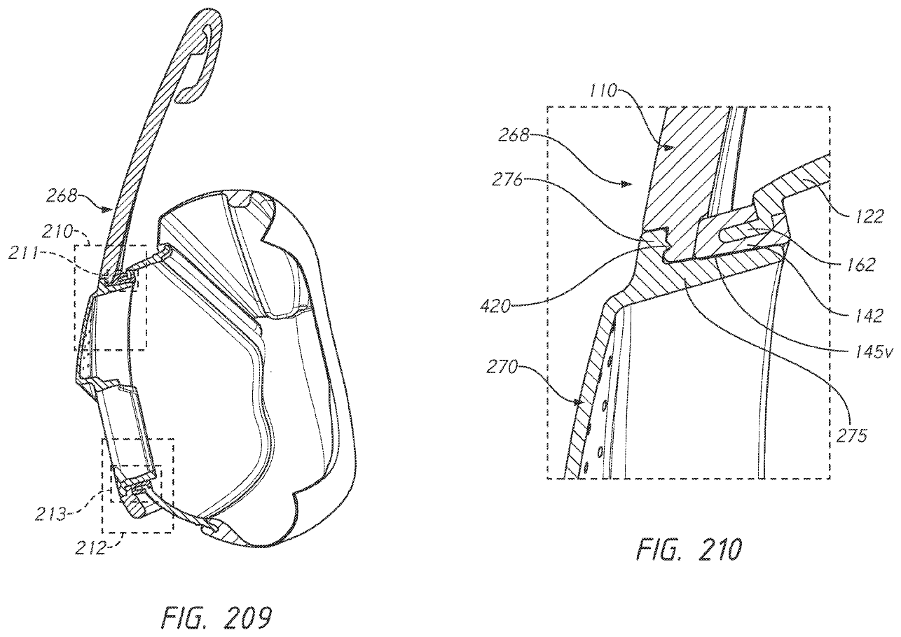

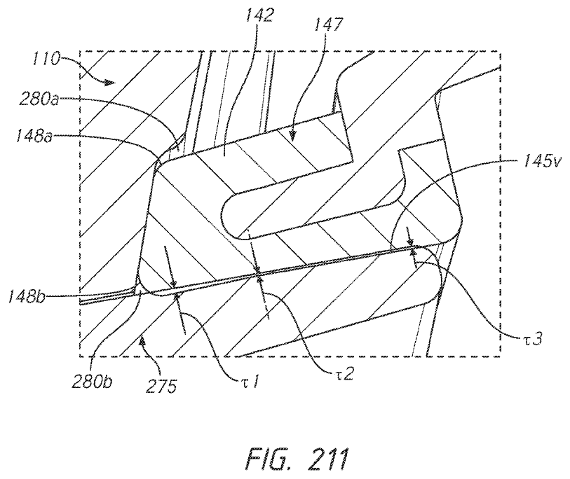

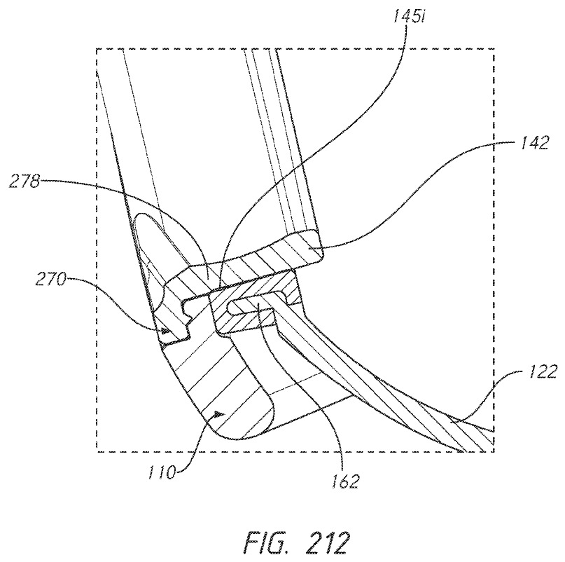

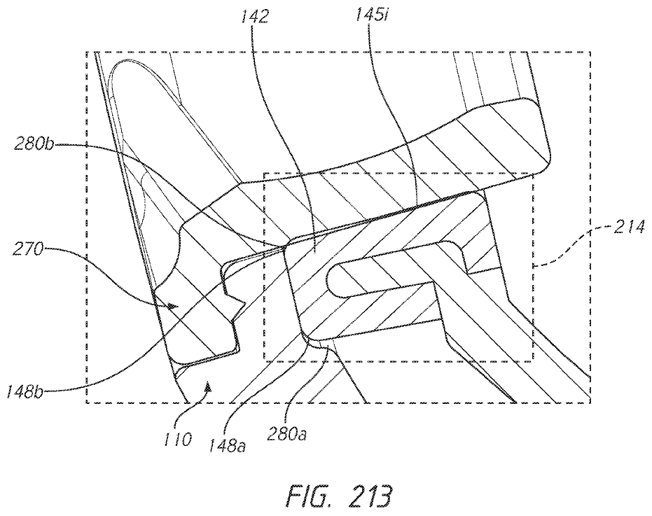

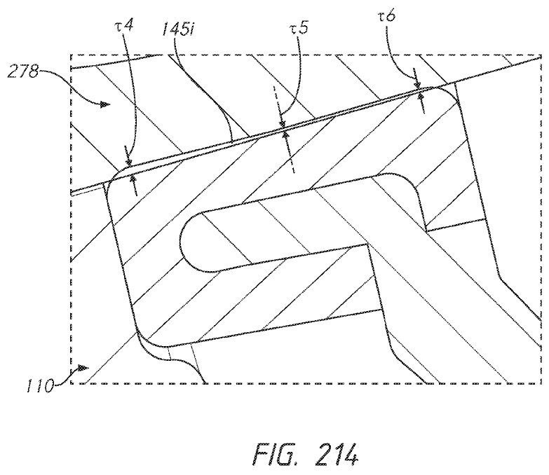

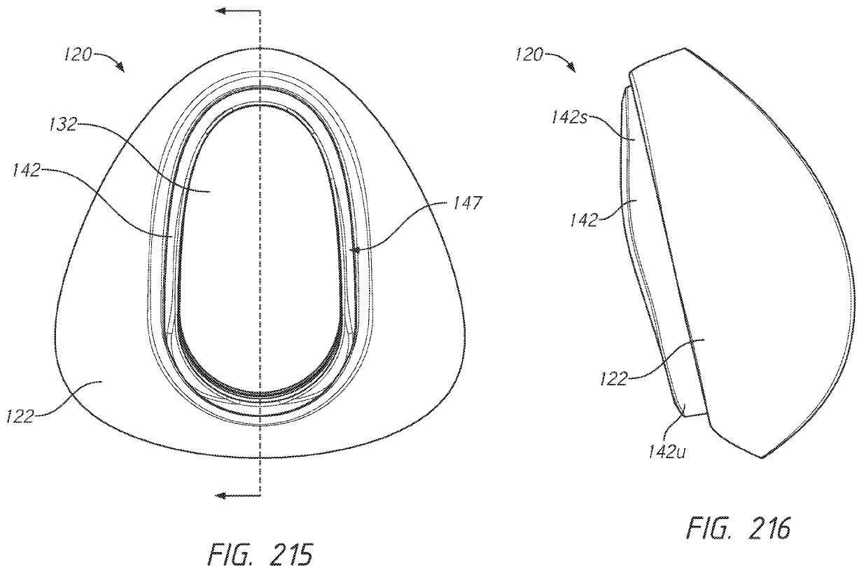

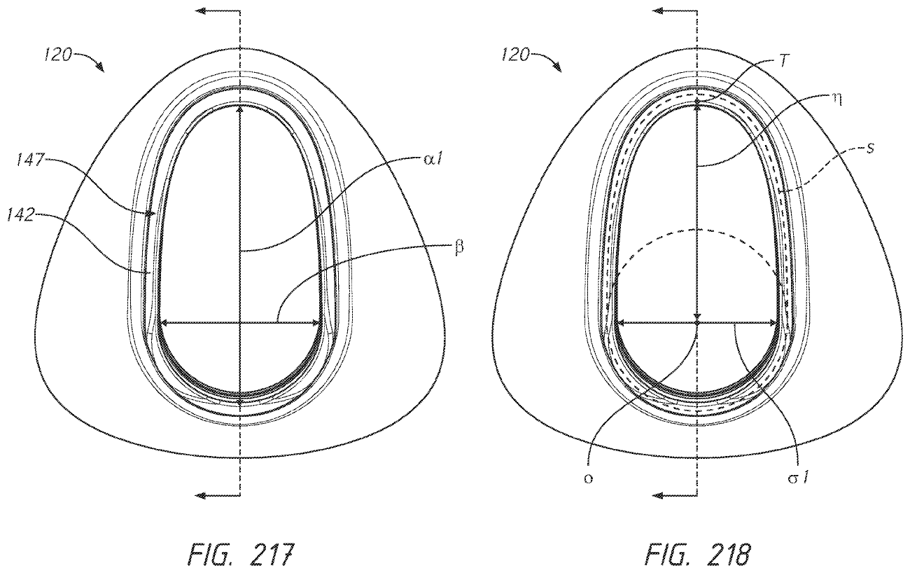

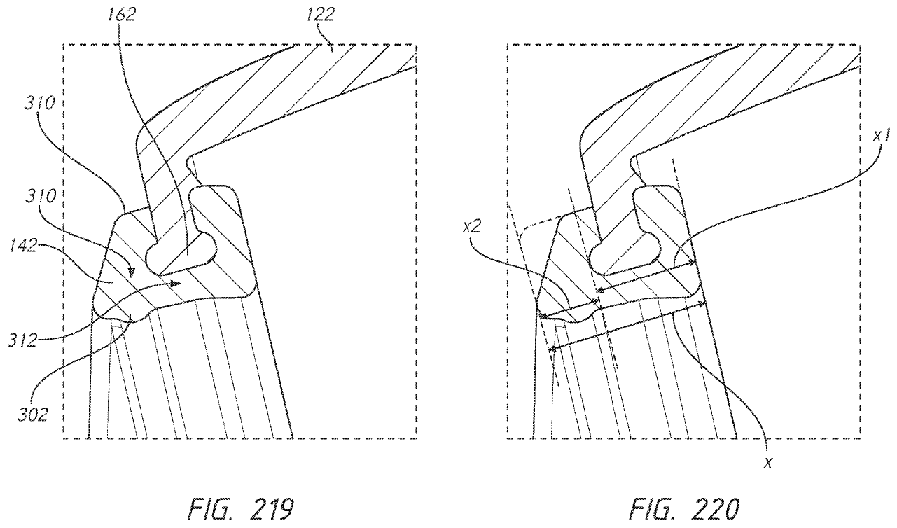

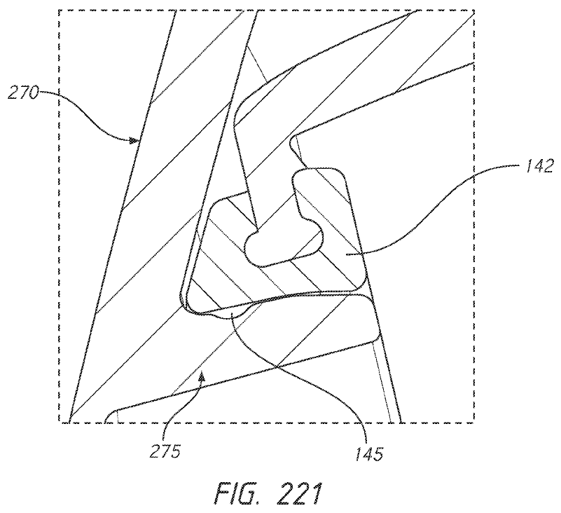

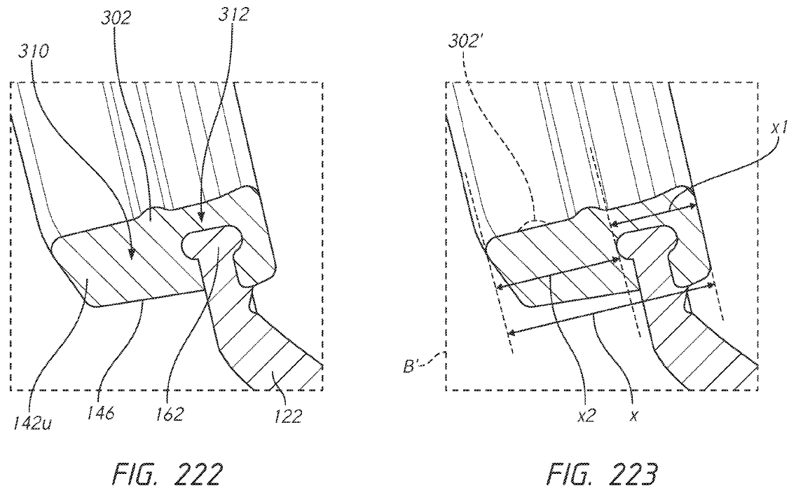

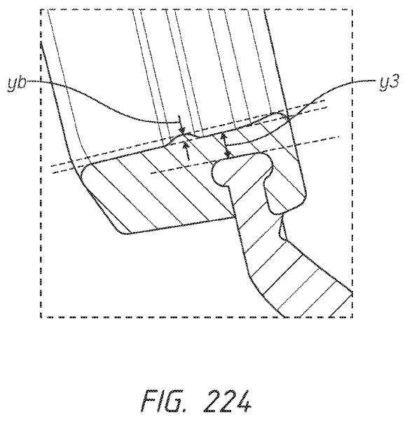

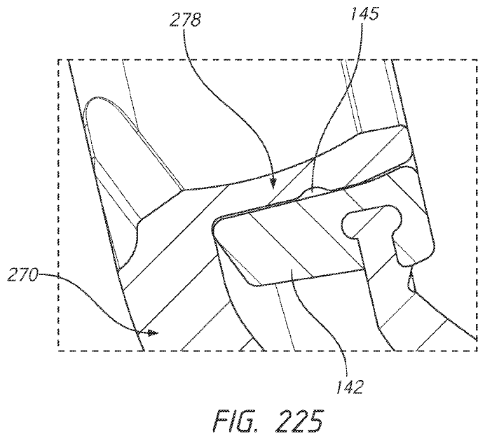

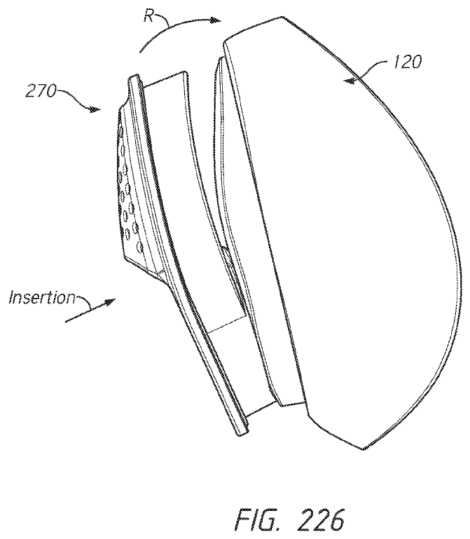

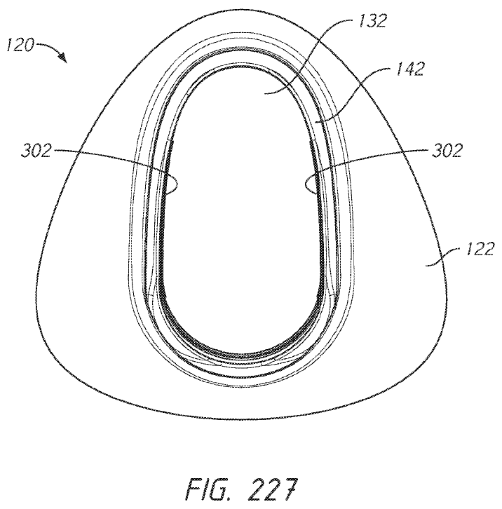

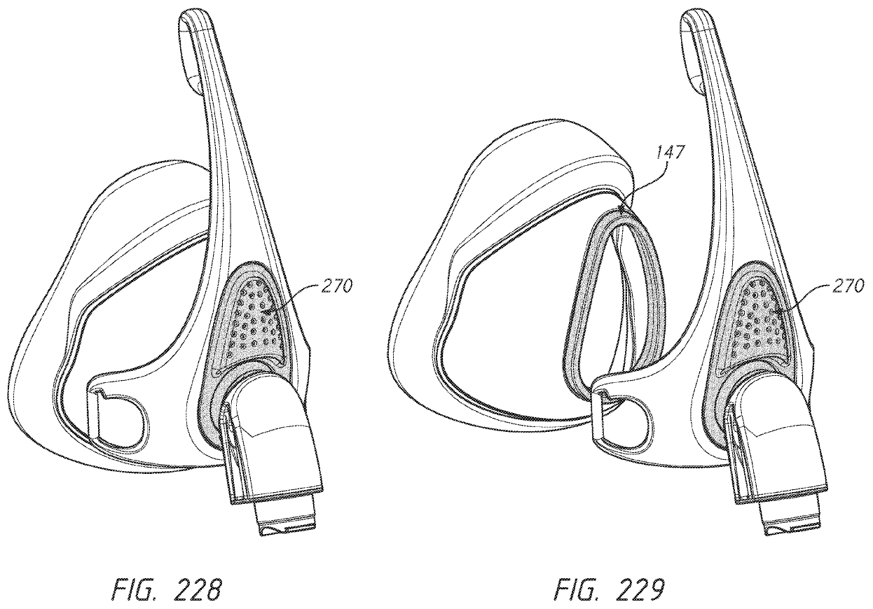

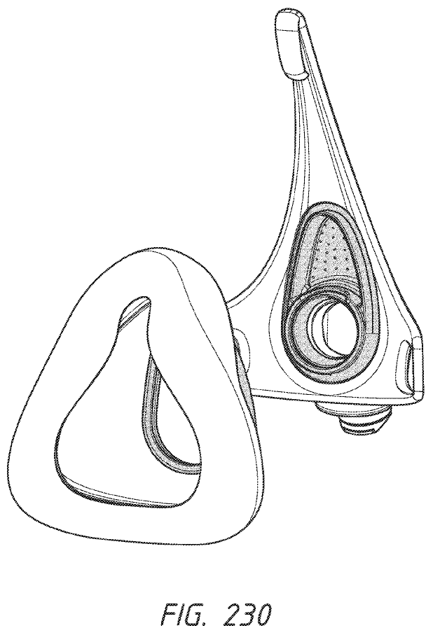

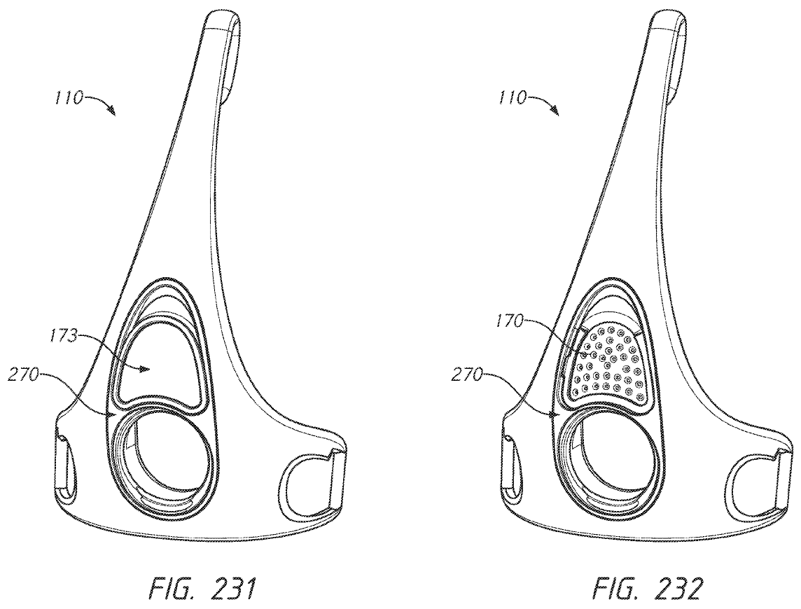

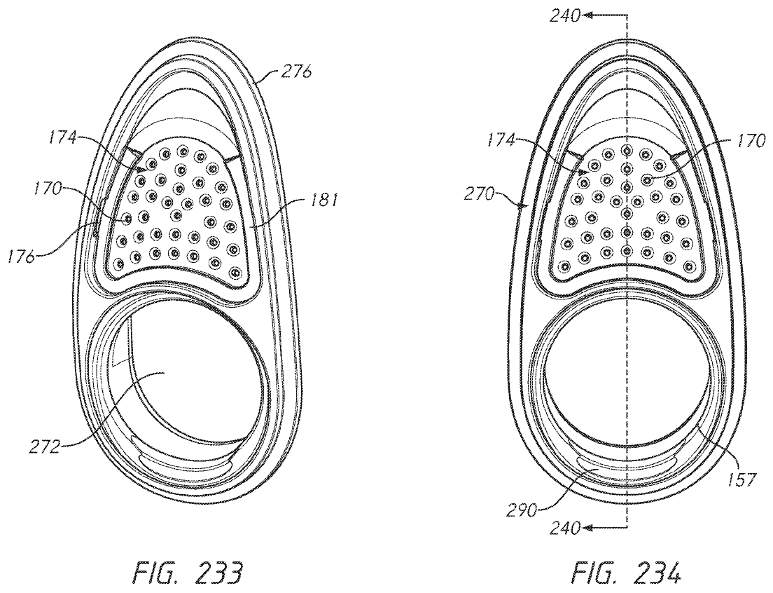

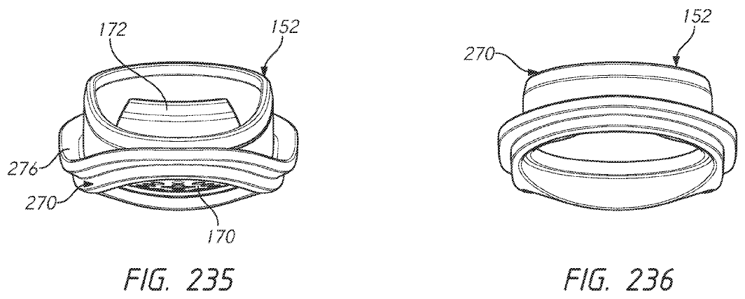

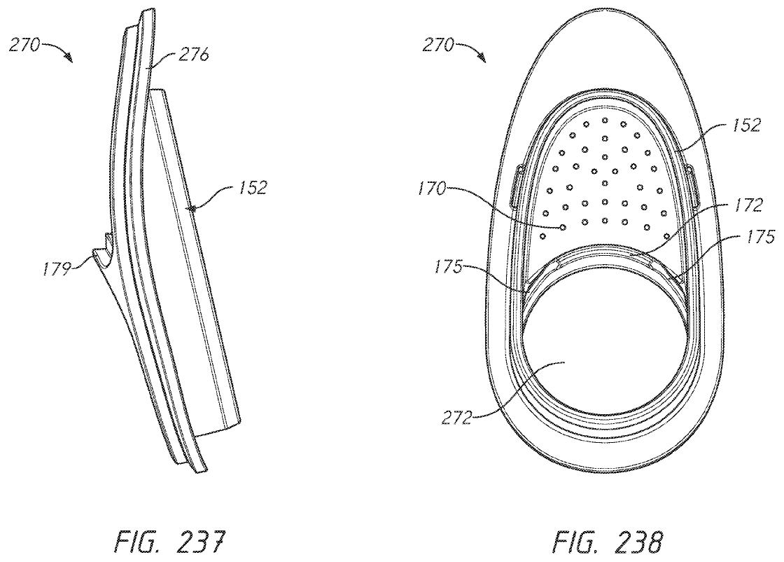

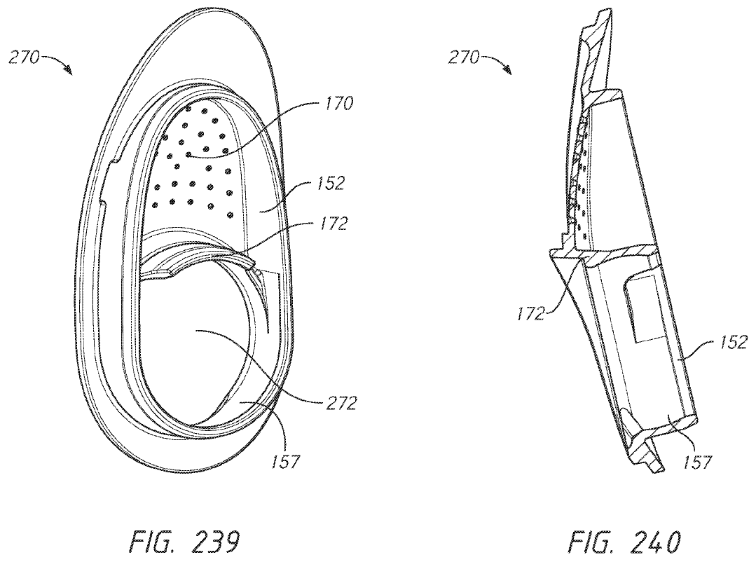

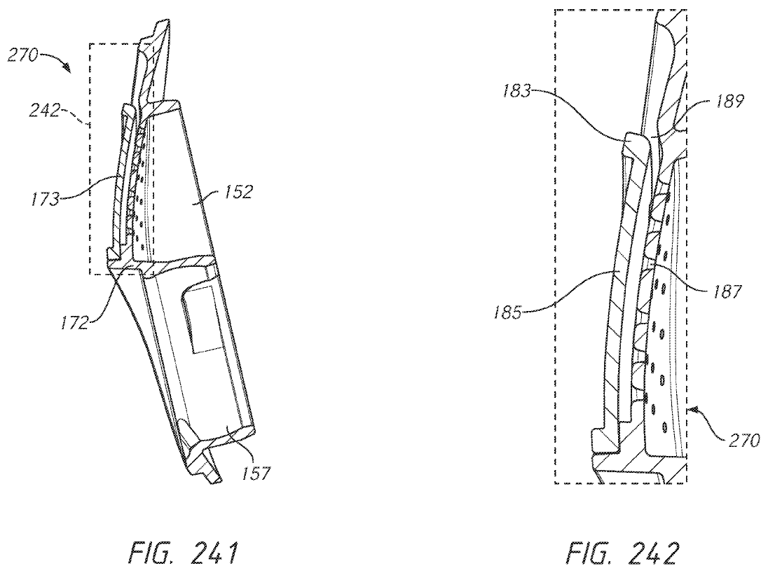

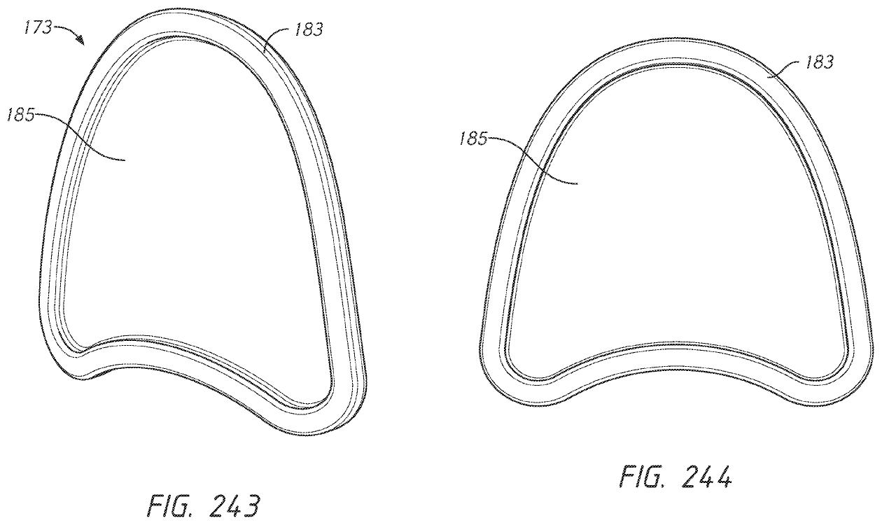

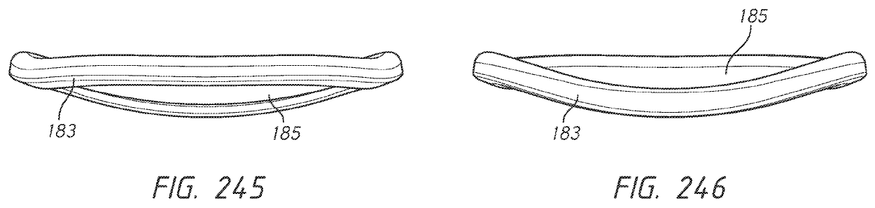

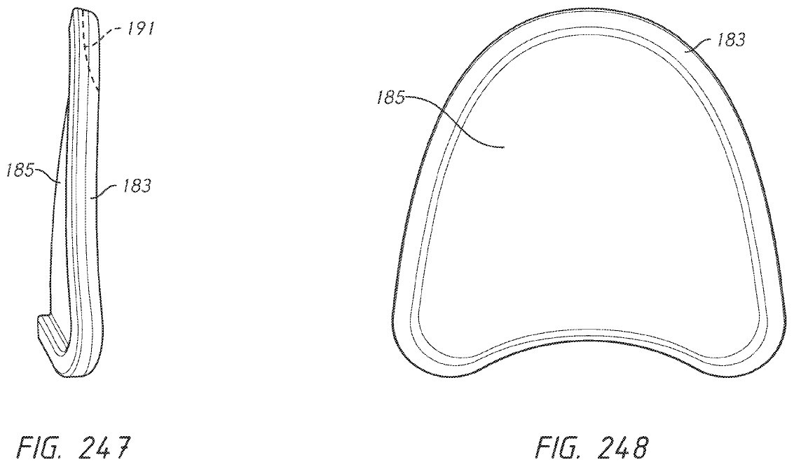

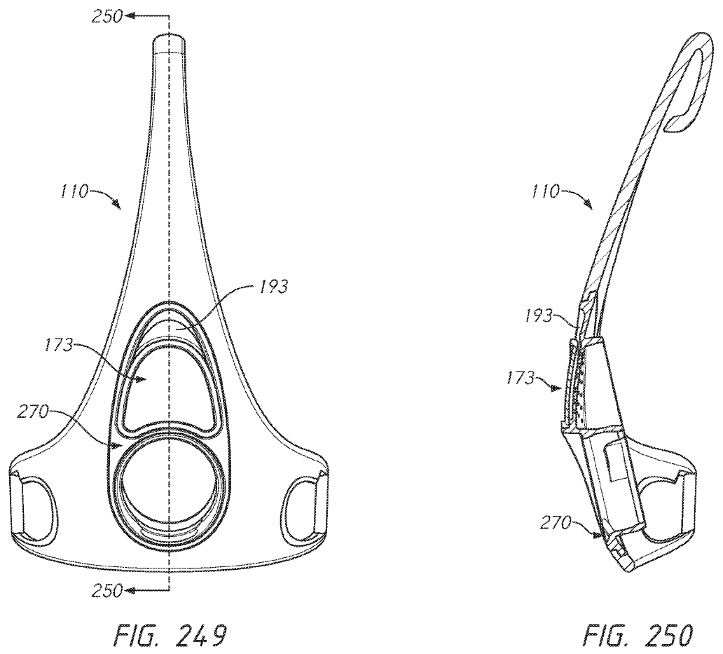

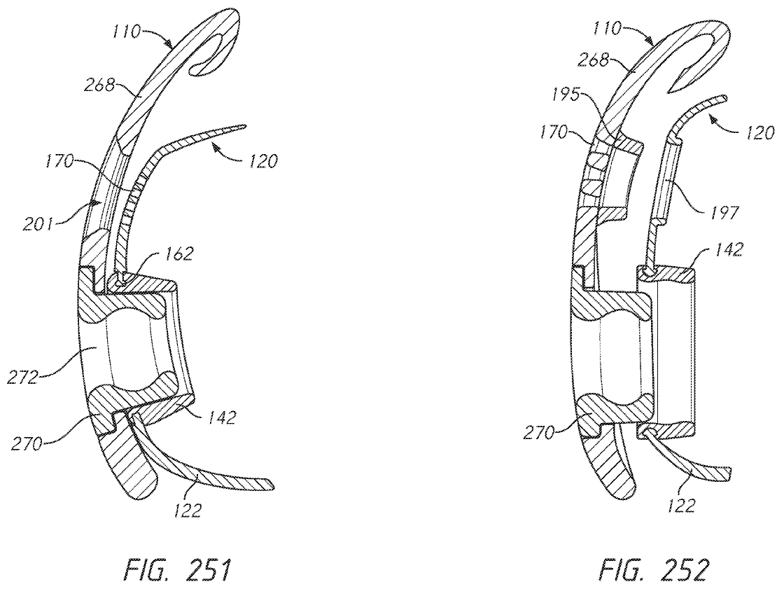

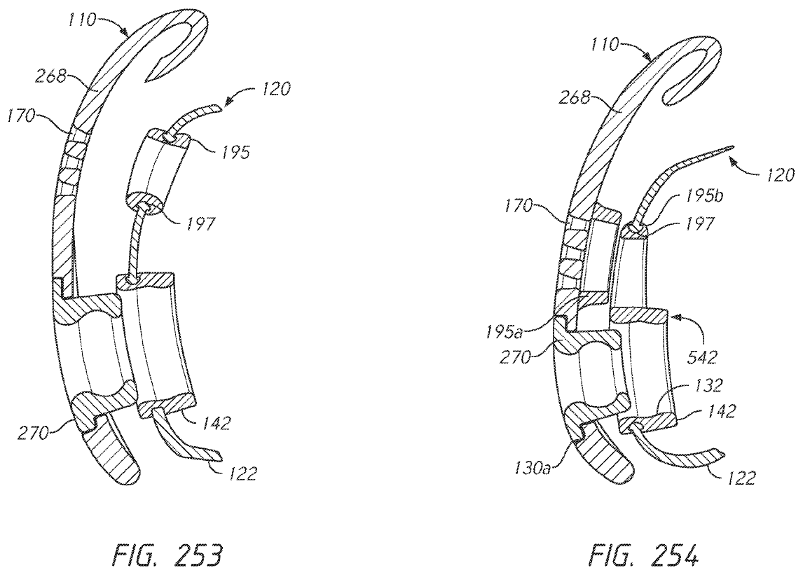

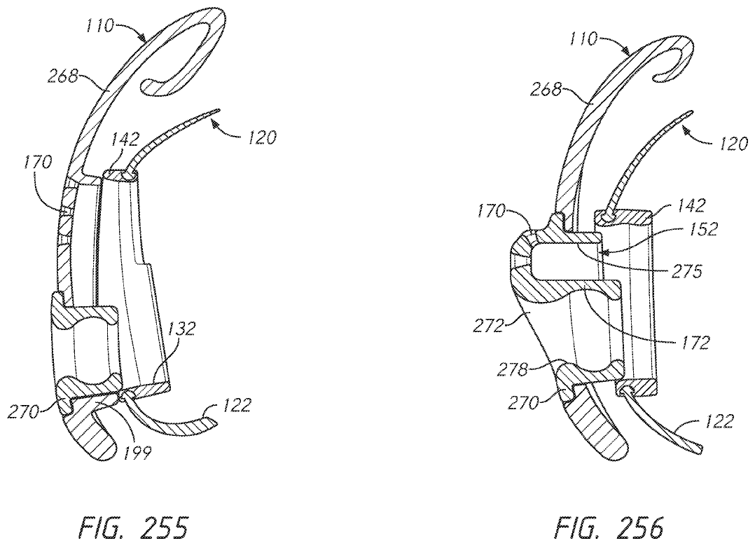

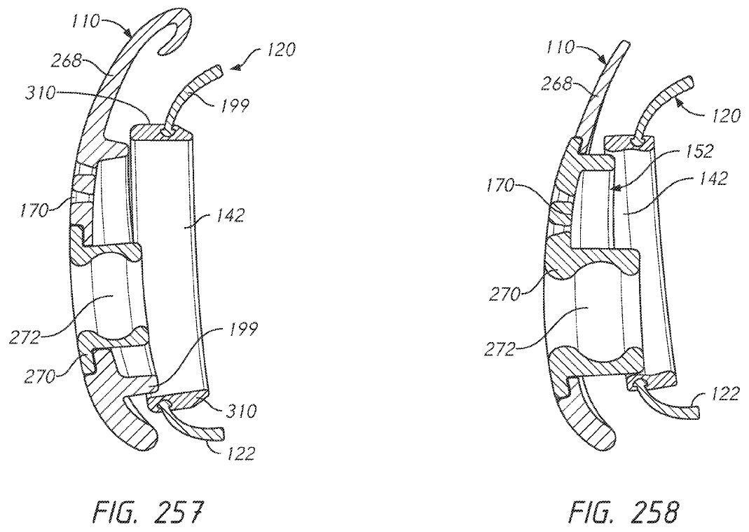

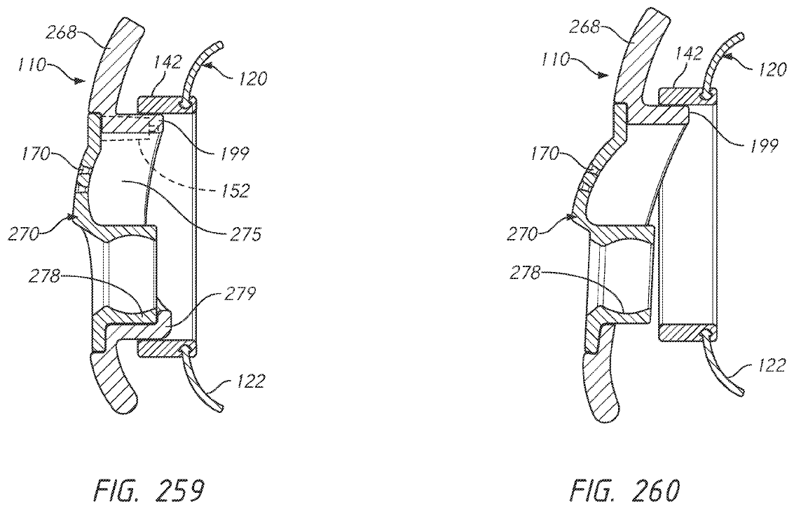

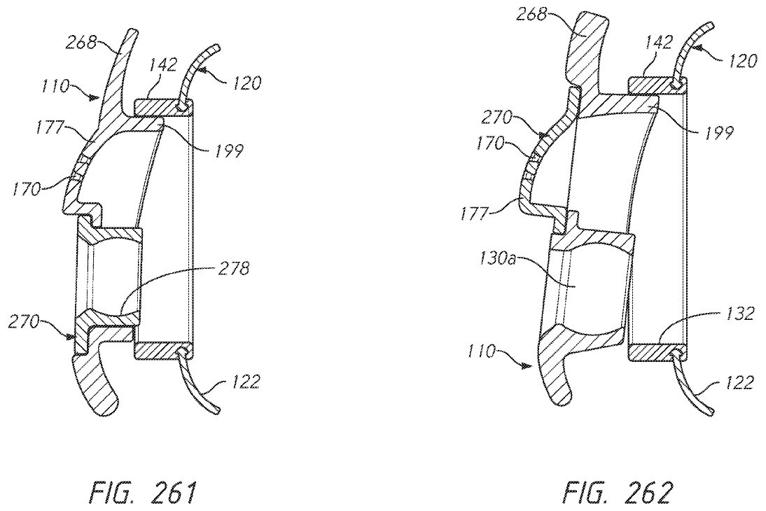

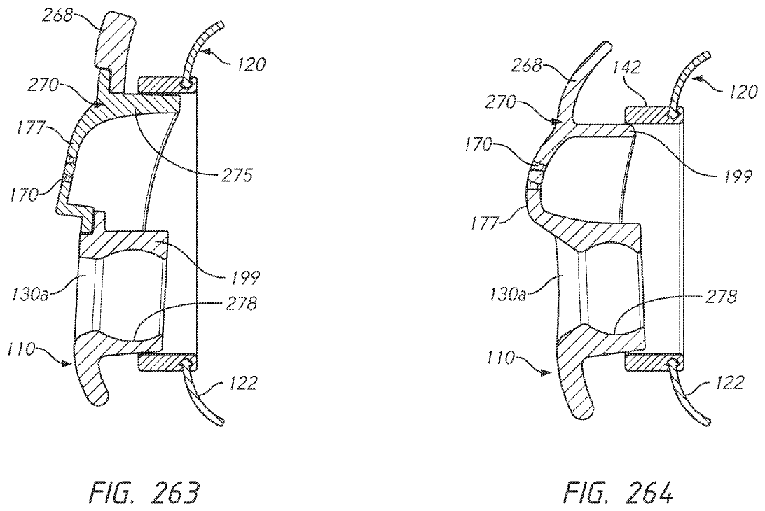

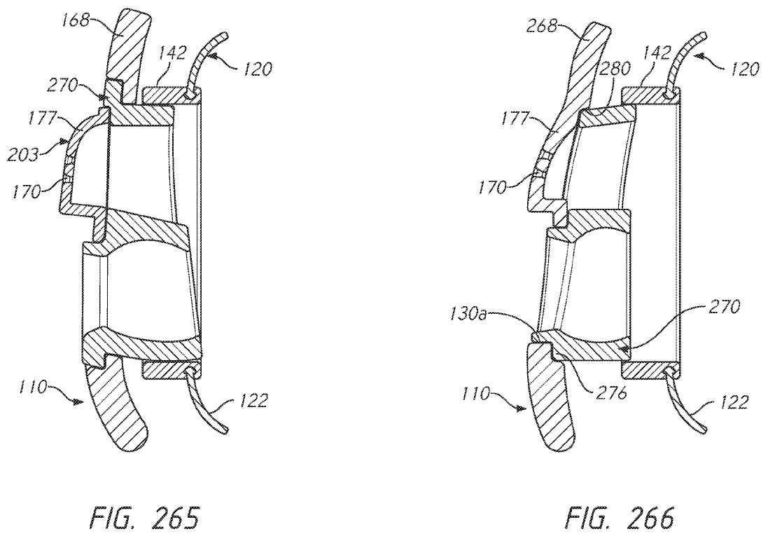

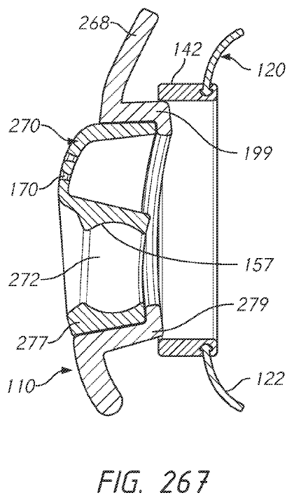

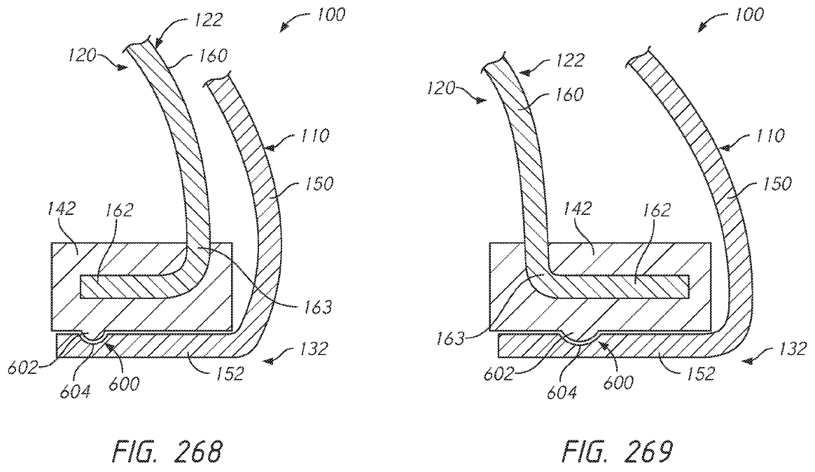

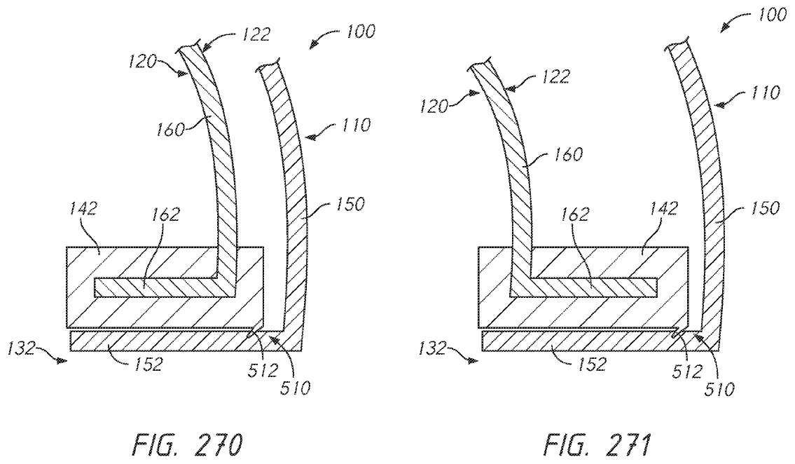

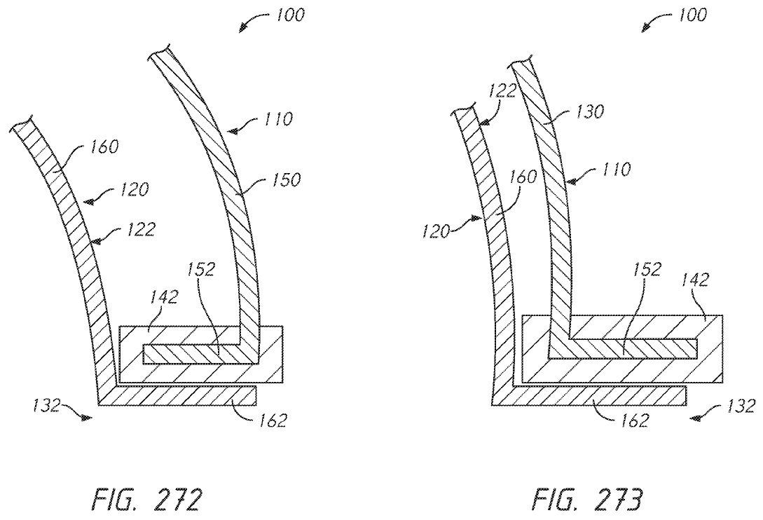

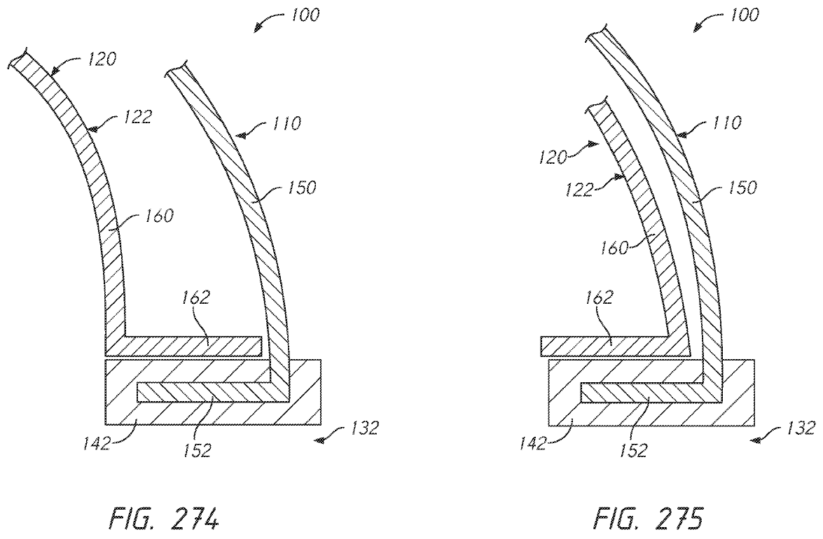

FIGS. 71-100 illustrate another patient interface 100 that is similar to the interface 100 of FIGS. 1-7 in some respects. Accordingly, the same reference numbers are used to refer to the same or similar features or components between the two interfaces 100. Any features or components of the patient interface 100 of FIGS. 71-100 that are not described in detail can be the same as or similar to the corresponding feature or component of the patient interface 100 of FIGS. 1-7, or can be of another suitable arrangement. The illustrated patient interface 100 includes a frame 110 and a cushion module 120 that includes a cushion or seal 124 coupled to a housing 122. In some configurations, for example as shown in FIGS. 72 and 74, the housing 122 can include one or more grips 123 that can provide the user a place to better or more easily grip the cushion module 120, for example, during assembly and/or disassembly with the frame 110. In the illustrated arrangement, the grips 123 are defined by recessed portions of the housing 122. In other arrangements, the grips 123 could be defined by protruding portions or by other arrangements that increase a user's grip on the cushion module 120 relative to a version lacking the grips 123. The connection opening 132 can be generally circular to accommodate a generally circular inlet collar 152 of the frame 110. A friction coupling 140 couples the cushion module 120 and the frame 110.

As described herein, the friction coupling 140 is or comprises an elastomeric friction member 142 coupled to, e.g., overmolded to, a support flange 162. The support flange 162 can be relatively more rigid than the elastomeric friction member 142. The elastomeric friction member 142 and support flange 162 together can be considered or called a cushion module flange 147. The cushion module flange 147 can define the connection opening 132. In the illustrated configuration, the elastomeric friction member 142 encloses the support flange 162. Therefore, the support flange 162 can be considered an internal flange. In some configurations, the cushion module flange 147 is designed to be backwards compatible such that the cushion module 120 can be coupled to other or prior models of the frame 110. The housing 122 comprises one or more apertures 166 through which the material of the elastomeric friction member 142 extends as a result of the over-molding process. In the illustrated arrangement, the apertures 166 are located in the support flange 162. At least a portion of the perimeter of the apertures 166 can be curved, which can help reduce stress concentration locations at the border(s) between the elastomeric friction member 142 and the support flange 162. The size of the apertures 166 can be selected based at least in part on the strength needed for the elastomeric friction member 142. The support flange 162 can include a region 159 of increased thickness. In the illustrated embodiment, the region 159 of increased thickness is disposed at or along the top of the support flange 162. The region 159 of increased thickness can serve as a gate used during injection molding of the housing 122.

As shown in, for example, FIG. 81, at least a portion of a front edge or perimeter of the support flange 162 can be a curved edge 168. The curved edge 168 can help reduce stress concentration locations at the border(s) between the elastomeric friction member 142 and the support flange 162. Reducing stress concentrations can help increase the lifespan and/or decrease wear of the elastomeric friction member 142 and/or the support flange 162.

In the illustrated configuration, a rearward-most extent of the elastomeric friction member 142 is at or forward of a portion of the main wall 160 (shown in FIG. 77) surrounding and/or adjacent to the connection opening 132. In other words, the support flange 162 and/or friction member 142 extend only outward from the main wall 160. Such an arrangement avoids the elastomeric friction member 142 extending into the breathing chamber 102 thereby providing more room and/or avoiding interference with the user's nose, for example in a nasal mask interface 100. As the user's nose typically fills a large portion of space within the breathing chamber 102 of a nasal mask, the housing 122 would need to be made larger to accommodate an inwardly projecting cushion module flange, which in some cases may not be desirable. An outwardly projecting cushion module flange 147 can help reduce hose-pull.

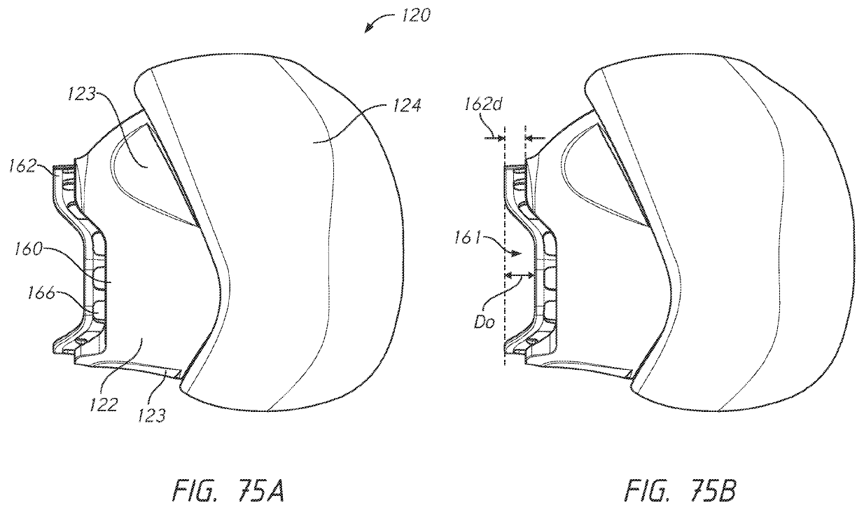

As shown in FIGS. 75A-75B, lateral sides or portions (e.g., arcs) of the support flange 162 are offset from upper and lower portions (e.g., arcs) in a direction of a longitudinal axis of the support flange 162 by an offset distance D_{O}. The offset forms recessed regions 161 along the lateral sides of the support flange 162. When the elastomeric friction member 142 is coupled (e.g., overmolded) to the support flange 162, portions of the elastomeric friction member 142 span the recessed regions 161. A portion of the elastomeric friction member 142 that is radially inward and/or outward (with respect to the connection opening 132) from the support wall 163 and/or the support flange 162 defines a supported portion 312 (shown in, e.g., FIG. 78) of the elastomeric friction member 142. The supported portion 312 is directly supported in a radial direction by the support wall 163 and/or the support flange 162. A portion of the elastomeric friction member 142 that does not extend radially outward or inward with respect to the support flange 162 and/or the support wall 163 defines an unsupported portion 310 (shown in, e.g., FIG. 78). In other words, the unsupported portion 310 is not directly supported in a radial direction by the support flange 162 (or the support wall 163). Thus, the unsupported portion 310 can deform radially outward or increase in diameter or circumference with less force than the supported portion 312. In some configurations, as described further herein, the unsupported portion 310 applies less radial force to the collar 152 than the supported portion 312.

The elastomeric friction member 142 can therefore comprise supported regions 142s. In at least one form, the supported regions 142s can be the regions of the elastomeric friction member 142 where the axial length of the supported portion 312 is greater than the axial length of the unsupported portion 310. In other words, the supported regions 142s can be regions where the second length 182 of the elastomeric friction member 142 (as described with reference to at least FIG. 3) is greater than the third length 184.

The elastomeric friction member 142 can also comprise unsupported regions 142u. In at least one form, the unsupported regions 142u can be the regions of the elastomeric friction member 142 where the axial length of the supported portion 312 is less than the axial length of the unsupported portion 310. In other words, the unsupported regions 142u can be regions where the second length 182 (as described with reference to at least FIG. 3) of the elastomeric friction member 142 is less than the third length 184.

The elastomeric friction member 142 therefore has supported regions 142s (that mostly overlie or surround the support flange 162) along the upper and lower portions of the elastomeric friction member 142, and unsupported regions 142u (that mostly span the recessed regions 161) along the lateral portions, as shown in FIG. 77. In some configurations, a dimension 162d (e.g., an axial length) of the support flange 162 is constant along an entirety of the support flange 162. In some configurations, the dimension 162d of the support flange 162 (shown in FIG. 75B) is about 3.4 mm. In some such configurations, the offset distance D_{O} is about 5 mm. In such configurations, a ratio of the offset distance D_{O} to the support flange dimension 162d is therefore 5:3.4 or 1.47:1.

In some configurations, one or more second flange portion(s) of the support flange 162 (which may be the one or more lateral sides or portions of the support flange 162 as in the configuration shown in, e.g., FIGS. 75A-75B) are offset from one or more first flange portion(s) of the support flange 162 (which may be the upper and/or lower portions of the support flange 162 as in the configuration shown in, e.g., FIGS. 75A-75B. The one or more first flange portion(s) surround a first perimeter portion of the connection opening 132. The one or more second flange portion(s) surround a second perimeter portion of the connection opening 132. The one or more first flange portion/s can be offset from the one or more second flange portion/s along the assembly axis of the patient interface 100. The one or more first flange portion/s can be offset from the one or more second flange portion/s by a distance approximately equal to the dimension 162 of the support flange 162. The first flange portion(s) have or define a first portion of a front edge or perimeter of the support flange 162. The second flange portion(s) have or define a second portion of the front edge or perimeter of the support flange 162. The second portion of the front edge of the support flange 162 can be offset from the first portion of the front edge of the support flange 162. In some configurations, the second portion of the front edge of the support flange 162 is rearwardly (along a longitudinal axis of the connection opening 132 or along a direction of assembly of the cushion module 120 to the frame 110) offset from the first portion of the front edge of the support flange 162.

In the supported regions 142s of the elastomeric friction member 142, the support flange 162 extends for a significant length of the total length of the elastomeric friction member 142, thereby supporting the elastomeric friction member 142 along a significant portion of its length. Therefore, a supported length x1, shown in FIG. 82, of the elastomeric friction member 142 (where the supported length x1 can be defined as an axial length from the base or rear edge of the elastomeric friction member 142 to the forward, free end of the support flange 162 as shown) is a significant portion of the total length of the elastomeric friction member 142. Both the supported regions 142s and unsupported regions 142u can include unsupported lengths, i.e., the length of the elastomeric friction member 142 that extends beyond the support flange 162, indicated by x2 in FIG. 82. However, in the supported regions 142s, the unsupported lengths are small relative to the total length of the elastomeric friction member 142. Therefore, if x1≥x2 for a given region of the elastomeric friction member 142, as shown in FIG. 82, that region is a supported region 142s. If x1<x2, that region of the elastomeric friction member 142 is an unsupported region 142u. The unsupported regions 142u can advantageously reduce the influence of manufacturing variations on the removal force between the cushion module 120 and frame 110. The unsupported regions 142u can flex, for example like cantilevers, along their length, thereby allowing for more compensation for manufacturing variations compared to a cushion module 120 with no unsupported regions.

As shown in FIG. 82, the elastomeric friction member 142 defines a first thickness 190 that extends in or substantially in a radial direction or perpendicular to the direction of assembly 104. The support flange 162 defines a second thickness 192 that extends in or substantially in a radial direction or perpendicular to the direction of assembly 104. A portion of the elastomeric friction member 142 located radially outside of the support flange 162 defines a third thickness 194 that extends in or substantially in a radial direction or perpendicular to the direction of assembly 104. A portion of the elastomeric friction member 142 located radially inside of the support flange 162 defines a fourth thickness 196 that extends in or substantially in a radial direction or perpendicular to the direction of assembly 104. In the illustrated configuration, the first thickness 190 equals the sum of the second thickness 192, the third thickness 194, and the fourth thickness 196.

In the illustrated configuration, the fourth thickness 196≥the second thickness 192, and the fourth thickness 196≥the third thickness 194. The third thickness 194 can result from the material that forms the elastomeric friction member 142 passing through the apertures 166 during overmolding to secure the elastomeric friction member 142 to the support flange 162. In some configurations, for example in which the elastomeric friction member 142 is secured to the support flange 162 via means other than overmolding, such as chemical bonding, the third thickness 194 can be zero or approach zero. In some configurations, the second thickness 192 is equal or approximately equal to the third thickness 194. In some configurations, the second thickness 192 and/or third thickness 194 is 1 mm or approximately 1 mm. The third thickness 194 can vary along the length of the elastomeric friction member 142 due to, for example, angled surfaces of the support flange 162 and/or expansion or shrinkage of the elastomeric friction member 142 after molding.

Similar to other configurations of patient interfaces 100 shown and described herein, the elastomeric friction member 142 includes a pair of protrusions 362, each configured to be received by one of a pair of recesses 360 (shown in FIG. 91) of the collar 152 when the cushion module 120 is assembled to the frame 110. Cooperation between the protrusions 362 and recesses 360 can advantageously inhibit relative motion (e.g., rotation) between the frame 110 and cushion module 120. In the illustrated configuration, the protrusions 362 and recesses 360 are generally triangular. The elastomeric friction member 142 can include a pair of recesses or indents 361 in the peripheral surface 146. The recesses 361 can cooperate with a rear side of the frame 110 to help with alignment between the cushion module 120 and the frame 110.

In the illustrated arrangement, the elastomeric friction member 142 comprises an engagement surface 144 that engages an engagement surface 154 of the collar 152. A friction-fit between the collar 152, e.g., the engagement surface 154, and the elastomeric friction member 142 secures the frame 110 and cushion module 120 together. In at least one configuration, the engagement surface 144 of the elastomeric friction member 142 is an interior or radially inward-facing surface of the elastomeric friction member 142. In at least one configuration, the engagement surface 154 of the collar 152 is an exterior or radially outward-facing surface of the collar 152. However, in other arrangements, the elastomeric friction member 142 could engage an interior surface of the collar 152. Preferably, with either arrangement the frame 110 defines at least a portion of the breathing chamber 102. For example, in the illustrated configuration, an interior space defined by the collar 152 defines a portion of the breathing chamber 102.

As described above, in the illustrated arrangement, the peripheral surface 146 is exposed or is not directly covered when the cushion module 120 is assembled to the frame 110. With such an arrangement, the elastomeric friction member 142 is compressed from a single surface (e.g., the engagement surface 144), or from the engagement surface 144 and a front surface of the elastomeric friction member 142 (that may abut a rear surface of the frame 110). Accordingly, dimensional variations as a result of the manufacturing process will not impact the assembly and removal force to as great an extent as a design in which both the interior and exterior surfaces of the elastomeric friction member 142 engage a surface of a cooperating structure. Moreover, having the peripheral surface 146 of the elastomeric friction member 142 exposed to atmosphere advantageously allows the cushion module 120 and/or frame 110 to be easier to clean than an arrangement having a channel, which can be difficult to access by the user.

The engagement surface 144 can have a first engaging surface 144a and a second engaging surface 144b, as shown in FIGS. 81-82. As shown in FIG. 82, the second engaging surface 144b can extend at an angle θ relative to the first engaging surface 144a. The angle θ can be selected based at least in part on the grade of silicone used for the elastomeric friction member 142 and/or the forces desired to be required to connect and/or disconnect the frame 110 and cushion module 120. For example, in some configurations in which the elastomeric friction member 142 is made of or includes LSR 4070 silicone, a θ of 3° may be selected. In some configurations, the entire first engaging surface 144a is part of the unsupported length x2 of the elastomeric friction member 142, as shown in FIG. 85 (which shows a section view of the unsupported regions 142u, as indicated by x1<x2). In the supported regions 142s, a portion of the first engaging surface 144a may be part of the supported length x1 of the elastomeric friction member 142.

As the frame 110 and cushion module 120 are coupled, an interference region 145 of the elastomeric friction member 142 compresses and flexes to accommodate the inlet collar 152 and create the friction fit between the frame 110 and cushion module 120. As shown in FIG. 96, the amount the elastomeric friction member 142 compresses can be considered a thickness Ti of the interference region 145 between the elastomeric friction member 142 and the inlet collar 152. A greater amount of compression or interference region thickness Ti results in a greater degree of or stronger friction or interference between the two components. The inlet collar 152 and cushion module flange 147 can be sized and/or otherwise designed so that the interference between the two is such that the force required to separate the frame 110 and cushion module 120 once connected is between about 10 N and 50 N during the intended lifespan of the patient interface 100.

In some configurations, the interference region thickness Ti decreases between the forward end of the cushion module flange 147 and rear end of the cushion module flange 147. In the illustrated configuration, the interference region thickness Ti is greatest or at a maximum at or near the forward end of the cushion module flange 147 (e.g., where Ti is labeled in FIG. 96) and decreases toward the base or rear end of the cushion module flange 147 to a small interference region thickness labeled Ti1 in FIG. 96. The interference between the frame cushion module flange 147 and inlet collar 152 is therefore biased (or greater) toward the front of the cushion module flange 147. Having a greater interference closer to the body and more vertical part of the frame 110 and farther away from the rear, free end of the inlet collar 152 can advantageously cause the inlet collar 152 to flex less and/or shifts more of the friction forces between the frame 110 and the cushion module 120 toward the base or forward end of the inlet collar 152. This can lessen the effects of rotation of a conduit connector coupled to the frame 110 as described herein. The variation in interference region thickness Ti can be at least partially the result of an angle of the inlet collar 152 relative to the cushion module flange 147 and/or the angle θ of the second engaging surface 144b relative to the first engaging surface 144a.

In some configurations, for example as shown in FIG. 100, in the unsupported regions 142u, the interference region 145 is entirely disposed in the unsupported length x2 of the elastomeric friction member 142. In some configurations or portions of the cushion module flange 147, e.g., in supported regions 142s, the interference region 145 can at least partially be in or overlap with the supported length x1 of the elastomeric friction member 142. In portions of the interference region 145 in the supported length x1, the elastomeric friction member 142 will compress (and may flex around the edges of the support flange 162) to accommodate the inlet collar 152. In portions of the interference region 145 in the unsupported length x2, the elastomeric friction member 142 can flex outward, for example like a cantilever, instead of or in addition to compressing. A larger thickness of the elastomeric friction member 142 can flex in the unsupported lengths x2 compared to a smaller thickness of the elastomeric friction member 142 that compresses in the supported lengths x1. The unsupported lengths x2, and therefore particularly the unsupported regions 142u in which the unsupported length x2 is greater than the supported length x1, can advantageously reduce the influence of manufacturing variations on the removal force between the cushion module 120 and frame 110. A given manufacturing tolerance (e.g., 0.1 mm) is a greater percentage of the interference region thickness Ti compared to the thickness of the unsupported length of the elastomeric friction member 142, which can flex.

In the illustrated arrangement, the frame 110 defines a socket 157 configured to receive a conduit connector, such as an elbow 158, configured to connect a breathing circuit to the patient interface 100. In the illustrated arrangement, the collar 152 defines at least a portion of the elbow socket 157. In some configurations, the conduit connector or elbow 158 is capable of pivoting or rotating about one or more axes relative to the frame 110. The conduit connector or elbow 158 may have one rotational degree of freedom. For example, the conduit connector or elbow 158 can have a swivel connection with the frame 110. Alternatively, the conduit connector or elbow 158 can have more than one rotational degree of freedom. For example, the conduit connector or elbow 158 may have more than one rotational degree of freedom. The elbow 158 can be removably coupled to the frame 110.

The vent 170 can be located in the elbow 158. In particular, the vent 170 can be located in a portion of an upper, outer, or patient-distal curve of the elbow 158, as shown in FIGS. 71 and 93-95. In the illustrated configuration, an elbow vent piece 155 is fitted over a portion of the elbow 158 including the vent 170. The elbow vent piece 155 can be removably coupled to the elbow 158. The elbow vent piece 155 includes one or more vent apertures 153 extending through the cover 155. In use, gases pass through the vent 170 and then through the one or more vent apertures 153 to atmosphere. In some configurations, a diffusing element is disposed between the vent 170 and the vent apertures 153. In some configurations, a diffusing element is disposed within the vent apertures 153.

FIGS. 101-138 illustrate another patient interface 100 that is similar to the patient interface 100 of FIGS. 71-100 in some respects. Accordingly, the same reference numbers are used to refer to the same or similar features or components between the two interfaces 100. Any features or components of the patient interface 100 of FIGS. 101-138 that are not described in detail can be the same as or similar to the corresponding feature or component of the patient interface 100 of FIGS. 71-100, or can be of another suitable arrangement. The illustrated patient interface 100 includes a frame 110 and a cushion module 120 that includes a cushion or seal 124 coupled to a housing 122. The connection opening 132 can be generally circular to accommodate a generally circular portion (e.g., insert collar 278 as described herein) of the frame 110. A friction coupling 140 couples the cushion module 120 and the frame 110.

As described herein, the friction coupling 140 is or comprises an elastomeric friction member 142 coupled to, e.g., overmolded to, a support flange 162. The support flange 162 can be relatively more rigid than the elastomeric friction member 142. The elastomeric friction member 142 and support flange 162 together can be considered or called a cushion module flange 147. The cushion module flange 147 can define the connection opening 132. In the illustrated configuration, the elastomeric friction member 142 encloses the support flange 162. Therefore, the support flange 162 can be considered an internal flange. The support flange 162 can include a region 159 of increased thickness. In the illustrated embodiment, the region 159 of increased thickness is disposed at or along the top of the support flange 162. The region 159 of increased thickness can serve as a gate used during injection molding of the housing 122.

The housing 122 comprises one or more apertures 166 through which the material of the elastomeric friction member 142 extends as a result of the over-molding process. In the illustrated arrangement, the apertures 166 are located in the support flange 162. The apertures 166 can be rectangular or trapezoidal. At least a portion of the perimeter of the apertures 166 can be curved, which can help reduce stress concentration locations at the border(s) between the elastomeric friction member 142 and the support flange 162. The size of the apertures 166 can be selected based at least in part on the strength needed for the elastomeric friction member 142. In some configurations, each aperture 166 can have a major dimension of about 3 mm. Each aperture 166 can have a minor dimension of about 1.8 mm. The apertures 166 can be separated from each other by a distance of about 3 mm along the perimeter of the support flange 162. In at least one configuration, an outer edge (defining the profile of the aperture on a radially outer surface of the support flange 162) of at least one of the apertures 166 can be rounded. In at least one configuration, an inner edge (defining the profile of the aperture on a radially inner surface of the support flange 162) of at least one of the apertures 166 can be rounded. In at least one configuration, both the outer edge and the inner edge of at least one of the apertures 166 can be rounded. Rounding the inner edge and/or the outer edge of one or more of the apertures 166 can help reduce stress concentrations in the elastomeric friction member 142 at or near the apertures 166, thereby increasing the life of the connection between the elastomeric friction member 142 and the housing 122.

As previously described, a portion of the elastomeric friction member 142 that is radially inward and/or (with respect to the connection opening 132) from the support wall 163 and/or the support flange 162 defines a supported portion 312 of the elastomeric friction member 142. A portion of the elastomeric friction member 142 that does not extend radially outward and/or inward with respect to the support flange 162 and/or the support wall 163 defines an unsupported portion 310. In the illustrated configuration, a supported length x1 of the elastomeric friction member 142 is greater than an unsupported length x2 of the elastomeric friction member 142 (as shown in FIG. 113) about the entire perimeter of the elastomeric friction member 142. The supported length x1 can correspond to the second length 182 as described with reference to FIG. 3. The unsupported length x2 can correspond to the third length 184 described with reference to FIG. 3. The supported length x1 of the elastomeric friction member 142 or cushion module flange 147 can be about 6.7 mm. The unsupported length x2 of the elastomeric friction member 142 or cushion module flange 147 can be about 1.8 mm. A total length x of the elastomeric friction member 142 or cushion module flange 147 can be about 8.5 mm. As shown, the unsupported length x2 can be less than the supported length x1. In some configurations, a ratio of the unsupported length x2 to the supported length x1 can be about 1:3.7. In use, for example, when the cushion module 120 is coupled or being coupled to the frame 110, the unsupported length x2 can elastically deform, compress, and/or flex outwardly, for example, like a cantilever, relatively more than the supported length x1.

As shown in FIG. 114, the elastomeric friction member 142 defines a first thickness 190 that extends in or substantially in a radial direction or perpendicular to the direction of assembly 104. The support flange 162 defines a second thickness 192 that extends in or substantially in a radial direction or perpendicular to the direction of assembly 104. A portion of the elastomeric friction member 142 located radially outside of the support flange 162 defines a third thickness 194 that extends in or substantially in a radial direction or perpendicular to the direction of assembly 104. A portion of the elastomeric friction member 142 located radially inside of the support flange 162 defines a fourth thickness 196 that extends in or substantially in a radial direction or perpendicular to the direction of assembly 104.

In the illustrated configuration, the first thickness 190 equals the sum of the second thickness 192, the third thickness 194, and the fourth thickness 196. As shown, the first thickness 190, or total thickness of the elastomeric friction member 142, can vary along its length. The second thickness 192, third thickness 194, and/or fourth thickness 196 can vary along their length(s). In the embodiment of FIG. 114, the fourth thickness has a distal thickness 196, 1, a central thickness 196, 2, and a proximal thickness 196, 3. The first thickness 190 can be about 3.5 mm. The second thickness 192 can be about 1 mm. The third thickness 194 can be about 1.5 mm. The distal thickness 196, 1 can be about 0.9 mm. The central thickness 196, 2 can be about 1.1 mm. The proximal thickness 196, 3 can be about 1.4 mm. In some cases, the various thickness of the elastomeric friction member 142 can vary due to, for example, angled surfaces of the support flange 162 and/or expansion or shrinkage of the elastomeric friction member 142 during or after molding.

As shown in, e.g., FIGS. 121-138, the frame 110 can include a frame insert 270. In the illustrated configuration, the frame insert 270 includes an abutment collar 274, an abutment flange 276, and an insert collar 278. The abutment flange 276 has an enlarged dimension, e.g., diameter, circumference, or perimeter, compared to the abutment collar 274 and insert collar 278. The abutment collar 274 extends forward (or away from the user in use) from the abutment flange 276. The insert collar 278 extends rearward (or toward the user in use) from the abutment flange 276. The abutment flange 276 can include an aligning feature 282. In the illustrated configuration, the aligning feature 282 is a projection or portion or the abutment flange 276 protruding radially outward from the remainder of the abutment flange 276. In the illustrated configuration, the aligning feature 282 is located at or along a bottom of the abutment flange 276. The insert collar 278 can include an insert collar recess 360 recessed from a free rear edge of the insert collar 278. In the illustrated configuration, the insert collar recess 360 is disposed in or along a top of the insert collar 278. The frame insert 270 has an insert opening 272 defining a gas flow path through the frame insert 270. As described herein, the cushion module 120 can be coupled to the frame 110 by sliding the cushion module flange 147 onto the insert collar 278. When the cushion module 120 and frame 110 are fully coupled, a leading surface or abutment surface 148 (shown in FIGS. 111-112) of the elastomeric friction member 142 abuts a rear surface of the abutment flange 276 of the frame insert 170.

A main body of the frame 110, shown in FIGS. 115-120, includes a frame inlet opening 130a that surrounds the gas inlet 130. When the frame insert 270 is assembled with the main body of the frame 110, the abutment collar 274 of the frame insert 270 extends into the frame inlet opening 130a, and at least a portion of the insert opening 272 defines the gas inlet 130, as shown in FIGS. 129-134. The main body of the frame 110 includes a rear peripheral recess or channel 280 in a rear or back surface of the frame 110 surrounding the perimeter of the frame inlet opening 130a. When the frame insert 270 is assembled with the main body of the frame 110, the abutment flange 276 of the frame insert 270 at least partially rests in the rear peripheral recess 280 of the main body of the frame 110. The rear peripheral recess 280 can include an extension 284 (shown in FIG. 118) that receives the aligning feature 282 of the abutment flange 276 when the frame insert 270 is coupled to the main body of the frame 110. The aligning feature 282 and extension 284 can help properly align the frame insert 270 relative to the main body of the frame 110 during assembly. In the illustrated configuration, the extension 284 if positioned at or along a bottom of the rear peripheral recess 280 to accommodate an aligning feature 282 positioned at or along the bottom of the abutment flange 266, as illustrated in FIGS. 121-128. A front surface of the abutment flange 276 can contact a surface of the rear peripheral recess 280 when the frame insert 270 and main body of the frame 110 are coupled. The frame insert 270 can be secured to the main body of the frame 110, for example, via welding (e.g., ultrasonic welding), adhesives, and/or other means along the abutting surfaces of the frame insert 270 (e.g., the front surface of the abutment flange 276) and the main body of the frame 110 (e.g., the rear surface of the rear peripheral recess 280). An energy concentrator 420 can be used to concentrate energy during an ultrasonic welding process to ensure a complete weld between the abutment flange 276 and rear peripheral recess 280. As shown in FIG. 128, the energy concentrator 420 can be located on the abutment flange 276. In the illustrated configuration, the energy concentrator 420 protrudes from a front surface of the abutment flange 276. The energy concentrator 420 can be generally triangular as shown.

The elastomeric friction member 142 can include a protrusion or projection 362 (shown in, e.g., FIG. 106) configured to be received by the insert collar recess 360 of the frame insert 270 (shown in, e.g., FIGS. 123 and 127), when the cushion module 120 is assembled to the frame 110. Cooperation between the protrusion 362 and recess 360 can advantageously inhibit relative motion (e.g., rotation) between the frame 110 and cushion module 120. The protrusion 362 can have tapered lateral edges such that a leading edge of the protrusion 362 as the cushion module 120 is coupled to the frame 110, e.g., the frame insert 270, is narrower than an opposite base of the protrusion 362. The tapered lateral edges can act as a lead in feature for the user when coupling the cushion module 120 to the frame 110 and/or can help the cushion module 120 and frame 110 self-align or correct slightly improper alignment during assembly. During assembly of the cushion module 120 and the frame 110, there is interference (indicated by region 294 in FIG. 138) between the lateral edges of the protrusion 362 and lateral edges of the corresponding recess 360 of the frame insert 270. The interference can provide tactile feedback, for example, via a resistance force, to the user that the cushion module 120 and frame 110 are properly coupled.

In the illustrated arrangement, the elastomeric friction member 142 comprises an engagement surface 144 that engages an engagement surface 154 of the insert collar 278. A friction-fit between the collar 278, e.g., the engagement surface 154, and the elastomeric friction member 142 secures the frame 110 and cushion module 120 together. In at least one configuration, the engagement surface 144 is an interior or radially inward-facing surface of the elastomeric friction member 142. In at least one configuration, the engagement surface 154 is an exterior or radially outward-facing surface of the collar 278. However, in other arrangements, the elastomeric friction member 142 could engage an interior surface of the collar 278. Preferably, with either arrangement the frame 110 defines at least a portion of the breathing chamber 102. For example, in the illustrated configuration, an interior space defined by the collar 278 defines a portion of the breathing chamber 102.

As described above, in the illustrated arrangement, the peripheral surface 146 (shown in, e.g., FIG. 108) is exposed or is not directly covered when the cushion module 120 is assembled to the frame 110. With such an arrangement, the elastomeric friction member 142 is compressed from a single surface (e.g., the engagement surface 144). Accordingly, dimensional variations as a result of the manufacturing process will not impact the assembly and removal force to as great an extent as a design in which both the interior and exterior surfaces of the elastomeric friction member 142 engage a surface of a cooperating structure. Moreover, having the peripheral surface 146 of the elastomeric friction member 142 exposed to atmosphere advantageously allows the cushion module 120 and/or frame 110 to be easier to clean than an arrangement having a channel, which can be difficult to access by the user.

The engagement surface 144 can have a first engaging surface 144a and a second engaging surface 144b, as shown in FIGS. 111-112. The second engaging surface 144b can extend at an angle relative to the first engaging surface 144a. The angle can be selected based at least in part on the grade of silicone used for the elastomeric friction member 142 and/or the forces desired to be required to connect and/or disconnect the frame 110 and cushion module 120.

As the frame 110 and cushion module 120 are coupled, an interference region 145 (shown in FIG. 135) of the elastomeric friction member 142 compresses and flexes to accommodate the inlet collar 152 and create the friction fit between the frame 110 and cushion module 120. As shown in FIG. 135, the amount the elastomeric friction member 142 compresses can be considered a thickness Ti of the interference region 145 between the elastomeric friction member 142 and the inlet collar 152. A greater amount of compression or interference region thickness Ti results in a greater degree of or stronger friction or interference between the two components. The inlet collar 152 and cushion module flange 147 can be sized and/or otherwise designed so that the interference between the two is such that the force required to separate the frame 110 and cushion module 120 once connected is between about 10 N and 50 N during the intended lifespan of the patient interface 100.

The angle between the first 144a and second 144b engaging surfaces causes there to be a different interference between the first engaging surface 144a and the insert collar 152 compared to the interference between the second engaging surface 144b and the insert collar 152. In some configurations, the interference region thickness Ti decreases between the forward end of the cushion module flange 147 and rear end of the cushion module flange 147 toward the rear end of the cushion module flange 147. In the illustrated configuration, the interference region thickness Ti is greatest or at a maximum at or near the forward end of the cushion module flange 147 (e.g., where Ti is labeled in FIG. 135) and decreases toward the base or rear end of the cushion module flange 147 to a small interference region thickness labeled Ti2 in FIG. 135. In other words, Ti>Ti2. The interference between the frame cushion module flange 147 and inlet collar 152 is therefore biased (or greater) toward the front of the cushion module flange 147. Having a greater interference closer to the body and more vertical part of the frame 110 and farther away from the rear, free end of the inlet collar 152 can advantageously cause the inlet collar 152 to flex less and/or shifts more of the friction forces between the frame 110 and the cushion module 120 toward the base or forward end of the inlet collar 152, which can lessen the effects of rotation of a conduit connector coupled to the frame 110 as described herein. The variation in interference region thickness Ti can be at least partially the result of an angle of the inlet collar 152 relative to the cushion module flange 147 and/or the angle of the second engaging surface 144b relative to the first engaging surface 144a. In some configurations, the thickness of a central portion or midpoint of the cushion module flange 147 (e.g., where Ti1 is labeled in FIG. 135), which can be located at or near a transition between the first engaging surface 144a and the second engaging surface 144b, can be equal to the thickness Ti2. In other words, the interference region can have a varying thickness in the region of the first engaging surface 144a and a constant thickness in the region of the second engaging surface 144b. In other configurations, central thickness Ti1 can be greater or less than Ti2.

In the illustrated arrangement, the frame insert 270 defines a socket 157 configured to receive a conduit connector, such as an elbow 158, configured to connect a breathing circuit to the patient interface 100. In the illustrated arrangement, the insert collar 278 includes and/or defines at least a portion of the elbow socket 157. The elbow socket 157 can include a cut-out 290, as shown in FIG. 121, which can assist with inserting and/or removing the elbow 158. The cut-out 290 can be located along an inner surface of a bottom portion of the abutment collar 274. The elbow socket 157 can include one or more recesses 292. The elbow socket recesses 292 can be regions of reduced thickness of the insert collar 278. The elbow socket recesses 292 can allow the insert collar 278 to flex, for example, when being removed from a mold tool during manufacturing and/or during assembly with other components, while reducing or minimizing deformation of the socket 157. In some configurations, the conduit connector or elbow 158 is capable of pivoting or rotating about one or more axes relative to the frame 110. For example, the conduit connector or elbow 158 can have a swivel connection with the frame 110. The elbow 158 can be removably coupled to the frame 110. The vent 170 can be located in the elbow 158. In particular, the vent 170 can be located in a portion of an upper, outer, or patient-distal curve of the elbow 158, as shown in FIG. 101.

FIGS. 139-167 illustrate another patient interface 100 that is similar to the patient interface 100 of FIGS. 15-20 in some respects. Accordingly, the same reference numbers are used to refer to the same or similar features or components between the two interfaces 100. Any features or components of the patient interface 100 of FIGS. 139-167 that are not described in detail can be the same as or similar to the corresponding feature or component of the interface 100 of FIGS. 15-20, or can be of another suitable arrangement. The illustrated patient interface 100 includes a frame 110 and a cushion module 120 that includes a cushion or seal 124 coupled to a housing 122. The seal 124 is configured to seal on a patient in use.

The housing 122 has an outer surface exposed to ambient pressure in use and an inner surface exposed to therapeutic pressure in use. The housing 122 has a first end and a second end. The first end of the housing 122 is coupled to the seal 124. The second end of the housing 122 can define, surround, or include the connection opening 132. The first end of the housing 122 has a first end perimeter defining a first opening. The second end of the housing 122 has a second end perimeter defining a second opening. The first end perimeter can be larger than the second end perimeter. The first opening can be larger than the second opening. The first opening can have a generally triangular shape. Generally triangular shapes include triangular shapes and shapes that resemble a triangle in appearance. Generally triangular shapes can include shapes having a first or base portion that is wider than an opposing portion. Generally triangular shapes can taper from one end to the other end. One or more of the “corners” of a generally triangular shape may be rounded. One or more sides of a generally triangular shape can be curved or non-linear. The first opening can have a first opening major dimension along a first opening major axis and a first opening minor dimension along a first opening minor axis. The first opening major axis can be a vertical axis. The first opening major axis can extend along a greatest dimension of the first opening in a first direction, e.g., along a vertical direction such that the first opening major dimension is a greatest height of the first opening. A remainder of the first opening can have a lesser height than the major dimension. The first opening minor axis can be a horizontal axis. The first opening minor axis can extend along a greatest dimension of the first opening in a second direction, which may be perpendicular to the first direction, e.g., along a horizontal direction such that the first opening minor dimension is a greatest width of the first opening. A remainder of the first opening can have a lesser width than the minor dimension. The first opening minor dimension and/or minor axis can be offset (e.g., offset below in some configurations) from a midpoint of the first opening major dimension. The second opening can have a generally triangular shape. The second opening can have a second opening major dimension along a second opening major axis and a second opening minor dimension along a second opening minor axis. The second opening major axis can be a vertical axis. The second opening major axis can extend along a greatest dimension of the second opening in a first direction, e.g., along a vertical direction such that the second opening major dimension is a greatest height of the second opening. A remainder of the second opening can have a lesser height than the minor dimension. The second opening minor axis can be a horizontal axis. The second opening minor axis can extend along a greatest dimension of the second opening in a second direction, which may be perpendicular to the first direction, e.g., along a horizontal direction such that the second opening minor dimension is a greatest width of the second opening. A remainder of the second opening can have a lesser width than the minor dimension. The second opening minor dimension and/or minor axis can be offset (e.g., offset below in some configurations) from a midpoint of the second opening major dimension. At least one of the first opening major dimension and the first opening minor dimension can be greater than at least one of the second opening major dimension and the second opening minor dimension, respectively. For example, the first opening major dimension can be greater than twice the second opening major dimension. The first opening minor dimension can be greater than twice the second opening minor dimension. In use, gases flow through the connection opening 132 and/or second opening of the second end of the housing 122, through the housing 122, through the first opening of the first end of the housing 122, and into or through a space bounded by the scal 124.

In the illustrated embodiment, the connection opening 132 is generally ovate, or obovate (or has a shape that can be described as a combination of a semicircle and two splines) to accommodate a generally ovate or obovate collar 152 of the frame 110. Such a non-circular and/or asymmetric shape can inhibit or prevent the cushion module 120 from rotating relative to the frame 110 and/or from being assembled to the frame 110 in an improper alignment or orientation. The illustrated shape advantageously does not include discontinuities, pinch points, or corners in the cushion module flange. The illustrated shape can allow for an elastomeric friction member 142 of consistent dimensions to be used across a range of seal sizes (e.g., small, medium, and/or large). The size and/or shape of the illustrated connection opening 132 can accommodate both the gas inlet 130 and vent 170 of the frame 110. Such an arrangement can allow for a relatively large area for the vent 170.

In some configurations, the connection opening 132 has a major dimension α along a major axis (shown in FIG. 149). The major dimension α can be about 57 mm. The connection opening 132 can have a minor dimension β along a minor axis (shown in FIG. 149). The minor dimension β can be about 30 mm. In other words, the major dimension α is larger than the minor dimension β. The values of the major dimension α and the minor dimension β can vary depending on the size of the cushion module 120. However, in some configurations, regardless of the size of the cushion module 120 or exact values of the major dimension α and the minor dimension β, a ratio of α:β can be about 57:30, about 1.9:1 or about 2:1. The connection opening 132 is symmetric about the major axis. The connection opening is asymmetric about the minor axis. The major axis is perpendicular to the minor axis. The major axis corresponds to a vertical axis of the cushion module 120. The major dimension α is therefore a vertical dimension. The minor axis corresponds to a horizontal axis of the cushion module 120. The minor dimension β is therefore a horizontal dimension.

As described above, in some configurations, the connection opening 132 can be considered to have a shape that is a combination of a semicircle and a spline, for example as shown in FIG. 150. As shown, the semi-circle, or at least a portion of a semi-circle can form a lower extreme or portion of the connection opening 132. The semi-circle can have a diameter B and an origin O. A spline S can extend upwards from each end of the semi-circle. The splines S can meet at a point T that is positioned vertically above origin O and forms an uppermost extreme or point of the connection opening 132. A distance between origin O and point T is indicated by η. The major dimension α is the distance between T and the bottommost extreme or point of the semi-circle. In other words, α=η+β/2. In some configurations, η is about 42 mm. In some configurations, a ratio of η:α is 42:57 or about 1:1.35.

A friction coupling 140 couples the cushion module 120 and the frame 110. As described herein, the friction coupling 140 is or comprises an elastomeric friction member 142. The elastomeric friction member 142 can be connected to or adjacent the second end of the housing. In the illustrated configuration, the elastomeric friction member 142 is coupled to, e.g., overmolded to, a support flange 162. The support flange 162 can define, be disposed at or adjacent, and/or extend from the second end of the housing 122. The support flange 162 can at least partially surround the second opening. The elastomeric friction member 142 and support flange 162 together can be considered or called a cushion module flange. The cushion module flange can define the connection opening 132. In some embodiments, the connection opening 132 can be defined by the housing 122, for example the support flange 162. In other embodiments, the connection opening 132 can be defined by the elastomeric friction member 142. In the illustrated configuration, the elastomeric friction member 142 encloses the support flange 162. Therefore, the support flange 162 can be considered an internal flange. In other words, the support flange 162 is embedded within the elastomeric friction member 142.

The length of the support flange 162 (in a longitudinal or axial direction) can vary about the perimeter of the support flange 162. As shown in, e.g., FIGS. 143 and 146, the support flange 162 is longer (in a longitudinal or axial direction) at a bottom (or in a lower portion) of the cushion module 120 than at a top (or in an upper portion) of the cushion module 120. The upper portion is located above the lower portion. The upper portion can include a portion or an entirety of the upper half of the support flange 162 and/or connection opening 132. The lower portion can include a portion or an entirety of the lower half of the support flange 162 and/or connection opening 132. The upper portion can include an apex of the support flange 162 and/or connection opening 132. The upper portion can be disposed vertically above the gas inlet 130 of the frame 110 when the cushion module 120 is coupled to the frame 110. The upper portion can be disposed at least partially above and/or partially surround the vent arrangement 170 when the cushion module 120 is coupled to the frame 110. The upper portion can include, for example, an upper third of the support flange 162. The lower portion can be disposed at least partially below and/or partially surround the gas inlet 130 of the frame 110 when the cushion module 120 is coupled to the frame 110. The lower portion can include, for example, a lower third of the support flange 162. A relatively longer support flange 162 (and therefore elastomeric friction member 142) can provide greater rotational stability to the connection between the frame 110 and the cushion module 120. A relatively longer support flange 162 and elastomeric friction member 142 along the bottom or lower portion of the cushion module 120 can allow the frame 110 to be more easily connected to the cushion module 120. As shown in FIG. 167, this arrangement can allow the frame 110 to be rolled or pivoted into place on the cushion module 120. In other words, the bottoms of the frame 110 and cushion module 120 can be coupled or engaged along the direction of arrow I in FIG. 167, then the top of the frame 110 can be rolled, pivoted, or rotated toward the cushion module 120, as indicated by arrow R in FIG. 167. When the top portion of the frame 110 engages the top portion of the elastomeric friction member 142, the shorter support flange 162 and elastomeric friction member 142 in that portion allow for a smaller interference region of the elastomeric friction member 142 for easier coupling or engagement of the frame 110 and cushion module 120 at this portion. The frame 110 can be more easily disengaged by rotating it away from the cushion module 120 in an opposing direction to that which it was connected.

The housing 122 of the illustrated cushion module 120 includes a main wall 160 and the support flange 162. In the illustrated configuration, the support flange 162 extends rearwardly (posteriorly or toward the user in an assembled and fitted in use orientation). The main wall 160 and/or the support flange 162 surrounds and/or defines the connection opening 132. The housing 122 also includes a support wall 163 that surrounds and is adjacent the connection opening 132 and/or the support flange 162. The support wall 163 can be a portion of the main wall 160. The support wall 163 can connect the support flange 162 to a remainder of the main wall 160. The support wall 163 can be a portion of the main wall 160 that is embedded within the elastomeric friction member 142. Alternatively, the support wall 163 could be separate from the main wall 160, such as spaced forwardly or rearwardly from the main wall 160. The support flange 162 defines a perimeter shape that preferably corresponds to the perimeter shape of the collar 152, but preferably is somewhat larger to accommodate a portion of the friction member 142 therebetween. The support flange 162 of the housing 122 could extend in a forward direction (away from the user) in addition or in the alternative of the rearward-extending support flange 162.

The housing 122 comprises one or more apertures 166 through which the material of the elastomeric friction member 142 extends as a result of the over-molding process. In the illustrated arrangement, the apertures 166 are located in the support wall 163. The apertures 166 can be rectangular or trapezoidal. At least a portion of the perimeter of the apertures 166 can be curved, which can help reduce stress concentration locations at the border(s) between the elastomeric friction member 142 and the support flange 162 and/or support wall 163. The size of the apertures 166 can be selected based at least in part on the strength needed for the elastomeric friction member 142. In some configurations, each aperture 166 can have a major dimension of about 3 mm. Each aperture 166 can have a minor dimension of about 1.8 mm. The apertures 166 can be separated from each other by a distance of about 3 mm along the perimeter of the support wall 163. In at least one configuration, an outer edge (defining the profile of the aperture on a radially outer surface of the support wall 163) of at least one of the apertures 166 can be rounded. In at least one configuration, an inner edge (defining the profile of the aperture on a radially inner surface of the support wall 163) of at least one of the apertures 166 can be rounded. In at least one configuration, both the outer edge and the inner edge of at least one of the apertures 166 can be rounded. Rounding the inner edge and/or the outer edge of one or more of the apertures 166 can help reduce stress concentrations in the elastomeric friction member 142 at or near the apertures 166, thereby increasing the life of the connection between the elastomeric friction member 142 and the housing 122. In the illustrated arrangement, the elastomeric friction member 142 comprises an engagement surface 144 (shown in, e.g., FIG. 152) that engages an engagement surface 154 (shown in, e.g., FIG. 156) of the frame collar 152. A friction-fit between the collar 152, e.g., the engagement surface 154, and the elastomeric friction member 142 secures the frame 110 and cushion module 120 together. In at least one configuration, the engagement surface 144 is an interior or radially inward-facing surface of the elastomeric friction member 142. In at least one configuration, the engagement surface 154 is an exterior or radially outward-facing surface of the collar 152. However, in other arrangements, the elastomeric friction member 142 could engage an interior surface of the collar 152. Preferably, with either arrangement the frame 110 can define at least a portion of the breathing chamber 102. For example, in the illustrated configuration, an interior space defined by the collar 152 defines a portion of the breathing chamber 102.

As described above, in the illustrated arrangement, the peripheral surface 146 (shown in, e.g., FIG. 152) is exposed or is not directly covered when the cushion module 120 is assembled to the frame 110. With such an arrangement, the elastomeric friction member 142 is compressed from a single surface (e.g., the engagement surface 144), or from the engagement surface 144 and a front or leading surface of the elastomeric friction member 142, for example as shown in FIGS. 164 and 165. Accordingly, dimensional variations as a result of the manufacturing process will not impact the assembly and removal force to as great an extent as a design in which both the interior and exterior surfaces of the elastomeric friction member 142 engage a surface of a cooperating structure. Moreover, having the peripheral surface 146 of the elastomeric friction member 142 exposed to atmosphere advantageously allows the cushion module 120 and/or frame 110 to be easier to clean than an arrangement having a channel, which can be difficult to access by the user.

In some configurations, the housing 122 includes a rim 232, as shown in FIGS. 151-152. The rim 232 is a portion of the main wall 160 that projects rearwardly or inward into the breathing chamber of the cushion module 120. In other words, the rim 232 has a thickness greater than a thickness of an adjacent portion of the housing 122. In the illustration configuration, the rim 232 is or extends adjacent the elastomeric friction member 142. The rim 232 is therefore positioned just outside or adjacent the mold tool during overmolding of the elastomeric friction member 142 on the support flange 162. The rim 232 can provide improved manufacturing yields. Arrows 234 in FIG. 152 indicate draw directions for the mold tools used when overmolding the elastomeric friction member 142 to the support flange 162. Without an enlarged rim 232, during the molding process, the pressure of the molten plastic may cause the housing 122 to deform along the length of the support flange 162 and/or main wall 160. Such deformation could create small gaps in areas intended to be sealed by the mold tool. The rim 232 can help prevent or inhibit such deformation. If the housing 122 tries to deform during overmolding of the elastomeric friction member 142, the rim 232 presses against the mold tool to provide a reactive force to help reduce or minimize deformation of the housing 122. This can help improve yields by reducing cases of flashing and/or reducing variability of the position of the support flange 162 within the elastomeric friction member 142, which could result from deformation of the support flange 162 under pressure.

The elastomeric friction member 142 can have a rounded front edge or surface 244, as shown in FIG. 151. A thickness Ti (as measured from the support flange 162 to an exterior edge or surface of the elastomeric friction member 142 as shown) of a front portion 246 of the elastomeric friction member 142 can be greater than a thickness T2 of a more rearward portion of the elastomeric friction member 142 as shown in FIG. 151. In other words, an exterior surface of the front portion 246 is farther away from the support flange 162 than the exterior surface of the more rearward portion. Such an arrangement can concentrate the interference, indicated by region 298 in FIGS. 164 and 165, between the elastomeric friction member 142 and the frame collar 152 towards the front of the elastomeric friction member 142. This concentration of interference toward the front of the elastomeric friction member 142 can result in a greater retaining force between the elastomeric friction member 142 and frame collar 152 at the distal or forward end of the elastomeric friction member 142 compared to the proximal or rear end of the elastomeric friction member 142. The concentration of interference toward the front of the elastomeric friction member 142 can reduce deformation of the support flange 162 and/or elastomeric friction member 142 along their length and/or reduce deformation of the frame collar 152, which can improve rotatability of the elbow 158. In the case of the frame collar 152, the main wall of the frame 110 provides strength to the frame 110 so the frame 110 can better support interference forces.

In the illustrated configuration, the support flange 162 extends rearwardly from or relative to the main wall 160 and/or support wall 163 of the housing 122 into the breathing chamber. As shown in, e.g., FIGS. 152 and 165, a transition region 262 of the housing 122 is formed where the support flange 162 meets the main wall 160 or the support wall 163 (in other words, where a front surface of the main wall 160 or support wall 163 turns inwardly to form an inward facing surface of the support flange 162). In the illustrated configuration, the transition region 262 can have a relatively large radius. In other words, the transition region 262, which is disposed between and connects the main wall 160 or support wall 163 (specifically, the front surface) and the support flange 162 (specifically, the inward facing surface), can be curved or rounded rather than sharp. In some configurations, during assembly of the cushion module 120 with the frame 110, a compressive force on the elastomeric friction member 142 compresses the elastomeric friction member 142 against the support wall 163 and the transition region 262. The large radius of the transition region 262 can reduce stress concentrations in the elastomeric friction member 142 around the front portion 246. As the interference between the elastomeric friction member 142 and frame 110 and the deformation of the elastomeric friction member 142 are concentrated toward the front of the elastomeric friction member 142, the large radius of the transition region 262 can help reduce or prevent stress concentrations in portions of the elastomeric friction member 142 under greater stress. Reduction of such stress concentrations can help improve the longevity of the elastomeric friction member 142.

In the illustrated arrangement, the frame 110 defines a socket 157 (shown in FIG. 156) configured to receive a conduit connector, such as an elbow 158. The elbow 158 can be configured to connect a breathing circuit to the patient interface 100. In the illustrated arrangement, the collar 152 defines at least a portion of the elbow socket 157. In some configurations, the conduit connector or elbow 158 is capable of pivoting or rotating about one or more axes relative to the frame 110. For example, the conduit connector or elbow 158 can have a swivel connection with the frame 110. Alternatively, the conduit connector or elbow 158 can form a ball-jointed connection with the frame 110. The elbow 158 can be removably coupled to the frame 110.

The vent 170 can be located and/or integrated in the frame 110. In the illustrated embodiment, the vent 170 is located in a portion of the frame 110 that overlies the connection opening 132. In other words, the frame collar 152 encircles the vent 170 and the frame opening 130 as shown. Such an arrangement can allow for a relatively large area for the vent 170, which can allow the bias flow holes of the vent 170 to be spread out, while still accommodating the gas inlet 130 of the frame 110. A diffuser 173 can be disposed over a front of the vent 170 as shown in FIG. 153. The diffuser 173 can diffuse air vented through the bias flow holes of the vent 170 to help reduce the noise of the air flow through the vent 170 and/or to help reduce draft produced by the air flow through the vent 170. As shown in FIG. 156, the frame 110 can include a divider wall 172 that is positioned between and/or separates the vent 170 and the gas inlet 130. The divider wall 172 can partially define the elbow socket 157. To provide an elbow socket 157 that allows for multi-directional rotation of the elbow 158, the interior surface of the elbow socket 157, defined partially by the frame collar 152 and partially by the divider wall 172, is spherical. To create such a spherical socket 157, a spherical tool (or spherical portion) is used during injection molding of this portion of the frame 110. After molding, to remove the frame 110, or portion thereof, from the mold tool, the frame 110 or frame portion is pulled in a direction parallel or substantially parallel to the central axis of the frame opening 130. This motion causes the frame collar 152 and divider wall 172 to flex outwards as the rear edges of the frame collar 152 and divider wall 172 pass over the spherical portion of the mold tool. Such flexing can stretch the rear sections of the frame collar 152 and result in a deformed collar 152 and elbow socket 157, which can hinder performance of the elbow 158 and/or introduce a leak. In some configurations, the divider wall 172 includes one or more divider wall recesses 175, as shown in FIGS. 156-157, which allow the divider wall 172 and collar 152 to elastically flex around the spherical tool portion during removal of the frame 110. This can advantageously reduce permanent deformation of the frame collar 152 and/or divider wall 172. In the illustrated configuration, the divider wall 172 includes two recesses 175, one on each lateral side of the divider wall 172 such that a gap exists between each distal or lateral end of the divider wall 172 and the frame collar 152.

FIG. 166 shows a variation of the cushion module 120 in which the elastomeric friction member 142 includes a lateral projection of each lateral side to help improve the grip between the elastomeric friction member 142 and the frame collar 152. In the illustrated configuration, the lateral projections are in the form of lateral beads 302. The lateral beads 302 span a portion of the internal perimeter of the elastomeric friction member 142. When forming the elastomeric friction member 142, manufacturing variations between different elastomeric friction members 142 can cause variations in the removal force (the force required to remove the frame 110 from the cushion module 120 once the frame 110 is coupled to the cushion module 120) between the different cushion modules 120. Including lateral beads 302 (or other lateral projections) on only lateral portions of the elastomeric friction member 142 can help reduce this variance in removal force among different cushion modules 120 and therefore allow for more consistent performance of the elastomeric friction member 142. In some configurations, the elastomeric friction member 142 can include more than one lateral bead 302 on each lateral side. In other configurations, the elastomeric friction member 142 can include a single bead 302 spanning an entire perimeter of the elastomeric friction member 142.

FIGS. 168-214 illustrate another patient interface 100 that is similar to the patient interfaces 100 of FIGS. 15-20 and 139-167 in some respects. The illustrated patient interface 100 includes a frame 110, which includes a frame main body 268 and a frame insert 270, and a cushion module 120, which includes a cushion or seal 124 coupled to a housing 122. The seal 124 is configured to seal on a patient in use. In the patient interface 100 of FIGS. 168-214, the support flange 162 of the cushion module 120 extends forwardly, anteriorly, or away from the user in an assembled and fitted in-use orientation similar to the support flange 162 of the patient interface 100 of FIGS. 15-20 (in contrast, the support flange 162 of the cushion module 120 of the patient interface 100 of FIGS. 139-167 extends rearwardly, posteriorly, or toward the user in an assembled and fitted-in-use orientation).

In some configurations, a majority or entirety of the elastomeric friction member 142 can be considered a supported region 142s, as an axial length or a supported length x1 (shown in FIG. 185) of the supported portion 312 is greater than an axial length or unsupported length x2 (shown in FIG. 185) of the unsupported portion 310 about a majority or entirety of the perimeter of the elastomeric friction member 142, for example as shown in FIGS. 182 and 185. In some configurations, a first length or total length x (shown in FIG. 185) of the elastomeric friction member 142 is about 7 mm. The supported length x1 of the supported portion 312 can be about 5 mm. The unsupported length x2 of the unsupported portion 310 can be about 2 mm. A ratio of x2:x1 can be about 5:2.

As shown in FIG. 186, the elastomeric friction member 142 defines a first thickness 190 that extends in or substantially in a radial direction or perpendicular to the direction of assembly 104. The support flange 162 defines a second thickness 192 that extends in or substantially in a radial direction or perpendicular to the direction of assembly 104. A portion of the elastomeric friction member 142 located radially outside of the support flange 162 defines a third thickness 194 that extends in or substantially in a radial direction or perpendicular to the direction of assembly 104 (FIG. 179). A portion of the elastomeric friction member 142 located radially inside of the support flange 162 defines a fourth thickness 196 that extends in or substantially in a radial direction or perpendicular to the direction of assembly 104.

In the illustrated configuration, the first thickness 190 equals the sum of the second thickness 192, the third thickness 194, and the fourth thickness 196. As shown, the first thickness 190, or total thickness of the elastomeric friction member 142, can vary along its length. The second thickness 192, third thickness 194, and/or fourth thickness 196 can vary along their length(s). In the embodiment of FIG. 186, the fourth thickness has a distal thickness 196, 1, a central thickness 196, 2, and a proximal thickness 196, 3. The first thickness 190 can be about 2.86 mm. The second thickness 192 can be about 1 mm. The third thickness 194 can be about 1.1 mm. The distal thickness 196, 1 can be about 0.75 mm. The central thickness 196, 2 can be about 0.79 mm. The proximal thickness 196, 3 can be about 1.07 mm. In other words, the proximal thickness 196, 3 can be greater than the central thickness 196, 2 and/or the distal thickness 196, 1. In some cases, the various thickness of the elastomeric friction member 142 can vary due to, for example, angled surfaces of the support flange 162 and/or expansion or shrinkage of the elastomeric friction member 142 during or after molding. In at least one configuration, the central thickness 196, 2 can be greater than the distal thickness 196, 1 and/or the proximal thickness 196, 3. This can concentrate interference between the elastomeric friction member 142 and the collar 152 near the central thickness 196, 2. In at least one configuration, the distal thickness 196, 1 can be greater than the central thickness 196, 2 and/or the proximal thickness 196, 3. This can concentrate interference between the elastomeric friction member 142 and the collar 152 near the distal thickness 196, 1.

In some configurations, the connection opening 132 has a major dimension α1 along a major axis (shown in FIG. 178). As shown, the major dimension α1 is measured from an inner surface of a distal end of the elastomeric flange 142 at a top of the connection opening 132 to an inner surface of a distal end of the elastomeric flange 142 at a bottom of the connection opening 132. The major dimension α1 can be about 60 mm. The connection opening 132 can have a minor dimension β along a minor axis (shown in FIG. 178). As shown, the minor dimension β is measured from an inner surface of a lateral side of the elastomeric flange 142 at the left most lateral point of the connection opening 132 to an inner surface of a lateral side of the elastomeric flange 142 at a right most lateral point of the connection opening 132. The minor dimension β can be about 32 mm. In other words, the major dimension α1 is larger than the minor dimension β. The values of the major dimension α1 and the minor dimension β can vary depending on the size of the cushion module 120. However, in some configurations, regardless of the size of the cushion module 120 or exact values of the major dimension α1 and the minor dimension β, a ratio of α1:β can be about 60:32, about 1.9:1 or about 2:1. In the illustrated arrangement, the connection opening 132 is symmetric about the major axis and asymmetric about the minor axis. The major axis is perpendicular to the minor axis. The major axis corresponds to a vertical axis of the cushion module 120. The major dimension α1 is therefore a vertical dimension. The minor axis corresponds to a horizontal axis of the cushion module 120. The minor dimension β is therefore a horizontal dimension. The minor axis is disposed within a lower third of the connection opening 132. In other words, the minor axis is disposed within a lower third of the major dimension α1. In at least one form, the minor axis can be disposed within a lower half of the connection opening 130. The minor axis can therefore be disposed within a lower half of the major dimension α1. In at least one configuration, the minor axis can be disposed within an upper third, or an upper half of the connection opening 132, and/or the major dimension α1.

A thickness γ of the elastomeric flange 142 at its distal end, shown in FIGS. 178 and 179, can be approximately constant around the perimeter of the elastomeric flange 142. In some configurations, the thickness γ is about 3 mm (e.g., 3.03 mm). A width of the housing 122 at its widest point, indicated by δ in FIG. 178, can be about 80-85 mm (e.g., 81.42 mm), for example, for a medium sized seal. An overall width of the cushion module 120, which in the illustrated configuration corresponds to a width of the seal 124 at its widest point, indicated by ε in FIG. 178, can be about 90-100 mm (e.g., 94.5 mm), for example, for a medium sized seal. An overall height of the cushion module 120, which in the illustrated configuration corresponds to a height of the seal 124 at its tallest point (or point with the greatest height), indicated by ζ in FIG. 179, can be about 105-110 mm (e.g., 106.5 mm), for example, for a medium sized seal. In the illustrated configuration, the height of the seal ζ is greater than the sum of the major dimension α1 and the minor dimension β. The width of the housing δ is more than twice the minor dimension β. The height of the seal ζ is less than twice the major dimension α1.

In some configurations, the connection opening 132 can be considered to have a shape that is a combination of a semicircle and a at least one spline, for example as shown in FIG. 180. As shown, the semi-circle, or at least a portion of a semi-circle can form a lowermost extreme or lowermost portion of the connection opening 132. The semi-circle can have a diameter σ1 and an origin O. A spline S can extend upwards from each end of the semi-circle. The splines S can meet at a point T that is positioned vertically above origin O and forms an uppermost extreme or uppermost point of the connection opening 132. A distance between origin O and point T is indicated by n. The major dimension α1 is the distance between T and the lowermost extreme or lowermost point of the semi-circle. In other words, α1=η+σ1/2. In some configurations, η is about 45-50 mm (e.g., 46 mm). In some configurations, σ1 is about 32-38 mm (e.g., 34.6 mm).

As shown in FIG. 181, the elastomeric flange 142 can be curved when viewed from the side such that lateral portions of the elastomeric flange 142 extend or project forward to a greater extent than the uppermost and lowermost points or extremes of the elastomeric flange 142. In some configurations, the semicircle and spline are aligned with or extend along a midpoint of the thickness and depth of the elastomeric flange 142, as shown in FIG. 181. An angle θ2 is therefore formed between the semicircle and the major dimension α1. The angle θ2 can be about 9°.

In the illustrated arrangement, the elastomeric friction member 142 comprises an engagement surface 144 (shown in, e.g., FIG. 175) that engages an engagement surface 154 (shown in, e.g., FIG. 198) of the insert collar 152. A friction-fit between the collar 152, e.g., the engagement surface 154, and the elastomeric friction member 142 secures the frame 110 and cushion module 120 together. In at least one configuration, the engagement surface 144 is an interior or radially inward-facing surface of the elastomeric friction member 142. In at least one configuration, the engagement surface 154 is an exterior or radially outward-facing surface of the collar 152. However, in other arrangements, the elastomeric friction member 142 could engage an interior surface of the collar 152. Preferably, with either arrangement the frame 110 can define at least a portion of the breathing chamber 102. For example, in the illustrated configuration, an interior space defined by the collar 152 defines a portion of the breathing chamber 102.

An abutment surface 148 (FIG. 182) of the elastomeric friction member 142 abuts or contacts the frame 110 when the cushion module 120 is coupled to the frame 110. The abutment surface 148 is a forward-facing surface of the friction member 142. The abutment surface 148 is configured to contact a rearward-facing surface of the frame 110 surrounding and/or adjacent the collar 152. For example, the abutment surface 148 can contact a rear peripheral recess 280 of the frame 110 as shown in FIGS. 189-190 and described herein. As shown in FIG. 182, the abutment surface 148 extends at an angle Φ2 relative to the engaging surface 144 (e.g., a first engaging surface 144a as described herein) of the elastomeric friction member 142. Angle Φ2 can be about 70°. Angle Φ2 can be selected to reduce or minimize the curve of the rear peripheral recess 280 of the frame 110.

The engagement surface 144 can have a first engaging surface 144a and a second engaging surface 144b, as shown in FIG. 182. The second engaging surface 144b can extend at an angle Φ1 relative to the first engaging surface 144a. The angle Φ1 can be selected based at least in part on the grade of silicone used for the elastomeric friction member 142 and/or the forces desired to be required to connect and/or disconnect the frame 110 and cushion module 120. For example, in some configurations in which the elastomeric friction member 142 is made of or includes LSR 4070 silicone, an angle Φ1 of 3° may be selected. As shown in FIG. 182, the elastomeric friction member 142 can be angled inward towards the center of the inlet 132 as the elastomeric friction member 142 extends from the housing 122 toward the front end or edge of the elastomeric friction member 142. This angle can accommodate a cooperating shape of the insert collar 152. The first engaging surface 144a can be tapered or angled more than the second engaging surface 144b as shown. A greater interference or stronger friction fit is therefore formed between the first engaging surface 144a (or portion of the elastomeric friction member 142 including the first engaging surface 144a) and frame 110. Angle Φ1 can be varied depending on the difference in interference desired between the first 144a and second 144b engaging surfaces.

As described herein, the elastomeric friction member 142 can be coupled to the support flange 162 via an overmolding process. FIGS. 183 and 184 illustrate the housing 122 disposed in an example mold tool assembly. The mold tool assembly includes a first mold tool component 502, a second mold tool component 504, and a third mold tool component 506. FIG. 184 shows a void 508 for injection molding of the elastomeric friction member 142 within the mold tool assembly. FIG. 184 also shows a shut off 512 for the second mold tool component 405 and a shut off 514 for the third mold tool component 506. As shown, shut off 512 and shut off 514 are not aligned with each other. In other words, shut off 512 and shut off 514 do not directly oppose each other. The offset shut offs 512, 514 allow the size of the cushion module inlet to be maximized, at least for the illustrated configuration. To have the shut offs 512, 514 be aligned, the housing 122 and elastomeric friction member 142 would need to be displaced to the right in the orientation of FIG. 184, thereby increasing the length of the horizontal (in the orientation of FIG. 184) portion of the housing 122 and decreasing the size of the cushion module inlet. Decreasing the size of the cushion module inlet can reduce the surface area available for vent holes, which can lead to louder venting in use. In other configurations, the shuts offs 512, 514 may be aligned, which can reduce shear force exerted on the housing 122 when the mold tool assembly closes and clamps onto the housing 122 and/or reduce or prevent leaking (flash) of the injection molding material (e.g., silicone) from the void 508.

As shown in, e.g., FIGS. 192-199, the frame 110 can include a frame insert 270. In the illustrated configuration, the frame insert 270 includes a vent wall 275, an abutment flange 276, and an insert wall, which can also be referred to herein as an insert collar 278. In some configurations, the insert wall 278 is a closed loop or a substantial portion of a closed loop that forms or approximates a collar and, thus, is referred to as an insert collar 278. However, in other configurations, the insert wall 278 may be shorter in length and may not approximate a collar. Thus, the instances of “insert collar” herein may be replace with “insert wall.” The vent wall 275 and insert collar 278 can together form or be considered an insert collar, a vent collar or, when assembled to the frame main body 268, a frame collar 152 (FIG. 196). The abutment flange 276 has an enlarged dimension, e.g., diameter, circumference, or perimeter, compared to the vent wall 275 and insert collar 278. The vent wall 275 and insert collar 278 extend rearward (or toward the user in use) from the abutment flange 276. The vent wall 275 extends upward from lateral sides or portions of the insert collar 278. When the cushion module 120 is coupled to the frame insert 270, a lower portion (e.g., a lower half) of the insert collar 278 corresponds to and/or contacts the portion of the elastomeric flange 142 surrounding the semicircle of the connection opening 132, and the vent wall 275 corresponds to and/or contacts the portion of the elastomeric flange 142 surrounding the splines S of the connection opening 132. The insert collar 278 can include or define an insert collar recess 360 recessed from a free rear edge of the insert collar 278. In the illustrated configuration, the insert collar recess 360 is disposed in or along a top of the insert collar 278. The frame insert 270 has an insert opening 272 defining a gas flow path through the frame insert 270. In the illustrated configuration, the insert collar 278 (e.g., an inner surface of the insert collar 278) defines the insert opening 272.

The cushion module 120 can be coupled to the frame 110 by sliding the cushion module flange 147 onto the insert collar 278 and vent wall 275. In some configurations, the frame insert 270 and elastomeric friction member 142 are colored with the same color to indicate to the user that those two parts should be joined together. In FIGS. 228-230, for example, the frame insert 270 and elastomeric friction member 142 are shaded to indicate the color blue.

As shown in FIG. 211, the frame 110 (or frame 110 and frame insert 270 combination), includes a rear peripheral recess outer deflection portion 280a extending along a radially outer edge or portion of the rear peripheral recess 280 and a rear peripheral recess inner deflection portion 280b extending along a radially inner edge or portion of the rear peripheral recess 280. The elastomeric friction member 142 includes an outer elastomeric flange curve 148a forming a transition between the abutment surface 148 and the peripheral surface 146, and an inner elastomeric flange curve 148b forming a transition between the abutment surface 148 and the engaging surface 144. When the cushion module 120 is coupled to the frame 110, the elastomeric friction member 142 can deform such that portions of the friction member 142, e.g., portions at or near the outer elastomeric flange curve 148a and inner elastomeric flange curve 148b, can at least partially fill the rear peripheral recess outer deflection portion 280a and rear peripheral recess inner deflection portion 280b, respectively. This deformation can advantageously allow for a better fit between the elastomeric friction member 142 and the frame 110.

FIGS. 210 and 211 show an interference region 145v between the elastomeric friction member 142 and the vent wall 275. A portion or entirety of one or both of the vent wall 275 and the elastomeric friction member 142 deflects or deforms to allow the components to fit together. FIGS. 212-214 shows an interference region 145i between the elastomeric friction member 142 and the insert collar 278. Similarly, a portion or entirety of one or both of the insert collar 278 and the elastomeric friction member 142 deflects or deforms to allow the components to fit together. Because of the variable thickness of the portion of the elastomeric friction member 142 located radially inside of the support flange 162 and/or the angle of the first engaging surface 144a relative to the second engaging surface 144b, the thickness τ of the interference region 145 (145i and/or 145v) varies along the axial length of the elastomeric friction member 142.

As shown in FIG. 211, in the illustrated configuration, the interference region 145v is thicker along the length of the first engaging surface 144a relative to along the second engaging surface 144b. The interference, and therefore retaining force between the elastomeric friction member 142 and vent wall 275, is therefore greater at or near the distal or front end of the cushion module flange than at or near the proximal or rear end of the cushion module flange. The interference region 145v has a thickness τ1 proximate the front or patient-distal end of the elastomeric friction member 142, a thickness τ3 proximate a rear or patient-proximal end of the elastomeric friction member 142 and/or a rear or free end of the vent wall 275, and a thickness τ2 between thickness τ1 and thickness τ3. As shown, thickness τ2 may be at a transition between the first engaging surface 144a and the second engaging surface 144b. In the illustrated configuration, the interference region 145v has a constant thickness along the second engaging surface 144b. In some configurations, τ1>τ2 and τ1>τ3, where τ2=τ3, τ2>τ3, or τ2<τ3.

Similar to interference region 145v, the interference region 145i shown in FIGS. 213 and 214 is thicker along the length of the first engaging surface 144a relative to along the second engaging surface 144b. The interference, and therefore retaining force between the elastomeric friction member 142 and insert collar 278, is therefore greater at or near the distal or front end of the cushion module flange than at or near the proximal or rear end of the cushion module flange. The interference region 145i has a thickness τ4 proximate the front or patient-distal end of the elastomeric friction member 142, a thickness τ6 proximate a rear or patient-proximal end of the elastomeric friction member 142, and a thickness τ5 between thickness τ4 and thickness τ6. As shown, thickness τ5 may be at a transition between the first engaging surface 144a and the second engaging surface 144b. In the illustrated configuration, the interference region 145i has a constant thickness along the second engaging surface 144b. In some configurations, τ4>τ5 and τ4>τ6, where τ5=τ6, τ5>τ6, or τ5<τ6.

The illustrated frame insert 270 includes a vent arrangement 170 disposed in a projecting surface 177 that extends or projects forward (or away from the user in use) from and/or relative to the abutment flange 276. As shown, the projecting surface 177 can project forward from and/or relative to a front surface of a portion of the abutment flange 276 from which the vent wall 275 projects from a rear surface. The vent arrangement 170 and/or projecting surface 177 can be bounded by the vent wall 275 and an upper portion of the insert collar 278. The projecting surface 177 and vent arrangement 170 are therefore positioned generally above the insert collar 278 and insert opening 272.

The vent arrangement 170 can have various vent hole arrangements. Different vent hole arrangements can allow for different air flow patterns through the vent arrangement 170. For example, all vent holes can share or be aligned with respective parallel axes, as shown by arrows V1 (the left 3 arrows) in FIG. 194. In such an arrangement, air is directed parallel out of all of the holes. As another example, the vent holes can be aligned at angles relative to each other, as shown by arrows V2 (the right 3 arrows) in FIG. 194, such that air is vented along non-parallel paths through different vent holes. The configuration indicated by arrows V2 can advantageously reduce vent noise and/or reduce draft produced by the vent arrangement 170 by more effectively diffusing air through the vent holes.

The frame main body 268, shown in FIGS. 187-191, includes a frame inlet opening 130a that surrounds the gas inlet 130 and vent arrangement 170 when the frame insert 270 is assembled with the frame main body 268. When the frame insert 270 is assembled with the frame main body 268, at least a portion of the insert opening 272 defines the gas inlet 130. The frame main body 268 includes a rear peripheral recess or channel 280 in a rear surface of the frame main body 268 surrounding the perimeter of the frame inlet opening 130a. When the cushion module 120 and frame main body 268 are fully coupled, a leading surface or the abutment surface 148 of the elastomeric friction member 142 fits into the rear peripheral recess 280 and abuts a rear surface of the frame 110. The frame main body 268 includes a front peripheral recess or channel 281 in a front surface of the frame main body 268 surrounding the perimeter of the frame inlet opening 130a. When the frame insert 270 is assembled with the main body of the frame 110, the frame insert 270 is inserted into the frame inlet opening 130a and a rear surface of the abutment flange 276 of the frame insert 270 at least partially rests in the front peripheral recess 281 of the frame main body 268. The frame insert 270 can be secured to the frame main body 268, for example, via welding (e.g., ultrasonic welding), adhesives, molding, and/or other means along the abutting surfaces of the frame insert 270 (e.g., the rear surface of the abutment flange 276) and the frame main body 268 (e.g., the front surface of the front peripheral recess 281). An energy concentrator 420 (FIG. 210) can be used to concentrate energy during an ultrasonic welding process to ensure a complete weld between the abutment flange 276 and front peripheral recess 281. The energy concentrator 420 can be located on or in the abutment flange 276 or the frame main body 268 (e.g., the front peripheral recess 281). In at least one configuration, the energy concentrator 420 can be in the form of a projection from a surface of the abutment flange 276. In the illustrated configuration, the energy concentrator 420 protrudes from a rear surface of the abutment flange 276. The energy concentrator 420 can be generally triangular in cross-section as shown.

In the illustrated arrangement, the frame insert 270 defines a socket 157 (shown in FIGS. 192-193) configured to receive a conduit connector, such as an elbow 158. The elbow 158 can be configured to connect a breathing circuit to the patient interface 100. In the illustrated arrangement, the insert collar 278 defines at least a portion of the elbow socket 157. In some configurations, the conduit connector or elbow 158 is capable of pivoting or rotating about one or more axes relative to the frame 110. For example, the conduit connector or elbow 158 can have a swivel connection with the frame 110. Alternatively, the conduit connector or elbow 158 can form a ball-and-socket connection with the frame 110. The elbow 158 can be removably coupled to the frame 110. The elbow socket 157 can include a cut-out 290, as shown in FIGS. 192-193, which can assist with inserting and/or removing the elbow 158. The cut-out 290 can be located along an inner surface of a bottom portion of the abutment flange 276.

To allow for multi-directional rotation of the elbow 158 relative to the frame 110, the interior surface of the elbow socket 157 is spherical. A spherical mold tool is used to injection mold the spherical socket 157. After molding, the frame insert 270 is removed from the mold tool by pulling in a direction parallel to the central axis of the insert opening 272, thereby flexing the insert collar 278 outward as the rear edge of the insert collar 278 passes over the spherical portion of the mold tool. The rear edge may be unlikely to revert back to its unstretched or unflexed state, which can result in a deformed insert collar 278 and elbow socket 157. This deformation can reduce performance of the elbow 158 and/or introduce a leak. The insert collar recess 360 allows the insert collar 278 to elastically flex around the spherical mold tool during removal, which can reduce deformation of the insert collar 278 and/or elbow socket 157.

Some users prefer to position the elbow 158 such that the conduit passes over their forehead and behind them in use (e.g., into a position indicated by 158U in FIG. 201, compared to a position in which the elbow 158 and conduit extend downward as indicated by 158D in FIG. 201). When rotated in this manner, the underside of the elbow 158 may be positioned close to the vent arrangement 170, which can increase noise as gases vented through the vent arrangement 170 blows over the elbow 158. To reduce this effect (the increase in noise), a bottom surface of the projecting surface 177 can project (e.g., can be sized and shaped) to an extent or in a way that limits rotation of the elbow 158 toward the frame insert 270 (in the direction indicated by arrows 158r in FIG. 201) when the elbow 158 is in the 158U position thereby providing a minimum spacing of the elbow 158 from the vent arrangement 170. This can help reduce noise produce by venting air hitting the elbow 158 by increasing the distance between the vent arrangement 170 and the elbow 158.

FIGS. 202 and 203 illustrate a variation of the frame insert 270 in which the front surface of the vent arrangement 170 does not project forward as much or to as great an extent. In this configuration, a lip 179 projects forward or outward from or near a bottom of the vent arrangement 170. The lip 179 acts as an anti-rotation feature or physical barrier limiting rotation of the elbow 158 toward the frame insert 270 (in the direction indicted by arrow 158r in FIG. 203). FIG. 204 illustrates another variation of the frame insert 270 that includes a central projecting surface 177. A bottom surface of the projecting surface 177 can act to limit rotation of the elbow 158 toward the frame insert 270. In this configuration, the vent arrangement 270 is split with vent holes positioned in side surfaces 178 of the frame insert 270 that extend from the projecting surface 177 toward or to the front surface of the abutment flange 276. Positioning the vent holes in the side surfaces 178 can advantageously reduce discomfort or disruption for a user's bed partner.

FIGS. 215-226 illustrate a variation of the cushion module 120 of FIGS. 168-214. In the configuration of FIGS. 215-226, the elastomeric friction member 142 includes an unsupported region 142u. In the illustrated configuration, the unsupported region 142u is disposed along a bottom portion or region of the elastomeric friction member 142. In the configuration of FIGS. 215-226, the shape and dimensions of the connection opening 132 are varied relative to those of the cushion module 120 of FIGS. 168-214 such that the connection opening 132 of FIGS. 215-226 has a reduced vertical (or major) dimension and an increased lateral dimension in the upper portion of the connection opening 132.

The connection opening 132 has a major dimension α1 along a major axis (shown in FIG. 217). As shown, the major dimension α1 is measured from an inner surface of a distal end of the elastomeric flange 142 at a top of the connection opening 132 to an inner surface of a distal end of the elastomeric flange 142 at a bottom of the connection opening 132. The major dimension α1 can be about 56 mm. The connection opening 132 can have a minor dimension β along a minor axis (shown in FIG. 217). As shown, the minor dimension β is measured from an inner surface of a lateral side of the elastomeric flange 142 at the left most lateral point of the connection opening 132 to an inner surface of a lateral side of the elastomeric flange 142 at a right most lateral point of the connection opening 132. The minor dimension β can be about 30 mm. In other words, the major dimension α1 is larger than the minor dimension β. The values of the major dimension α1 and the minor dimension β can vary depending on the size of the cushion module 120. However, in some configurations, regardless of the size of the cushion module 120 or exact values of the major dimension α1 and the minor dimension β, a ratio of α1:β can be about 56:30, about 1.87:1, about 1.9:1, or about 2:1. In the illustrated arrangement, the connection opening 132 is symmetric about the major axis and asymmetric about the minor axis. The major axis is perpendicular to the minor axis. The major axis corresponds to a vertical axis of the cushion module 120. The major dimension α1 is therefore a vertical dimension. The minor axis corresponds to a horizontal axis of the cushion module 120. The minor dimension β is therefore a horizontal dimension.

In some configurations, the connection opening 132 can be considered to have a shape that is a combination of a semicircle and at least one spline, for example as shown in FIG. 218. As shown, the semi-circle, or at least a portion of a semi-circle can form a lowermost extreme or lowermost portion of the connection opening 132. The semi-circle can have a diameter σ1 and an origin O. A spline S can extend upwards from each end of the semi-circle. The splines S can meet at a point T that is positioned vertically above origin O and forms an uppermost extreme or uppermost point of the connection opening 132. A distance between origin O and point T is indicated by η. The major dimension α1 is the distance between T and the lowermost extreme or lowermost point of the semi-circle. In other words, α1=η+σ1/2. In some configurations, η is about 45-50 mm (e.g., 46 mm). In some configurations, σ1 is about 32-38 mm (e.g., 33 mm). A ratio of η:α1 can be 46:56, or about 1:1.2. Such a non-circular and/or asymmetric shape can inhibit or prevent the cushion module 120 from rotating relative to the frame 110 and/or from being assembled to the frame 110 in an improper alignment or orientation. The illustrated shape advantageously does not include discontinuities, pinch points, or corners in the cushion module flange. The illustrated shape can allow for an elastomeric friction member 142 of consistent dimensions to be used across a range of scal sizes (e.g., small, medium, and/or large).

As shown in FIGS. 219-220, a top portion of the elastomeric friction member 142 is defined as a supported region 142s because the length x1 of the supported portion 312 is greater than the length x2 of the unsupported portion 310. In some configurations, the length x of the elastomeric friction member 142 is about 5.35 mm. The length x1 of the supported portion 312 can be about 3.4 mm. The length x2 of the unsupported portion 310 can be about 2.0 mm. Therefore, x2<x1. In some configurations, a ratio of x2:x1 is about 2:3.4 or about 1:1.7. In at least one configuration, the length x1 of the supported portion 312 can be equal to, and/or comparable to the length x2 of the unsupported portion 310.

The unsupported portion 310 can include a projection(s) to help improve the grip between the elastomeric friction member 142 and the frame 110 or frame insert 270. In the illustrated configuration, the projection is in the form of a bead 302. The bead 302 spans a portion or an entirety of the internal perimeter of the elastomeric friction member 142. The bead 302 can have a rounded (e.g., semi-circular), squared, triangular, or any other suitable profile. The illustrated bead 302 has a triangular profile.

FIG. 221 illustrates the interference region 145 between the elastomeric friction member 142 and the frame 110 (and/or frame insert 270) in the top portion or supported region 142s of the elastomeric friction member 142. Because of the bead 302 and/or the variable taper along the length of the elastomeric friction member 142, the interference region 145 is thicker along the unsupported portion 310 than along the supported portion 312. Therefore, the interference, and therefore the retaining force between the cushion module 120 and the frame 110 (and/or frame insert 270), is greater along the unsupported portion 310. In the illustrated configuration, the interference forces are greatest close to, or proximate, the vertical section (e.g., abutment flange 276) of the frame 110 or frame insert 270. This is beneficial because the frame insert 270 collars 275, 278 are stronger and more resistant to deformation by forces applies closer to the vertical section compared to at or near the free (rear or patient-proximal) end of the collars 275, 278 (e.g., due to lever mechanics of the cantilever collars 275, 278). If forces were applied near the free end of the insert collar 278, the collar 278 could deform, which could change the shape of the elbow socket 157, which could then inhibit or prevent the elbow 158 from rotating freely, properly, or at all.

As shown in FIGS. 222-223, a bottom portion of the elastomeric friction member 142 is defined as an unsupported region 142u (FIG. 216) because the length x1 of the supported portion 312 is less than the length x2 of the unsupported portion 310. In some configurations, the length x of the elastomeric friction member 142 is about 8.2 mm. The length x1 of the supported portion 312 can be about 3.35 mm. The length x2 of the unsupported portion 310 can be about 4.85 mm. Therefore, x2>x1. In some configurations, a ratio of x2:x1 is about 4.85:3.35 or about 1.45:1 or 1.5:1.

The unsupported portion 310 can include a projection(s) to help improve the grip between the elastomeric friction member 142 and the frame 110 or frame insert 270. In the illustrated configuration, the projection is in the form of a bead 302. The bead 302 spans a portion or entirety of the internal perimeter of the elastomeric friction member 142. The bead 302 can have a rounded (e.g., semi-circular), squared, triangular, or any other suitable profile. FIGS. 222-223 show the bead 302 positioned in the unsupported portion 310, but adjacent the supported portion 312. Alternatively, the bead 302 could be positioned closer to the front of the elastomeric friction member 142, as indicated by outline 302′ in FIG. 223. As shown in FIG. 224, the portion of the elastomeric friction member 142 adjacent the engaging surface 144 has a thickness y3. The bead 302 has a thickness y_{b}. In some configurations, y3 is about 1 mm and y_{b} is about 0.5 mm. The ratio of y_{b}:y3 can therefore be about 1:2. In other configurations, the ratio of y_{b}:y3 is in the range of about 0.3:1 to 0.5:1.

FIG. 225 illustrates the interference region 145 between the elastomeric friction member 142 and the frame 110 (or frame insert 270) in the bottom portion or unsupported region 142u of the elastomeric friction member 142. Because of the bead 302 and/or the variable taper along the length of the elastomeric friction member 142, the interference region 145 is thicker along the unsupported portion 310 than along the supported portion 312. Therefore, the interference, and therefore the retaining force between the cushion module 120 and the frame 110, is greater along the unsupported portion 310.

As shown and described herein, the length x of the elastomeric friction member 142 is greater at or near a bottom of the elastomeric friction member 142 (shown in FIGS. 222-223) compared to at or near a top of the elastomeric friction member 142 (shown in FIGS. 219-220). The length x2 of the unsupported region 310 is also greater at or near the bottom of the elastomeric friction member 142. Such a configuration allows the frame 110 or frame insert 270 to be more easily rolled into place when coupled to the cushion module 120, as indicated by arrow R in FIG. 226. The longer elastomeric friction member 142 and/or unsupported region 310 at the bottom of the elastomeric friction member 142 provides a more pronounced cantilever projection at the bottom of the elastomeric friction member 142. Although the cushion module 120 is rolled onto the frame 110 or the frame insert 270, or vice-versa, the assembly still occurs generally along an assembly direction as indicated by the insertion arrow in FIG. 226. The assembly direction can be used to provide a frame of reference by distinguishing the assembly direction from directions that are substantially perpendicular to the assembly direction (e.g., lateral direction or vertical direction).

FIG. 227 shows a variation of the cushion module 120 including two projections or beads 302. The illustrated cushion module 120 includes at least one bead 302 extending along at least a portion of each lateral side of the elastomeric friction member 142. The lateral beads 302 span a portion of the internal perimeter of the elastomeric friction member 142. When forming the elastomeric friction member 142, manufacturing variations between different elastomeric friction members 142 can cause variations in the removal force (the force required to remove the frame 110 from the cushion module 120 once the frame 110 is coupled to the cushion module 120) between different cushion modules 120 manufactured to the same specification. Including lateral beads 302 (or other lateral projections) on only lateral portions of the elastomeric friction member 142 can help reduce this variance in removal force among different cushion modules 120 and therefore allow for more consistent performance of the elastomeric friction member 142. In some configurations, the elastomeric friction member 142 can include more than one lateral bead 302 on each lateral side.

FIGS. 231-250 illustrate a variation of the frame main body 268 and the frame insert 270. In this configuration, a diffuser 173 is disposed over a front of the vent 170. The diffuser 173 can diffuse air vented through the bias flow holes of the vent 170 to help reduce the noise of the air flow through the vent 170 and/or to help reduce draft produced by the air flow through the vent 170. In the illustrated configuration, the diffuser 173 is removably coupled to the frame insert 270. In other configurations, the diffuser 173 can be integrally formed with or permanently coupled to the frame insert 270.

The vent arrangement 170 can be recessed rearwardly from the front surface of the frame insert 270 in a vent arrangement recess 174, as shown in, for example, FIGS. 233-234. Therefore, in some configurations, when the diffuser 173 is coupled to the frame insert 270, the front of the diffuser 173 can lie flush or approximately flush with the front surface of the frame insert 270. Alternatively, if the diffuser 173 is relatively thick, the vent arrangement recess 174 reduces the extent to which the diffuser 173 projects from the front surface of the frame insert 270. The illustrated frame insert 270 includes two diffuser clip recesses 176, one disposed in each side of the vent arrangement recess 174. The diffuser clip recesses 176 cooperate with or receive (e.g., via a snap fit), corresponding projections extending from sides of the diffuser 173 to couple the diffuser 173 to the frame insert 270. Alternatively, the diffuser 173 can be coupled to the frame insert 270 via a magnetic connection (e.g. with at least one magnet on one of the diffuser 173 and the frame insert 270, and at least one magnet or ferrous metallic portion on the other of the diffuser 173 and the frame insert 270), a friction fit arrangement (e.g., via an elastomeric friction member similar to the connection between the cushion module 120 and frame insert 270), chemical bonding, adhesive(s), and/or other suitable means. A diffuser spacing flange 181 at least partially surrounds the vent arrangement 170 and projects forward relative to the vent arrangement 170. When the diffuser 173 is coupled to the frame insert 270, the diffuser 173 abuts the diffuser spacing flange 181 so that there is a spacing or distance (e.g., equal to the thickness of the diffuser spacing flange 181 or distance the diffuser spacing flange 181 projects relative to the vent arrangement 170) between the vent arrangement 170 and the diffuser 173.

The frame insert 270 of FIGS. 231-250 includes an insert collar 152. The insert collar 152 surrounds the gas inlet 130 and the vent arrangement 170. In some configurations, the frame insert 270 includes a divider wall 172 between the vent 170 and the gas inlet 130. The divider wall 172 can be positioned between and/or at least partially separate the vent 170 from the gas inlet 130. In some configurations, the divider wall 172 includes one or more divider wall recesses 175, as shown in FIG. 238, which allow the divider wall 172 and collar 152 to elastically flex around the spherical mold tool portion during removal of the frame insert 270 from the mold tool after injection molding of the frame insert 270 during manufacturing. This can advantageously reduce permanent deformation of the frame collar 152 and/or divider wall 172. In the illustrated configuration, the divider wall 172 includes two recesses 175, one on each lateral side of the divider wall 172 such that a gap exists between each distal or lateral end of the divider wall 172 and the frame collar 152.

The frame insert 270 can include a rotation limiting feature 179 (FIG. 237) disposed along a portion of the frame insert 270 defining or surrounding a top portion of the insert opening 272 and projecting forward relative to adjacent portions of the frame insert 270, as shown in FIG. 237. The feature 179 can act as an anti-rotation feature or physical barrier limiting rotation of the elbow 158 toward the frame insert 270 if the elbow 158 is rotated, for example, toward position 158U shown in FIG. 201.

As shown in FIGS. 241-242, the diffuser 173 includes a diffuser rim 183 surrounding a diffusing element 185. In use, gases vent through the vent holes 187 in the vent arrangement 170 of the frame insert 270, then are directed through the diffusing element 185. The frame insert 270 and diffuser 173 assembly comprises a diffuser bypass channel 189. The diffuser bypass channel 189 is positioned above the diffuser 173 or between the diffuser 173 and frame insert 270 at or near a top of the vent arrangement 170. The diffuser bypass channel 189 allows gases to vent from the vent holes 187 if the diffuser 173 is blocked or covered with water (e.g., condensate). In some configurations, the diffuser rim 183 includes a scallop 191 at or along a top portion of the diffuser 173, as shown in FIG. 247. The scallop 191 can allow the user to remove the diffuser 173 from the frame insert 270 more easily. The scallop 191 can form a portion of the boundary of the diffuser bypass channel 189. The size of the scallop 191 can be selected to control the amount of gases flow that can be vented from the diffuser bypass channel 189. Increasing the size of the scallop 191 increases the size of the diffuser bypass channel 189. In some configurations, the frame insert 270 has a vertical dimension selected or sized to provide a diffuser grip region 193, as shown in FIGS. 249-250. The diffuser grip region 193 is a recessed area of the frame insert 270 above the diffuser 173 that provides a space for the user to maneuver his or her finger behind the diffuser 173 to more easily remove the diffuser 173.

FIGS. 251-267 illustrate cross-sections of variations of the patient interfaces 100 of FIGS. 168-250. In the configuration of FIG. 251, the elastomeric friction member 142 projects rearwardly from the housing 122 rather than forwardly. The support flange 162 is substantially perpendicular to the long axis of the elastomeric friction member 142. In other configurations, the support flange 162 can extend parallel to the long axis of the elastomeric friction member 142. The length of the unsupported portion of the elastomeric friction member 142 is much longer than the length of the supported portion. The housing 122 of the cushion module 120 includes the vent arrangement 170 (instead of the frame main body 268 or frame insert 270), and the vent arrangement 170 vents through a hole 201 in the frame 110 that is aligned with or overlies the vent arrangement 170 when the cushion module 120 is coupled to the frame 110.

In the configuration of FIG. 252, the frame 110 includes the vent arrangement 170. In particular, the frame main body 268 includes the vent arrangement 170.

The frame main body 268 includes a rearwardly projecting elastomeric vent flange 195 projecting from a portion of the frame 110 surrounding the vent arrangement 170. The elastomeric vent flange 195 forms a friction fit with a corresponding vent hole 197 in the housing 122 (e.g., similar to the friction fit between the elastomeric friction member 142 and the frame insert 270). In use, gases vent through the vent hole 197 and the vent arrangement 170.

The frame 110 also includes the vent arrangement 170 in the configuration of FIG. 253. In particular, the frame main body 268 includes the vent arrangement 170. However, in this configuration, the housing 122 includes the elastomeric vent flange 195 disposed about the perimeter of the vent hole 197 in the housing 122. When the frame 110 and cushion module 120 are coupled, the elastomeric vent flange 195 forms a seal with the frame 110 around the perimeter of the vent arrangement 170 so that gases vent through the vent arrangement 170 from the housing vent hole 197. The elastomeric friction member 142 projects inwardly from the housing 122 and includes a relatively large unsupported portion.

In the configuration of FIG. 254, the frame 110 includes the vent arrangement 170 and a rigid vent flange 195a projecting rearwardly from the frame main body 268 around a perimeter of the vent arrangement 170. The housing 122 includes an elastomeric portion 542 that includes the elastomeric friction member 142 surrounding the connection opening 132 and an elastomeric vent flange 195b surrounding the housing opening 197. The frame insert 270 is coupled to the frame main body 268 (e.g., inserted into the frame inlet opening 130a), and the frame 110 is coupled to the cushion module 120 via a friction fit. The frame insert 270 forms a friction fit with the elastomeric friction member 142, and the rigid vent flange 195a forms a friction fit with the elastomeric vent flange 195b.

In the configuration of FIG. 255, the frame main body 268 includes the vent arrangement 170 and a frame flange or collar 199 that surrounds the frame insert 270 and the vent arrangement 170. The frame flange 199 can be rigid or elastomeric. The frame flange 199 engages (e.g., via a friction fit) the elastomeric friction member 142 of the cushion module 120 when the cushion module 120 and frame 110 are coupled. The elastomeric friction member 142 projects rearwardly (or inwardly into the breathing cavity) at the bottom of the connection opening 132 and projects forwardly (or outwardly from or relative to the breathing cavity) at the top of the connection opening 132.

In the configuration of FIGS. 256, the frame insert 270 includes the vent arrangement 170. The vent arrangement 170 is separated from the inlet 272 of the frame insert 270 by the upper wall or upper portion of the insert wall or collar 278. In one form, this could be a divider wall 172. The elastomeric friction member 142 of the cushion module 120 engages (e.g., via a friction fit) a collar 152 (e.g., a vent collar or frame insert collar) of the frame insert 270 projecting rearwardly from a top portion of the vent arrangement 170 and a bottom portion of the insert collar 278. In other words, the frame insert collar 152 can include a vent wall 275 and the insert wall or collar 278. The vent arrangement 170 (or portion of the frame insert 270 including the vent arrangement 170) projects or protrudes forward from or relative to the frame main body 268. In the illustrated configuration, the surface of the frame insert 270 in which the vent holes are formed is curved, which can help improve diffusion of gases vented through the vent holes.

In the configuration of FIG. 257, the elastomeric friction member 142 of the cushion module 120 projects forward or outwardly with respect to the housing 122 at or along a top portion of the elastomeric friction member 142 and projects rearward or inwardly at or along a bottom portion of the elastomeric friction member 142. The elastomeric friction member 142 has a relatively large unsupported portion 310. The frame main body 268 includes the vent arrangement 170. The frame insert 270 defines the frame inlet 272. The elastomeric friction member 142 engages (e.g., via a friction fit) a frame flange 199 that extends around or surrounds the frame insert 270 and vent arrangement 170 in the frame 110. In one form, the frame flange 199 can be rigid. In at least one form, the frame flange 199 can be elastomeric.

In the configuration of FIG. 258, the frame insert 270 includes the vent arrangement 170. A frame insert collar 152 surrounds the frame inlet 272 and the vent arrangement 170. The frame insert collar 152 includes a bottom portion of the frame insert 270 (that forms or defines a bottom portion of the frame inlet 272) and a portion of the frame insert 270 projecting rearwardly relative to the frame 110 and disposed above the vent arrangement 170. The elastomeric friction member 142 of the cushion modules engages (e.g., via a friction fit) the frame insert collar 152.

In some configurations, the frame insert 270 has at least three important or critical dimensions: the number and position of the vent holes; the dimensions of the elbow socket 157; and/or the dimensions of the insert collar 278 and/or vent wall 275. Even small variations or errors in these dimensions can significantly impair the performance of the frame insert 270. The configuration of FIG. 259 can eliminate, or reduce or minimize the criticality or importance of the dimensions of the insert collar 278 and/or optional vent wall 275. In this configuration, the elastomeric friction member 142 of the cushion module 120 engages (e.g., via a friction fit) a frame flange or collar 199 projecting rearwardly from the frame 110 and generally surrounding or following the perimeter of the frame insert 270. Because the elastomeric friction member 142 engages the frame collar 199, the critical dimension is transferred from the frame insert 270 to the frame collar 199, which can reduce manufacturing complexity of the frame insert 270. In the illustrated configuration, a rear or free end portion of the frame collar 199 at or along a bottom portion of the frame collar 199 curves upward (forming a curved section 279) to engage a rear free end of a bottom portion of the insert collar 278. The curved section 279 allows for an additional weld location between the curved section 279 and the rear free end of the insert collar 278. This weld can prevent or inhibit a gap between the insert collar 278 and the frame collar 199 from becoming a dirt trap. As indicated by the dashed lines in FIG. 259, the frame insert 270 could include a frame insert collar 152 that engages or is positioned adjacent to the frame flange or collar 199.

In the configuration of FIG. 260, the frame collar 199 lacks or does not include a bottom portion. The cushion module 120 therefore engages the frame collar 199 along the top and/or sides of the frame collar 199. A bottom portion of the cushion module 120 engages the insert collar 278. The frame collar 199 and insert collar 278 meet at a transition point or portion, which can be at or near a middle of lateral sides of the frame 110, frame main body 268, and/or frame insert 270. This configuration can reduce or eliminate any dirt trap between the frame collar 199 and insert collar 278.

In the configuration of FIG. 261, the projecting surface 177, which includes the vent arrangement 170, is integrated with or included in the frame main body 268 (rather than the frame insert 270). The cushion module engages (e.g., via a friction fit) the frame collar 199 around its entire perimeter. The frame collar 199 surrounds the vent arrangement 170 and insert collar 278. In the illustrated arrangement, the insert collar 278 extends rearwardly or inwardly beyond the rear boundary or edge of the lower or bottom portion of the frame collar 199.

In the configuration of FIG. 262, the frame inlet opening 130a is defined by a collar 199 formed by the frame main body 268 (rather than an insert 270). The projecting surface 177 is formed or defined by, and the vent arrangement 170 is formed in, a separate component 270 that is coupled to the frame main body 268. The cushion module 120 engages (e.g., via a friction fit) the frame collar 199 (which includes the portion of the frame main body 268 defining a bottom portion of the inlet opening 130a) around the entire perimeter of the cushion module connection opening 132 or entire inner perimeter of the elastomeric friction member 142.

In the configuration of FIG. 263, the frame inlet opening 130a is again defined by the frame main body 268, e.g., the frame collar 199. The frame insert 270 defines or includes the projecting surface 177, vent arrangement 170 and a vent wall 275. The cushion module 120 engages the vent collar 275 and the frame collar 199 to form the friction fit with the frame 110.

In the configuration of FIG. 264, the frame inlet opening 130a, vent arrangement 170 and projecting surface 177 are formed in or by, defined by, and/or integrated into the frame main body 268. The frame 110 is therefore a single piece (without a frame insert 270). The cushion module 120 engages (e.g., via a friction fit) the frame collar 199.

In the configuration of FIG. 265, the projecting surface 177 is or is defined by a separate component that is coupled, e.g., welded, to the frame insert 270. In one form, the separate component is in the form of a vent connector 203. The vent connector 203 is configured to connect to the frame insert 270. This arrangement removes or reduces the criticality of dimensions of the vent holes of the vent arrangement 170 from the frame insert 270. This can advantageously simplify manufacturing of the frame insert 270.

In the configuration of FIG. 266, the projecting surface 177 and vent arrangement 170 are formed in or by, defined by, and/or incorporated into the frame main body 268 (rather than the frame insert 270), which can simplify manufacturing of the frame insert 270. The frame main body 268 comprises a rear peripheral recess 280. The rear peripheral recess 280 is in a rear surface of the frame main body 268 surrounding the perimeter of the frame inlet opening 130a. When the frame insert 270 is assembled with the frame main body 268, a front surface (for example, part of an abutment flange 276) at least partially rests in the rear peripheral recess 280 of the frame main body 268. The frame insert 270 can be secured to the frame main body 268, for example, via welding, adhesives, molding, and/or other means along the abutting surfaces of the frame insert 270 and the frame main body 268.

In the configuration of FIG. 267, the frame insert 270 includes the inlet 272, elbow socket 157, and vent arrangement 170. The frame main body 268 includes the frame collar 199. The frame collar 199 includes a curved section 279 that curves or extends around a rear end or edge of the insert collar 278, which provides a weld point or surface between the frame main body 268 and frame insert 270. This weld can prevent or inhibit a gap between the frame main body 268 and frame insert 270, that would otherwise be accessible from inside the breathing chamber, from becoming a dirt trap. The cushion module 120 is configured to connect to the frame 110 with a friction fit between the elastomeric friction member 142 and the frame collar 199.

FIGS. 268-280 illustrate portions of several patient interfaces 100, each having a version of a friction coupling 140. The patient interfaces 100 and illustrated friction couplings 140 can share many similarities with the other interfaces and friction couplings disclosed herein. Accordingly, the same reference characters can be used to refer to the same or similar components or features and the following description is focused on the unique features of the interfaces 100 and/or friction couplings 140 relative to other interfaces disclosed herein. Accordingly, the interfaces 100 and/or friction couplings 140 can incorporate features of the other interfaces disclosed herein. In addition, features that are not disclosed in detail can be the same as or similar to other interfaces disclosed herein, or can be of another suitable arrangement.

The patient interfaces 100 of FIGS. 268-280 each includes a frame 110 and a cushion module 120. Only a portion of the patient interface 100 incorporating the friction coupling 140 is shown in each of FIGS. 268-280. Moreover, only a portion of the friction coupling 140 is illustrated in the figures. In particular, each figure illustrates a section view of only an upper portion of the friction coupling 140. It will be understood that the friction coupling 140 forms a closed loop, which may be circular or non-circular. The closed loop of the friction coupling 140 may have the same or substantially the same shape as illustrated throughout an entirety of the closed loop. In other arrangements, the closed loop of the friction coupling 140 can vary at different locations along the closed loop. For example, certain features may be provided once or intermittently along the closed loop. In some arrangements, a length, a thickness or other dimensions of the friction coupling 140 can vary around the closed loop, many examples of which have been disclosed herein.

As with other of the frames described herein, the frame 110 is configured to be connected to headgear, which holds the patient interface 100 in a sealed relation to the user's face. The frame 110 can include any suitable number or type of headgear mounts, as described herein. The cushion module 120 defines at least a portion of the breathing chamber 102 of the patient interface 100. The cushion module 120 can be removably coupled to the frame 110. The illustrated cushion module 120 includes a housing 122 and a cushion or seal (124—not shown in FIGS. 268-280). As described above, portions of the frame 110 and/or cushion module 120 not specifically illustrated can be the same as or substantially similar to any of the other frames or cushion modules disclosed herein, or can be of another suitable arrangement. Thus, the features illustrated in and described with respect to FIGS. 268-280 can be implemented in any of the patient interfaces 100 disclosed herein.

FIGS. 268 and 269 illustrate a patient interface 100 having a friction coupling 140 that incorporates a user feedback arrangement 600. The user feedback arrangement 600 can be configured to provide feedback (e.g., haptic feedback) to a user indicating complete engagement between the cushion module 120 and the frame 110. In the illustrated arrangements, the user feedback arrangement 600 is a detent that provides at least tactile feedback to the user that indicates complete or sufficient connection between the cushion module 120 and the frame 110. The user feedback arrangement 600 may also or instead be configured to provide auditory, visual or other feedback to the user.

In the illustrated arrangements, an elastomeric friction member 142 is carried by the housing 122 of the cushion module and engages a collar 152 of the frame 110. This arrangement could, however, be reversed and the elastomeric friction member 142 could be carried by the frame 110 instead. The housing 122 includes a main wall 160 and a support flange 162. The support flange 162 surrounds and/or defines a connection opening 132 and supports the elastomeric friction member 142. A support wall 163 surrounds and is adjacent the connection opening and/or the support flange 162. The support wall 163 can define a curved transition between the main wall 160 and the support flange 162, as is described herein with reference to, for example, FIG. 152. The support flange 162 can provide support for the elastomeric friction member 142, as described herein, which can enhance the operation of the user feedback arrangement 600. However, in other arrangements, the support flange 162 can be omitted.

In the illustrated arrangement, the elastomeric friction member 142 includes a projection 502 and the collar 152 of the frame 110 includes a recess 604; however, this arrangement could be reversed. The movement of the projection 602 into the recess 604 can provide haptic or tactile feedback to the user indicating that the cushion module 120 and frame 110 are properly coupled. The projection 602 can be a discrete structure located along only a portion of the closed loop of the friction coupling 140. Alternatively, the projection 602 can comprise a plurality of discrete structures located along the closed loop. Alternatively, the projection 602 can extend around an entirety of the closed loop. If desired, multiple projections 602 could be provided lengthwise in an assembly direction. Preferably, corresponding recesses 604 are provided for each projection 602.

In the patient interface 100 of FIG. 268, the support flange 162 of the cushion module 120 extends in a rearward direction from the main wall 160 of the housing 122 into the breathing chamber 102. In such an arrangement, the projection 602 can be located on a rearward portion or a free end portion of the elastomeric friction member 142 and the recess 604 can be located on a rearward portion or a free end portion of the collar 152. With such an arrangement, the projection 502 is located away from the location of initial engagement between the elastomeric friction member 142 and the collar 152. Accordingly, the cushion module 120 can be moved onto the collar 152 a significant distance before the projection 302 engages the collar 152, at which point resistance to relative movement of the cushion module 120 and the frame 110 my increase.

In the illustrated arrangement, the projection 602 is located close to the free end of the elastomeric friction member 142 such that a significant overlap between the elastomeric friction member 142 and the collar 152 is achieved before the projection engages the collar 152. In addition, the recess 604 is also located close to the free end of the collar 152 so that once the projection 602 engages the collar 152, the cushion module 120 is relatively close to full connection and does not need to be moved very far before the projection 602 engages the recess 604. In some configurations, the projection 602 and/or the recess 604 are located on a rearward one-half, one-quarter, one-eighth or one-tenth of the elastomeric friction member 142 or collar 152. This assumes the connection direction is with a forward end of the elastomeric friction member 142 making initial contact with a rearward end of the collar 152. In any event, as described above, it can be desirable for the projection 602 to be located away from the end or portion of the elastomeric friction member 142 that makes initial contact with the collar 152. For example, if the elastomeric friction member 142 were carried by the collar 152 of the frame 110, the projection 602 and recess 604 preferably would be located closer to a forward end/supported end of the elastomeric friction member 142 and support flange 162, respectively.

FIG. 269 illustrates a modification of the patient interface 100 of FIG. 268. In the arrangement of FIG. 269, the support flange 162 of the cushion module 120 extends in a forward direction from the main wall 160 of the housing 122 away from the user and the breathing chamber 102 and towards the frame 110. In such an arrangement, the projection 602 can be located on a rearward portion of the elastomeric friction member 142, which is a fixed or supported end of the elastomeric friction member 142/support flange 162. In the illustrated arrangement, the collar 152 extends in a rearward direction from a front wall or main body 150 of the frame 110. Accordingly, the recess 304 is located on a rearward portion or free end of the collar 152, similar to the cushion module 120 of FIG. 268. In some configurations, the projection 602 and/or the recess 604 are located on a rearward one-half, one-quarter, one-eighth or one-tenth of the elastomeric friction member 142 or collar 152, which corresponds to a fixed or supported end of the support flange 162 and a free end of the collar 152. As described above, a user feedback arrangement 600 similar to that of FIG. 268 or 269 can be incorporated into any of the patient interface disclosed herein.

FIGS. 270 and 271 illustrate patient interfaces 100 each having a friction coupling 140 incorporating an enhanced sealing feature 510 configured to provide an enhanced seal between the frame 110 and the cushion module 120, such as an improved seal, increased seal consistency or increased seal longevity. The enhanced sealing feature 510 comprises an angled flap or lip 512 extending from the elastomeric friction member 142. Preferably, the angled lip 512 extends around an entirety of a closed loop perimeter of the elastomeric friction member 142. In the illustrated arrangement, the angled lip 512 extends inwardly from an inner surface of the elastomeric friction member 142 and engages an outer surface of the collar 152 of the frame 110. In other words, the enhanced sealing feature 510 can extend radially inward from the elastomeric friction member 142. In other arrangements in which the elastomeric friction member 142 is located inside of the collar 152, the lip 512 could extend from an outer surface of the elastomeric friction member 142 and contact an inner surface of the collar 152. In other words, the enhanced sealing feature 510 can extend radially outward from the elastomeric friction member 142.

The angled lip 512 has a base that is connected to a main body portion of the elastomeric friction member 142. The angled lip 512 extends from the base toward the breathing chamber 102 at an oblique angle relative to a longitudinal axis of the elastomeric friction member 142. Thus, the angled lip 512 extends in a rearward direction such that the free end or edge is located rearward of the base of the angled lip 512. With such an arrangement, pressurized gas within the breathing chamber 102 may exert a force on the angle lip 512 tending to straighten the lip 512 or move the free end or edge of lip 512 in a forward direction. As a result, the pressurized gas may force the angled lip 512 tightly against the collar 152. The force biasing the angled lip 512 against the collar 152 may vary with the pressure in the breathing chamber 102. Other arrangements are also possible. For example, the lip 512 could be oriented generally perpendicular to an interior surface of the elastomeric friction member 142 and could be moved to an angled orientation in use, such as a result of the assembly movement of the cushion module 120 onto the frame 110.

In the illustrated arrangement, the angled lip 512 is located on a forward end of the elastomeric friction member 142. However, other axial locations along the elastomeric friction member 142 are also possible. For example, the angled lip 512 could be positioned in an intermediate portion of the elastomeric friction member 142 or at or near a rearward end of the elastomeric friction member 142. Moreover, multiple lips 512 could be provided.

FIG. 270 illustrates an arrangement in which the support flange 162 extends in a rearward direction from the main wall 160 of the housing 122. FIG. 271 illustrates an arrangement in which the support flange 162 extends in a forward direction from the main wall 160 of the housing 122. In an alternative arrangement, the elastomeric friction member 142 can be carried by the collar 152 of the frame 110. In such an arrangement, the angled lip 512 can extend from an outer surface of the elastomeric friction member 142 and engage an inner surface of the support flange 162 of the housing 122 of the cushion module 120.

FIGS. 272-275 illustrate patient interfaces 100 having a frame 110 and a cushion module 120. The frame 110 includes a collar 152 and the cushion module 120 includes a support flange 162. In the interfaces 100 of FIGS. 272-275, the elastomeric friction member 142 is carried by the frame 110 and releasably engages the cushion module 120. In the illustrated arrangements, the elastomeric friction member 142 is carried by the collar 152 of the frame 110 such that the collar 152 is partially or wholly embedded in the elastomeric friction member 142.

In the arrangement of FIG. 272, the collar 152 of the frame 110 extends in a rearward direction from the front wall 150 toward the user and the breathing chamber 102 of the cushion module 120. In the arrangement of FIG. 273, the collar 152 of the frame 110 extends in a forward direction away from the user and the breathing chamber 102 of the cushion module 120. In each arrangement, an inner surface of the elastomeric friction member 142 engages an outer surface of the support flange 162. In each arrangement, the support flange 162 extends in a forward direction away from the user and the breathing chamber 102.

In the arrangements of FIGS. 274 and 275, the elastomeric friction member 142 is received within the support flange 162 of the cushion module 120 such that an outer surface of the elastomeric friction member 142 engages an inner surface of the collar 152 of the frame 110. In the arrangement of FIG. 274, the support flange 162 of the cushion module 120 extends in a forward direction away from the user. In the arrangement of FIG. 275, the support flange 162 of the cushion module 120 extends in a rearward direction toward the user and away from the frame 110. In each of the arrangements of FIGS. 274 and 275, the collar 152 of the frame 110 extends in a rearward direction toward the user and the breathing chamber 102. In some configurations, the arrangements of FIGS. 274 and 275 can include a user feedback arrangement configured to provide feedback (e.g., haptic feedback) to a user of complete engagement between the cushion module 120 and the frame 110, such as the user feedback arrangement 600 shown in and described with respect to FIGS. 268 and 269.

Arrangements such as those illustrated in FIGS. 272-275 can advantageously reduce the manufacturing cost and/or case the manufacturing process of the patient interface 100. For example, cushion modules 120 are often provided in multiple sizes, such as two-four sizes. Frames 110 configured for use with these multiple sizes of cushion module 120, in contrast, are usually provided in a single size. Including the elastomeric friction member 142 on the frame 110 reduces the complexity of the mold tools and/or manufacturing process for the multiple sizes of the cushion module 120. Moreover, the increased complexity of the mold tool and/or manufacturing process for the frame 110 with the elastomeric friction member 142 can be required for only a single size of frame 110- or at least for a smaller number of frame sizes than cushion module sizes. Thus, the increased complexity of the mold tool/process for the single frame size (or lower number of frame sizes) is more than offset by the decreased complexity of the multiple mold tools/processes for the multiple cushion module sizes.

As described herein, each of the patient interfaces 100 (or portions thereof, e.g., frames 110 or cushion modules 120) can be modified so that the elastomeric friction member 142 is carried by the frame 110 rather than the cushion module 120. Thus, although FIGS. 272-275 illustrate relatively basic arrangements in which the elastomeric friction member 142 is carried by the frame 110 and releasably engages the cushion module 120, additional embodiments are contemplated in which the structural features disclosed with the various embodiments herein are implemented in the manner of FIGS. 272-275 with the elastomeric friction member 142 carried by the frame 110 instead of the cushion module 120.

For example, the patient interface 100 of FIGS. 139-165 can be modified so that the elastomeric friction member 142 is carried by the frame 110. The frame 110 can be modified to include features of the cushion module 120 and the cushion module 120 can be modified to include features of the frame 110. In such an arrangement, the elastomeric friction member 142 can be omitted from at least a portion of an inner surface of the collar 152 so that the conduit elbow 158 can still be accommodated by the frame 110.

Similarly, the patient interface 100 of FIGS. 21-45 can be modified so that the elastomeric friction member 142 is carried by the frame 110. The frame 110 can be modified to include features of the cushion module 120 and the cushion module 120 can be modified to include features of the frame 110. In such an arrangement, the elastomeric friction member 142 can be omitted from at least a portion of an inner surface of the collar 152 so that the conduit elbow 158 can still be accommodated by the frame 110.

The patient interface 100 of FIGS. 46-69 can be modified so that the elastomeric friction member 142 is carried by the frame 110. In such an arrangement, the frame 110 can incorporate a structure similar to the support flange 162 that is embedded in the elastomeric friction member 142. The cushion module 120 can incorporate a structure similar to the collar 152 that can be engaged by the elastomeric friction member 142.

The patient interface 100 of FIGS. 71-100 can be modified so that the elastomeric friction member 142 is carried by the frame 110. The frame 110 can be modified to include features of the cushion module 120 and the cushion module 120 can be modified to include features of the frame 110. In such an arrangement, the elastomeric friction member 142 can be omitted from at least a portion of an inner surface of the collar 152 so that the conduit elbow 158 can still be accommodated by the frame 110.

The patient interface 100 of FIGS. 101-138 can be modified so that the elastomeric friction member 142 is carried by the frame 110. The frame 110 can be modified to include features of the cushion module 120 and the cushion module 120 can be modified to include features of the frame 110. In such an arrangement, the elastomeric friction member 142 can be omitted from at least a portion of an inner surface of the collar 152 so that the conduit elbow 158 can still be accommodated by the frame 110.

FIGS. 276 and 277 illustrate patient interfaces 100 including a frame 110 and a cushion module 120. The cushion module 120 includes a support flange 162. The illustrated frame 110 includes a support wall 163 that surrounds the connection opening 132 of the patient interface 100. In the interfaces 100 of FIGS. 276 and 277, the elastomeric friction member 142 is carried by the frame 110 and releasably engages the cushion module 120. In the illustrated arrangements, the support wall 163 is partially or wholly embedded in the elastomeric friction member 142. In the illustrated arrangements, the elastomeric friction member 142 has a substantial unsupported length, similar to the arrangements shown in and described with respect to FIGS. 8-12.

In the arrangement of FIG. 276, the elastomeric friction member 142 extends in a rearward direction from the frame 110 toward the user and the breathing chamber 102. In the arrangement of FIG. 277, the elastomeric friction member 142 extends in a forward direction from the frame 110 away from the user and the breathing chamber 102. In each of the arrangements, the elastomeric friction member 142 surrounds the support flange 162 such that an inner surface of the elastomeric friction member 142 engages an outer surface of the support flange 162. In other arrangements, the elastomeric friction member 142 could be located on an interior of the support flange 162 such that an outer surface of the elastomeric friction member 142 engages an inner surface of the support flange 162.

FIGS. 278-280 illustrate additional patient interfaces 100 having a frame 110 and a cushion module 120. The cushion modules 120 each includes a support flange 162 and the frames 110 each includes a collar 152 that surrounds the connection opening 132 of the patient interface 100. In each of the interfaces 100 of FIGS. 278-280, the elastomeric friction member 142 is carried by the frame 110 and releasably engages the cushion module 120.

In the arrangement of FIG. 278, the collar 152 of the frame 110 extends in both a forward and a rearward direction from the front wall 150 of the frame 110. Such an arrangement is similar to the patient interface 100 illustrated in FIGS. 52-69 except that the elastomeric friction member 142 is carried by the frame 110 instead of the cushion module 120. The collar 152 is wholly embedded within the elastomeric friction member 142. However, in other arrangements, the elastomeric friction member 142 could be provided only on the side of the collar 152 (e.g., the inside) that faces the support flange 162 of the housing 122 of the cushion module 120. In such an arrangement, the elastomeric friction member 142 could also be provided on an end of the collar 152 that faces the housing 122 to provide soft contact with the housing 122.

In the arrangement of FIG. 279, the elastomeric friction member 142 is provided on only a side of the collar 152 of the frame 110 that faces the support flange 162 of the cushion module 120. In the illustrated arrangement, the elastomeric friction member 142 is provided on an inside surface of the collar 152. However, in other arrangements, the elastomeric friction member 142 could be located on an outside surface of the collar 152, which could be located within the support flange 162 when the cushion module 120 is attached to the frame 110. Furthermore, although the collar 152 extends only in a rearward direction from the front wall 150 of the frame, in other arrangements the collar 152 could extend only in a forward direction or could extend in both a forward direction and a rearward direction from the front wall 150.

In the arrangement of FIG. 280, the cushion module 120 defines a recess 400 configured to receive a portion or an entirety of the elastomeric friction member 142. In the illustrated arrangement, an inner surface of the elastomeric friction member 142 engages an outer surface of the support flange 162 of the cushion module 120. The arrangement of FIG. 280 is similar to the arrangement of the patient interface 100 illustrated in FIGS. 52-69 except the elastomeric friction member 142 is carried by the frame 110 instead of the cushion module 120. Accordingly, structural details shown in and described with respect to FIGS. 52-69 can be incorporated into the patient interface of 280. In addition, in the illustrated arrangement of FIG. 280, the collar 152 is completely embedded in the elastomeric friction member 142. In alternative arrangements, the elastomeric friction member 142 could cover only the side of the collar 152 that faces the support flange 162 of the cushion module. The elastomeric friction member 142 could also be provided on the end facing the housing 122 of the cushion module 120 to provide soft contact with the cushion module 120. In addition, the collar 152 could extend in a forward direction in addition to a rearward direction from the front wall 150 of the frame 110.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”. Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

The term “plurality” refers to two or more of an item. Recitations of quantities, dimensions, sizes, formulations, parameters, shapes and other characteristics should be construed as if the term “about” or “approximately” precedes the quantity, dimension, size, formulation, parameter, shape or other characteristic. The terms “about” or “approximately” mean that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. Recitations of quantities, dimensions, sizes, formulations, parameters, shapes and other characteristics should also be construed as if the term “substantially” precedes the quantity, dimension, size, formulation, parameter, shape or other characteristic. Unless otherwise defined, the term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “1 to 3,” “2 to 4” and “3 to 5,” etc. This same principle applies to ranges reciting only one numerical value (e.g., “greater than 1”) and should apply regardless of the breadth of the range or the characteristics being described.

A plurality of items may be presented in a common list for convenience.

However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

Reference to any prior art in this specification is not, and should not be taken as, an acknowledgement or any form of suggestion that that prior art forms part of the common general knowledge in the field of endeavour in any country in the world.

Where, in the foregoing description reference has been made to integers or components having known equivalents thereof, those integers are herein incorporated as if individually set forth.

The invention may also be said broadly to consist in the parts, elements and features referred to or indicated in the specification of the application, individually or collectively, in any or all combinations of two or more of said parts, elements or features.

It should be noted that various changes and modifications to the presently preferred embodiments described herein will be apparent to those skilled in the art. Such changes and modifications may be made without departing from the spirit and scope of the invention and without diminishing its attendant advantages. For instance, various components may be repositioned as desired. It is therefore intended that such changes and modifications be included within the scope of the invention. Moreover, not all of the features, aspects and advantages are necessarily required to practice the present invention. Accordingly, the scope of the present invention is intended to be defined only by the claims that follow.

What is claimed is:

1. A cushion module for a respiratory mask, the cushion module configured for attachment to a frame, the frame having a front wall and a collar extending from the front wall, the cushion module comprising:
a housing;
a cushion; and
an elastomeric friction member comprising a linear frictional surface;
wherein the cushion defines a face-contacting surface and the housing defines a connection opening and supports the elastomeric friction member, and
wherein the housing comprises a wall portion, at least a portion of which is external to the elastomeric friction member and extends between the elastomeric friction member and the cushion,
wherein the elastomeric friction member is configured to engage the collar with a friction fit and compress when the cushion module is coupled to the frame;
wherein the linear frictional surface is configured to apply a retention force determined in part by an amount of compression of the elastomeric friction member along an assembly axis of the respiratory mask; and
wherein the linear friction surface defines an entire length of contact between the elastomeric friction member and the collar.

2. The cushion module of claim 1, wherein a portion of the housing defining the connection opening is embedded within the elastomeric friction member to couple the elastomeric friction member to the housing.

3. The cushion module of claim 1, wherein the housing of the cushion module further comprises a support flange, wherein the support flange at least partially defines the connection opening and extends within the elastomeric friction member substantially in a direction of assembly of the cushion module to the frame.

4. The cushion module of claim 3, wherein the support flange comprises a plurality of apertures and wherein the elastomeric friction member extends through each of the plurality of apertures.

5. The cushion module of claim 3, wherein the housing comprises a plurality of apertures at a junction between the support flange and an adjacent portion of the housing and wherein the elastomeric friction member extends through each of the apertures.

6. The cushion module of claim 1, wherein the wall portion of the housing comprises a plurality of apertures and wherein the elastomeric friction member extends through each of the apertures.

7. The cushion module of claim 1, wherein the elastomeric friction member defines an abutment surface configured to contact the front wall of the frame when the cushion module is coupled to the frame.

8. The cushion module of claim 1, wherein the elastomeric friction member and the cushion are constructed of the same material.

9. The cushion module of claim 1, wherein the elastomeric friction member is more rigid than the cushion.

10. The cushion module of claim 1, wherein the linear frictional surface is configured such that a relative angle is defined between the linear frictional surface and the collar.

11. The cushion module of claim 10, wherein the linear frictional surface is tapered along the assembly axis of the respiratory mask.

12. The cushion module of claim 10, wherein the relative angle is configured to apply a variable retention force along the assembly axis.

13. A respiratory mask, comprising:
a frame configured to directly connect to and receive headgear, the frame comprising a front wall at least partially defining a gas inlet opening, the frame further comprising a collar extending away from the front wall, the collar at least partially surrounding the gas inlet opening;
a cushion module comprising a housing, a cushion and an elastomeric friction member having a linear frictional surface, the cushion defining a face-contacting surface, the housing defining a connection opening, wherein a portion of the housing defining the connection opening is embedded within the elastomeric friction member to couple the elastomeric friction member to the housing, wherein the housing is made of a material more rigid than the cushion and forms at least a portion of a breathing chamber of the respiratory mask, the elastomeric friction member configured to selectively connect the cushion module to the collar of the frame with a friction fit so that a flow of gas can be delivered to the breathing chamber through the gas inlet opening of the frame and the connection opening of the cushion module, wherein the linear frictional surface exists in a condition when the cushion module is not connected to the collar of the frame;
wherein the elastomeric friction member engages an outer surface of the collar, wherein an outer surface of the elastomeric friction member is exposed to atmosphere, and wherein the elastomeric friction member is configured to compress when the cushion module is connected to the frame; and
wherein the linear frictional surface is configured to apply a retention force along an assembly axis of the respiratory mask.

14. The respiratory mask of claim 13, wherein the housing comprises a wall portion, at least a portion of which is external of the elastomeric friction member and extends between the elastomeric friction member and the cushion.

15. The respiratory mask of claim 13, wherein the front wall of the frame further comprises a vent, and wherein the collar surrounds the vent in addition to the gas inlet.

16. The respiratory mask of claim 13, wherein the housing of the cushion module further comprises a support flange, wherein the support flange at least partially defines the connection opening and extends within the elastomeric friction member substantially in a direction of assembly of the cushion module to the frame.

17. The respiratory mask of claim 16, wherein the support flange comprises a plurality of apertures and wherein the elastomeric friction member extends through each of the plurality of apertures.

18. The respiratory mask of claim 16, wherein a portion of a wall portion embedded within the elastomeric friction member comprises a plurality of apertures and wherein the elastomeric friction member extends through each of the apertures.

19. The respiratory mask of claim 16, wherein a portion of a wall portion embedded within the elastomeric friction member and the support flange each comprise a plurality of apertures and wherein the elastomeric friction member passes through each of the apertures.

20. The respiratory mask of claim 13, wherein the elastomeric friction member defines an abutment surface configured to contact the front wall of the frame when the cushion module is coupled to the frame.

21. The respiratory mask of claim 13, wherein the elastomeric friction member and the cushion are constructed of the same material.

22. The respiratory mask of claim 13, wherein the elastomeric friction member is more rigid than the cushion.

23. The cushion module of claim 13, wherein the frame and the housing are more rigid than the elastomeric friction member and the cushion.

24. The cushion module of claim 13, wherein the frame is less rigid than the friction member and the cushion.

25. The cushion module of claim 13, wherein the retention force is the only force that connects the cushion module to the frame.
